(19) United States
(12) Patent Application Publication
(10) Pub. No.: US 2026/0258048 A1
(43) Pub. Date: Sep. 3, 2026

(54) LACTAM-CONTAINING IMIDAZOPYRIDAZINE IL-17 INHIBITOR COMPOUNDS

(71) Applicant: Janssen Pharmaceutica NV, Beerse (BE)

(72) Inventors: Steven D. Goldberg, San Diego, CA (US); Deane Gordan, San Diego, CA (US); Luke E. Hanna, San Diego, CA (US); Steven A. Loskot, San Diego, CA (US); Anastassia Matviitsuk, San Diego, CA (US); Steven P. Meduna, Meridian, ID (US)

(73) Assignee: Janssen Pharmaceutica NV, Beerse (BE)

(21) Appl. No.: 19/469,476

(22) PCT Filed: Mar. 27, 2024
(86) PCT No.: PCT/IB2024/052955

§ 371 Date: Sep. 26, 2025

Related U.S. Application Data
(60) Provisional application No. 63/492,553, filed on Mar. 28, 2023.

(51) Int. Cl. C07D 487/04; A61K 31/5025; A61K 31/55; A61P 29/00; C07D 519/00

(52) U.S. Cl. CPC C07D 487/04; A61K 31/5025; A61K 31/55; A61P 29/00; C07D 519/00

(57) ABSTRACT

The present application discloses compounds having the following formula (I), or pharmaceutically acceptable salts thereof, wherein R^{1a}, R^{1b}, R^{1c}, R^{1d}, R^{2a}, R^{2b}, R³, R⁴, n, and m are defined in the specification, as well as methods of making and using the compounds disclosed herein for treating or ameliorating an IL-17 mediated syndrome, disorder and/or disease.

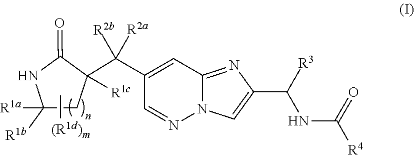

### REFERENCE TO SEQUENCE LISTING SUBMITTED ELECTRONICALLY

This application contains a sequence listing, which is submitted electronically via EFS-Web as an ST.26 XML formatted sequence listing with a file name “PRD4255WOPCT1-SeqListing.xml”, creation date of Mar. 12, 2024, and having a size of 3 KB. The sequence listing submitted via EFS-Web is part of the specification and is herein incorporated by reference in its entirety.

### BACKGROUND

Interleukin-17 (“IL-17”), also known as IL-17A and CT A-8, is produced mainly by CD4+Th17 cells, and also by other immune cells such as CD8+ T cells, γδ T cells, NK cells, NKT cells, and innate lymphoid cells (ILCs). IL-17A exists as a homodimer (A/A) or as a heterodimer (ATF) with IL-17F and signals through binding to dimeric receptor complex IL-17RA and IL-17RC. IL-17RA is ubiquitously expressed at particularly high levels by haematopoietic cell types, whereas IL-17RC is preferentially expressed by non-haematopoietic cells (Gaffen, S. Structure and signaling in the IL-17 receptor family. Nat. Rev. Immunol. 2009, 9, 556-567). IL-17A/IL-17R signaling induces de novo gene transcription by triggering NT-kB, C/EBP and MAPK pathways through ACT1-TRAF6-TRAF4. It can also stabilize target mRNA transcripts through the ACT1-TRAF2-TRAF5 complex (Amatya N. et al., Trends in Immunology, 2017, 38, 310-322). IL-17A stimulates the release of inflammatory mediators including IL-6, IL-S, G-CSF, TNF-α, and IL-1β that recruit and activate lymphocytes to the site of injury or inflammation and maintain a proinflammatory state.

As discussed in the present disclosure, preclinical and clinical data have demonstrated the significant pathological role of IL-17A in multiple autoimmune and inflammatory diseases.

In summary, animal and human studies have shown that IL-17A plays a crucial role in pathogenesis of the multiple diseases and/or conditions discussed above. The significance of targeting IL-17A has been demonstrated by the transformational efficacy of injectable IL-17A neutralizing antibodies in patients.

Despite the advances achieved with injectable IL-17A antagonist antibodies, there is a long-felt need for the development of an oral small molecule IL-17A inhibitor as it may broaden treatment options for many patients without access to biologics. In addition, a safe and efficacious small molecule IL-17A inhibitor may offer significant benefits to patients over the injectable IL-17A neutralizing antibodies such as convenient dosing regimens and cost savings, which in turn may provide effective long-term disease management.

However, the development of an oral small molecule treatment has remained challenging. For example, no oral small molecule IL-17A inhibitor has progressed into late-stage clinical trials yet, and only two oral small molecule IL-17A inhibitors have progressed into phase I clinical trials (NCT04586920 and NCT04883333) as of Sep. 28, 2021. Additionally, as of December 2021, one of these clinical trials (NCT04586920) was suspended due to safety review. Accordingly, there is a need for new small molecule IL-17A modulators (e.g., inhibitors).

### SUMMARY

The present application discloses a compound of Formula I:

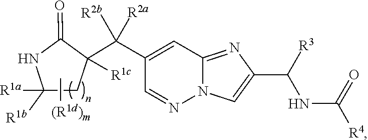

or a pharmaceutically acceptable salt thereof, wherein R^{1a}, R^{1b}, R^{1c}, R^{1d}, R^{2a}, R^{2b}, R³, R⁴, n, and m are as defined herein. In some embodiments, the compound of Formula I, as described and provided herein, has a formula of Formula Ig-1:

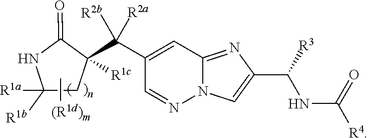

In some embodiments, the compound of Formula I, as described and provided herein, has a formula of Formula Ib-8:

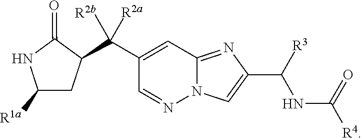

The present application also discloses a pharmaceutical composition comprising a compound of Formula I, or a pharmaceutically acceptable salt thereof, and a pharmaceutically acceptable carrier.

The present application also discloses a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof. In some embodiments, the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus.

### DETAILED DESCRIPTION

### Definitions

Discussion of documents, acts, materials, devices, articles or the like which has been included in the present specification is for the purpose of providing context. Such discussion is not an admission that any or all of these matters form part of the prior art with respect to any compounds or methods disclosed or claimed.

Unless defined otherwise, all technical and scientific terms used herein have the same meaning as commonly understood to one of ordinary skill in the art to which this disclosure pertains. Otherwise, certain terms used herein have the meanings as set forth in the specification.

It must be noted that as used herein and in the appended claims, the singular forms “a,” “an,” and “the” include plural reference unless the context clearly dictates otherwise.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

The term “administering” with respect to the methods of the present disclosure, means a method for therapeutically or prophylactically preventing, treating or ameliorating a syndrome, disorder or disease as described herein by using a compound of the disclosure, or pharmaceutically acceptable salt thereof, composition thereof, or medicament thereof. Such methods include administering a therapeutically effective amount of a compound of the disclosure, or pharmaceutically acceptable salt thereof, composition thereof, or medicament thereof, at different times during the course of a therapy or concurrently or sequentially as a combination therapy.

The term “subject” refers to a patient, which may be an animal, preferably a mammal, most preferably a human, whom will be or has been treated by a method according to an embodiment of the application. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

The term “therapeutically effective amount” or “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

The term “pharmaceutically acceptable” means approved or approvable by a regulatory agency of Federal or a state government or the corresponding agency in countries other than the United States, or that is listed in the U S. Pharmacopoeia or other generally recognized pharmacopoeia for use in animals, and more particularly, in humans.

A “pharmaceutically acceptable salt” is intended to mean a salt of a free acid or base of a compound disclosed herein that is non-toxic, biologically tolerable, or otherwise biologically suitable for administration to the subject. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A compound of the disclosure may possess a sufficiently acidic group, a sufficiently basic group, or both types of functional groups, and accordingly react with a number of inorganic or organic bases, and inorganic and organic acids, to form a pharmaceutically acceptable salt.

As used herein, “IL-17” or “IL-17A” refers to interleukin 17A. It is also named IL17, CTLA8, CTLA-8. Interleukin 17A is a pro-inflammatory cytokine. This cytokine is produced by a group of immune cells in response to their stimulation. An exemplary amino acid sequence of human IL-17 is represented in GenBank Accession No. NP_002181.1, which can be encoded by a nucleic acid sequence such as that of GenBank Accession No. NM_002190.3.

The term “modulator” as used herein refers to any agents or molecules that can bind to IL-17, including small molecule compounds.

As used herein, the term “composition” is intended to encompass a product comprising the specified ingredients in the specified amounts, as well as any product which results, directly or indirectly, from combinations of the specified ingredients in the specified amounts.

As used herein, the term “treat,” “treating,” or “treatment” of any disease, condition, syndrome or disorder refers, in one embodiment, to ameliorating the disease, condition, syndrome or disorder (i.e., slowing or arresting or reducing the development of the disease or at least one of the clinical symptoms thereof). In another embodiment, “treat,” “treating,” or “treatment” refers to alleviating or ameliorating at least one physiological or biochemical parameter associated with or causative of the disease, condition, syndrome or disorder, including those which may not be discernible by the patient. In a further embodiment, “treat,” “treating,” or “treatment” refers to modulating the disease, condition, syndrome or disorder either physically (e.g. stabilization of a discernible symptom), physiologically, (e.g. stabilization of a physical parameter), or both.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease. In an embodiment, “prevent” or “prevention” refers to preventing or delaying the onset or development or progression of the disease, condition, syndrome or disorder.

The term “alkyl” is a straight or branched saturated hydrocarbon. For example, an alkyl group can have 1 to 12 carbon atoms (i.e., (C₁-C₁₂)alkyl) or 1 to 6 carbon atoms (i.e., (C₁-C₆)alkyl). Examples of alkyl groups include, but are not limited to, methyl (Me, —CH₃), ethyl (Et, —CH₂CH₃), 1-propyl (n-Pr, n-propyl, —CH₂CH₂CH₃), isopropyl (i-Pr, i-propyl, —CH(CH₂)₂), 1-butyl (n-bu, n-butyl, —CH₂CH₂CH₂CH₃), 2-butyl (s-bu, s-butyl, —CH(CH₃)CH₂CH₃), tert-butyl (t-bu, t-butyl, —CH(CH₃)₃), 1-pentyl (n-pentyl, —CH₂CH₂CH₂CH₂CH₁), 2-pentyl (—CH(CH₃) CH₂CH₂CH₃), neopentyl (—CH₂C(CH₃)₃), 1-hexyl (—CH₂CH₂CH₂CH₂CH₂CH₃), 2-hexyl (—CH(CH₃)CH₂CH₂CH₂CH₃), heptyl (—(CH₂)₆CH₃), octyl (—(CH₂)₇CH₃), 2,2,4-trimethylpentyl (—CH₂C(CH₃)₂CH₂CH(CH₃)₂), nonyl (—(CH₂)₈CH₃), decyl (—(CH₂)₉CH₃), undecyl (—(CH)₁₀CH₃), and dodecyl (—(CH₂)₁₁CH₃). Any alkyl group may be unsubstituted or substituted.

The term “C_{(a-b)}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₍₁₋₄₎ denotes a radical containing 1, 2, 3 or 4 carbon atoms.

The term “alkylene” refers to a linear or branched saturated divalent hydrocarbon moiety derived from an alkane having 1 to 12 carbon atoms (i.e., (C₁-C₁₂)alkylene), 1 to 6 carbon atoms (i.e., (C₁-C₆)alkylene), 1 to 4 carbon atoms (i.e., (C₁-C₄)alkylene), or 1 to 3 carbon atoms (i.e., (C₁-C₃)alkylene). Examples of alkylene groups include, but are not limited to, methylene (—CH₂—), ethylene (—CH₂CH₂—), —C(CH₃)H—, propylene (—CH₂CH₂CH₂—), isopropylene (—CH(CH₃)CH₂—), and —CH₂CH(CH₃)—. In an embodiment, alkylene refers to C₍₁₋₄₎alkylene. In another embodiment, alkylene refers to C₍₁₋₂₎alkylene.

The term “cycloalkyl” refers to a saturated or partially unsaturated all carbon ring system having 3 to 8 carbon atoms (i.e., C₍₃₋₈₎cycloalkyl), and preferably 3 to 6 carbon atoms (i.e., C₍₃₋₆₎cycloalkyl), wherein the cycloalkyl ring system has a single ring or multiple rings in a spirocyclic or bicyclic form. Exemplary cycloalkyls include, but are not limited to, cyclopropyl, cyclobutyl, cyclopentyl, cyclohexyl, cycloheptyl, and cyclooctyl. Unless otherwise stated specifically in the specification, a cycloalkyl group may be unsubstituted or substituted. Some cycloalkyl groups may exist as spirocycloalkyls, wherein two cycloalkyl rings are fused through a single carbon atom; for example and without limitation, an example of a spiropentyl group is

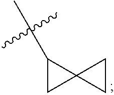 [SMILES: *C1CC12CC2]

for example and without limitation, examples of spirohexyl groups include

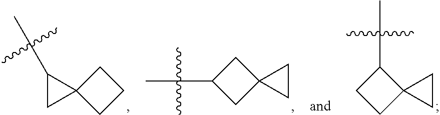 [SMILES: *C1CC12CCC2 ; *C1CC2(CC2)C1 ; *C1CCC12CC2]

for example and without limitation examples of cycloheptyl groups include

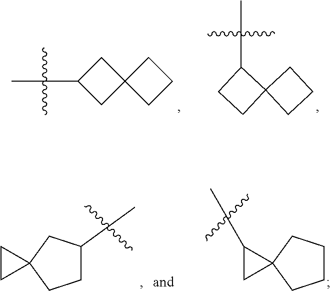 [SMILES: *C1CC2(CCC2)C1 ; *C1CCC12CCC2 ; *C1CCC2(CC2)C1 ; *C1CC12CCCC2]

for example and without limitation examples of cyclooctyl groups include

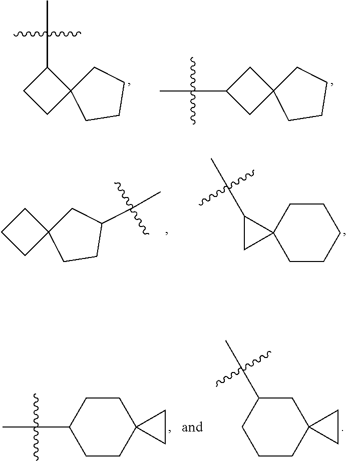 [SMILES: *C1CCC12CCCC2 ; *C1CC2(CCCC2)C1 ; *C1CCC2(CCC2)C1 ; *C1CC12CCCCC2 ; *C1CCC2(CC1)CC2 ; *C1CCCC2(CC2)C1]

Unless otherwise stated specifically in the specification, a siprocycloalkyl group may be unsubstituted or substituted. Bicyclic cycloalkyl ring systems also

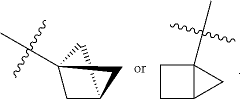

The term “heterocyclyl” refers to a single saturated or partially unsaturated ring having 3 to 12 ring members, 3 to 10 ring members, 3 to 8 ring members, or 3 to 6 ring members and which contains carbon atoms and at least one atom other than carbon in the ring, wherein the atom is selected from the group consisting of N, O, and S. The terms “heterocyclyl” and “heterocycloalkyl” include cyclic esters (e.g., lactones) and cyclic amides (e.g., lactams). Exemplary heterocycles include, but are not limited to oxetanyl, aziridinyl, azetidinyl, pyrrolidinyl, pyrrolidonyl, piperidinyl, piperazinyl, morpholinyl, tetrahydropyranyl, tetrahydrofuranyl, and thiomorpholinyl. Unless otherwise noted, the heterocyclyl group is attached to its pendant group at any heteroatom or carbon atom that results in a stable structure. In an embodiment, heterocyclyl refers to 3- to 10-membered heterocyclyl. In another embodiment, heterocyclyl refers to 3- to 8-membered heterocyclyl. In another embodiment, heterocyclyl refers to 3- to 6-membered heterocyclyl.

The term “heteroaryl” refers to a single aromatic ring that has at least one atom other than carbon in the ring, wherein the atom is selected from the group consisting of oxygen, nitrogen and sulfur. The term “heteroaryl” includes single aromatic rings of from 1 to 6 carbon atoms and 1 to 4 heteroatoms selected from the group consisting of oxygen, nitrogen and sulfur. Exemplary heteroaryl ring systems include but are not limited to pyridinyl, pyrimidinyl, pyrazinyl, pyridazinyl, pyrimidinyl, pyrazolyl, oxazolyl, oxadiazolyl, isoxazolyl, triazolyl, imidazolyl, tetrazolyl, thienyl, thiazolyl, isothiazolyl, thiadiazolyl, or furyl.

The term “halogen” or “halo” refers to bromo (—Br), chloro (—Cl), fluoro (—F) or iodo (—I).

Where the compounds disclosed herein have at least one stereocenter, they may accordingly exist as enantiomers or diastereomers. It is to be understood that all such isomers and mixtures thereof are encompassed within the scope of the present disclosure.

“Diastereoisomers” are stereoisomers that have at least two asymmetric atoms, but which are not mirror images of each other.

“Enantiomers” are a pair of stereoisomers that are non-superimposable mirror images of each other. A “racemic” mixture is a 1:1 mixture of a pair of enantiomers. A “scalemic” mixture of enantiomers is mixture of enantiomers at a ratio other than 1:1.

Where the processes for the preparation of the compounds according to the present disclosure give rise to mixture of stereoisomers, these isomers may be separated by conventional techniques such as preparative chromatography. The compounds may be prepared in racemic form, a scalemic mixture, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral column via HPLC or SFC. In some instances rotamers of compounds may exist which are observable by ¹H NMR leading to complex multiplets and peak integration in the ¹H NMR spectrum.

The absolute stereochemistry is specified according to the Cahn-Ingold-Prelog R-S system. Chiral centers, of which the absolute configurations are known, are labelled by prefixes R and S, assigned by the standard sequence-rule procedure, and preceded when necessary by the appropriate locants (Pure & Appl. Chem. 45, 1976, 11-30). Certain examples contain chemical structures that are depicted or labelled as an (R*) or (S*). When (R*) or (S*) is used in the name of a compound or in the chemical representation of the compound, it is intended to convey that the compound is a pure single isomer at that stereocenter; however, absolute configuration of that stereocenter has not been established. Thus, a compound designated as (R*) refers to a compound that is a pure single isomer at that stereocenter with an absolute configuration of either (R) or (S), and a compound designated as (S*) refers to a compound that is a pure single isomer at that stereocenter with an absolute configuration of either (R) or (S). For example, 4-cyclopropyl-N—((S)-(7-(((R*)-5,5-difluoro-2-oxopiperidin-3-yl)methyl)imidazo[1,2-b]pyridazin-2-yl)(4,4-difluorocyclohexyl)methyl)-1,2,5-oxadiazole-3-carboxamide:

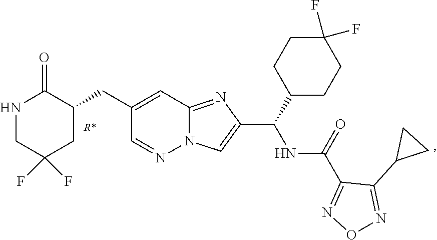 [SMILES: O=C(N[C@H](c1cn2ncc(C[C@@H]3CC(F)(F)CNC3=O)cc2n1)C1CCC(F)(F)CC1)c1nonc1C1CC1]

refers to a compound that is either:

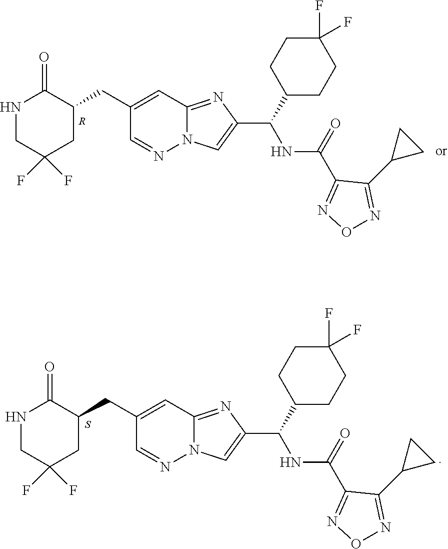

During any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Furthermore, it is intended that within the scope of the present disclosure, any element, in particular when mentioned in relation to a compound of the disclosure, or pharmaceutically acceptable salt thereof, shall comprise all isotopes and isotopic mixtures of said element, either naturally occurring or synthetically produced, either with natural abundance or in an isotopically enriched form. For example, a reference to hydrogen includes within its scope ¹H, ²H (i.e., deuterium or D), and ¹H (i.e., tritium or T). In some embodiments, the compounds described herein include a ²H (i.e., deuterium) isotope. By way of example, the group denoted —C₍₁₋₆₎alkyl includes not only —CH₃, but also —CD₃; not only —CH₂CH₃, but also —CD₂CD, etc. Similarly, references to carbon and oxygen include within their scope respectively ¹²C, ¹³C and ¹⁴C and ¹⁵O and ¹⁶O and ¹⁷O and ¹⁸O. The isotopes may be radioactive or non-radioactive. Radiolabelled compounds of the disclosure may include a radioactive isotope selected from the group comprising ³H, ¹¹C, ¹⁸F, ³⁵S, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

### Compounds

The present application discloses a compound of Formula I:

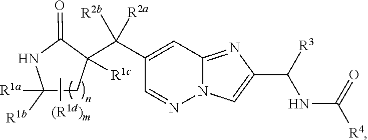

or a pharmaceutically acceptable salt thereof,
wherein:
R^{1a} and R^{1b} are each independently H, —C₍₁₋₃₎alkyl, —C₍₃₋₅₎cycloalkyl, —C₍₁₋₃₎alkyl-O—C₍₁₋₃₎alkyl or —C₍₁₋₃₎alkyl-N(C₍₁₋₃₎alkyl)₂, wherein the —C₍₁₋₃₎alkyl is unsubstituted or substituted with one to six R^{1aa} groups; or R^{1a} and R^{1b} together with the carbon atom to which the are attached form a spiro C₍₃₋₅₎cycloalkyl or a spiro heterocyclyl selected from

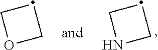

wherein · indicates the carbon atom to which R^{1a} and R^{1b} are attached; or R^{1a} and R^{1c} are taken together to form a C₍₁₋₂₎alkylenyl bridge;
R^{1c} is H, halo, —OH, —C₍₁₋₃₎alkyl, —C₍₁₋₃₎alkyl-CN, —C₍₃₋₅₎cycloalkyl, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups;
R^{1d} is independently for each occurrence —OH, halo, —C₍₁₋₃₎alkyl, —C₍₃₋₅₎cycloalkyl, —C₍₁₋₃₎alkyl-O—C₍₁₋₃₎alkyl or —C₍₁₋₃₎alkyl-N—(C₍₁₋₃₎alkyl)₂ wherein the —C₍₁₋₃₎alkyl is unsubstituted or substituted with one to six R^{1aa} groups;
R^{1aa} independently for each occurrence is halo;
R^{2a} is H, —OH, —C₍₁₋₃₎alkyl, —C₍₃₋₅₎cycloalkyl, —C₍₁₋₃₎alkyl-O—C₍₁₋₃₎alkyl, —C₍₁₋₃₎alkyl-O—C₍₃₋₅₎ cycloalkyl, or 4- to 6-membered heterocyclyl, wherein the —C₍₁₋₃₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one to six R^{2aa} groups;
R^{2aa} independently for each occurrence is halo, —OH, or —CN;
R^{2b} is H or —C₍₁₋₃₎alkyl; or R^{2a} and R^{2b} are taken together with the carbon atom to which they are attached to form a C═O;
R³ is —C₍₁₋₁₀₎alkyl, —C₍₁₋₆₎alkyl-O—C₍₁₋₆₎alkyl, —C₍₁₋₆₎alkyl-O—C₍₃₋₅₎cycloalkyl, —C₍₁₋₆₎alkyl-O—C₍₃₋₅₎cycloalkyl-C₍₁₋₃₎alkyl, —C₍₃₋₈₎cycloalkyl, or —C₍₁₋₃₎alkyl-(C₍₃₋₅₎cycloalkyl)₁₋₂, each of which is unsubstituted or substituted with one to six halo atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, —C₍₁₋₆₎alkyl-O—C₍₁₋₆₎alkyl, and —C₍₀₋₂₎alkyl-C₍₃₋₆₎cycloalkyl are unsubstituted or substituted with one to six substituents independently selected from halo, —CH₃, —CH₂F, —CHF₂, and —CF₃;
n is 1, 2 or 3; and
m is 0, 1, 2, 3, or 4.

The present application also discloses a compound of Formula I:

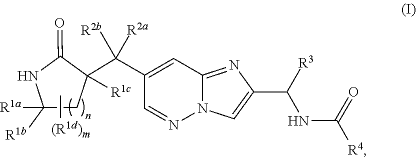

or a pharmaceutically acceptable salt thereof,
wherein:
R^{1a} and R^{1b} are each independently H, —C₍₁₋₃₎alkyl, —C₍₃₋₅₎cycloalkyl, —C₍₁₋₃₎alkyl-O—C₍₁₋₃₎alkyl or —C₍₁₋₃₎alkyl-N(C₍₁₋₃₎alkyl)₂, wherein the —C₍₁₋₃₎alkyl is unsubstituted or substituted with one to six R^{1aa} groups; or R^{1a} and R^{1b} together with the carbon atom to which they are attached form a spiro C₍₃₋₅₎cycloalkyl or a spiro heterocyclyl selected from

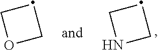

wherein · indicates the carbon atom to which R^{1a} and R^{1b} are attached; or R^{1a} and R^{1c} are taken together to form a C₍₁₋₂₎alkylenyl bridge;
R^{1c} is H, halo, —OH, —C₍₁₋₃₎alkyl, —C₍₁₋₃₎alkyl-CN, —C₍₃₋₅₎cycloalkyl, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups;
R^{1d} is independently for each occurrence —OH, halo, —C₍₁₋₃₎alkyl, —C₍₃₋₅₎cycloalkyl, —C₍₁₋₃₎alkyl-O—C₍₁₋₃₎alkyl or —C₍₁₋₃₎alkyl-N—(C₍₁₋₃₎alkyl)₂ wherein the —C₍₁₋₃₎alkyl is unsubstituted or substituted with one to six R^{1aa} groups;
R^{1aa} independently for each occurrence is halo;
R^{2a} is H, —OH, —C₍₁₋₃₎alkyl, —C₍₃₋₅₎cycloalkyl, —C₍₁₋₃₎alkyl-O—C₍₁₋₃₎alkyl, —C₍₁₋₃₎alkyl-O—C₍₃₋₅₎ cycloalkyl, or 4- to 6-membered heterocyclyl, wherein the —C₍₁₋₃₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one to six R^{2aa} groups;
R^{2aa} independently for each occurrence is halo, —OH, or —CN;
R^{2b} is H or —C₍₁₋₃₎alkyl; or R^{2a} and R^{2b} are taken together with the carbon atom to which they are attached to form a C═O;
R³ is —C₍₁₋₁₀₎alkyl, —C₍₁₋₆₎alkyl-O—C₍₁₋₆₎alkyl, —C₍₁₋₆₎alkyl-O—C₍₃₋₅₎cycloalkyl, —C₍₁₋₆₎alkyl-O—C₍₃₋₅₎cycloalkyl-C₍₁₋₃₎alkyl, or —C₍₃₋₆₎cycloalkyl, each of which is unsubstituted or substituted with one to six halo atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, —C₍₁₋₆₎alkyl-O—C₍₁₋₆₎alkyl, and —C₍₀₋₂₎alkyl-C₍₃₋₆₎ cycloalkyl are unsubstituted or substituted with one to six substituents independently selected from halo, —CH₃, —CH₂F, —CHF₂, and —CF₃;
n is 1, 2 or 3; and
m is 0, 1, 2, 3, or 4.

In some embodiments, disclosed is a compound of Formula I, as described and provided herein, or a pharmaceutically acceptable salt thereof, have a formula of Formula Ig-1:

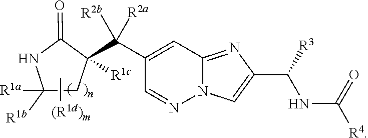

In some embodiments, disclosed is a compound of Formula I, as described and provided herein, or a pharmaceutically acceptable salt thereof, have a formula of Formula Ib-8:

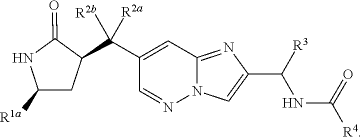

In some embodiments, disclosed herein is a compound of Formula I, or a pharmaceutically acceptable salt thereof, wherein:
R^{1a} and R^{1b} are each independently H, —C₍₁₋₃₎alkyl, or —C₍₃₋₅₎cycloalkyl, wherein the —C₍₁₋₃₎alkyl is unsubstituted or substituted with one to six R^{1aa} groups; or R^{1a} and R^{1b} together with the carbon atom to which they are attached form a spiro C₍₃₋₅₎cycloalkyl or a spiro heterocyclyl selected from

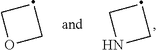

wherein · indicates the carbon atom to which R^{1a} and R^{1b} are attached; or R^{1a} and R^{1c} are taken together to form a C₍₁₋₂₎alkylenyl bridge;
R^{1c} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups;
R^{1d} is independently for each occurrence halo, —C₍₁₋₃₎alkyl, or —C₍₃₋₅₎cycloalkyl, wherein the —C₍₁₋₃₎alkyl is unsubstituted or substituted with one to six R^{1aa} groups;
R^{1aa} independently for each occurrence is fluorine;
R^{2a} is H, —OH, or —C₍₁₋₃₎alkyl, wherein the —C₍₁₋₃₎alkyl is unsubstituted or substituted with one to six R^{2aa} groups; or R^{2a} and R^{1c} are taken together with the carbon atoms to which they are attached to form a C₍₃₋₅₎cycloalkyl or a 4- to 6-membered heterocycle;
R^{2aa} independently for each occurrence is fluorine, —OH, or —CN;
R^{2b} is H or —C₍₁₋₃₎alkyl; or R^{2a} and R^{2b} are taken together with the carbon atom to which they are attached to form a C═O;
R³ is —C₍₁₋₁₀₎alkyl, —C₍₁₋₆₎alkyl-O—C₍₁₋₆₎alkyl, —C₍₁₋₆₎alkyl-O—C₍₃₋₅₎cycloalkyl, —C₍₁₋₆₎alkyl-O—C₍₃₋₅₎cycloalkyl-C₍₁₋₃₎alkyl, —C₍₃₋₈₎cycloalkyl, or —C₍₁₋₃₎alkyl-(C₍₃₋₅₎cycloalkyl)₁₋₂, each of which is unsubstituted or substituted with one to six fluorine atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, —C₍₁₋₆₎alkyl-O—C₍₁₋₆₎alkyl, and —C₍₀₋₂₎alkyl-C₍₃₋₆₎ cycloalkyl are unsubstituted or substituted with one to six substituents independently selected from fluorine, —CH₃, —CH₂F, —CHF₂, and —CF₃;
n is 1, 2 or 3; and
m is 0, 1, 2, 3, or 4.

In some embodiments, disclosed herein is a compound of Formula I, or a pharmaceutically acceptable salt thereof, wherein:
R^{1a} is H, —C₍₁₋₃₎alkyl, or —C₍₃₋₅₎cycloalkyl, wherein the —C₍₁₋₃₎alkyl is unsubstituted or substituted with one to six R^{1aa} groups; or R^{1a} and R^{1c} are taken together to form a C₍₁₋₂₎alkylenyl bridge;
R^{1b} is H or —C₍₁₋₃₎alkyl, wherein the —C₍₁₋₃₎alkyl is unsubstituted or substituted with one to six R^{1aa} groups; or R^{1a} and R^{1b} together with the carbon atom to which they are attached form a spiro C₍₃₋₅₎cycloalkyl or a spiro heterocyclyl selected from

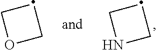

wherein · indicates the carbon atom to which R^{1a} and R^{1b} are attached;
R^{1c} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups;
R^{1d} is independently for each occurrence halo, —C₍₁₋₃₎alkyl, or —C₍₃₋₅₎cycloalkyl, wherein the —C₍₁₋₃₎alkyl is unsubstituted or substituted with one to six R^{1aa} groups;
R^{1aa} independently for each occurrence is fluorine;
R^{2a} is H, —OH, or —C₍₁₋₃₎alkyl, wherein the —C₍₁₋₃₎alkyl is unsubstituted or substituted with one to six R^{2aa} groups; or R^{2a} and R^{1c} are taken together with the carbon atoms to which they are attached to form a C₍₃₋₅₎cycloalkyl or a 4- to 6-membered heterocycle;
R^{2aa} independently for each occurrence is fluorine or —OH;
R^{2b} is H or —C₍₁₋₃₎alkyl; or R^{2a} and R^{2b} are taken together with the carbon atom to which they are attached to form a C═O;
R³ is —C₍₁₋₁₀₎alkyl, —C₍₁₋₆₎alkyl-O—C₍₁₋₆₎alkyl, —C₍₁₋₆₎alkyl-O—C₍₃₋₅₎cycloalkyl, or —C₍₃₋₅₎ cycloalkyl, each of which is unsubstituted or substituted with one to six fluorine atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎ cycloalkyl, wherein the —C₍₁₋₆₎alkyl, —O—C₍₁₋₆₎alkyl, —C₍₁₋₆₎alkyl-O—C₍₁₋₆₎alkyl, and —C₍₀₋₂₎alkyl-C₍₃₋₆₎ cycloalkyl are unsubstituted or substituted with one to six substituents independently selected from fluorine, —CH₃, —CH₂F, —CHF₂, and —CF₃;
n is 1, 2 or 3; and
m is 0, 1, 2, 3, or 4.

In some embodiments, disclosed herein is a compound of Formula I, or a pharmaceutically acceptable salt thereof, wherein:
R^{1a} is H, —C₍₁₋₃₎alkyl, or —C₍₃₋₅₎cycloalkyl, wherein the —C₍₁₋₃₎alkyl is unsubstituted or substituted with one to six R^{1aa} groups; or R^{1a} and R^{1c} are taken together to form a C₍₁₋₂₎alkylenyl bridge;
R^{1b} is H or —C₍₁₋₃₎alkyl, wherein the —C₍₁₋₃₎alkyl is unsubstituted or substituted with one to six R^{1aa} groups; or R^{1a} and R^{1b} together with the carbon atom to which they are attached form a spiro C₍₃₋₅₎cycloalkyl or a spiro heterocyclyl selected from

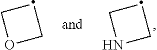

wherein · indicates the carbon atom to which R^{1a} and R^{1b} are attached;
R^{1c} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups;
R^{1d} is independently for each occurrence halo or —C₍₁₋₃₎alkyl, wherein the —C₍₁₋₃₎alkyl is unsubstituted or substituted with one to six R^{1aa} groups;
R^{1aa} independently for each occurrence is fluorine;
R^{2a} is H, —OH, or —C₍₁₋₃₎alkyl, wherein the —C₍₁₋₃₎alkyl is unsubstituted or substituted with one to six R^{2aa} groups; or R^{2a} and R^{1c} are taken together with the carbon atoms to which they are attached to form a C₍₃₋₅₎cycloalkyl or a 4- to 6-membered heterocycle;
R^{2aa} independently for each occurrence is fluorine or —OH;
R^{2b} is H or —C₍₁₋₃₎alkyl; or R^{2a} and R^{2b} are taken together with the carbon atom to which they are attached to form a C═O;
R³ is —C₍₁₋₁₀₎alkyl or —C₍₃₋₈₎cycloalkyl, each of which is unsubstituted or substituted with one to six fluorine atoms; or R³ is —C₍₁₋₁₀₎alkyl or —C₍₃₋₆₎cycloalkyl, each of which is unsubstituted or substituted with one to six fluorine atoms;
R⁴ is a 5-membered heteroaryl that is unsubstituted or substituted with one to two R^{4a} groups;
R⁴ is —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎cycloalkyl;
n is 1, 2 or 3; and
m is 0, 1, or 2.

In some embodiments, disclosed herein is a compound of Formula I, or a pharmaceutically acceptable salt thereof, wherein:
R^{1a} is H, —C₍₁₋₃₎alkyl, or —C₍₃₋₅₎cycloalkyl, wherein the —C₍₁₋₃₎alkyl is unsubstituted or substituted with one to six R^{1aa} groups;
R^{1b} is H or —C₍₁₋₃₎alkyl, wherein the —C₍₁₋₃₎alkyl is unsubstituted or substituted with one to six R^{2aa} groups;
R^{1c} is H, halo, —OH, —C₍₁₋₆₎alkyl, —C₍₁₋₃₎alkyl-CN, —C₍₁₋₃₎alkyl-O—C₍₁₋₃₎alkyl, or —C₍₁₋₆₎alkyl-N(C₍₁₋₆₎alkyl)₂, wherein the —C₍₁₋₃₎alkyl is unsubstituted or substituted with one to six R^{1aa} groups;
R^{1d} is independently for each occurrence halo or —C₍₁₋₆₎alkyl, wherein the —C₍₁₋₃₎alkyl is unsubstituted or substituted with one to six R^{1aa} groups;
R^{1aa} independently for each occurrence is fluorine;
R^{2a} is H, —OH, or —C₍₁₋₃₎alkyl, wherein the —C₍₁₋₃₎alkyl is unsubstituted or substituted with one to six R^{2aa} groups;
R^{2aa} independently for each occurrence is fluorine;
R^{2b} is H or —C₍₁₋₃₎alkyl; or R^{2a} and R^{2b} are taken together with the carbon atom to which they are attached to form a C═O;
R³ is —C₍₄₋₁₀₎alkyl or cyclohexyl, each of which is unsubstituted or substituted with one to six fluorine atoms;
R⁴ is a 5-membered heteroaryl that is unsubstituted or substituted with one to two R⁴, groups;
R^{4a} is —C₍₁₋₆₎alkyl, —O—C₍₁₋₆₎alkyl, —C₍₁₋₆₎alkyl-O—C₍₁₋₆₎alkyl, or —C₍₃₋₆₎cycloalkyl;
n is 1, 2 or 3; and
m is 0, 1, or 2.

In some embodiments, disclosed herein is a compound of Formula I, or a pharmaceutically acceptable salt thereof, wherein:
R^{1a} is H or —C₍₁₋₃₎alkyl, wherein the —C₍₁₋₃₎alkyl is unsubstituted or substituted with one to six R^{1aa} groups;
R^{1b} is H or —C₍₁₋₃₎alkyl, wherein the —C₍₁₋₃₎alkyl is unsubstituted or substituted with one to six R^{1aa} groups;
R^{1c} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂;
R^{1d} is independently for each occurrence halo or —C₍₁₋₃₎alkyl, wherein the —C₍₁₋₃₎alkyl is unsubstituted or substituted with one to six R^{1aa} groups;
R^{1aa} independently for each occurrence is fluorine;
R^{2a} is H, —OH, or —C₍₁₋₃₎alkyl;
R^{2b} is H or —C₍₁₋₃₎alkyl;
R³ is —C₍₄₋₁₀₎alkyl or cyclohexyl, each of which is substituted with one to six fluorine atoms;
R⁴ is a 5-membered heteroaryl that is unsubstituted or substituted with one to two R^{4a} groups;
R^{4a} is —C₍₁₋₆₎alkyl, —O—C₍₁₋₆₎alkyl, —C₍₁₋₆₎alkyl-O—C₍₁₋₆₎alkyl, or —C₍₃₋₆₎cycloalkyl;
n is 1 or 2; and
m is 0, 1, or 2.

In some embodiments, disclosed herein is a compound of Formula I, or a pharmaceutically acceptable salt thereof, wherein:
R^{1a} is —C₍₁₋₃₎alkyl, wherein the —C₍₁₋₃₎alkyl is unsubstituted or substituted with one to three R^{1aa} groups;
R^{1b} is H or —C₍₁₋₃₎alkyl, wherein the —C₍₁₋₃₎alkyl is unsubstituted or substituted with one to three R^{1aa} groups;
R^{1aa} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂;
R^{1aa} independently for each occurrence is fluorine;
R^{2a} is H, or —OH;
R^{2b} is H;
R³ is —C₍₄₋₈₎alkyl substituted with one to three fluorine atoms or

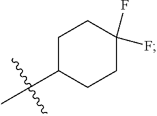 [SMILES: *C1CCC(F)(F)CC1]

R⁴ is

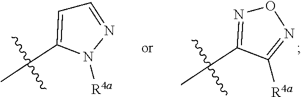

R^{4a} is —C₍₁₋₆₎alkyl, —O—C₍₁₋₆₎alkyl, or —C₍₃₋₆₎cycloalkyl;
n is 1; and
m is 0.

In some embodiments, disclosed herein is a compound of Formula I, or a pharmaceutically acceptable salt thereof, wherein:
R^{1a} is —C₍₁₋₃₎alkyl, wherein the —C₍₁₋₃₎alkyl is unsubstituted or substituted with one to three R^{1aa} groups;
R^{1b} is H or —C₍₁₋₃₎alkyl;
R^{1c} is —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂;
R^{1aa} independently for each occurrence is fluorine;
R^{2a} is H, or —OH;
R^{2b} is H;
R³ is —C₍₄₋₈₎alkyl substituted with one to three fluorine atoms or

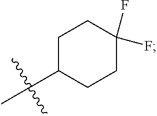 [SMILES: *C1CCC(F)(F)CC1]

R⁴ is

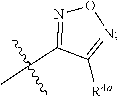

R^{4a} is —C₍₁₋₆₎alkyl, —O—C₍₁₋₆₎alkyl, or —C₍₃₋₆₎cycloalkyl;
n is 1; and
m is 0.

In some embodiments, disclosed herein is a compound of Formula I, or a pharmaceutically acceptable salt thereof, having a formula according to Formula Ia-1:

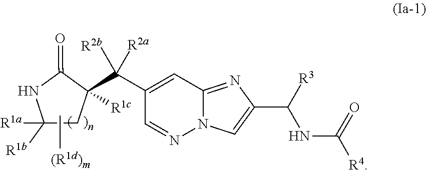

In some embodiments, disclosed herein is a compound of Formula I, or a pharmaceutically acceptable salt thereof, having a formula according to Formula Ia-2:

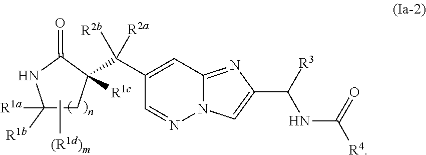

In some embodiments, disclosed herein is a compound of Formula I, or a pharmaceutically acceptable salt thereof, wherein n is 1.

In some embodiments, disclosed herein is a compound of Formula I, or a pharmaceutically acceptable salt thereof, having a formula selected from Formulas Ib-1 to Ib-7:

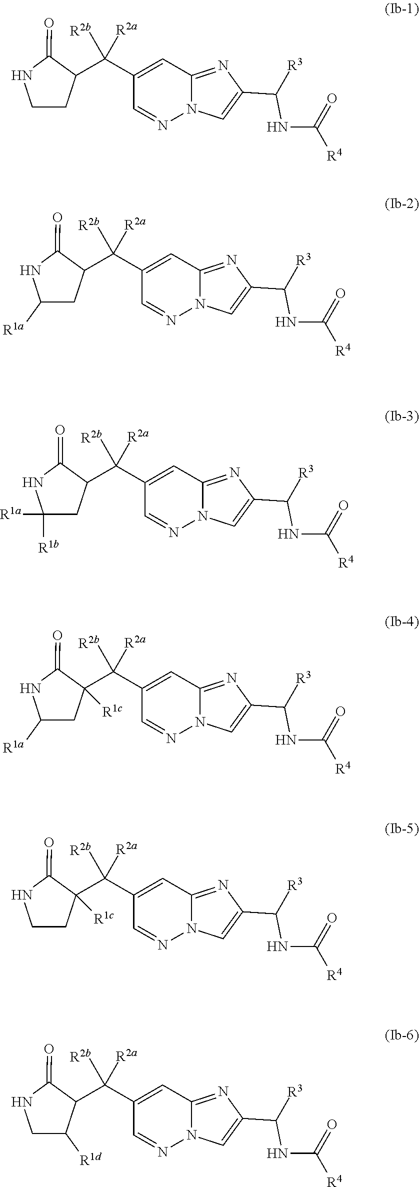

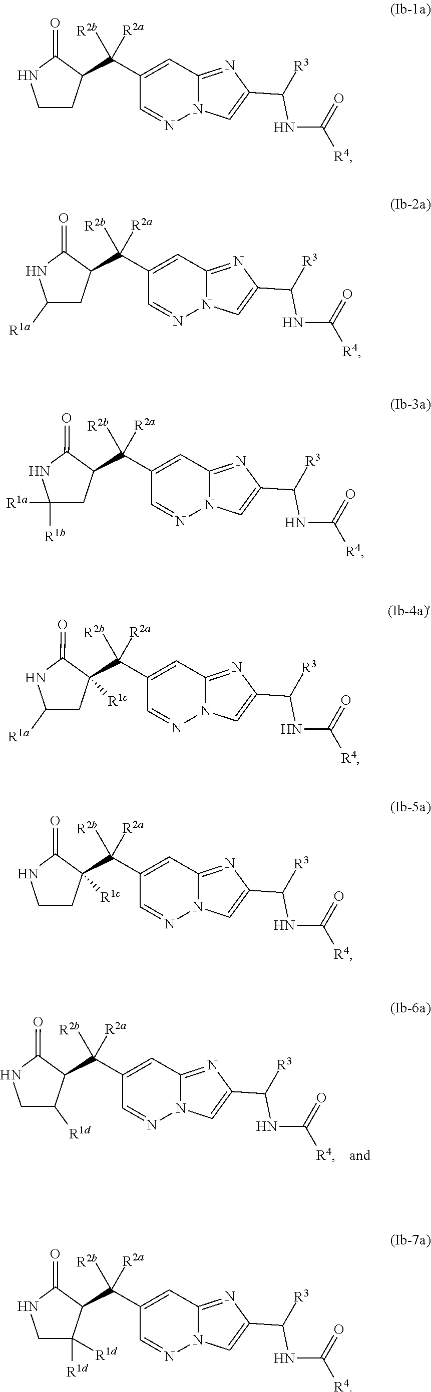

In some embodiments, disclosed herein is a compound of Formula I, or a pharmaceutically acceptable salt thereof, having a formula selected from Formulas Ib-1a to Ib-7a:

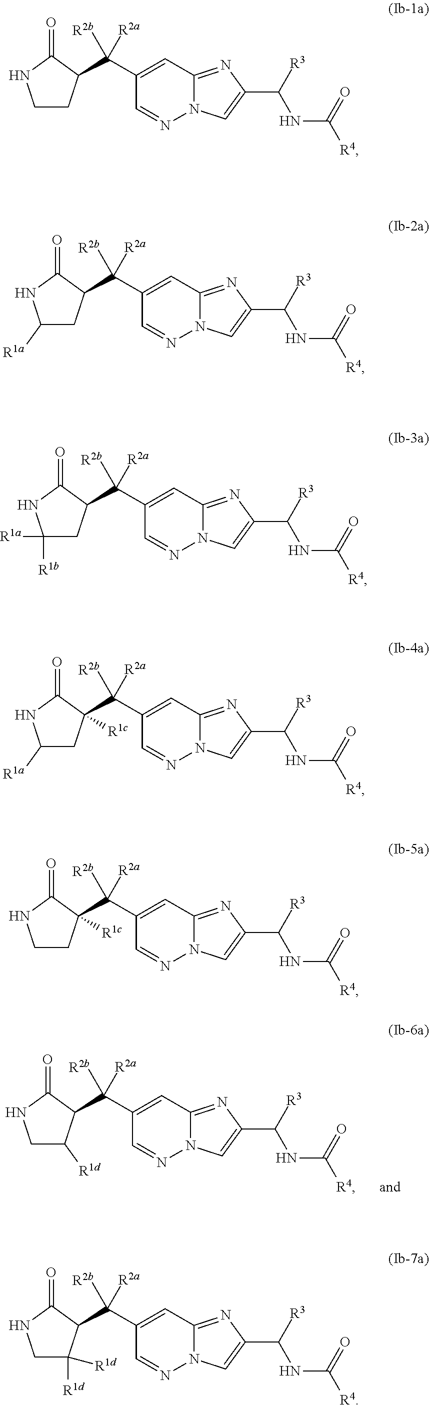

In some embodiments, disclosed herein is a compound of Formula I, or a pharmaceutically acceptable salt thereof, having a formula of Formula Ib-2:

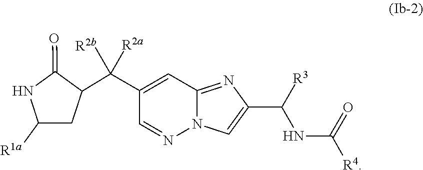

In some embodiments, disclosed herein is a compound of Formula I, or a pharmaceutically acceptable salt thereof, having a formula of Formula Ib-3:

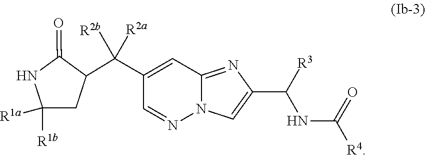

In some embodiments, disclosed herein is a compound of Formula I, or a pharmaceutically acceptable salt thereof, having a formula of Formula Ib-4:

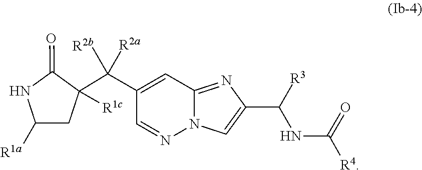

In some embodiments, disclosed herein is a compound of Formula I, or a pharmaceutically acceptable salt thereof, having a formula of Formula Ib-8:

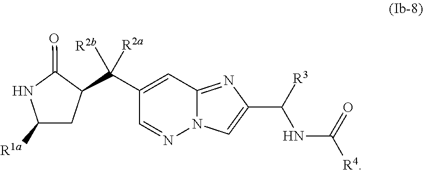

In some embodiments, disclosed herein is a compound of Formula I, or a pharmaceutically acceptable salt thereof, wherein n is 2.

In some embodiments, disclosed herein is a compound of Formula I, or a pharmaceutically acceptable salt thereof, having a formula selected from Formulas Ic-1 to Ic-7:

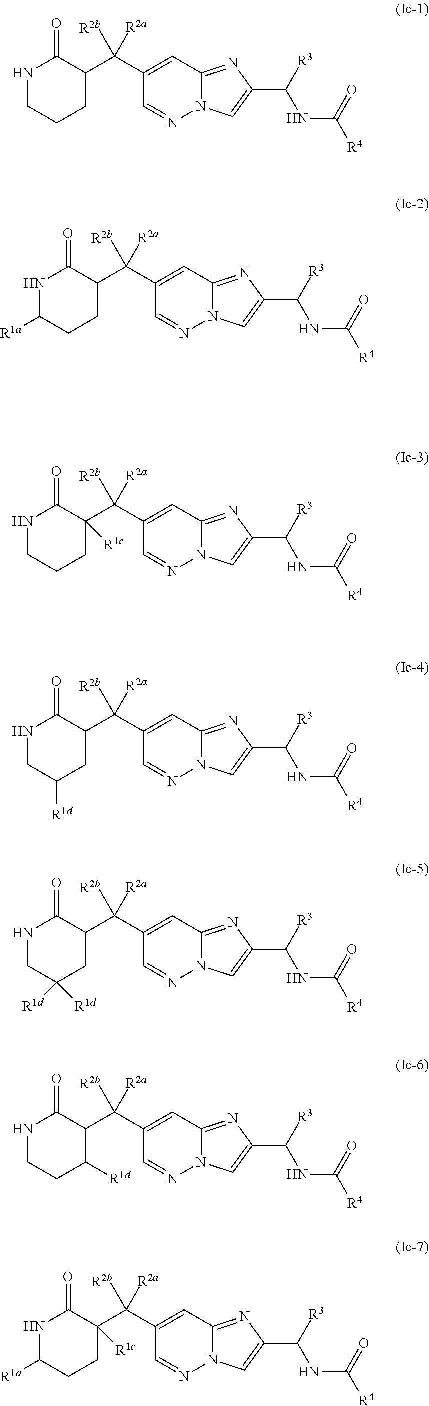

In some embodiments, disclosed herein is a compound of Formula I, or a pharmaceutically acceptable salt thereof, having a formula selected from Formulas Ic-1a to Ic-7a:

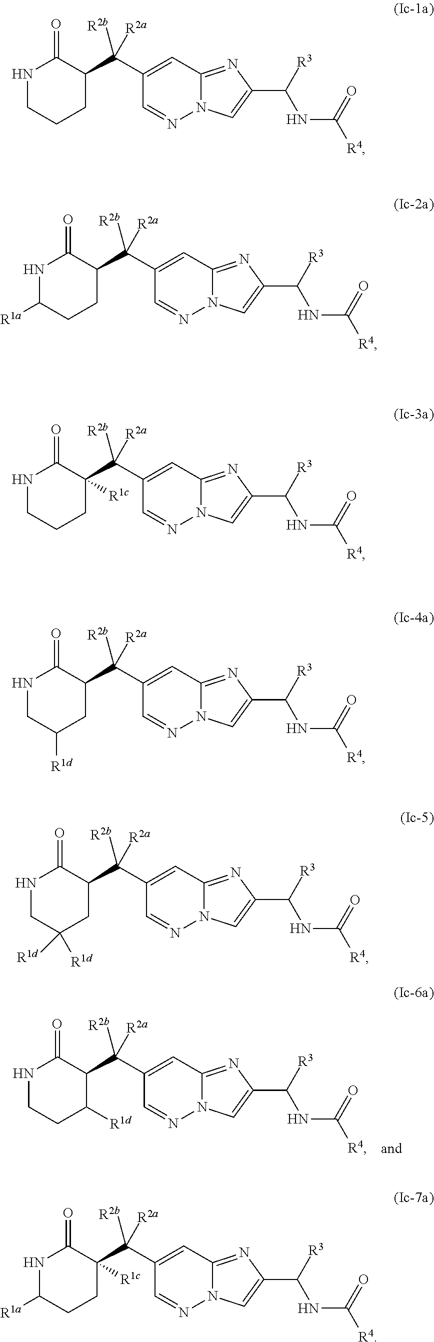

In some embodiments, disclosed herein is a compound of Formula I, or a pharmaceutically acceptable salt thereof, having a formula of Formula Ic-2:

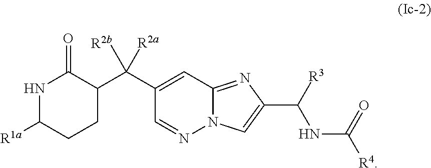

In some embodiments, disclosed herein is a compound of Formula I, or a pharmaceutically acceptable salt thereof, having a formula of Formula Ic-5:

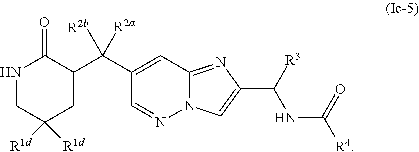

In some embodiments, disclosed herein is a compound of Formula I, or a pharmaceutically acceptable salt thereof, having a formula of Formula Ic-7:

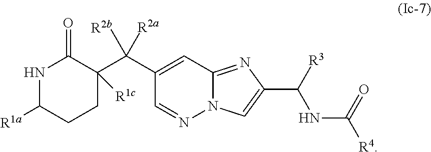

In some embodiments, disclosed herein is a compound of Formula I, or a pharmaceutically acceptable salt thereof, wherein n is 3.

In some embodiments, disclosed herein is a compound of Formula I, or a pharmaceutically acceptable salt thereof, having a formula selected from Formulas Id-1 to Id-2:

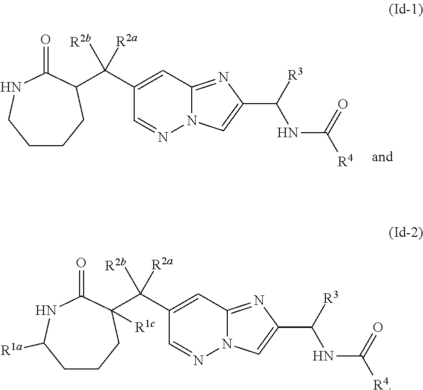

In some embodiments, disclosed herein is a compound of Formula I, or a pharmaceutically acceptable salt thereof, having a formula selected from Formulas Id-1a to Id-2a:

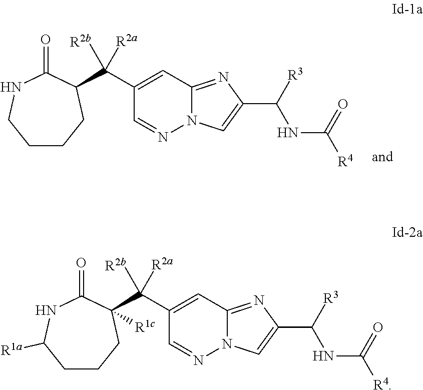

In some embodiments, disclosed herein is a compound of Formula I, or a pharmaceutically acceptable salt thereof, wherein R^{1c} is H, —C₍₁₋₃₎alkyl, or —C₍₃₋₅₎cycloalkyl, wherein the —C₍₁₋₃₎alkyl is unsubstituted or substituted with one to six R^{1aa} groups. In some embodiments, disclosed herein is a compound of Formula I, or a pharmaceutically acceptable salt thereof, wherein R^{1a} is H, —C₍₁₋₆₎alkyl, or —C₍₃₋₅₎cycloalkyl, wherein the —C₍₁₋₃₎alkyl is unsubstituted or substituted with one to six fluorine atoms.

In some embodiments, disclosed herein is a compound of Formula I, or a pharmaceutically acceptable salt thereof, wherein R^{1a} is H or —C₍₁₋₃₎alkyl, wherein the —C₍₁₋₃₎alkyl is unsubstituted or substituted with one to six R^{1aa} groups. In some embodiments, disclosed herein is a compound of Formula I, or a pharmaceutically acceptable salt thereof, wherein R^{1a} is H or —C₍₁₋₃₎alkyl, wherein the —C₍₁₋₃₎alkyl is unsubstituted or substituted with one to six fluorine atoms.

In some embodiments, disclosed herein is a compound of Formula I, or a pharmaceutically acceptable salt thereof, wherein R^{1a} is H or —C₍₁₋₃₎alkyl, wherein the —C₍₁₋₃₎alkyl is unsubstituted or substituted with one to three R^{1aa} groups. In some embodiments, disclosed herein is a compound of Formula I, or a pharmaceutically acceptable salt thereof, wherein R^{1a} is H or —C₍₁₋₃₎alkyl, wherein the —C₍₁₋₃₎alkyl is unsubstituted or substituted with one to three fluorine atoms.

In some embodiments, disclosed herein is a compound of Formula I, or a pharmaceutically acceptable salt thereof, wherein R^{1a} is —C₍₁₋₃₎alkyl, wherein the —C₍₁₋₃₎alkyl is unsubstituted or substituted with one to three R^{1aa} groups. In some embodiments, disclosed herein is a compound of Formula I, or a pharmaceutically acceptable salt thereof, wherein R^{1a} is —C₍₁₋₃₎alkyl, wherein the —C₍₁₋₃₎alkyl is unsubstituted or substituted with one to three fluorine atoms.

In some embodiments, disclosed herein is a compound of Formula I, or a pharmaceutically acceptable salt thereof, wherein R^{1c} is H, —CH₃, —CH₂CH₃, —CH(CH₃)₂, —CF₃, or cyclopropyl. In some embodiments, disclosed herein is a compound of Formula I, or a pharmaceutically acceptable salt thereof, wherein R^{1a} is H, —CH₃, or —CF₃. In some embodiments, disclosed herein is a compound of Formula I, or a pharmaceutically acceptable salt thereof, wherein R^{1a} is —CF₃.

In some embodiments, disclosed herein is a compound of Formula I, or a pharmaceutically acceptable salt thereof, wherein R^{1b} is H, —C₍₁₋₃₎alkyl, or —C₍₃₋₅₎cycloalkyl, wherein the —C₍₁₋₃₎alkyl is unsubstituted or substituted with one to six R^{1aa} groups. In some embodiments, disclosed herein is a compound of Formula I, or a pharmaceutically acceptable salt thereof, wherein R^{1b} is H, —C₍₁₋₆₎alkyl, or —C₍₃₋₅₎cycloalkyl, wherein the —C₍₁₋₃₎alkyl is unsubstituted or substituted with one to six fluorine atoms.

In some embodiments, disclosed herein is a compound of Formula I, or a pharmaceutically acceptable salt thereof, wherein R^{1b} is H or —C₍₁₋₃₎alkyl, wherein the —C₍₁₋₃₎alkyl is unsubstituted or substituted with one to six R^{1aa} groups. In some embodiments, disclosed herein is a compound of Formula I, or a pharmaceutically acceptable salt thereof, wherein R^{1b} is H or —C₍₁₋₃₎alkyl, wherein the —C₍₁₋₃₎alkyl is unsubstituted or substituted with one to six fluorine atoms.

In some embodiments, disclosed herein is a compound of Formula I, or a pharmaceutically acceptable salt thereof, wherein R^{1b} is H or —C₍₁₋₆₎alkyl, wherein the —C₍₁₋₃₎alkyl is unsubstituted or substituted with one to three R^{1aa} groups. In some embodiments, disclosed herein is a compound of Formula I, or a pharmaceutically acceptable salt thereof, wherein R^{1b} is H or —C₍₁₋₃₎alkyl, wherein the —C₍₁₋₃₎alkyl is unsubstituted or substituted with one to three fluorine.

In some embodiments, disclosed herein is a compound of Formula I, or a pharmaceutically acceptable salt thereof, wherein R^{1b} is H, —CH₃, or —CF₃. In some embodiments, disclosed herein is a compound of Formula I, or a pharmaceutically acceptable salt thereof, wherein R^{1b} is H or —CH₃. In some embodiments, disclosed herein is a compound of Formula I, or a pharmaceutically acceptable salt thereof, wherein R^{1b} is H. In some embodiments, disclosed herein is a compound of Formula I, or a pharmaceutically acceptable salt thereof, wherein R^{1b} is —CH₃.

In some embodiments, disclosed herein is a compound of Formula I, or a pharmaceutically acceptable salt thereof, wherein R^{1a} and R^{1b} together with the carbon atom to which they are attached form a spiro C₍₃₋₅₎cycloalkyl or a spiro heterocyclyl selected from

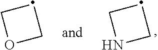

wherein · indicates the carbon atom to which R^{1a} and R^{1b} are attached.

In some embodiments, disclosed herein is a compound of Formula I, or a pharmaceutically acceptable salt thereof, wherein R^{1a} and R^{1b} together with the carbon atom to which they are attached form a spirocyclopropyl or

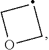

wherein · indicates the carbon atom to which R^{1a} and R^{1b} are attached.

In some embodiments, disclosed herein is a compound of Formula I, or a pharmaceutically acceptable salt thereof, wherein R^{1a} and R^{1c} are taken together to form a C₍₁₋₂₎alkylenyl bridge. In some embodiments, disclosed herein is a compound of Formula I, or a pharmaceutically acceptable salt thereof, wherein R^{1a} and R^{1c} are taken together to form a —CH₂— bridge. In some embodiments, disclosed herein is a compound of Formula I, or a pharmaceutically acceptable salt thereof, wherein R^{1a} and R^{1c} are taken together to form a —CH₂CH₂— bridge.

In some embodiments, disclosed herein is a compound of Formula I, or a pharmaceutically acceptable salt thereof, wherein R^{1a} is H, —C₍₁₋₃₎alkyl, or —C₍₃₋₅₎cycloalkyl, wherein the —C₍₁₋₃₎alkyl is unsubstituted or substituted with one to six fluorine atoms; and
R^{1b} is H or —C₍₁₋₃₎alkyl, wherein the —C₍₁₋₃₎alkyl is unsubstituted or substituted with one to six fluorine atoms.

In some embodiments, disclosed herein is a compound of Formula I, or a pharmaceutically acceptable salt thereof, wherein R^{1a} is H, —CH₃, —CH₂CH₃, —CH(CH₃)₂, —CF₃, or cyclopropyl; and R^{1b} is H, —CH₃, or —CF₃.

In some embodiments, disclosed herein is a compound of Formula I, or a pharmaceutically acceptable salt thereof, wherein R^{1a} is H, —CH₃, or —CF₃; and R^{1b} is H or —CH₃.

In some embodiments, disclosed herein is a compound of Formula I, or a pharmaceutically acceptable salt thereof, wherein R^{1c} is halo, —OH, —C₍₁₋₃₎alkyl, —C₍₁₋₆₎alkyl-CN, —C₍₃₋₅₎cycloalkyl, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups.

In some embodiments, disclosed herein is a compound of Formula I, or a pharmaceutically acceptable salt thereof, wherein R^{1c} is halo, —OH, —C₍₁₋₃₎alkyl, —C₍₁₋₃₎alkyl-CN, —C₍₃₋₅₎cycloalkyl, —C₍₁₋₃₎alkyl-O—C₍₁₋₃₎alkyl, or —C₍₁₋₃₎alkyl-N(C₍₁₋₃₎alkyl)₂.

In some embodiments, disclosed herein is a compound of Formula I, or a pharmaceutically acceptable salt thereof, wherein R^{1c} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups.

In some embodiments, disclosed herein is a compound of Formula I, or a pharmaceutically acceptable salt thereof, wherein R^{1c} is H, halo, —OH, —C₍₁₋₃₎alkyl, —C₍₁₋₆₎alkyl-CN, —C₍₁₋₃₎alkyl-O—C₍₁₋₃₎alkyl, or —C₍₁₋₃₎alkyl-N(C₍₁₋₃₎alkyl)₂.

In some embodiments, disclosed herein is a compound of Formula I, or a pharmaceutically acceptable salt thereof, wherein R^{1c} is H, halo, —OH, —C₍₁₋₆₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂.

In some embodiments, disclosed herein is a compound of Formula I, or a pharmaceutically acceptable salt thereof, wherein R^{1c} is halo, —OH, —C₍₁₋₃₎alkyl, —C₍₁₋₃₎alkyl-CN, —C₍₁₋₃₎alkyl-O—C₍₁₋₃₎ alkyl, or —C₍₁₋₃₎alkyl-N(C₍₁₋₃₎alkyl)₂.

In some embodiments, disclosed herein is a compound of Formula I, or a pharmaceutically acceptable salt thereof, wherein R^{1c} is H, —OH, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂.

In some embodiments, disclosed herein is a compound of Formula I, or a pharmaceutically acceptable salt thereof, wherein R^{1c} is —OH, —C₍₁₋₃₎alkyl-CN, —C₍₁₋₃₎alkyl-O—C₍₁₋₃₎alkyl, or —C₍₁₋₃₎ alkyl-N(C₍₁₋₃₎alkyl)₂.

In some embodiments, disclosed herein is a compound of Formula I, or a pharmaceutically acceptable salt thereof, wherein R^{1c} is H, —C₍₁₋₃₎alkyl-CN, or —C₍₁₋₃₎alkyl-O—C₍₁₋₃₎alkyl. In some embodiments, disclosed herein is a compound of Formula I, or a pharmaceutically acceptable salt thereof, wherein R^{1c} is —C₍₁₋₃₎alkyl-CN or —C₍₁₋₃₎alkyl-O—C₍₁₋₃₎alkyl.

In some embodiments, disclosed herein is a compound of Formula I, or a pharmaceutically acceptable salt thereof, wherein R^{1c} is H, fluorine, —OH, —CH₃, —CH₂CN, —CH₂OCH₃, or —CH₂N(CH₃)₂. In some embodiments, disclosed herein is a compound of Formula I, or a pharmaceutically acceptable salt thereof, wherein R^{1c} is fluorine, —OH, —CH₃, —CH₂CN, —CH₂OCH₃, or —CH₂N(CH₃)₂.

In some embodiments, disclosed herein is a compound of Formula I, or a pharmaceutically acceptable salt thereof, wherein R^{1c} is H. In some embodiments, disclosed herein is a compound of Formula I, or a pharmaceutically acceptable salt thereof, wherein R^{1c} is halo. In some embodiments, disclosed herein is a compound of Formula I, or a pharmaceutically acceptable salt thereof, wherein R^{1c} is fluorine. In some embodiments, disclosed herein is a compound of Formula I, or a pharmaceutically acceptable salt thereof, wherein R^{1c} is —OH. In some embodiments, disclosed herein is a compound of Formula I, or a pharmaceutically acceptable salt thereof, wherein R^{1c} is —C₍₁₋₃₎alkyl. In some embodiments, disclosed herein is a compound of Formula I, or a pharmaceutically acceptable salt thereof, wherein R^{1c} is —CH₃. In some embodiments, disclosed herein is a compound of Formula I, or a pharmaceutically acceptable salt thereof, wherein R^{1c} is —C₍₁₋₃₎alkyl-CN. In some embodiments, disclosed herein is a compound of Formula I, or a pharmaceutically acceptable salt thereof, wherein R^{1c} is —CH₂CN. In some embodiments, disclosed herein is a compound of Formula I, or a pharmaceutically acceptable salt thereof, wherein R^{1c} is —C₍₁₋₃₎alkyl-O—C₍₁₋₃₎alkyl. In some embodiments, disclosed herein is a compound of Formula I, or a pharmaceutically acceptable salt thereof, wherein R^{1c} is —CH₂OCH₃. In some embodiments, disclosed herein is a compound of Formula I, or a pharmaceutically acceptable salt thereof, wherein R^{1c} is —C₍₁₋₃₎alkyl-N(C₍₁₋₃₎alkyl)₂. In some embodiments, disclosed herein is a compound of Formula I, or a pharmaceutically acceptable salt thereof, wherein R^{1c} is —CH₂N(CH₃)₂.

In some embodiments, disclosed herein is a compound of Formula I, or a pharmaceutically acceptable salt thereof, wherein R^{1d} is independently for each occurrence halo, —C₍₁₋₃₎alkyl, or —C₍₃₋₅₎ cycloalkyl, wherein the —C₍₁₋₃₎alkyl is unsubstituted or substituted with one to six R^{1aa} groups. In some embodiments, disclosed herein is a compound of Formula I, or a pharmaceutically acceptable salt thereof, wherein R^{1d} is independently for each occurrence halo, —C₍₁₋₃₎alkyl, or —C₍₃₋₅₎cycloalkyl, wherein the —C₍₁₋₃₎alkyl is unsubstituted or substituted with one to six fluorine atoms.

In some embodiments, disclosed herein is a compound of Formula I, or a pharmaceutically acceptable salt thereof, wherein R^{1d} is independently for each occurrence halo or —C₍₁₋₃₎alkyl, wherein the —C₍₁₋₃₎alkyl is unsubstituted or substituted with one to six R^{1aa} groups. In some embodiments, disclosed herein is a compound of Formula I, or a pharmaceutically acceptable salt thereof, wherein R^{1d} is independently for each occurrence halo or —C₍₁₋₃₎alkyl, wherein the —C₍₁₋₃₎alkyl is unsubstituted or substituted with one to three fluorine atoms.

In some embodiments, disclosed herein is a compound of Formula I, or a pharmaceutically acceptable salt thereof, wherein R^{1d} is independently for each occurrence F, —CH₃, or —CF₃.

In some embodiments, disclosed herein is a compound of Formula I, or a pharmaceutically acceptable salt thereof, wherein R^{1d} is F. In some embodiments, disclosed herein is a compound of Formula I, or a pharmaceutically acceptable salt thereof, wherein R^{1d} is —CH₃. In some embodiments, disclosed herein is a compound of Formula I, or a pharmaceutically acceptable salt thereof, wherein R^{1d} is —CF₃.

In some embodiments, disclosed herein is a compound of Formula I, or a pharmaceutically acceptable salt thereof, wherein R^{1aa} independently for each occurrence is fluorine.

In some embodiments, disclosed herein is a compound of Formula I, or a pharmaceutically acceptable salt thereof, wherein n is 1, 2, or 3. In some embodiments, disclosed herein is a compound of Formula I, or a pharmaceutically acceptable salt thereof, wherein n is 1. In some embodiments, disclosed herein is a compound of Formula I, or a pharmaceutically acceptable salt thereof, wherein n is 2. In some embodiments, disclosed herein is a compound of Formula I, or a pharmaceutically acceptable salt thereof, wherein n is 3.

In some embodiments, disclosed herein is a compound of Formula I, or a pharmaceutically acceptable salt thereof, wherein m is 0, 1, or 2. In some embodiments, disclosed herein is a compound of Formula I, or a pharmaceutically acceptable salt thereof, wherein m is 0. In some embodiments, disclosed herein is a compound of Formula I, or a pharmaceutically acceptable salt thereof, wherein m is 1. In some embodiments, disclosed herein is a compound of Formula I, or a pharmaceutically acceptable salt thereof, wherein m is 2.

In some embodiments, disclosed herein is a compound of Formula I, or a pharmaceutically acceptable salt thereof, having a formula according to Formula Ie-1:

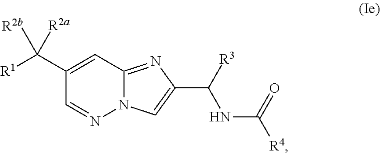

wherein R¹ is selected from the group consisting of:

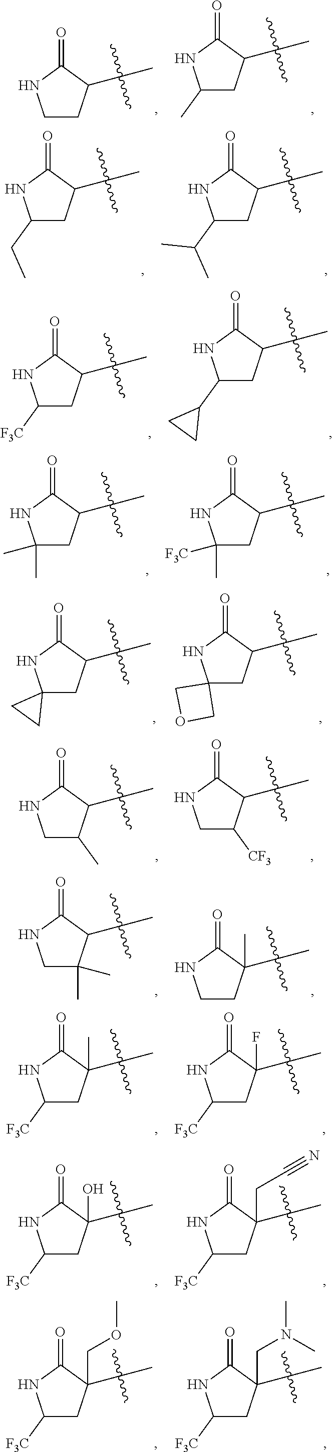

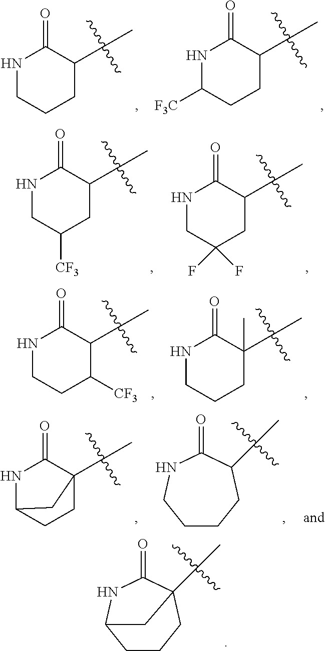

In some embodiments, disclosed herein is a compound of Formula I, or a pharmaceutically acceptable salt thereof, having a formula according to Formula Ie:

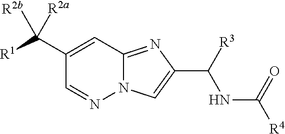

wherein R¹ is selected from the group consisting of:

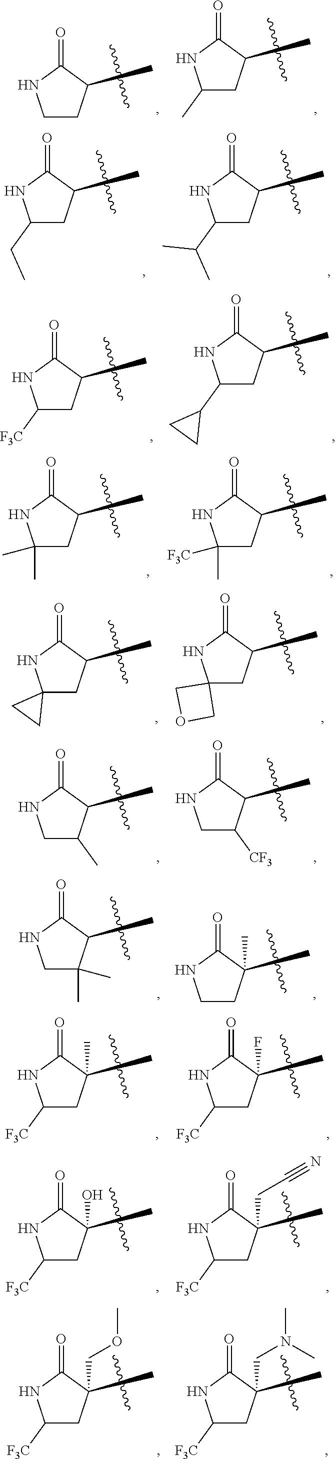

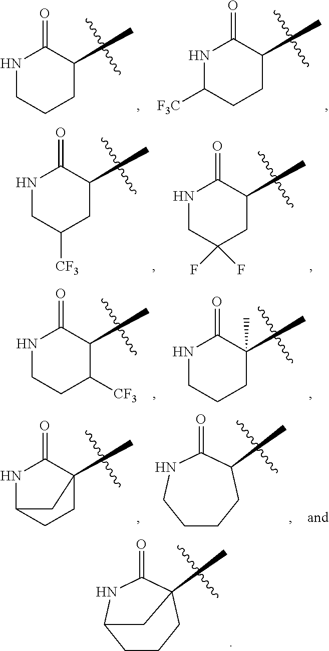

In some embodiments, disclosed herein is a compound of Formula Ie, or a pharmaceutically acceptable thereof, wherein R¹ is selected from the group consisting of:

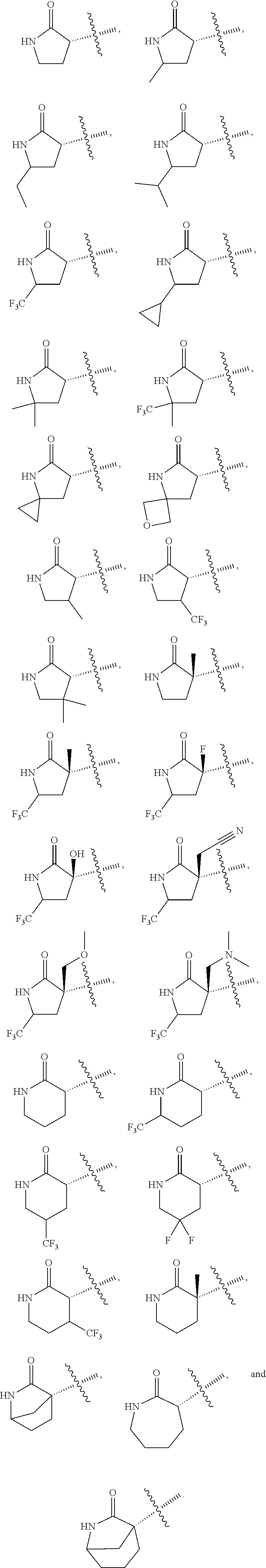

In some embodiments, disclosed herein is a compound of Formula Ie, or a pharmaceutically acceptable salt thereof, wherein R¹ is selected from the group consisting of:

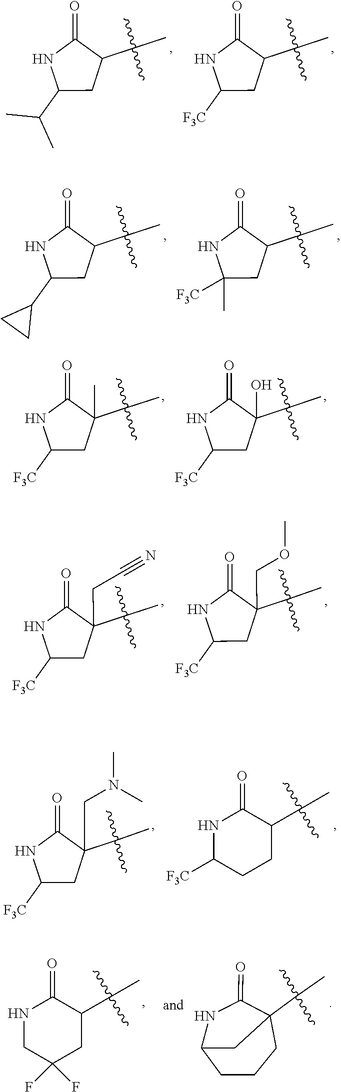

In some embodiments, disclosed herein is a compound of Formula Ie, or a pharmaceutically acceptable salt thereof, wherein R¹ is selected from the group consisting of:

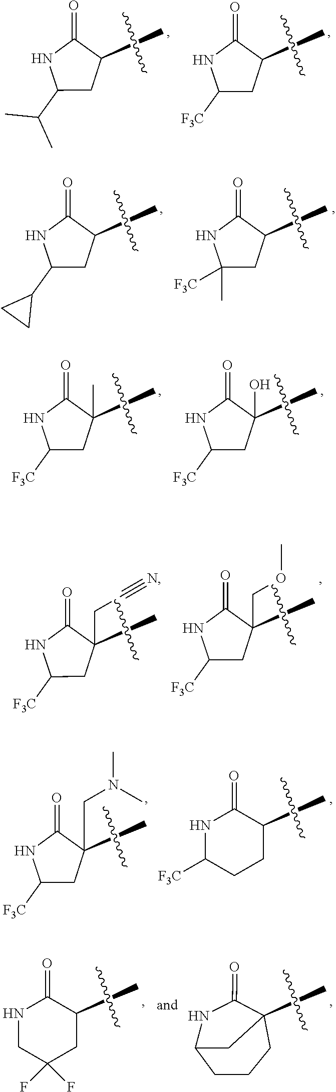

In some embodiments, disclosed herein is a compound of Formula Ie, or a pharmaceutically acceptable salt thereof, wherein R¹ is selected from the group consisting of:

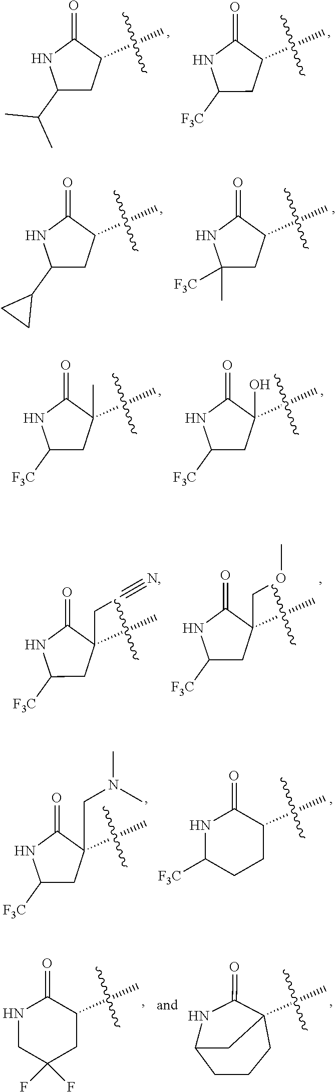

In some embodiments, disclosed herein is a compound of Formula Ie, or a pharmaceutically acceptable salt thereof, wherein R¹ is selected from the group consisting of:

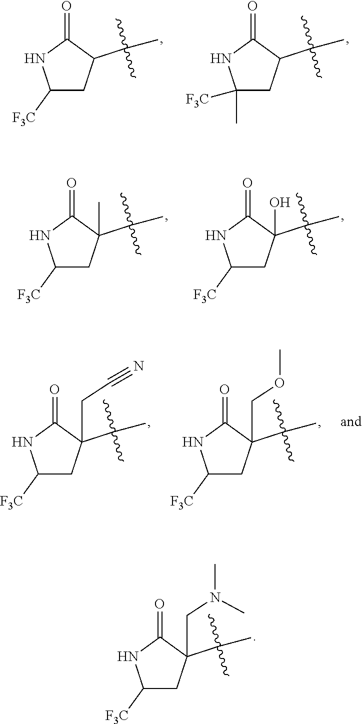

In some embodiments, disclosed herein is a compound of Formula Ie, or a pharmaceutically acceptable salt thereof, wherein R¹ is selected from the group consisting of:

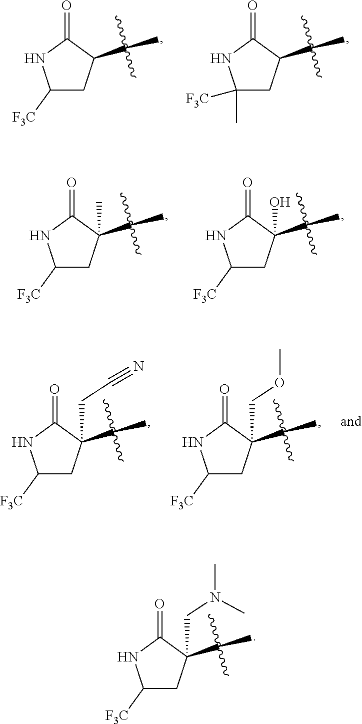

In some embodiments, disclosed herein is a compound of Formula Ie, or a pharmaceutically acceptable salt thereof, wherein R¹ is selected from the group consisting of:

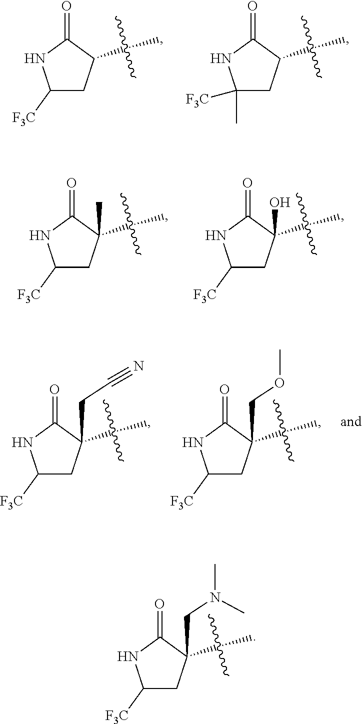

In some embodiments, disclosed herein is a compound of Formula I, or a pharmaceutically acceptable salt thereof, wherein R^{2a} is H, —OH, or —C₍₁₋₃₎alkyl, wherein the —C₍₁₋₃₎alkyl is unsubstituted or substituted with one to six R² groups. In some embodiments, disclosed herein is a compound of Formula I, or a pharmaceutically acceptable salt thereof, wherein R^{2a} is H, —OH, or —C₍₁₋₃₎alkyl. In some embodiments, disclosed herein is a compound of Formula I, or a pharmaceutically acceptable salt thereof, wherein R^{2a} is H or —OH.

In some embodiments, disclosed herein is a compound of Formula I, or a pharmaceutically acceptable salt thereof, wherein R^{2a} is H. In some embodiments, disclosed herein is a compound of Formula I, or a pharmaceutically acceptable salt thereof, wherein R^{2a} is —OH. In some embodiments, disclosed herein is a compound of Formula I, or a pharmaceutically acceptable salt thereof, wherein R^{2a} is —C₍₁₋₃₎alkyl. In some embodiments, disclosed herein is a compound of Formula I, or a pharmaceutically acceptable salt thereof, wherein R² is —CH₃. In some embodiments, disclosed herein is a compound of Formula I, or a pharmaceutically acceptable salt thereof, wherein R² is —CH₂CH₃.

In some embodiments, disclosed herein is a compound of Formula I, or a pharmaceutically acceptable salt thereof, wherein R^{2a} and R^{1c} are taken together with the carbon atoms to which they are attached to form a C₍₃₋₅₎cycloalkyl or a 4- to 6-membered heterocycle. In some embodiments, disclosed herein is a compound of Formula I, or a pharmaceutically acceptable salt thereof, wherein R^{2a} and R^{1c} are taken together with the carbon atoms to which they are attached to form a C₍₃₋₅₎cycloalkyl. In some embodiments, disclosed herein is a compound of Formula I, or a pharmaceutically acceptable salt thereof, wherein R^{2a} and R^{1c} are taken together with the carbon atoms to which they are attached to form a cyclopropyl.

In some embodiments, disclosed herein is a compound of Formula I, or a pharmaceutically acceptable salt thereof, wherein R^{2aa} independently for each occurrence is fluorine or —OH. In some embodiments, disclosed herein is a compound of Formula I, or a pharmaceutically acceptable salt thereof, wherein R^{2aa} independently for each occurrence is fluorine.

In some embodiments, disclosed herein is a compound of Formula I, or a pharmaceutically acceptable salt thereof, wherein R^{2b} is H. In some embodiments, disclosed herein is a compound of Formula I, or a pharmaceutically acceptable salt thereof, wherein R^{2b} is —C₍₁₋₃₎alkyl. In some embodiments, disclosed herein is a compound of Formula I, or a pharmaceutically acceptable salt thereof, wherein R^{2b} is —CH₃. In some embodiments, disclosed herein is a compound of Formula I, or a pharmaceutically acceptable salt thereof, wherein R^{2b} is —CH₂CH₃.

In some embodiments, disclosed herein is a compound of Formula I, or a pharmaceutically acceptable salt thereof, wherein R^{2a} and R^{2b} are taken together with the carbon atom to which they are attached to form a C═O.

In some embodiments, disclosed herein is a compound of Formula I, or a pharmaceutically acceptable salt thereof, wherein R^{2a} is H, —OH, or —C₍₁₋₃₎alkyl; and R^{2b} is H or —C₍₁₋₃₎alkyl; or R^{2a} and R^{2b} are taken together with the carbon atom to which they are attached to form a C═O.

In some embodiments, disclosed herein is a compound of Formula I, or a pharmaceutically acceptable salt thereof, wherein R^{2a} is H and R^{2b} is H; R^{2a} is —OH and R^{2b} is H; R^{2a} is —CH₃ and R^{2b} is H; R^{2a} is —CH₂CH₃ and R^{2b} is H; R^{2a} is —OH and R^{2b} is —CH₃; or R^{2a} and R^{2b} are taken together with the carbon atom to which they are attached to form a C═O.

In some embodiments, disclosed herein is a compound of Formula I, or a pharmaceutically acceptable salt thereof, wherein R^{2a} is H or —OH, and R^{2b} is H.

In some embodiments, disclosed herein is a compound of Formula I, or a pharmaceutically acceptable salt thereof, having a formula according to Formula If:

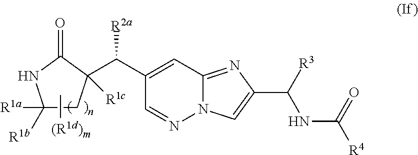

In some embodiments, disclosed herein is a compound of Formula I, or a pharmaceutically acceptable salt thereof, wherein R³ is —C₍₁₋₁₀₎alkyl, —C₍₁₋₆₎alkyl-O—C₍₁₋₆₎alkyl, —C₍₁₋₆₎alkyl-O—C₍₃₋₅₎ cycloalkyl, —C₍₁₋₆₎alkyl-O—C₍₃₋₅₎cycloalkyl-C₍₁₋₃₎alkyl, —C₍₃₋₈₎cycloalkyl, or —C₍₁₋₃₎alkyl-(C₍₃₋₅₎ cycloalkyl)₁₋₂, each of which is unsubstituted or substituted with one to six fluorine atoms.

In some embodiments, disclosed herein is a compound of Formula I, or a pharmaceutically acceptable salt thereof, wherein R³ is —C₍₁₋₁₀₎alkyl, —C₍₁₋₆₎alkyl-O—C₍₁₋₆₎alkyl, —C₍₁₋₆₎alkyl-O—C₍₃₋₅₎cycloalkyl, or —C₍₃₋₈₎cycloalkyl, each of which is unsubstituted or substituted with one to six fluorine atoms.

In some embodiments, disclosed herein is a compound of Formula I, or a pharmaceutically acceptable salt thereof, wherein R³ is —C₍₁₋₁₀₎alkyl or —C₍₃₋₈₎cycloalkyl, each of which is unsubstituted or substituted with one to six fluorine atoms.

In some embodiments, disclosed herein is a compound of Formula I, or a pharmaceutically acceptable salt thereof, wherein R³ is —C₍₄₋₁₀₎alkyl or cyclohexyl, each of which is unsubstituted or substituted with one to six fluorine atoms.

In some embodiments, disclosed herein is a compound of Formula I, or a pharmaceutically acceptable salt thereof, wherein R³ is —C₍₄₋₁₀₎alkyl or cyclohexyl, each of which is substituted with one to six fluorine atoms. In some embodiments, disclosed herein is a compound of Formula I, or a pharmaceutically acceptable salt thereof, wherein R³ is —C₍₄₋₈₎alkyl or cyclohexyl, each of which is substituted with one to six fluorine atoms.

In some embodiments, disclosed herein is a compound of Formula I, or a pharmaceutically acceptable salt thereof, wherein R³ is —C₍₄₋₈₎alkyl substituted with one to three fluorine atoms or

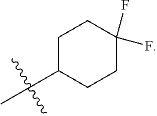 [SMILES: *C1CCC(F)(F)CC1]

In some embodiments, disclosed herein is a compound of Formula I, or a pharmaceutically acceptable salt thereof, wherein R³ is:

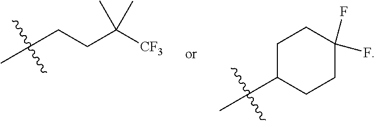

In some embodiments, disclosed herein is a compound of Formula I, or a pharmaceutically acceptable salt thereof, wherein R³ is

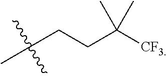 [SMILES: *CCC(C)(C)C(F)(F)F]

In some embodiments, disclosed herein is a compound of Formula I, or a pharmaceutically acceptable salt thereof, wherein R³ is

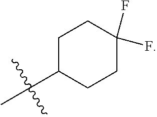 [SMILES: *C1CCC(F)(F)CC1]

In some embodiments, disclosed herein is a compound of Formula I, or a pharmaceutically acceptable salt thereof, having a formula according to Formula Ig:

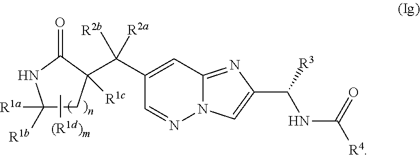

In some embodiments, disclosed herein is a compound of Formula I, or a pharmaceutically acceptable salt thereof, having a formula according to Formula Ig-1:

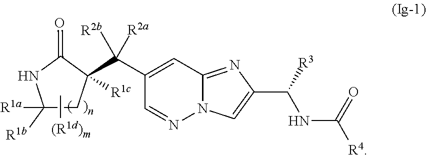

In some embodiments, disclosed herein is a compound of Formula I, or a pharmaceutically acceptable salt thereof, having a formula according to Formula Ig-2:

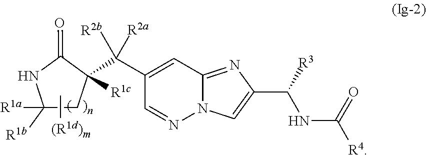

In some embodiments, disclosed herein is a compound of any one of the preceding Formula I, Formulas Ia-1-Ia-7, Formulas Ib-1-Ib-8, Formulas Ia-1a-Ia-7a, Formulas Ic-1-Ic-7, Formulas Ic-1a-Ic-7a. Formulas Id-1-Id-2, Formulas Id-1a-Id-2a, Formulas Ie-1-Ie-2, Formula Ic-1, Formula Ic-2, Formula Ic-3, Formula Ic-4, Formula Ic-5, Formula Ic-6, Formula Ic-7, Formula Id-1, Formula Id-2, Formula Ie, Formula If, Formula Ig, Formula Ig-1, and Formula Ig-2, Formula Ih, Formula Ih-1, and Formula Ih-2, Formula Ih-3, Formula II, Formula II-1, and Formula II-2, Formula II-3, Formula Ij, Formula Ij-1, and Formula Ij-2, Formula Ij-3, Formula Ik, Formula Ik-1, Formula Ik-2, Formula Ik-3, Formula Im, Formula Im-1, Formula Im-2, and Formula Im-3, as described and provided herein, or a pharmaceutically acceptable salt thereof, wherein R³ is —C₍₃₋₆₎cycloalkyl, each of which is unsubstituted or substituted with one to six fluorine atoms.

In some embodiments, disclosed herein is a compound of any one of the preceding Formula I, Formulas Ia-1-Ia-7, Formulas Ib-1-Ib-8, Formulas Ia-1a-Ia-7a, Formulas Ic-1-Ic-7, Formulas Ic-1a-Ic-7a, Formulas Id-1-Id-2, Formulas Id-1a-Id-2a, Formulas Ie-1-Ie-2, Formula Ic-1, Formula Ic-2, Formula Ic-3, Formula Ic-4, Formula Ic-5, Formula Ic-6, Formula Ic-7, Formula Id-1, Formula Id-2, Formula Ie, Formula If, Formula Ig, Formula Ig-1, and Formula Ig-2, Formula Ih, Formula Ih-1, and Formula Ih-2, Formula Ih-3, Formula II, Formula II-1, and Formula II-2, Formula II-3, Formula Ij, Formula Ij-1, and Formula Ij-2, Formula Ij-3, Formula Ik, Formula Ik-1, Formula Ik-2, Formula Ik-3, Formula Im, Formula Im-1, Formula Im-2, and Formula Im-3, or a pharmaceutically acceptable salt thereof, wherein R³ is —C₍₃₋₅₎cycloalkyl, each of which is unsubstituted or substituted with one to six fluorine atoms.

In some embodiments, disclosed herein is a compound of any one of the preceding Formula I, Formulas Ia-1-Ia-7, Formulas Ib-1-Ib-8, Formulas Ia-1a-Ia-7a, Formulas Ic-1-Ic-7, Formulas Ic-1a-Ic-7a, Formulas Id-1-Id-2, Formulas Id-1a-Id-2a, Formulas Ie-1-Ie-2, Formula Ic-1, Formula Ic-2, Formula Ic-3, Formula Ic-4, Formula Ic-5, Formula Ic-6, Formula Ic-7, Formula Id-1, Formula Id-2, Formula Ie, Formula If, Formula Ig, Formula Ig-1, and Formula Ig-2, Formula Ih, Formula Ih-1, and Formula Ih-2, Formula Ih-3, Formula Ii, Formula Ii-1, and Formula Ii-2, Formula Ii-3, Formula Ij, Formula Ij-1, and Formula Ij-2, Formula Ij-3, Formula Ik, Formula Ik-1, Formula Ik-2, Formula Ik-3, Formula Im, Formula Im-1, Formula Im-2, and Formula Im-3, or a pharmaceutically acceptable salt thereof, wherein R³ is —C₍₃₋₅₎cycloalkyl, each of which is unsubstituted or substituted with one to six fluorine atoms.

In some embodiments, disclosed herein is a compound of any one of the preceding Formula I, Formulas Ia-1-Ia-7, Formulas Ib-1-Ib-8, Formulas Ia-1a-Ia-7a, Formulas Ic-1-Ic-7, Formulas Ic-1a-Ic-7a, Formulas Id-1-Id-2, Formulas Id-1a-Id-2a, Formulas Ie-1-Ie-2, Formula Ic-1, Formula Ic-2, Formula Ic-3, Formula Ic-4, Formula Ic-5, Formula Ic-6, Formula Ic-7, Formula Id-1, Formula Id-2, Formula Ie, Formula If, Formula Ig, Formula Ig-1, and Formula Ig-2, Formula Ih, Formula Ih-1, and Formula Ih-2, Formula Ih-3, Formula Ii, Formula Ii-1, and Formula Ii-2, Formula Ii-3, Formula Ij, Formula Ij-1, and Formula Ij-2, Formula Ij-3, Formula Ik, Formula Ik-1, Formula Ik-2, Formula Ik-3, Formula Im, Formula Im-1, Formula Im-2, and Formula Im-3, or a pharmaceutically acceptable salt thereof, wherein R³ is cyclohexyl, each of which is unsubstituted or substituted with one to six fluorine atoms.

In some embodiments, disclosed herein is a compound of any one of the preceding Formula I, Formulas Ia-1-Ia-7, Formulas Ib-1-Ib-8, Formulas Ia-1a-Ia-7a, Formulas Ic-1-Ic-7, Formulas Ic-1a-Ic-7a, Formulas Id-1-Id-2, Formulas Id-1a-Id-2a, Formulas Ie-1-Ie-2, Formula Ic-1, Formula Ic-2, Formula Ic-3, Formula Ic-4, Formula Ic-5, Formula Ic-6, Formula Ic-7, Formula Id-1, Formula Id-2, Formula Ie, Formula If, Formula Ig, Formula Ig-1, and Formula Ig-2, Formula Ih, Formula Ih-1, and Formula Ih-2, Formula Ih-3, Formula Ii, Formula Ii-1, and Formula Ii-2, Formula Ii-3, Formula Ij, Formula Ij-1, and Formula Ij-2, Formula Ij-3, Formula Ik, Formula Ik-1, Formula Ik-2, Formula Ik-3, Formula Im, Formula Im-1, Formula Im-2, and Formula Im-3, or a pharmaceutically acceptable salt thereof, wherein R³ is cyclopentyl, each of which is unsubstituted or substituted with one to six fluorine atoms.

In some embodiments, disclosed herein is a compound of any one of the preceding Formula I, Formulas Ia-1-Ia-7, Formulas Ib-1-Ib-8, Formulas Ia-1a-Ia-7a, Formulas Ic-1-Ic-7, Formulas Ic-1a-Ic-7a, Formulas Id-1-Id-2, Formulas Id-1a-Id-2a, Formulas Ie-1-Ie-2, Formula Ic-1, Formula Ic-2, Formula Ic-3, Formula Ic-4, Formula Ic-5, Formula Ic-6, Formula Ic-7, Formula Id-1, Formula Id-2, Formula Ie, Formula If, Formula Ig, Formula Ig-1, and Formula Ig-2, Formula Ih, Formula Ih-1, and Formula Ih-2, Formula Ih-3, Formula Ii, Formula Ii-1, and Formula Ii-2, Formula Ii-3, Formula Ij, Formula Ij-1, and Formula Ij-2, Formula Ij-3, Formula Ik, Formula Ik-1, Formula Ik-2, Formula Ik-3, Formula Im, Formula Im-1, Formula Im-2, and Formula Im-3, or a pharmaceutically acceptable salt thereof, wherein R³ is cyclobutyl, each of which is unsubstituted or substituted with one to six fluorine atoms.

In some embodiments, disclosed herein is a compound of any one of the preceding Formula I, Formulas Ia-1-Ia-7, Formulas Ib-1-Ib-8, Formulas Ia-1a-Ia-7a, Formulas Ic-1-Ic-7, Formulas Ic-1a-Ic-7a, Formulas Id-1-Id-2, Formulas Id-1a-Id-2a, Formulas Ie-1-Ie-2, Formula Ic-1, Formula Ic-2, Formula Ic-3, Formula Ic-4, Formula Ic-5, Formula Ic-6, Formula Ic-7, Formula Id-1, Formula Id-2, Formula Ie, Formula If, Formula Ig, Formula Ig-1, and Formula Ig-2, Formula Ih, Formula Ih-1, and Formula Ih-2, Formula Ih-3, Formula Ii, Formula Ii-1, and Formula Ii-2, Formula Ii-3, Formula Ij, Formula Ij-1, and Formula Ij-2, Formula Ij-3, Formula k, Formula Ik-1, Formula Ik-2, Formula Ik-3, Formula Im, Formula Im-1, Formula Im-2, and Formula Im-3, or a pharmaceutically acceptable salt thereof, wherein R³ is cyclopropyl, each of which is unsubstituted or substituted with one to five fluorine atoms.

In some embodiments, disclosed herein is a compound of Formula I, or a pharmaceutically acceptable salt thereof, having a formula according to Formula Ih:

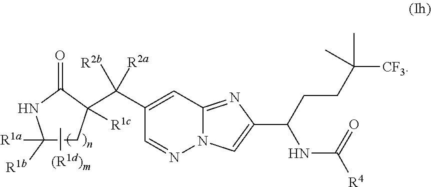

In some embodiments, disclosed herein is a compound of Formula I, or a pharmaceutically acceptable salt thereof, having a formula according to Formula Ih-1:

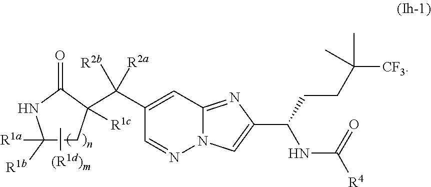

In some embodiments, disclosed herein is a compound of Formula I, or a pharmaceutically acceptable salt thereof, having a formula according to Formula Ih-2:

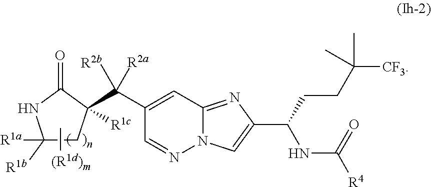

In some embodiments, disclosed herein is a compound of Formula I, or a pharmaceutically acceptable salt thereof, having a formula according to Formula Ih-3:

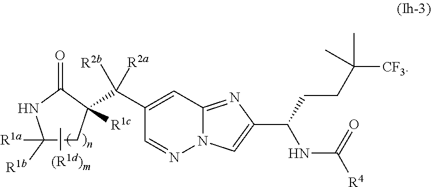

In some embodiments, disclosed herein is a compound of Formula I, or a pharmaceutically acceptable salt thereof, having a formula according to Formula Ii:

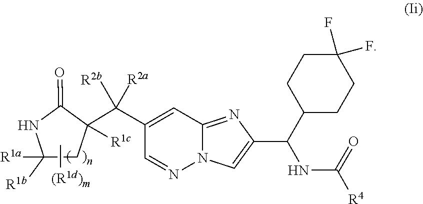

In some embodiments, disclosed herein is a compound of Formula I, or a pharmaceutically acceptable salt thereof, having a formula according to Formula Ii-1:

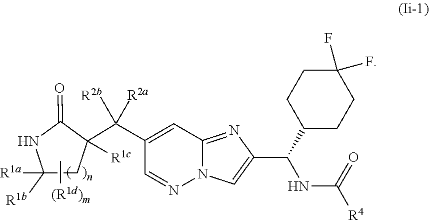

In some embodiments, disclosed herein is a compound of Formula I, or a pharmaceutically acceptable salt thereof, having a formula according to Formula Ii-2:

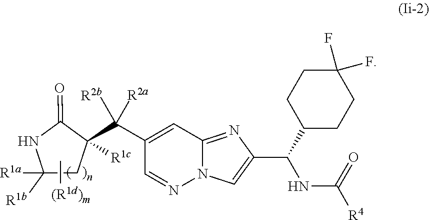

In some embodiments, disclosed herein is a compound of Formula I, or a pharmaceutically acceptable salt thereof, having a formula according to Formula Ii-3:

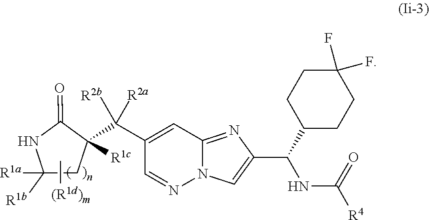

In some embodiments, disclosed herein is a compound of Formula I, or a pharmaceutically acceptable salt thereof, wherein R⁴ is a 5-membered heteroaryl that is substituted with one or two R¹ groups.

In some embodiments, disclosed herein is a compound of Formula I, or a pharmaceutically acceptable salt thereof, wherein R⁴ is a 5-membered heteroaryl comprising one to three heteroatoms selected from O and N, wherein the 5-membered heteroaryl is unsubstituted or substituted with one to two R^{4a} groups. In some embodiments, disclosed herein is a compound of Formula I, or a pharmaceutically acceptable salt thereof, wherein R^{4a} is a 5-membered heteroaryl comprising one to three heteroatoms selected from O and N, wherein the 5-membered heteroaryl is substituted with one R¹ groups.

In some embodiments, disclosed herein is a compound of Formula I, or a pharmaceutically acceptable salt thereof, wherein R⁴ is pyrrolyl, pyrazolyl, imidazolyl, triazolyl, oxazolyl, isoxazolyl, or oxadiazolyl, each of which is unsubstituted or substituted with one to two R^{4a} groups. In some embodiments, disclosed herein is a compound of Formula I, or a pharmaceutically acceptable salt thereof, wherein R⁴ is pyrazolyl, triazolyl, isoxazolyl, or oxadiazolyl, each of which is unsubstituted or substituted with one to two R^{4a} groups. In some embodiments, disclosed herein is a compound of Formula I, or a pharmaceutically acceptable salt thereof, wherein R⁴ is pyrazolyl or oxadiazolyl, each of which is unsubstituted or substituted with one to two R^{4a} groups. In some embodiments, disclosed herein is a compound of Formula I, or a pharmaceutically acceptable salt thereof, wherein R⁴ is pyrazolyl or oxadiazolyl, each of which is substituted with one R^{4a} group.

In some embodiments, disclosed herein is a compound of Formula I, or a pharmaceutically acceptable salt thereof, wherein R⁴ is pyrrolyl, pyrazolyl, imidazolyl, 1,2,3-triazolyl, 1,2,4-triazolyl, oxazolyl, isoxazolyl, 1,2,3-oxadiazolyl, 1,2,4-oxadizaolyl, 1,2,5-oxadiazolyl, or 1,3,4-oxadiazolyl, each of which is unsubstituted or substituted with one to two R^{4a} groups. In some embodiments, disclosed herein is a compound of Formula I, or a pharmaceutically acceptable salt thereof, wherein R⁴ is pyrazolyl or 1,2,5-oxadiazolyl, each of which is unsubstituted or substituted with one to two R^{4a} groups. In some embodiments, disclosed herein is a compound of Formula I, or a pharmaceutically acceptable salt thereof, wherein R⁴ is pyrazolyl or 1,2,5-oxadiazolyl, each of which is substituted with one R^{4a} group.

In some embodiments, disclosed herein is a compound of Formula I, or a pharmaceutically acceptable salt thereof, wherein R⁴ is:

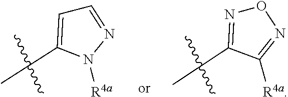

In some embodiments, disclosed herein is a compound of Formula I, or a pharmaceutically acceptable salt thereof, wherein R⁴ is:

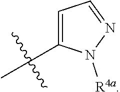

In some embodiments, disclosed herein is a compound of Formula I, or a pharmaceutically acceptable salt thereof, wherein R⁴ is:

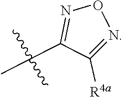

In some embodiments, disclosed herein is a compound of Formula I, or a pharmaceutically acceptable salt thereof, wherein R^{4a} is halo, —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, —C₍₁₋₆₎alkyl-O—C₍₁₋₆₎alkyl, and —C₍₀₋₂₎alkyl-C₍₃₋₆₎cycloalkyl are unsubstituted or substituted with one to six substituents independently selected from fluorine, —CH₃, —CH₂F, —CHF₂, and —CF₃.

In some embodiments, disclosed herein is a compound of Formula I, or a pharmaceutically acceptable salt thereof, wherein R^{4a} is halo, —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎cycloalkyl.

In some embodiments, disclosed herein is a compound of Formula I, or a pharmaceutically acceptable salt thereof, wherein R^{4a} is —C₍₁₋₆₎alkyl, —O—C₍₁₋₆₎alkyl, —C₍₁₋₆₎alkyl-O—C₍₁₋₆₎alkyl, or —C₍₀₋₂₎ alkyl-C₍₃₋₆₎cycloalkyl. In some embodiments, disclosed herein is a compound of Formula I, or a pharmaceutically acceptable salt thereof, wherein R^{4a} is —C₍₁₋₆₎alkyl, —O—C₍₁₋₆₎alkyl, —C₍₁₋₆₎alkyl-O—C₍₁₋₆₎alkyl, or —C₍₃₋₆₎cycloalkyl.

In some embodiments, disclosed herein is a compound of Formula I, or a pharmaceutically acceptable salt thereof, wherein R^{4a} is —C₍₁₋₆₎alkyl, —O—C₍₁₋₆₎alkyl, or —C₍₃₋₆₎cycloalkyl. In some embodiments, disclosed herein is a compound of Formula I, or a pharmaceutically acceptable salt thereof, wherein R^{4a} is —C₍₁₋₆₎alkyl. In some embodiments, disclosed herein is a compound of Formula I, or a pharmaceutically acceptable salt thereof, wherein R^{4a} is —O—C₍₁₋₆₎alkyl. In some embodiments, disclosed herein is a compound of Formula I, or a pharmaceutically acceptable salt thereof, wherein R^{4a} is —C₍₃₋₆₎cycloalkyl.

In some embodiments, disclosed herein is a compound of Formula I, or a pharmaceutically acceptable salt thereof, wherein R^{4a} is —CH₃, —CH(CH₃)₂, —OCH₃, or cyclopropyl. In some embodiments, disclosed herein is a compound of Formula I, or a pharmaceutically acceptable salt thereof, wherein R^{4a} is —CH₃. In some embodiments, disclosed herein is a compound of Formula I, or a pharmaceutically acceptable salt thereof, wherein R^{4a} is —CH(CH₃)₂. In some embodiments, disclosed herein is a compound of Formula I, or a pharmaceutically acceptable salt thereof, wherein R^{4a} is —OCH₃. In some embodiments, disclosed herein is a compound of Formula I, or a pharmaceutically acceptable salt thereof, wherein R^{4a} is cyclopropyl.

In some embodiments, disclosed herein is a compound of Formula I, or a pharmaceutically acceptable salt thereof, wherein R⁴ is selected from the group consisting of:

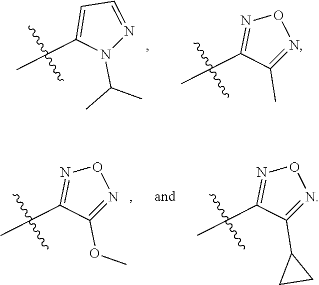

In some embodiments, disclosed herein is a compound of Formula I, or a pharmaceutically acceptable salt thereof, wherein R⁴ is:

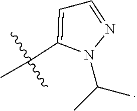 [SMILES: *c1ccnn1C(C)C]

In some embodiments, disclosed herein is a compound of Formula I, or a pharmaceutically acceptable salt thereof, wherein R⁴ is:

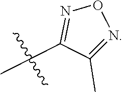 [SMILES: *c1nonc1C]

In some embodiments, disclosed herein is a compound of Formula I, or a pharmaceutically acceptable salt thereof, wherein R⁴ is:

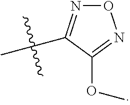 [SMILES: *c1nonc1OC]

In some embodiments, disclosed herein is a compound of Formula I, or a pharmaceutically acceptable salt thereof, wherein R⁴ is:

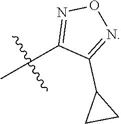 [SMILES: *c1nonc1C1CC1]

In some embodiments, disclosed herein is a compound of Formula I, or a pharmaceutically acceptable salt thereof, having a formula according to Formula Ij:

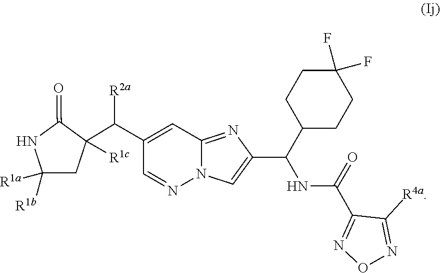

In some embodiments, disclosed herein is a compound of Formula I, or a pharmaceutically acceptable salt thereof, having a formula according to Formula Ij-1:

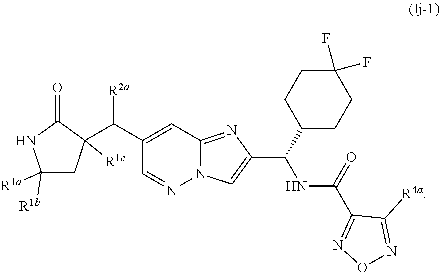

In some embodiments, disclosed herein is a compound of Formula I, or a pharmaceutically acceptable salt thereof, having a formula according to Formula Ij-2:

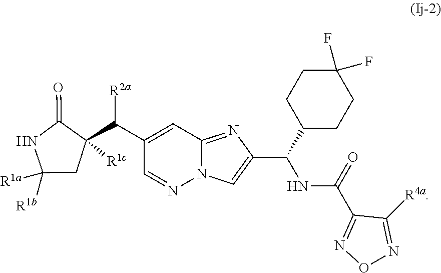

In some embodiments, disclosed herein is a compound of Formula I, or a pharmaceutically acceptable salt thereof, having a formula according to Formula Ij-3:

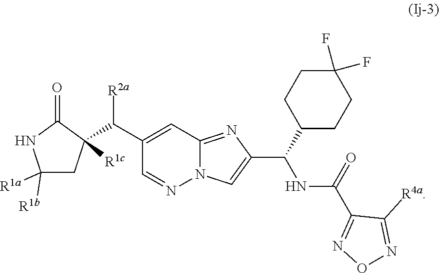

In some embodiments, disclosed herein is a compound of Formula I, or a pharmaceutically acceptable salt thereof, having a formula according to Formula Ik:

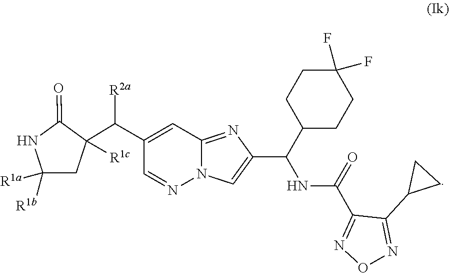

In some embodiments, disclosed herein is a compound of Formula I, or a pharmaceutically acceptable salt thereof, having a formula according to Formula Ik-1:

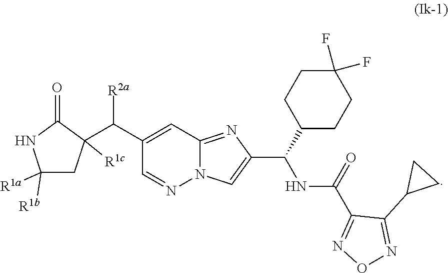

In some embodiments, disclosed herein is a compound of Formula I, or a pharmaceutically acceptable salt thereof, having a formula according to Formula Ik-2:

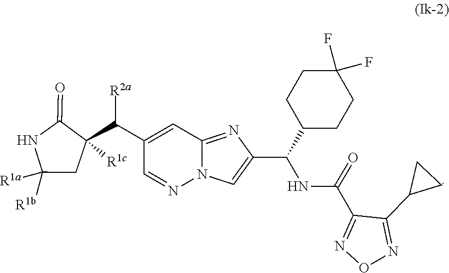

In some embodiments, disclosed herein is a compound of Formula I, or a pharmaceutically acceptable salt thereof, having a formula according to Formula Ik-3:

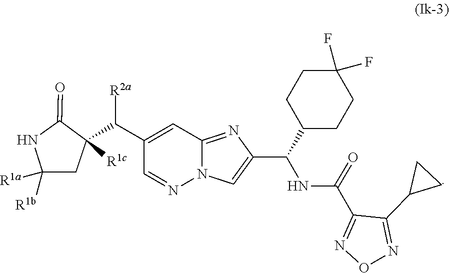

In some embodiments, disclosed herein is a compound of Formula I, or a pharmaceutically acceptable salt thereof, having a formula according to Formula Im:

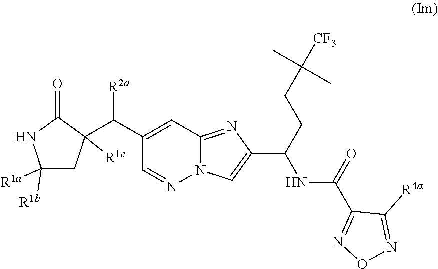

In some embodiments, disclosed herein is a compound of Formula I, or a pharmaceutically acceptable salt thereof, having a formula according to Formula Im-1:

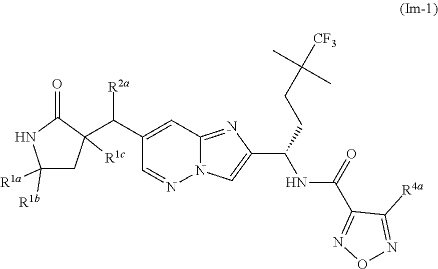

In some embodiments, disclosed herein is a compound of Formula I, or a pharmaceutically acceptable salt thereof, having a formula according to Formula Im-2:

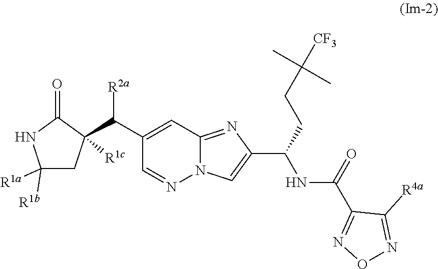

In some embodiments, disclosed herein is a compound of Formula I, or a pharmaceutically acceptable salt thereof, having a formula according to Formula Im-3:

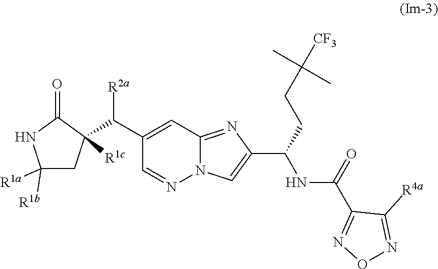

In some embodiments, disclosed herein is a compound of Formula I, or a pharmaceutically acceptable salt thereof, having a formula according to Formula In:

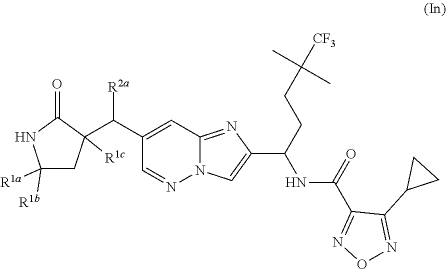

In some embodiments, disclosed herein is a compound of Formula I, or a pharmaceutically acceptable salt thereof, having a formula according to Formula In-1:

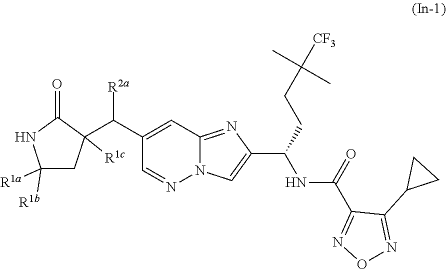

In some embodiments, disclosed herein is a compound of Formula I, or a pharmaceutically acceptable salt thereof, having a formula according to Formula In-2:

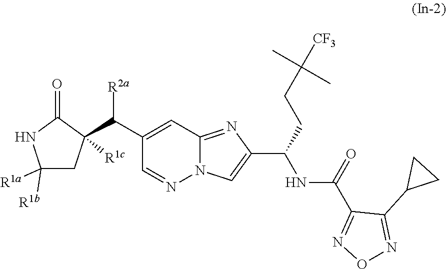

In some embodiments, disclosed herein is a compound of Formula I, or a pharmaceutically acceptable salt thereof, having a formula according to Formula In-3:

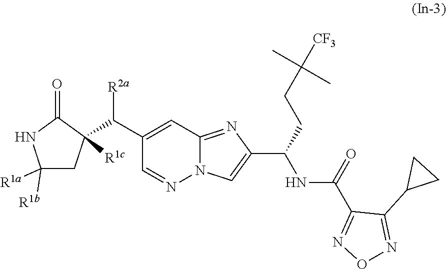

In some embodiments, disclosed herein is a compound of Formula I, or a pharmaceutically acceptable salt thereof, that is a deuterated isotope comprising one to thirty deuterium atoms. In some embodiments, disclosed herein is a compound of Formula I, or a pharmaceutically acceptable salt thereof, that is a deuterated isotope comprising one to fifteen deuterium atoms. In some embodiments, disclosed herein is a compound of Formula I, or a pharmaceutically acceptable salt thereof, that is a deuterated isotope comprising one to seven deuterium atoms.

In some embodiments, disclosed herein is a compound of Formula I having a formula as shown in Table 1, or a pharmaceutically acceptable salt thereof.

TABLE 1

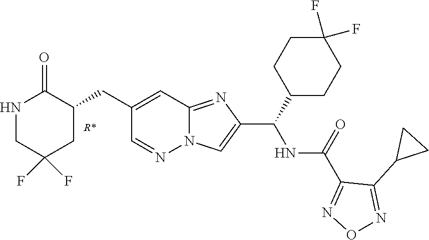 [SMILES: O=C(N[C@H](c1cn2ncc(C[C@@H]3CC(F)(F)CNC3=O)cc2n1)C1CCC(F)(F)CC1)c1nonc1C1CC1]

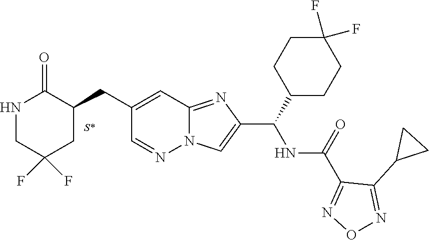 [SMILES: O=C(N[C@H](c1cn2ncc(C[C@H]3CC(F)(F)CNC3=O)cc2n1)C1CCC(F)(F)CC1)c1nonc1C1CC1]

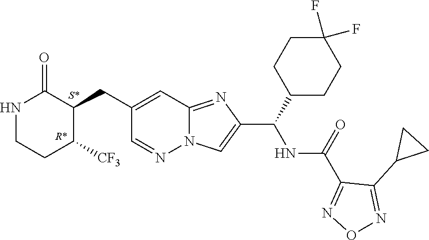 [SMILES: O=C(N[C@H](c1cn2ncc(C[C@@H]3C(=O)NCC[C@H]3C(F)(F)F)cc2n1)C1CCC(F)(F)CC1)c1nonc1C1CC1]

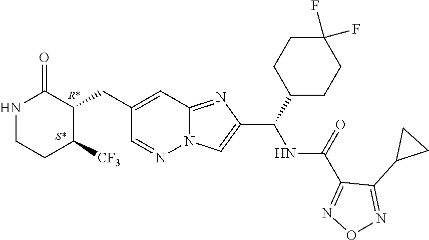 [SMILES: O=C(N[C@H](c1cn2ncc(C[C@H]3C(=O)NCC[C@@H]3C(F)(F)F)cc2n1)C1CCC(F)(F)CC1)c1nonc1C1CC1]

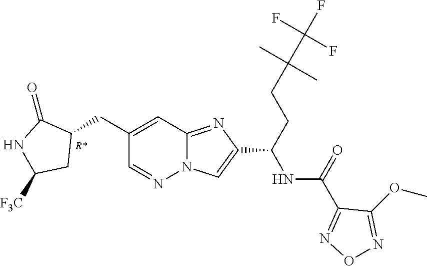 [SMILES: COc1nonc1C(=O)N[C@@H](CCC(C)(C)C(F)(F)F)c1cn2ncc(C[C@@H]3C[C@@H](C(F)(F)F)NC3=O)cc2n1]

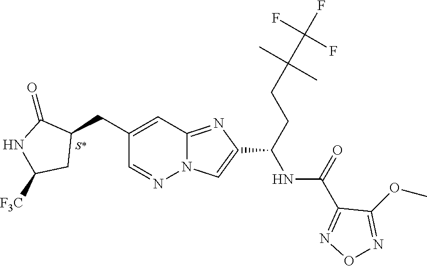 [SMILES: COc1nonc1C(=O)N[C@@H](CCC(C)(C)C(F)(F)F)c1cn2ncc(C[C@H]3C[C@@H](C(F)(F)F)NC3=O)cc2n1]

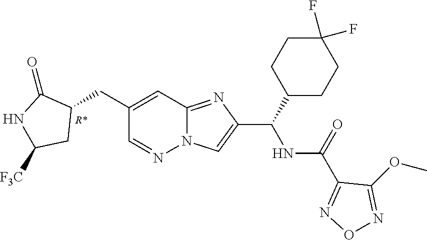 [SMILES: COc1nonc1C(=O)N[C@H](c1cn2ncc(C[C@@H]3C[C@@H](C(F)(F)F)NC3=O)cc2n1)C1CCC(F)(F)CC1]

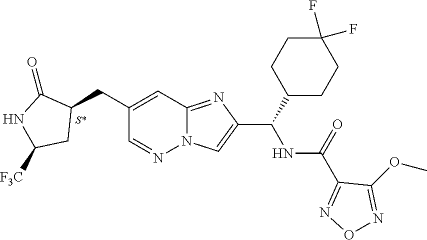 [SMILES: COc1nonc1C(=O)N[C@H](c1cn2ncc(C[C@H]3C[C@@H](C(F)(F)F)NC3=O)cc2n1)C1CCC(F)(F)CC1]

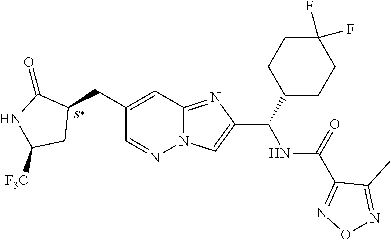 [SMILES: Cc1nonc1C(=O)N[C@H](c1cn2ncc(C[C@H]3C[C@@H](C(F)(F)F)NC3=O)cc2n1)C1CCC(F)(F)CC1]

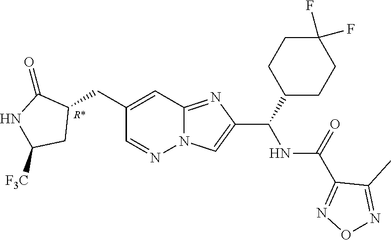 [SMILES: Cc1nonc1C(=O)N[C@H](c1cn2ncc(C[C@@H]3C[C@@H](C(F)(F)F)NC3=O)cc2n1)C1CCC(F)(F)CC1]

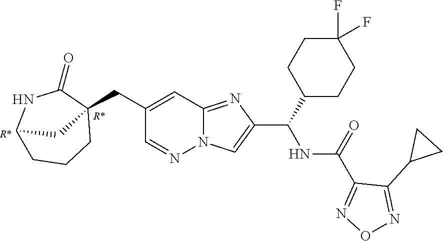 [SMILES: O=C(N[C@H](c1cn2ncc(C[C@@]34CCC[C@H](C3)NC4=O)cc2n1)C1CCC(F)(F)CC1)c1nonc1C1CC1]

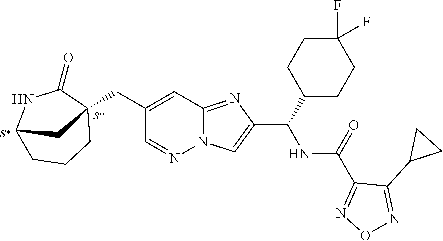 [SMILES: O=C(N[C@H](c1cn2ncc(C[C@]34CCC[C@@H](C3)NC4=O)cc2n1)C1CCC(F)(F)CC1)c1nonc1C1CC1]

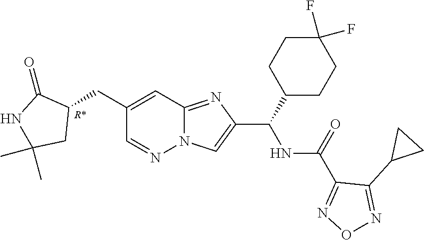 [SMILES: CC1(C)C[C@@H](Cc2cnn3cc([C@@H](NC(=O)c4nonc4C4CC4)C4CCC(F)(F)CC4)nc3c2)C(=O)N1]

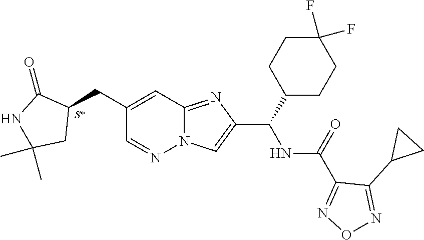 [SMILES: CC1(C)C[C@H](Cc2cnn3cc([C@@H](NC(=O)c4nonc4C4CC4)C4CCC(F)(F)CC4)nc3c2)C(=O)N1]

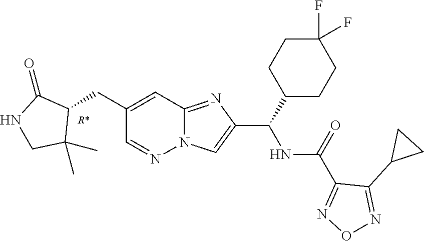 [SMILES: CC1(C)CNC(=O)[C@@H]1Cc1cnn2cc([C@@H](NC(=O)c3nonc3C3CC3)C3CCC(F)(F)CC3)nc2c1]

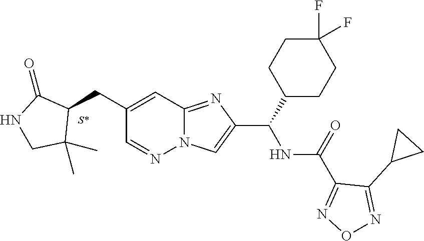 [SMILES: CC1(C)CNC(=O)[C@H]1Cc1cnn2cc([C@@H](NC(=O)c3nonc3C3CC3)C3CCC(F)(F)CC3)nc2c1]

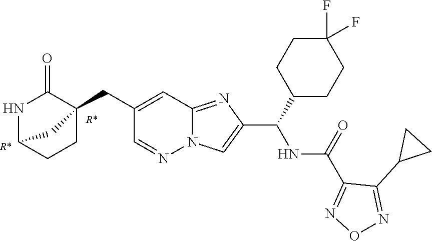 [SMILES: O=C(N[C@H](c1cn2ncc(C[C@@]34CC[C@H](C3)NC4=O)cc2n1)C1CCC(F)(F)CC1)c1nonc1C1CC1]

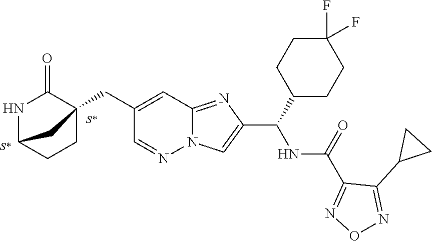 [SMILES: O=C(N[C@H](c1cn2ncc(C[C@]34CC[C@@H](C3)NC4=O)cc2n1)C1CCC(F)(F)CC1)c1nonc1C1CC1]

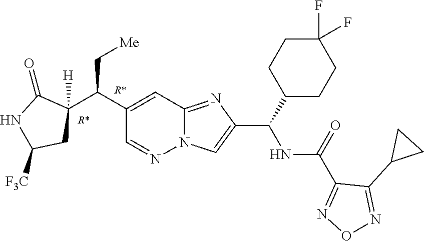 [SMILES: CC[C@@H](c1cnn2cc([C@@H](NC(=O)c3nonc3C3CC3)C3CCC(F)(F)CC3)nc2c1)[C@@H]1C[C@@H](C(F)(F)F)NC1=O]

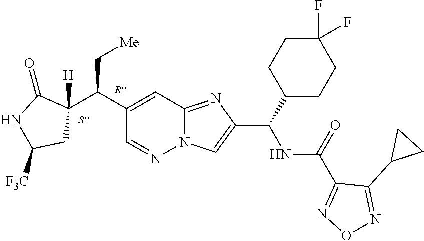 [SMILES: CC[C@@H](c1cnn2cc([C@@H](NC(=O)c3nonc3C3CC3)C3CCC(F)(F)CC3)nc2c1)[C@H]1C[C@@H](C(F)(F)F)NC1=O]

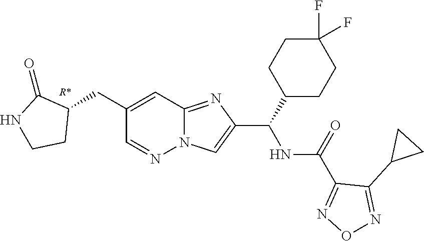 [SMILES: O=C(N[C@H](c1cn2ncc(C[C@@H]3CCNC3=O)cc2n1)C1CCC(F)(F)CC1)c1nonc1C1CC1]

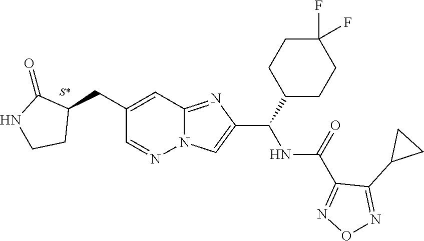 [SMILES: O=C(N[C@H](c1cn2ncc(C[C@H]3CCNC3=O)cc2n1)C1CCC(F)(F)CC1)c1nonc1C1CC1]

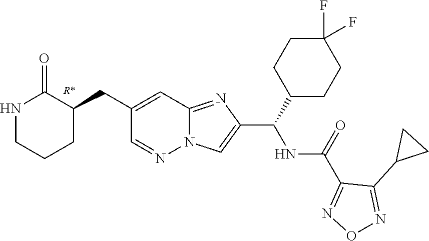 [SMILES: O=C(N[C@H](c1cn2ncc(C[C@H]3CCCNC3=O)cc2n1)C1CCC(F)(F)CC1)c1nonc1C1CC1]

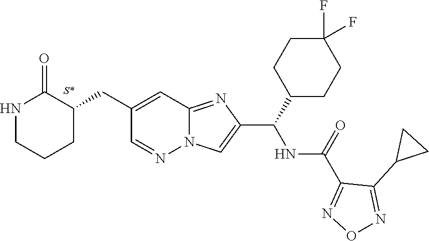 [SMILES: O=C(N[C@H](c1cn2ncc(C[C@@H]3CCCNC3=O)cc2n1)C1CCC(F)(F)CC1)c1nonc1C1CC1]

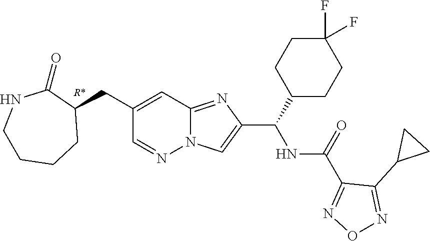 [SMILES: O=C(N[C@H](c1cn2ncc(C[C@H]3CCCCNC3=O)cc2n1)C1CCC(F)(F)CC1)c1nonc1C1CC1]

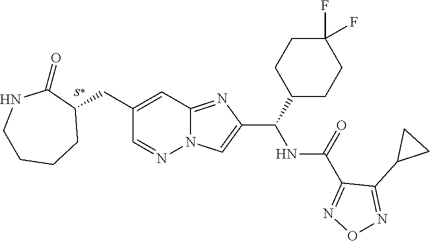 [SMILES: O=C(N[C@H](c1cn2ncc(C[C@@H]3CCCCNC3=O)cc2n1)C1CCC(F)(F)CC1)c1nonc1C1CC1]

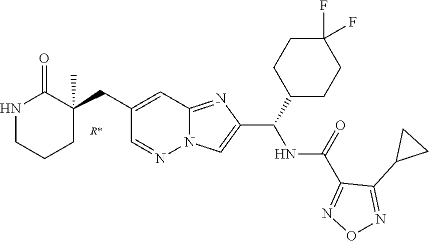 [SMILES: C[C@]1(Cc2cnn3cc([C@@H](NC(=O)c4nonc4C4CC4)C4CCC(F)(F)CC4)nc3c2)CCCNC1=O]

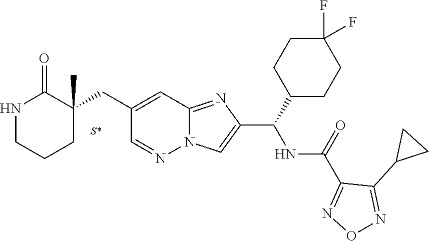 [SMILES: C[C@@]1(Cc2cnn3cc([C@@H](NC(=O)c4nonc4C4CC4)C4CCC(F)(F)CC4)nc3c2)CCCNC1=O]

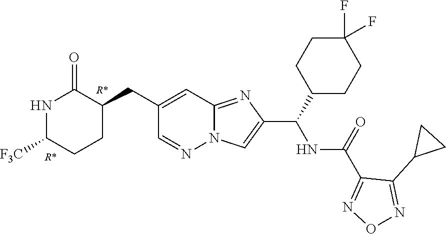 [SMILES: O=C(N[C@H](c1cn2ncc(C[C@H]3CC[C@H](C(F)(F)F)NC3=O)cc2n1)C1CCC(F)(F)CC1)c1nonc1C1CC1]

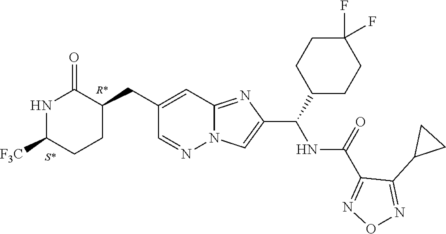 [SMILES: O=C(N[C@H](c1cn2ncc(C[C@H]3CC[C@@H](C(F)(F)F)NC3=O)cc2n1)C1CCC(F)(F)CC1)c1nonc1C1CC1]

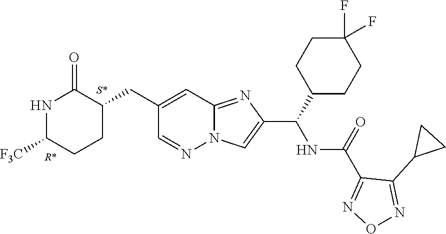 [SMILES: O=C(N[C@H](c1cn2ncc(C[C@@H]3CC[C@H](C(F)(F)F)NC3=O)cc2n1)C1CCC(F)(F)CC1)c1nonc1C1CC1]

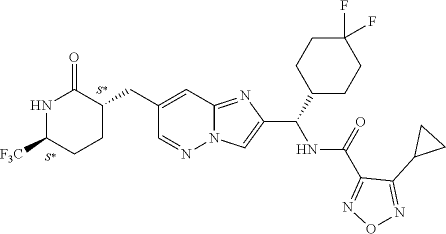 [SMILES: O=C(N[C@H](c1cn2ncc(C[C@@H]3CC[C@@H](C(F)(F)F)NC3=O)cc2n1)C1CCC(F)(F)CC1)c1nonc1C1CC1]

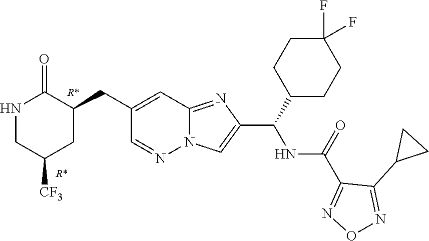 [SMILES: O=C(N[C@H](c1cn2ncc(C[C@H]3C[C@@H](C(F)(F)F)CNC3=O)cc2n1)C1CCC(F)(F)CC1)c1nonc1C1CC1]

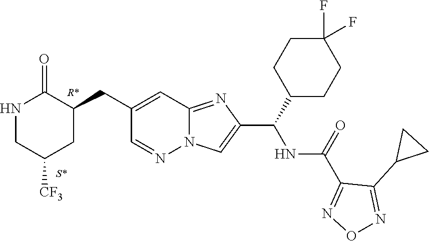 [SMILES: O=C(N[C@H](c1cn2ncc(C[C@H]3C[C@H](C(F)(F)F)CNC3=O)cc2n1)C1CCC(F)(F)CC1)c1nonc1C1CC1]

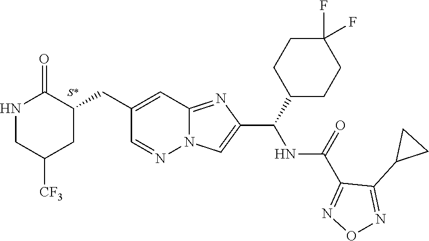 [SMILES: O=C(N[C@H](c1cn2ncc(C[C@@H]3CC(C(F)(F)F)CNC3=O)cc2n1)C1CCC(F)(F)CC1)c1nonc1C1CC1]

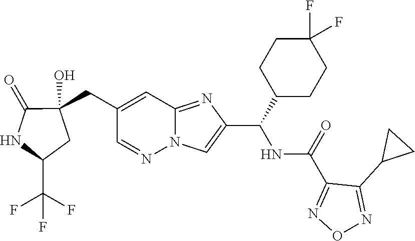 [SMILES: O=C(N[C@H](c1cn2ncc(C[C@@]3(O)C[C@@H](C(F)(F)F)NC3=O)cc2n1)C1CCC(F)(F)CC1)c1nonc1C1CC1]

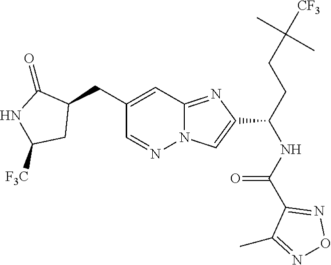 [SMILES: Cc1nonc1C(=O)N[C@@H](CCC(C)(C)C(F)(F)F)c1cn2ncc(C[C@H]3C[C@@H](C(F)(F)F)NC3=O)cc2n1]

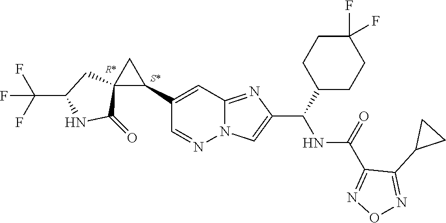 [SMILES: O=C(N[C@H](c1cn2ncc([C@@H]3C[C@@]34C[C@@H](C(F)(F)F)NC4=O)cc2n1)C1CCC(F)(F)CC1)c1nonc1C1CC1]

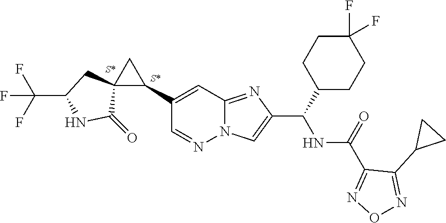 [SMILES: O=C(N[C@H](c1cn2ncc([C@@H]3C[C@]34C[C@@H](C(F)(F)F)NC4=O)cc2n1)C1CCC(F)(F)CC1)c1nonc1C1CC1]

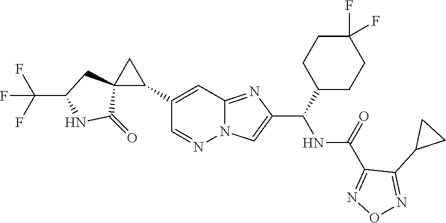 [SMILES: O=C(N[C@H](c1cn2ncc([C@H]3C[C@]34C[C@@H](C(F)(F)F)NC4=O)cc2n1)C1CCC(F)(F)CC1)c1nonc1C1CC1]

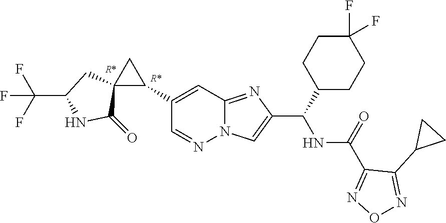 [SMILES: O=C(N[C@H](c1cn2ncc([C@H]3C[C@@]34C[C@@H](C(F)(F)F)NC4=O)cc2n1)C1CCC(F)(F)CC1)c1nonc1C1CC1]

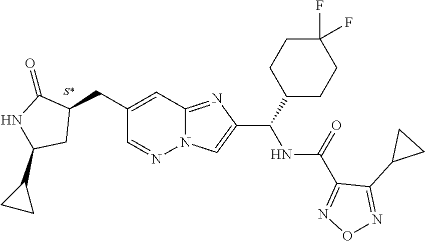 [SMILES: O=C(N[C@H](c1cn2ncc(C[C@H]3C[C@@H](C4CC4)NC3=O)cc2n1)C1CCC(F)(F)CC1)c1nonc1C1CC1]

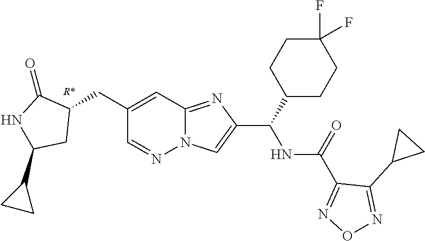 [SMILES: O=C(N[C@H](c1cn2ncc(C[C@@H]3C[C@@H](C4CC4)NC3=O)cc2n1)C1CCC(F)(F)CC1)c1nonc1C1CC1]

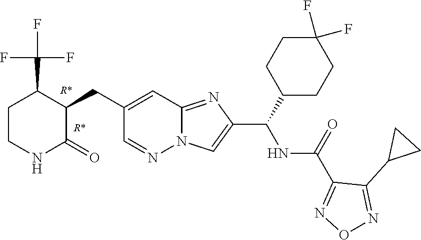 [SMILES: O=C(N[C@H](c1cn2ncc(C[C@H]3C(=O)NCC[C@H]3C(F)(F)F)cc2n1)C1CCC(F)(F)CC1)c1nonc1C1CC1]

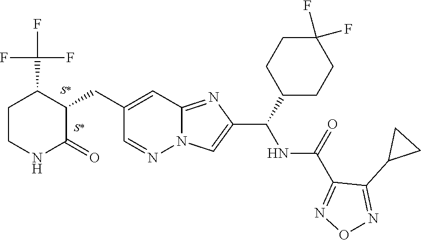 [SMILES: O=C(N[C@H](c1cn2ncc(C[C@@H]3C(=O)NCC[C@@H]3C(F)(F)F)cc2n1)C1CCC(F)(F)CC1)c1nonc1C1CC1]

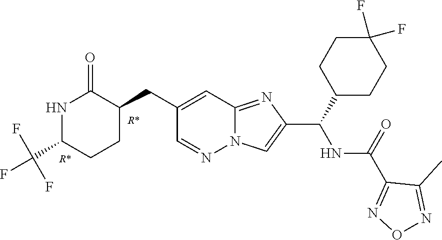 [SMILES: Cc1nonc1C(=O)N[C@H](c1cn2ncc(C[C@H]3CC[C@H](C(F)(F)F)NC3=O)cc2n1)C1CCC(F)(F)CC1]

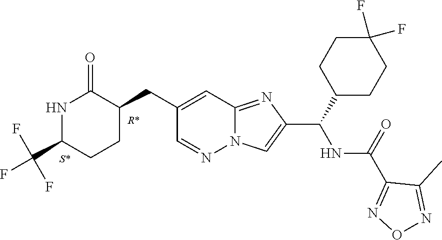 [SMILES: Cc1nonc1C(=O)N[C@H](c1cn2ncc(C[C@H]3CC[C@@H](C(F)(F)F)NC3=O)cc2n1)C1CCC(F)(F)CC1]

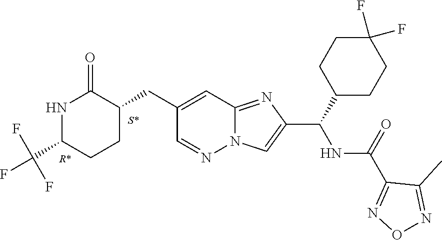 [SMILES: Cc1nonc1C(=O)N[C@H](c1cn2ncc(C[C@@H]3CC[C@H](C(F)(F)F)NC3=O)cc2n1)C1CCC(F)(F)CC1]

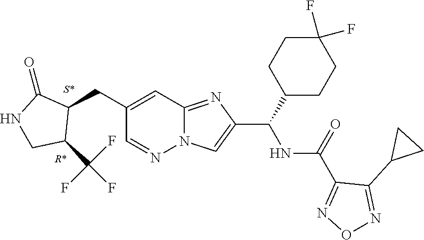 [SMILES: O=C(N[C@H](c1cn2ncc(C[C@@H]3C(=O)NC[C@@H]3C(F)(F)F)cc2n1)C1CCC(F)(F)CC1)c1nonc1C1CC1]

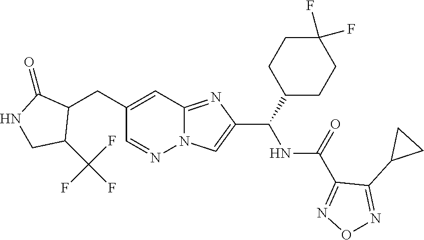 [SMILES: O=C(N[C@H](c1cn2ncc(CC3C(=O)NCC3C(F)(F)F)cc2n1)C1CCC(F)(F)CC1)c1nonc1C1CC1]

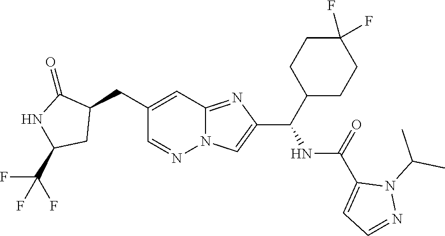 [SMILES: CC(C)n1nccc1C(=O)N[C@H](c1cn2ncc(C[C@H]3C[C@@H](C(F)(F)F)NC3=O)cc2n1)C1CCC(F)(F)CC1]

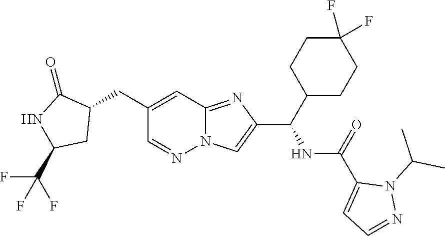 [SMILES: CC(C)n1nccc1C(=O)N[C@H](c1cn2ncc(C[C@@H]3C[C@@H](C(F)(F)F)NC3=O)cc2n1)C1CCC(F)(F)CC1]

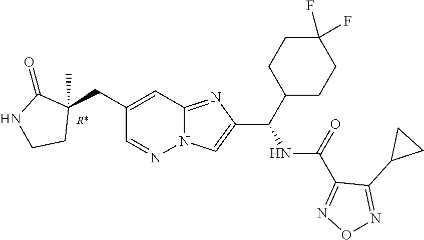 [SMILES: C[C@]1(Cc2cnn3cc([C@@H](NC(=O)c4nonc4C4CC4)C4CCC(F)(F)CC4)nc3c2)CCNC1=O]

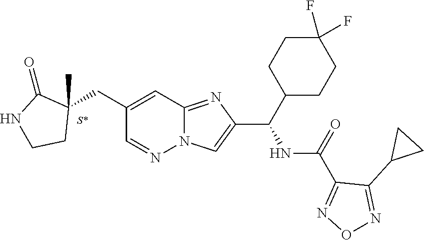 [SMILES: C[C@@]1(Cc2cnn3cc([C@@H](NC(=O)c4nonc4C4CC4)C4CCC(F)(F)CC4)nc3c2)CCNC1=O]

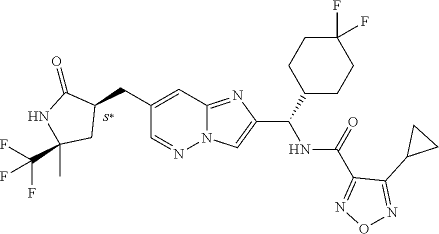 [SMILES: C[C@@]1(C(F)(F)F)C[C@H](Cc2cnn3cc([C@@H](NC(=O)c4nonc4C4CC4)C4CCC(F)(F)CC4)nc3c2)C(=O)N1]

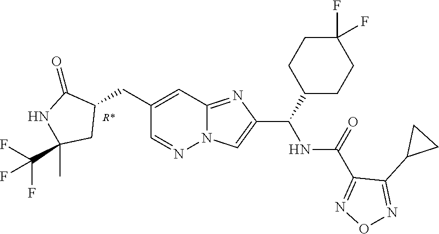 [SMILES: C[C@@]1(C(F)(F)F)C[C@@H](Cc2cnn3cc([C@@H](NC(=O)c4nonc4C4CC4)C4CCC(F)(F)CC4)nc3c2)C(=O)N1]

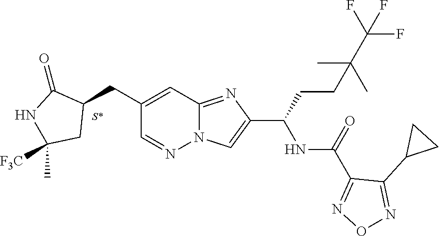 [SMILES: CC(C)(CC[C@H](NC(=O)c1nonc1C1CC1)c1cn2ncc(C[C@H]3C[C@@](C)(C(F)(F)F)NC3=O)cc2n1)C(F)(F)F]

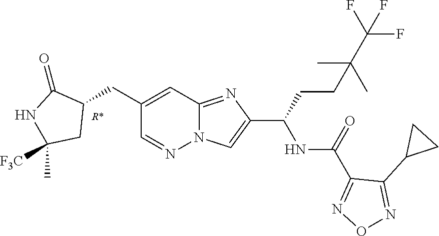 [SMILES: CC(C)(CC[C@H](NC(=O)c1nonc1C1CC1)c1cn2ncc(C[C@@H]3C[C@@](C)(C(F)(F)F)NC3=O)cc2n1)C(F)(F)F]

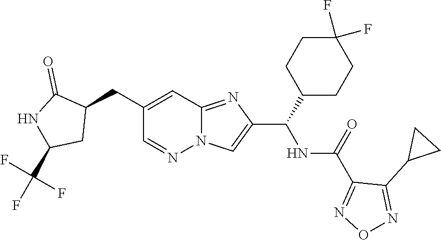 [SMILES: O=C(N[C@H](c1cn2ncc(C[C@H]3C[C@@H](C(F)(F)F)NC3=O)cc2n1)C1CCC(F)(F)CC1)c1nonc1C1CC1]

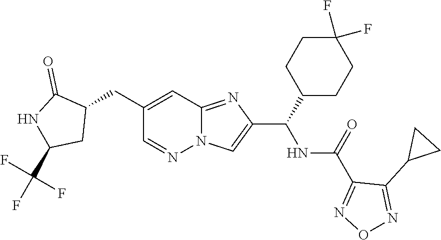 [SMILES: O=C(N[C@H](c1cn2ncc(C[C@@H]3C[C@@H](C(F)(F)F)NC3=O)cc2n1)C1CCC(F)(F)CC1)c1nonc1C1CC1]

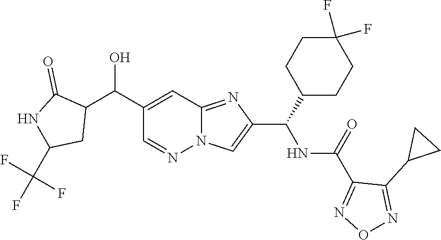 [SMILES: O=C(N[C@H](c1cn2ncc(C(O)C3CC(C(F)(F)F)NC3=O)cc2n1)C1CCC(F)(F)CC1)c1nonc1C1CC1]

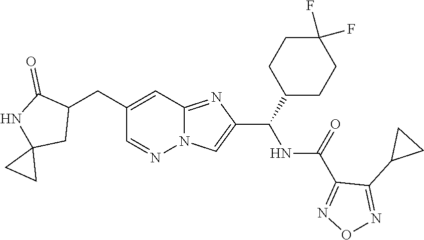 [SMILES: O=C(N[C@H](c1cn2ncc(CC3CC4(CC4)NC3=O)cc2n1)C1CCC(F)(F)CC1)c1nonc1C1CC1]

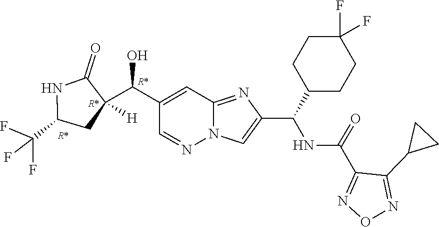 [SMILES: O=C(N[C@H](c1cn2ncc([C@H](O)[C@H]3C[C@H](C(F)(F)F)NC3=O)cc2n1)C1CCC(F)(F)CC1)c1nonc1C1CC1]

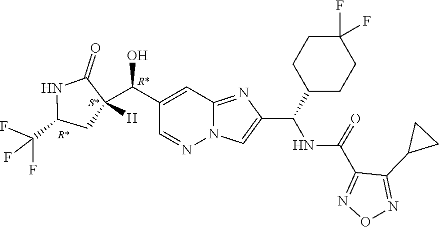 [SMILES: O=C(N[C@H](c1cn2ncc([C@H](O)[C@@H]3C[C@H](C(F)(F)F)NC3=O)cc2n1)C1CCC(F)(F)CC1)c1nonc1C1CC1]

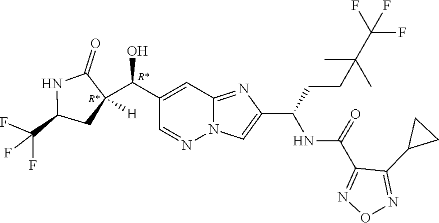 [SMILES: CC(C)(CC[C@H](NC(=O)c1nonc1C1CC1)c1cn2ncc([C@H](O)[C@H]3C[C@@H](C(F)(F)F)NC3=O)cc2n1)C(F)(F)F]

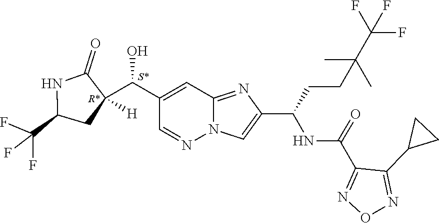 [SMILES: CC(C)(CC[C@H](NC(=O)c1nonc1C1CC1)c1cn2ncc([C@@H](O)[C@H]3C[C@@H](C(F)(F)F)NC3=O)cc2n1)C(F)(F)F]

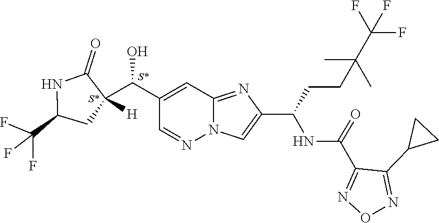 [SMILES: CC(C)(CC[C@H](NC(=O)c1nonc1C1CC1)c1cn2ncc([C@@H](O)[C@@H]3C[C@@H](C(F)(F)F)NC3=O)cc2n1)C(F)(F)F]

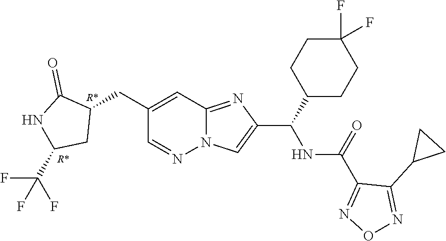 [SMILES: O=C(N[C@H](c1cn2ncc(C[C@@H]3C[C@H](C(F)(F)F)NC3=O)cc2n1)C1CCC(F)(F)CC1)c1nonc1C1CC1]

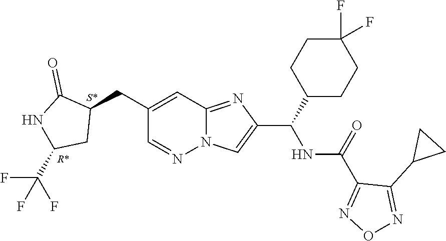 [SMILES: O=C(N[C@H](c1cn2ncc(C[C@H]3C[C@H](C(F)(F)F)NC3=O)cc2n1)C1CCC(F)(F)CC1)c1nonc1C1CC1]

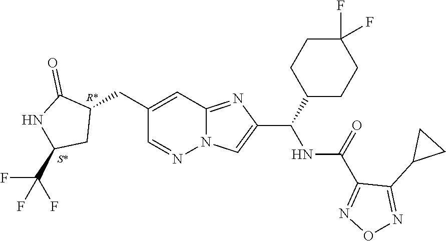 [SMILES: O=C(N[C@H](c1cn2ncc(C[C@@H]3C[C@@H](C(F)(F)F)NC3=O)cc2n1)C1CCC(F)(F)CC1)c1nonc1C1CC1]

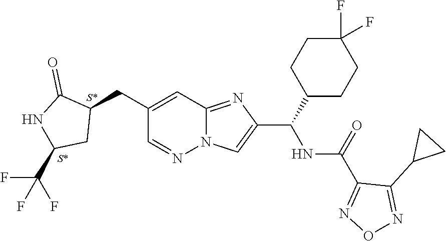 [SMILES: O=C(N[C@H](c1cn2ncc(C[C@H]3C[C@@H](C(F)(F)F)NC3=O)cc2n1)C1CCC(F)(F)CC1)c1nonc1C1CC1]

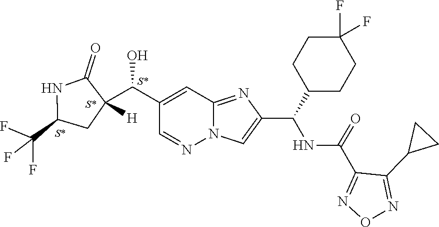 [SMILES: O=C(N[C@H](c1cn2ncc([C@@H](O)[C@@H]3C[C@@H](C(F)(F)F)NC3=O)cc2n1)C1CCC(F)(F)CC1)c1nonc1C1CC1]

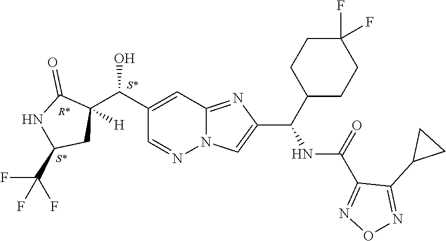 [SMILES: O=C(N[C@H](c1cn2ncc([C@@H](O)[C@H]3C[C@@H](C(F)(F)F)NC3=O)cc2n1)C1CCC(F)(F)CC1)c1nonc1C1CC1]

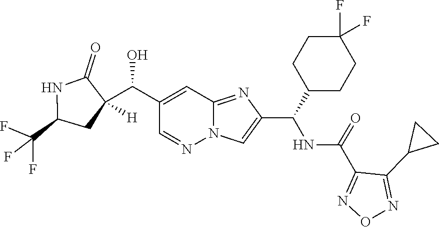 [SMILES: O=C(N[C@H](c1cn2ncc([C@@H](O)[C@H]3C[C@@H](C(F)(F)F)NC3=O)cc2n1)C1CCC(F)(F)CC1)c1nonc1C1CC1]

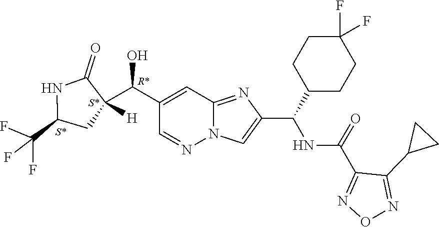 [SMILES: O=C(N[C@H](c1cn2ncc([C@H](O)[C@@H]3C[C@@H](C(F)(F)F)NC3=O)cc2n1)C1CCC(F)(F)CC1)c1nonc1C1CC1]

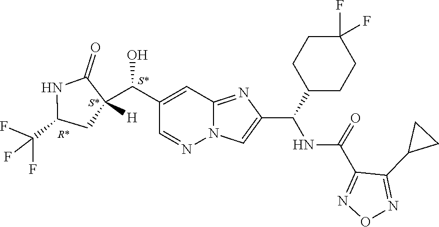 [SMILES: O=C(N[C@H](c1cn2ncc([C@@H](O)[C@@H]3C[C@H](C(F)(F)F)NC3=O)cc2n1)C1CCC(F)(F)CC1)c1nonc1C1CC1]

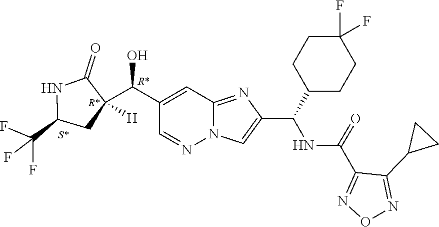 [SMILES: O=C(N[C@H](c1cn2ncc([C@H](O)[C@H]3C[C@@H](C(F)(F)F)NC3=O)cc2n1)C1CCC(F)(F)CC1)c1nonc1C1CC1]

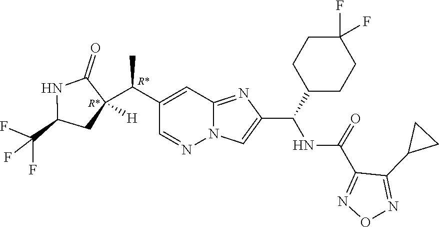 [SMILES: C[C@@H](c1cnn2cc([C@@H](NC(=O)c3nonc3C3CC3)C3CCC(F)(F)CC3)nc2c1)[C@H]1C[C@@H](C(F)(F)F)NC1=O]

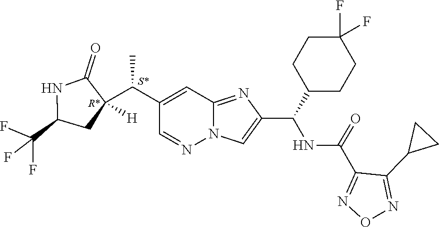 [SMILES: C[C@H](c1cnn2cc([C@@H](NC(=O)c3nonc3C3CC3)C3CCC(F)(F)CC3)nc2c1)[C@H]1C[C@@H](C(F)(F)F)NC1=O]

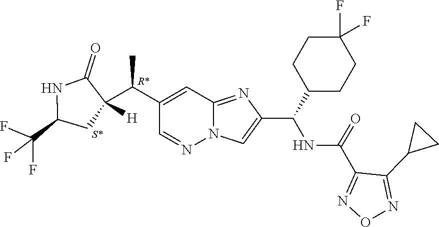 [SMILES: C[C@@H](c1cnn2cc([C@@H](NC(=O)c3nonc3C3CC3)C3CCC(F)(F)CC3)nc2c1)[C@@H]1C[C@@H](C(F)(F)F)NC1=O]

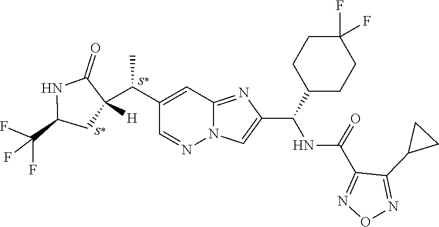 [SMILES: C[C@H](c1cnn2cc([C@@H](NC(=O)c3nonc3C3CC3)C3CCC(F)(F)CC3)nc2c1)[C@@H]1C[C@@H](C(F)(F)F)NC1=O]

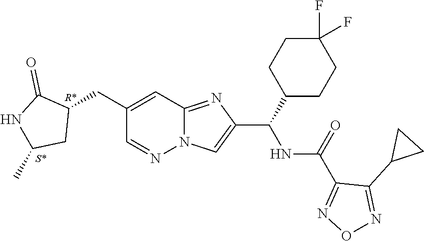 [SMILES: C[C@H]1C[C@@H](Cc2cnn3cc([C@@H](NC(=O)c4nonc4C4CC4)C4CCC(F)(F)CC4)nc3c2)C(=O)N1]

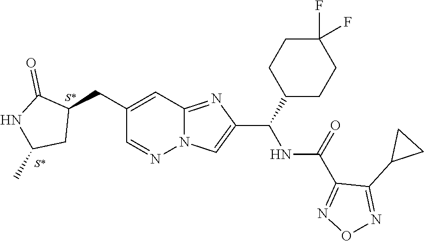 [SMILES: C[C@H]1C[C@H](Cc2cnn3cc([C@@H](NC(=O)c4nonc4C4CC4)C4CCC(F)(F)CC4)nc3c2)C(=O)N1]

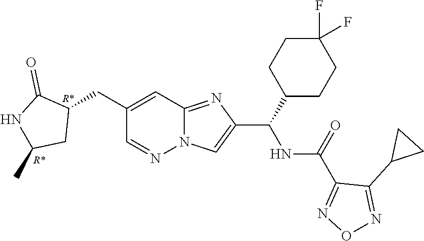 [SMILES: C[C@@H]1C[C@@H](Cc2cnn3cc([C@@H](NC(=O)c4nonc4C4CC4)C4CCC(F)(F)CC4)nc3c2)C(=O)N1]

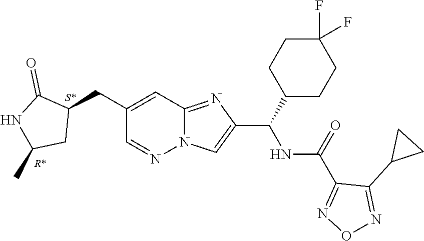 [SMILES: C[C@@H]1C[C@H](Cc2cnn3cc([C@@H](NC(=O)c4nonc4C4CC4)C4CCC(F)(F)CC4)nc3c2)C(=O)N1]

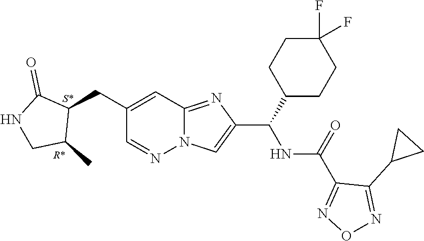 [SMILES: C[C@H]1CNC(=O)[C@H]1Cc1cnn2cc([C@@H](NC(=O)c3nonc3C3CC3)C3CCC(F)(F)CC3)nc2c1]

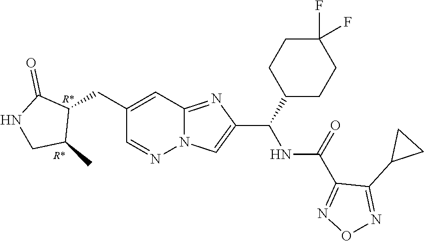 [SMILES: C[C@H]1CNC(=O)[C@@H]1Cc1cnn2cc([C@@H](NC(=O)c3nonc3C3CC3)C3CCC(F)(F)CC3)nc2c1]

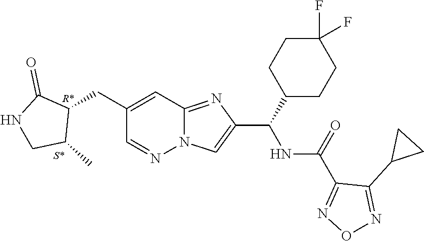 [SMILES: C[C@@H]1CNC(=O)[C@@H]1Cc1cnn2cc([C@@H](NC(=O)c3nonc3C3CC3)C3CCC(F)(F)CC3)nc2c1]

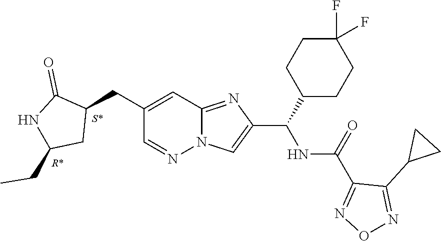 [SMILES: CC[C@@H]1C[C@H](Cc2cnn3cc([C@@H](NC(=O)c4nonc4C4CC4)C4CCC(F)(F)CC4)nc3c2)C(=O)N1]

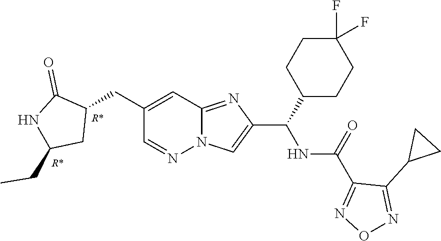 [SMILES: CC[C@@H]1C[C@@H](Cc2cnn3cc([C@@H](NC(=O)c4nonc4C4CC4)C4CCC(F)(F)CC4)nc3c2)C(=O)N1]

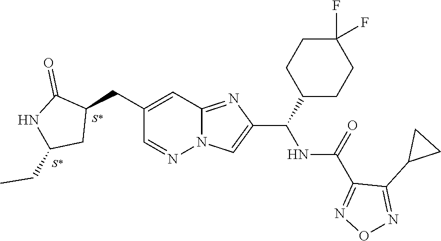 [SMILES: CC[C@H]1C[C@H](Cc2cnn3cc([C@@H](NC(=O)c4nonc4C4CC4)C4CCC(F)(F)CC4)nc3c2)C(=O)N1]

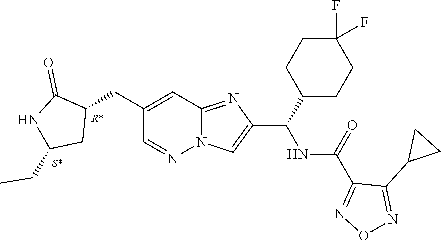 [SMILES: CC[C@H]1C[C@@H](Cc2cnn3cc([C@@H](NC(=O)c4nonc4C4CC4)C4CCC(F)(F)CC4)nc3c2)C(=O)N1]

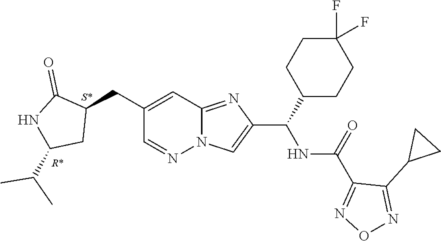 [SMILES: CC(C)[C@H]1C[C@H](Cc2cnn3cc([C@@H](NC(=O)c4nonc4C4CC4)C4CCC(F)(F)CC4)nc3c2)C(=O)N1]

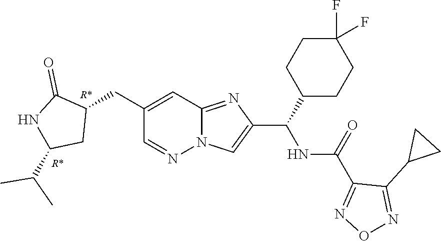 [SMILES: CC(C)[C@H]1C[C@@H](Cc2cnn3cc([C@@H](NC(=O)c4nonc4C4CC4)C4CCC(F)(F)CC4)nc3c2)C(=O)N1]

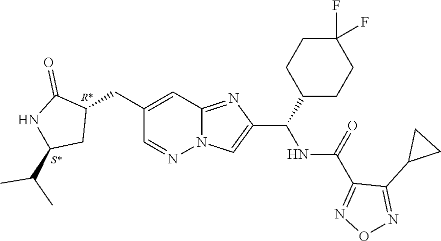 [SMILES: CC(C)[C@@H]1C[C@@H](Cc2cnn3cc([C@@H](NC(=O)c4nonc4C4CC4)C4CCC(F)(F)CC4)nc3c2)C(=O)N1]

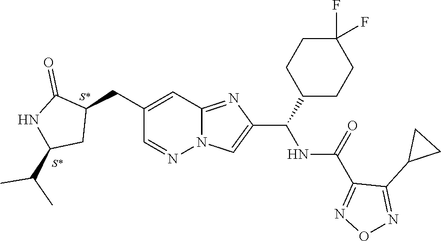 [SMILES: CC(C)[C@@H]1C[C@H](Cc2cnn3cc([C@@H](NC(=O)c4nonc4C4CC4)C4CCC(F)(F)CC4)nc3c2)C(=O)N1]

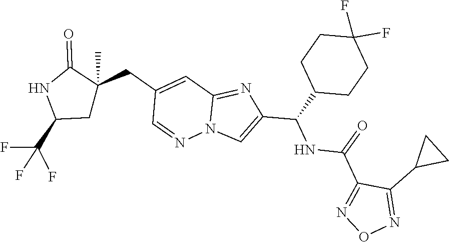 [SMILES: C[C@]1(Cc2cnn3cc([C@@H](NC(=O)c4nonc4C4CC4)C4CCC(F)(F)CC4)nc3c2)C[C@@H](C(F)(F)F)NC1=O]

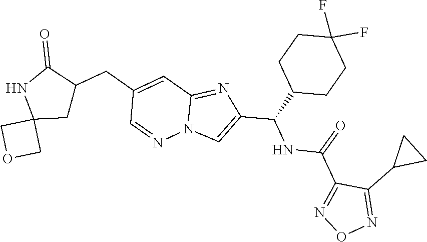 [SMILES: O=C(N[C@H](c1cn2ncc(CC3CC4(COC4)NC3=O)cc2n1)C1CCC(F)(F)CC1)c1nonc1C1CC1]

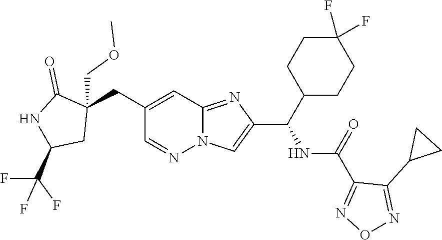 [SMILES: COC[C@]1(Cc2cnn3cc([C@@H](NC(=O)c4nonc4C4CC4)C4CCC(F)(F)CC4)nc3c2)C[C@@H](C(F)(F)F)NC1=O]

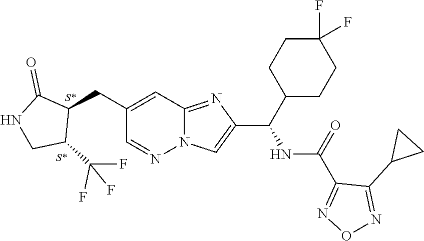 [SMILES: O=C(N[C@H](c1cn2ncc(C[C@@H]3C(=O)NC[C@H]3C(F)(F)F)cc2n1)C1CCC(F)(F)CC1)c1nonc1C1CC1]

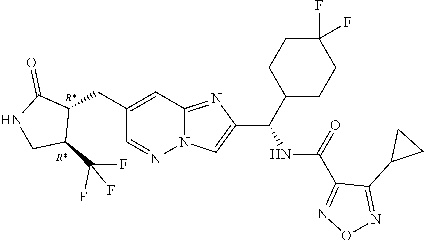 [SMILES: O=C(N[C@H](c1cn2ncc(C[C@H]3C(=O)NC[C@@H]3C(F)(F)F)cc2n1)C1CCC(F)(F)CC1)c1nonc1C1CC1]

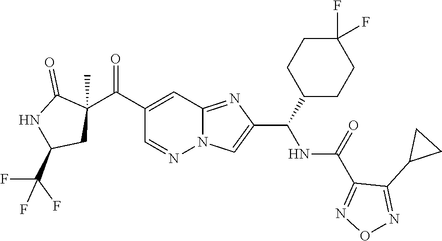 [SMILES: C[C@@]1(C(=O)c2cnn3cc([C@@H](NC(=O)c4nonc4C4CC4)C4CCC(F)(F)CC4)nc3c2)C[C@@H](C(F)(F)F)NC1=O]

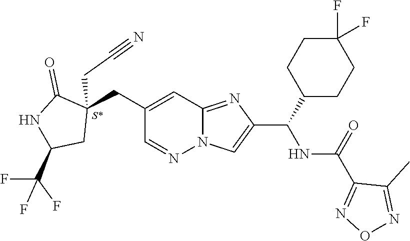 [SMILES: Cc1nonc1C(=O)N[C@H](c1cn2ncc(C[C@@]3(CC#N)C[C@@H](C(F)(F)F)NC3=O)cc2n1)C1CCC(F)(F)CC1]

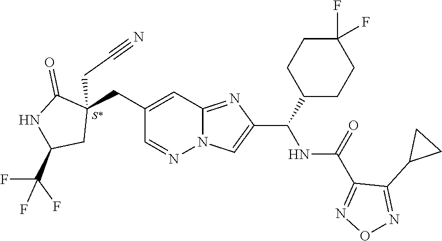 [SMILES: N#CC[C@]1(Cc2cnn3cc([C@@H](NC(=O)c4nonc4C4CC4)C4CCC(F)(F)CC4)nc3c2)C[C@@H](C(F)(F)F)NC1=O]

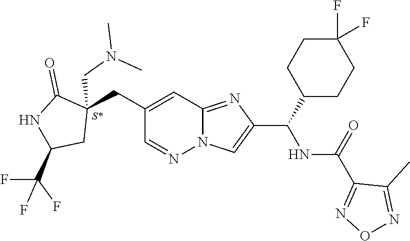 [SMILES: Cc1nonc1C(=O)N[C@H](c1cn2ncc(C[C@@]3(CN(C)C)C[C@@H](C(F)(F)F)NC3=O)cc2n1)C1CCC(F)(F)CC1]

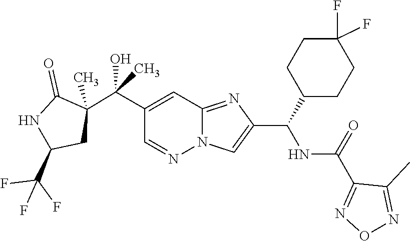 [SMILES: Cc1nonc1C(=O)N[C@H](c1cn2ncc([C@@](C)(O)[C@]3(C)C[C@@H](C(F)(F)F)NC3=O)cc2n1)C1CCC(F)(F)CC1]

In some embodiments, disclosed herein is a compound of Formula I, or a pharmaceutically acceptable salt thereof, selected from the group consisting of;

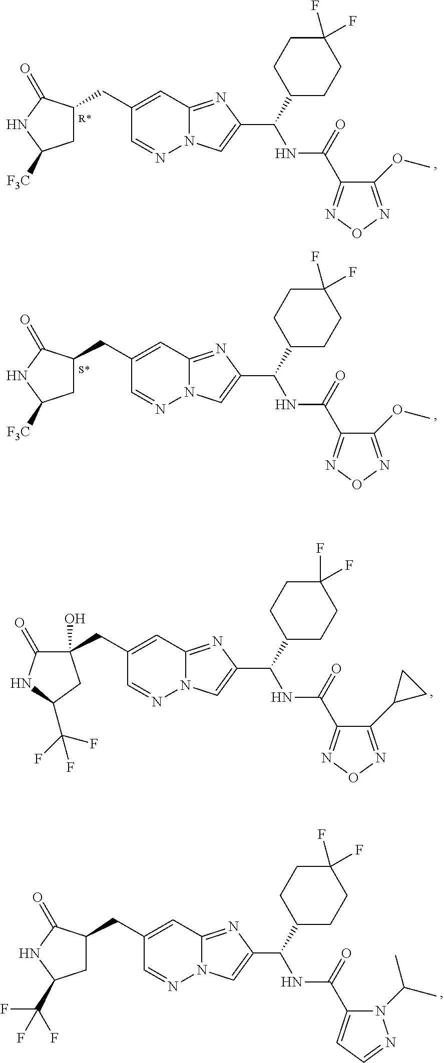

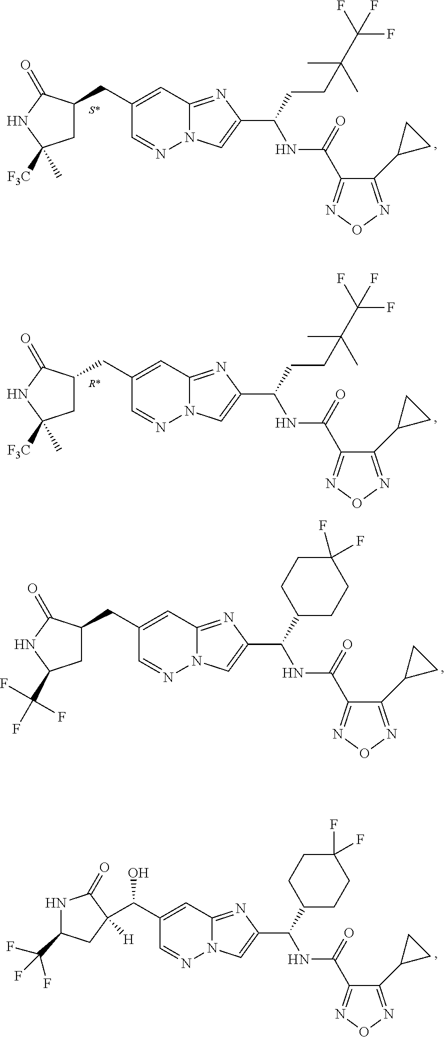

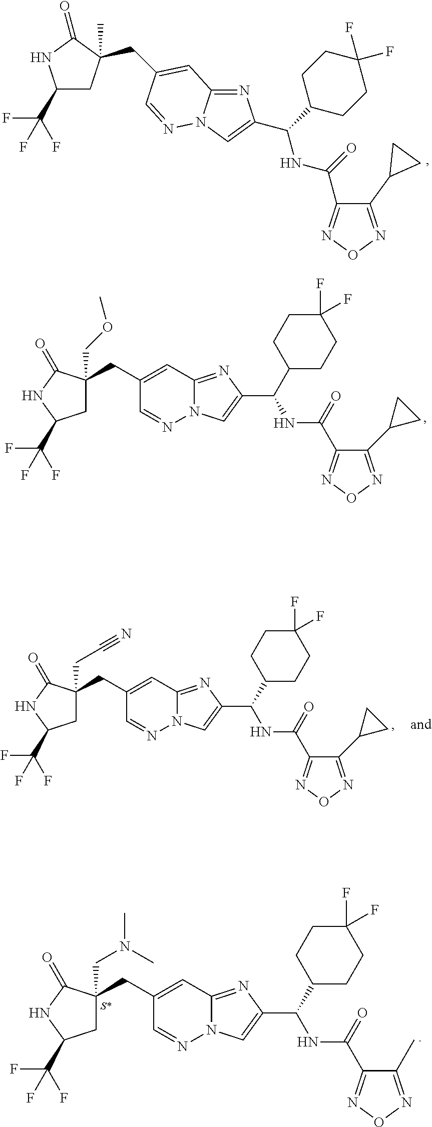

In some embodiments, disclosed herein is a compound of Formula I, or a pharmaceutically acceptable salt thereof, having the following formula:

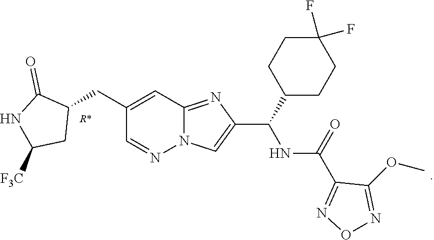 [SMILES: COc1nonc1C(=O)N[C@H](c1cn2ncc(C[C@@H]3C[C@@H](C(F)(F)F)NC3=O)cc2n1)C1CCC(F)(F)CC1]

In some embodiments, disclosed herein is a compound of Formula I, or a pharmaceutically acceptable salt thereof, having the following formula:

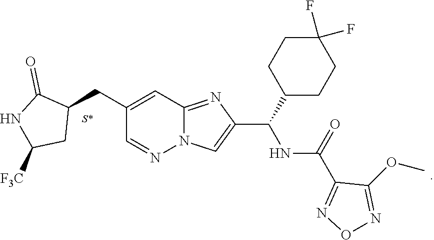 [SMILES: COc1nonc1C(=O)N[C@H](c1cn2ncc(C[C@H]3C[C@@H](C(F)(F)F)NC3=O)cc2n1)C1CCC(F)(F)CC1]

In some embodiments, disclosed herein is a compound of Formula I, or a pharmaceutically acceptable salt thereof, having the following formula:

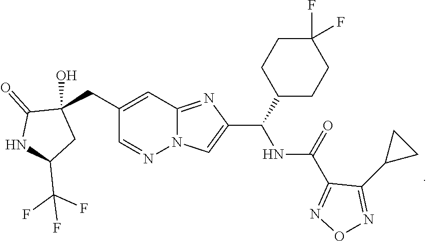 [SMILES: O=C(N[C@H](c1cn2ncc(C[C@@]3(O)C[C@@H](C(F)(F)F)NC3=O)cc2n1)C1CCC(F)(F)CC1)c1nonc1C1CC1]

In some embodiments, disclosed herein is a compound of Formula I, or a pharmaceutically acceptable salt thereof, having the following formula:

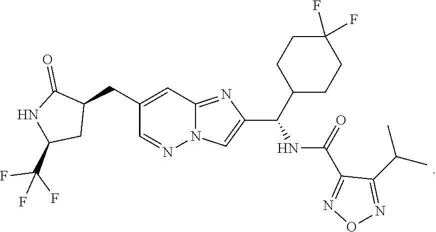 [SMILES: CC(C)c1nonc1C(=O)N[C@H](c1cn2ncc(C[C@H]3C[C@@H](C(F)(F)F)NC3=O)cc2n1)C1CCC(F)(F)CC1]

In some embodiments, disclosed herein is a compound of Formula I, or a pharmaceutically acceptable salt thereof, having the following formula:

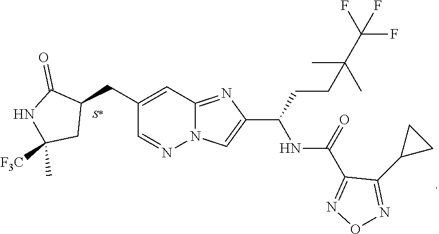 [SMILES: CC(C)(CC[C@H](NC(=O)c1nonc1C1CC1)c1cn2ncc(C[C@H]3C[C@@](C)(C(F)(F)F)NC3=O)cc2n1)C(F)(F)F]

In some embodiments, disclosed herein is a compound of Formula I, or a pharmaceutically acceptable salt thereof, having the following formula:

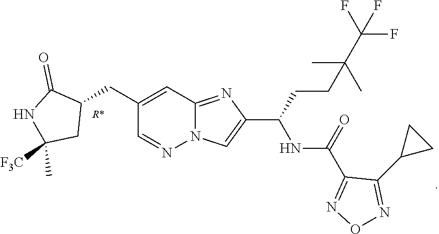 [SMILES: CC(C)(CC[C@H](NC(=O)c1nonc1C1CC1)c1cn2ncc(C[C@@H]3C[C@@](C)(C(F)(F)F)NC3=O)cc2n1)C(F)(F)F]

In some embodiments, disclosed herein is a compound of Formula I, or a pharmaceutically acceptable salt thereof, having the following formula:

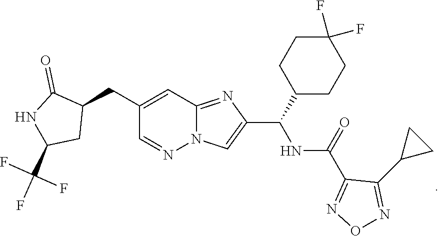 [SMILES: O=C(N[C@H](c1cn2ncc(C[C@H]3C[C@@H](C(F)(F)F)NC3=O)cc2n1)C1CCC(F)(F)CC1)c1nonc1C1CC1]

In some embodiments, disclosed herein is a compound of Formula I, or a pharmaceutically acceptable salt thereof, having the following formula:

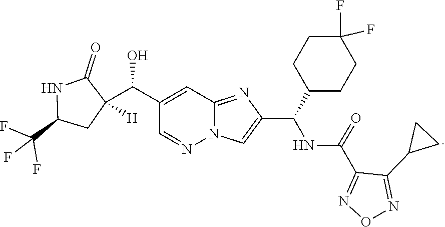 [SMILES: O=C(N[C@H](c1cn2ncc([C@@H](O)[C@H]3C[C@@H](C(F)(F)F)NC3=O)cc2n1)C1CCC(F)(F)CC1)c1nonc1C1CC1]

In some embodiments, disclosed herein is a compound of Formula I, or a pharmaceutically acceptable salt thereof, having the following formula:

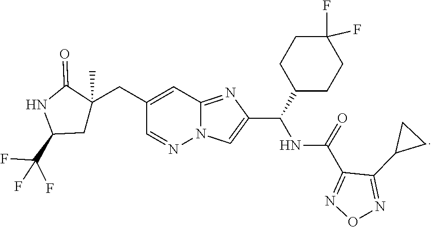 [SMILES: C[C@]1(Cc2cnn3cc([C@@H](NC(=O)c4nonc4C4CC4)C4CCC(F)(F)CC4)nc3c2)C[C@@H](C(F)(F)F)NC1=O]

In some embodiments, disclosed herein is a compound of Formula I, or a pharmaceutically acceptable salt thereof, having the following formula:

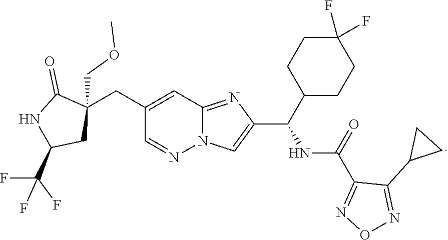 [SMILES: COC[C@@]1(Cc2cnn3cc([C@@H](NC(=O)c4nonc4C4CC4)C4CCC(F)(F)CC4)nc3c2)C[C@@H](C(F)(F)F)NC1=O]

In some embodiments, disclosed herein is a compound of Formula I, or a pharmaceutically acceptable salt thereof, having the following formula:

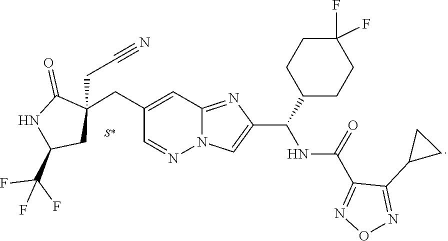 [SMILES: N#CC[C@@]1(Cc2cnn3cc([C@@H](NC(=O)c4nonc4C4CC4)C4CCC(F)(F)CC4)nc3c2)C[C@@H](C(F)(F)F)NC1=O]

In some embodiments, disclosed herein is a compound of Formula I, or a pharmaceutically acceptable salt thereof, having the following formula:

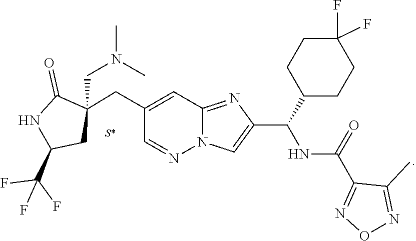 [SMILES: Cc1nonc1C(=O)N[C@H](c1cn2ncc(C[C@]3(CN(C)C)C[C@@H](C(F)(F)F)NC3=O)cc2n1)C1CCC(F)(F)CC1]

In some embodiments, disclosed herein is a compound of Formula I, or a pharmaceutically acceptable salt thereof, having a formula as shown in Table 2.

TABLE 2

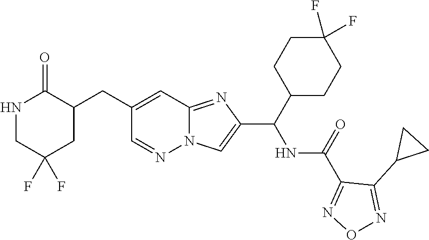 [SMILES: O=C(NC(c1cn2ncc(CC3CC(F)(F)CNC3=O)cc2n1)C1CCC(F)(F)CC1)c1nonc1C1CC1]

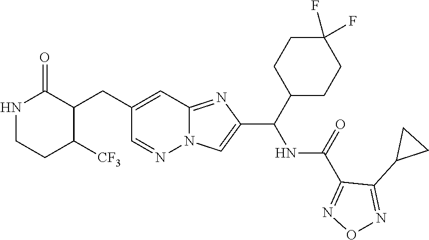 [SMILES: O=C(NC(c1cn2ncc(CC3C(=O)NCCC3C(F)(F)F)cc2n1)C1CCC(F)(F)CC1)c1nonc1C1CC1]

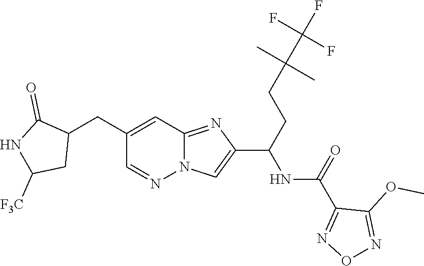 [SMILES: COc1nonc1C(=O)NC(CCC(C)(C)C(F)(F)F)c1cn2ncc(CC3CC(C(F)(F)F)NC3=O)cc2n1]

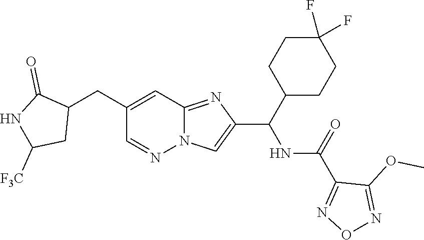 [SMILES: COc1nonc1C(=O)NC(c1cn2ncc(CC3CC(C(F)(F)F)NC3=O)cc2n1)C1CCC(F)(F)CC1]

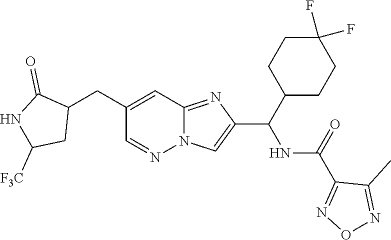 [SMILES: Cc1nonc1C(=O)NC(c1cn2ncc(CC3CC(C(F)(F)F)NC3=O)cc2n1)C1CCC(F)(F)CC1]

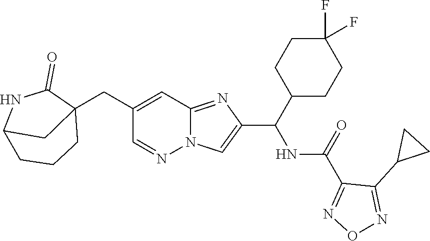 [SMILES: O=C(NC(c1cn2ncc(CC34CCCC(C3)NC4=O)cc2n1)C1CCC(F)(F)CC1)c1nonc1C1CC1]

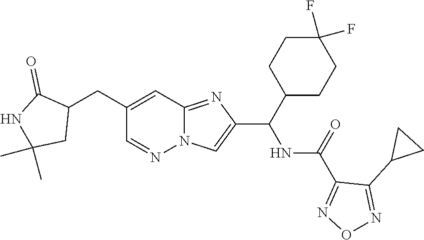 [SMILES: CC1(C)CC(Cc2cnn3cc(C(NC(=O)c4nonc4C4CC4)C4CCC(F)(F)CC4)nc3c2)C(=O)N1]

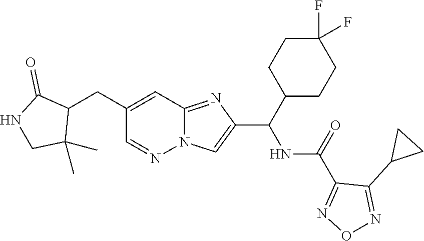 [SMILES: CC1(C)CNC(=O)C1Cc1cnn2cc(C(NC(=O)c3nonc3C3CC3)C3CCC(F)(F)CC3)nc2c1]

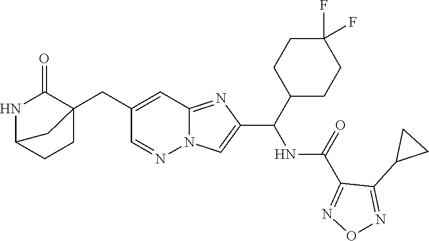 [SMILES: O=C(NC(c1cn2ncc(CC34CCC(C3)NC4=O)cc2n1)C1CCC(F)(F)CC1)c1nonc1C1CC1]

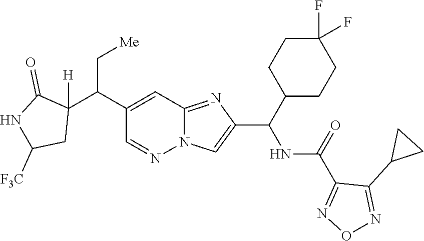 [SMILES: CCC(c1cnn2cc(C(NC(=O)c3nonc3C3CC3)C3CCC(F)(F)CC3)nc2c1)C1CC(C(F)(F)F)NC1=O]

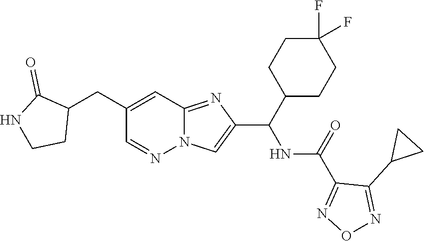 [SMILES: O=C(NC(c1cn2ncc(CC3CCNC3=O)cc2n1)C1CCC(F)(F)CC1)c1nonc1C1CC1]

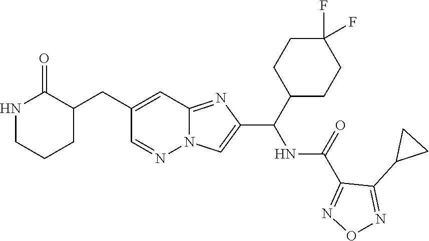 [SMILES: O=C(NC(c1cn2ncc(CC3CCCNC3=O)cc2n1)C1CCC(F)(F)CC1)c1nonc1C1CC1]

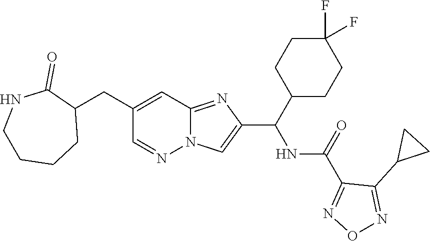 [SMILES: O=C(NC(c1cn2ncc(CC3CCCCNC3=O)cc2n1)C1CCC(F)(F)CC1)c1nonc1C1CC1]

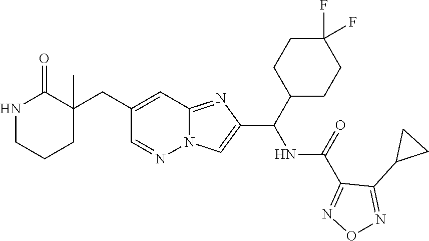 [SMILES: CC1(Cc2cnn3cc(C(NC(=O)c4nonc4C4CC4)C4CCC(F)(F)CC4)nc3c2)CCCNC1=O]

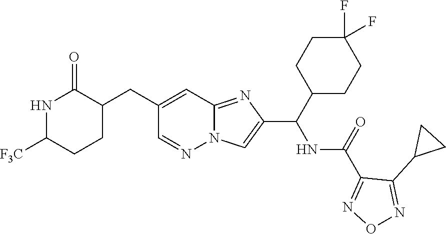 [SMILES: O=C(NC(c1cn2ncc(CC3CCC(C(F)(F)F)NC3=O)cc2n1)C1CCC(F)(F)CC1)c1nonc1C1CC1]

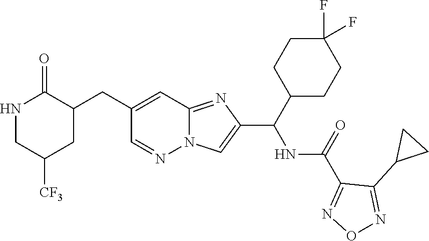 [SMILES: O=C(NC(c1cn2ncc(CC3CC(C(F)(F)F)CNC3=O)cc2n1)C1CCC(F)(F)CC1)c1nonc1C1CC1]

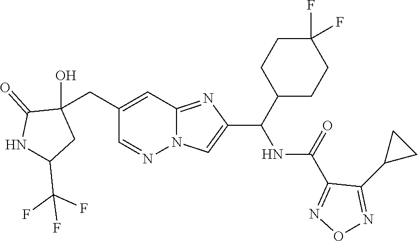 [SMILES: O=C(NC(c1cn2ncc(CC3(O)CC(C(F)(F)F)NC3=O)cc2n1)C1CCC(F)(F)CC1)c1nonc1C1CC1]

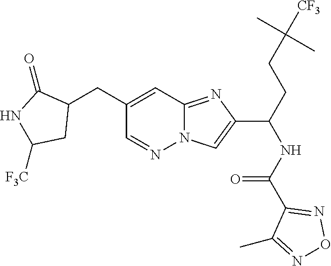 [SMILES: Cc1nonc1C(=O)NC(CCC(C)(C)C(F)(F)F)c1cn2ncc(CC3CC(C(F)(F)F)NC3=O)cc2n1]

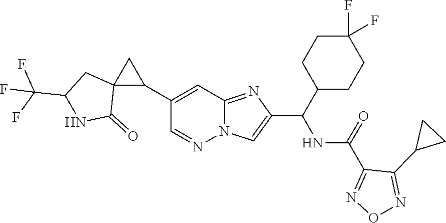 [SMILES: O=C(NC(c1cn2ncc(C3CC34CC(C(F)(F)F)NC4=O)cc2n1)C1CCC(F)(F)CC1)c1nonc1C1CC1]

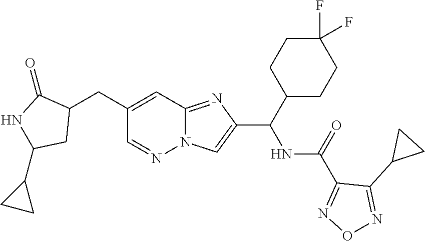 [SMILES: O=C(NC(c1cn2ncc(CC3CC(C4CC4)NC3=O)cc2n1)C1CCC(F)(F)CC1)c1nonc1C1CC1]

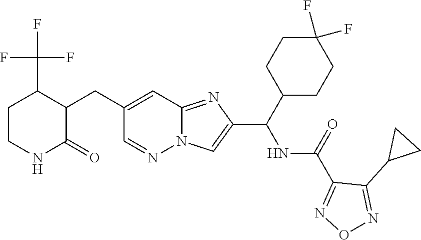 [SMILES: O=C(NC(c1cn2ncc(CC3C(=O)NCCC3C(F)(F)F)cc2n1)C1CCC(F)(F)CC1)c1nonc1C1CC1]

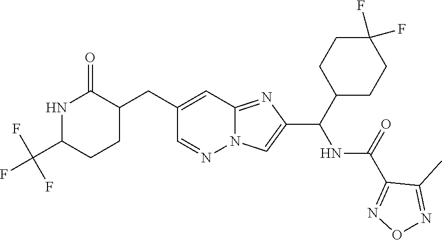 [SMILES: Cc1nonc1C(=O)NC(c1cn2ncc(CC3CCC(C(F)(F)F)NC3=O)cc2n1)C1CCC(F)(F)CC1]

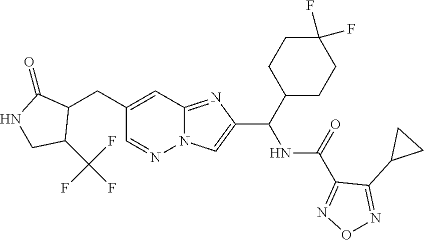 [SMILES: O=C(NC(c1cn2ncc(CC3C(=O)NCC3C(F)(F)F)cc2n1)C1CCC(F)(F)CC1)c1nonc1C1CC1]

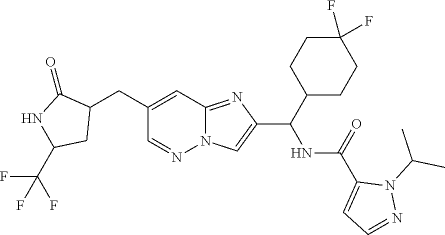 [SMILES: CC(C)n1nccc1C(=O)NC(c1cn2ncc(CC3CC(C(F)(F)F)NC3=O)cc2n1)C1CCC(F)(F)CC1]

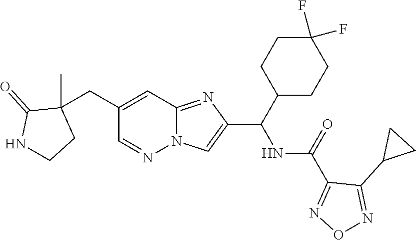 [SMILES: CC1(Cc2cnn3cc(C(NC(=O)c4nonc4C4CC4)C4CCC(F)(F)CC4)nc3c2)CCNC1=O]

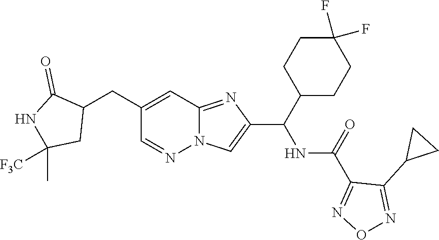 [SMILES: CC1(C(F)(F)F)CC(Cc2cnn3cc(C(NC(=O)c4nonc4C4CC4)C4CCC(F)(F)CC4)nc3c2)C(=O)N1]

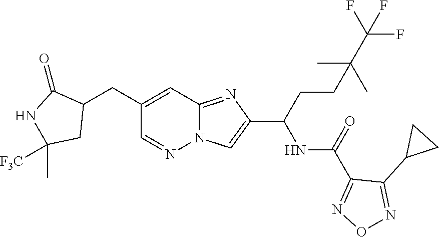 [SMILES: CC(C)(CCC(NC(=O)c1nonc1C1CC1)c1cn2ncc(CC3CC(C)(C(F)(F)F)NC3=O)cc2n1)C(F)(F)F]

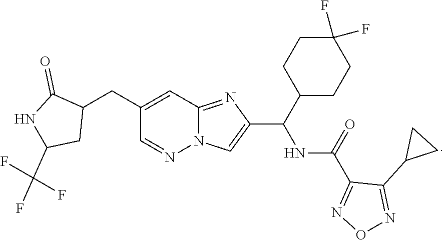 [SMILES: O=C(NC(c1cn2ncc(CC3CC(C(F)(F)F)NC3=O)cc2n1)C1CCC(F)(F)CC1)c1nonc1C1CC1]

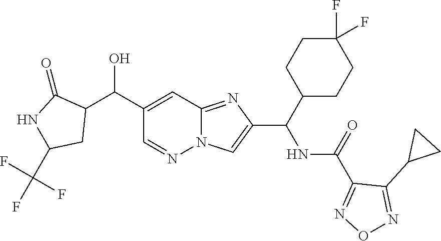 [SMILES: O=C(NC(c1cn2ncc(C(O)C3CC(C(F)(F)F)NC3=O)cc2n1)C1CCC(F)(F)CC1)c1nonc1C1CC1]

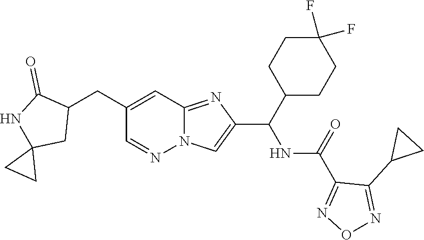 [SMILES: O=C(NC(c1cn2ncc(CC3CC4(CC4)NC3=O)cc2n1)C1CCC(F)(F)CC1)c1nonc1C1CC1]

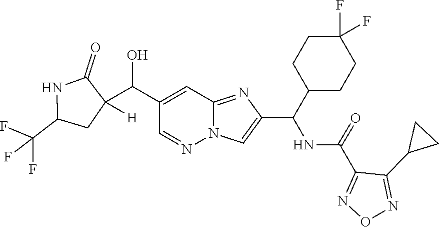 [SMILES: O=C(NC(c1cn2ncc(C(O)C3CC(C(F)(F)F)NC3=O)cc2n1)C1CCC(F)(F)CC1)c1nonc1C1CC1]

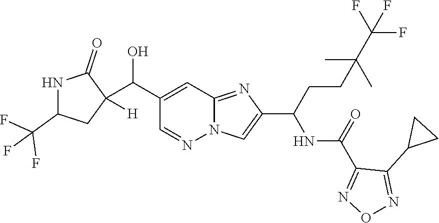 [SMILES: CC(C)(CCC(NC(=O)c1nonc1C1CC1)c1cn2ncc(C(O)C3CC(C(F)(F)F)NC3=O)cc2n1)C(F)(F)F]

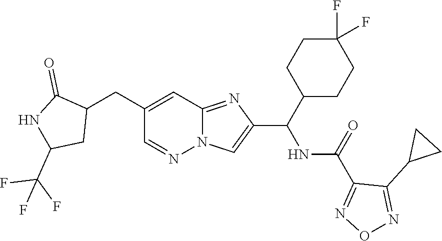 [SMILES: O=C(NC(c1cn2ncc(CC3CC(C(F)(F)F)NC3=O)cc2n1)C1CCC(F)(F)CC1)c1nonc1C1CC1]

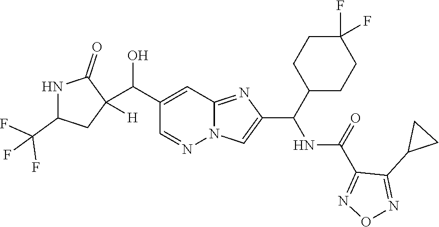 [SMILES: O=C(NC(c1cn2ncc(C(O)C3CC(C(F)(F)F)NC3=O)cc2n1)C1CCC(F)(F)CC1)c1nonc1C1CC1]

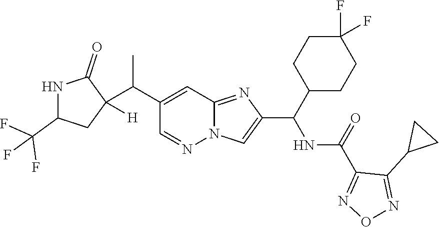 [SMILES: CC(c1cnn2cc(C(NC(=O)c3nonc3C3CC3)C3CCC(F)(F)CC3)nc2c1)C1CC(C(F)(F)F)NC1=O]

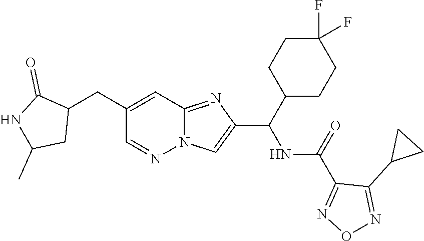 [SMILES: CC1CC(Cc2cnn3cc(C(NC(=O)c4nonc4C4CC4)C4CCC(F)(F)CC4)nc3c2)C(=O)N1]

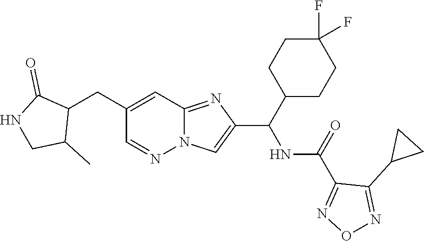 [SMILES: CC1CNC(=O)C1Cc1cnn2cc(C(NC(=O)c3nonc3C3CC3)C3CCC(F)(F)CC3)nc2c1]

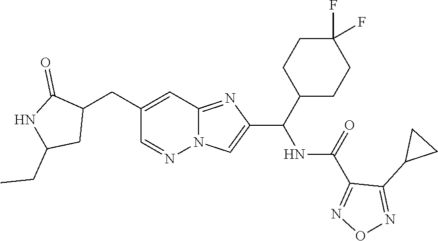 [SMILES: CCC1CC(Cc2cnn3cc(C(NC(=O)c4nonc4C4CC4)C4CCC(F)(F)CC4)nc3c2)C(=O)N1]

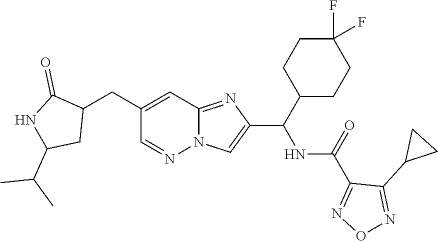 [SMILES: CC(C)C1CC(Cc2cnn3cc(C(NC(=O)c4nonc4C4CC4)C4CCC(F)(F)CC4)nc3c2)C(=O)N1]

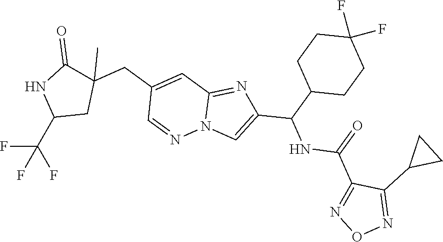 [SMILES: CC1(Cc2cnn3cc(C(NC(=O)c4nonc4C4CC4)C4CCC(F)(F)CC4)nc3c2)CC(C(F)(F)F)NC1=O]

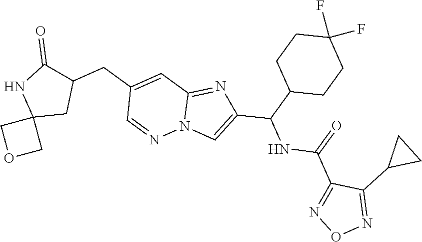 [SMILES: O=C(NC(c1cn2ncc(CC3CC4(COC4)NC3=O)cc2n1)C1CCC(F)(F)CC1)c1nonc1C1CC1]

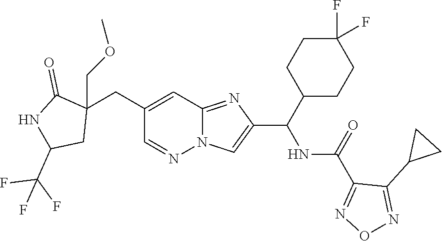 [SMILES: COCC1(Cc2cnn3cc(C(NC(=O)c4nonc4C4CC4)C4CCC(F)(F)CC4)nc3c2)CC(C(F)(F)F)NC1=O]

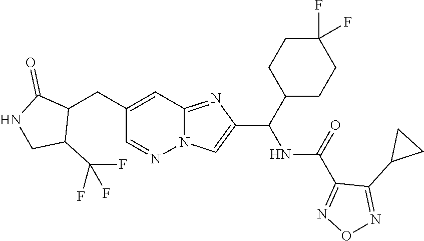 [SMILES: O=C(NC(c1cn2ncc(CC3C(=O)NCC3C(F)(F)F)cc2n1)C1CCC(F)(F)CC1)c1nonc1C1CC1]

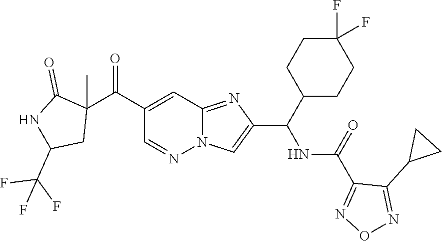 [SMILES: CC1(C(=O)c2cnn3cc(C(NC(=O)c4nonc4C4CC4)C4CCC(F)(F)CC4)nc3c2)CC(C(F)(F)F)NC1=O]

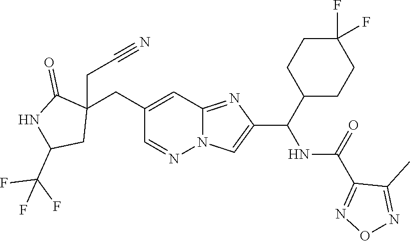 [SMILES: Cc1nonc1C(=O)NC(c1cn2ncc(CC3(CC#N)CC(C(F)(F)F)NC3=O)cc2n1)C1CCC(F)(F)CC1]

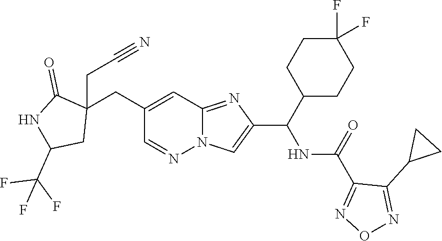 [SMILES: N#CCC1(Cc2cnn3cc(C(NC(=O)c4nonc4C4CC4)C4CCC(F)(F)CC4)nc3c2)CC(C(F)(F)F)NC1=O]

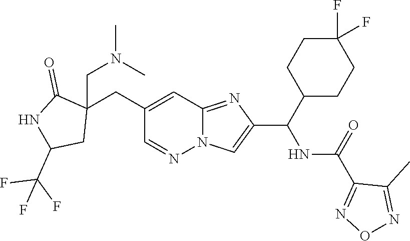 [SMILES: Cc1nonc1C(=O)NC(c1cn2ncc(CC3(CN(C)C)CC(C(F)(F)F)NC3=O)cc2n1)C1CCC(F)(F)CC1]

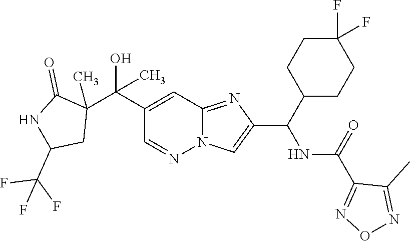 [SMILES: Cc1nonc1C(=O)NC(c1cn2ncc(C(C)(O)C3(C)CC(C(F)(F)F)NC3=O)cc2n1)C1CCC(F)(F)CC1]

In some embodiments, disclosed herein is a pharmaceutical composition, comprising a compound of Formula I, or a pharmaceutically acceptable salt thereof, and a pharmaceutically acceptable carrier. In some embodiments, the pharmaceutical composition is formulated for oral administration (e.g., a tablet or capsule).

In some embodiments, disclosed herein is a pharmaceutical composition made by mixing a compound of Formula I, or a pharmaceutically acceptable salt thereof, and a pharmaceutically acceptable carrier.

In some embodiments, disclosed herein is a process for making a pharmaceutical composition comprising mixing a compound of Formula I, or a pharmaceutically acceptable salt thereof, and a pharmaceutically acceptable carrier.

### Therapeutic Use

Preclinical and clinical data have demonstrated the significant pathological role of IL-17A in multiple autoimmune and inflammatory diseases.

For psoriasis: IL-17A mRNA and/or protein levels are elevated in the lesional skin and blood of patients with psoriasis and correlate with disease severity. IL-17A acts directly in synergy with other cytokines (such as TNFα, IFNγ or IL-22) on keratinocytes triggering a self-amplifying inflammatory response in the skin and leading to the formation of psoriatic plaques. The blockade of IL-17A by means of antibodies to IL-17A or IL-23 results in complete reversal of the molecular and clinical disease features in majority of psoriasis patients, manifesting the significant role of IL-17A and IL-17-producing T-cells in the immunopathogenesis of psoriasis. (Hawkes et al., Psoriasis Pathogenesis and the Development of Novel, Targeted Immune Therapies. J Allergy Clin Immunol. 2017, 140(3): 645-653). The development and approval of IL-17 monoclonal antibodies such as secukinumab, ixekizumab, and brodalumab and their transformational efficacy for psoriasis have demonstrated IL-17A as a valid target for psoriasis treatments. (Blauvelt A. and Chiricozzi A. The Immunologic Role of IL-17 in Psoriasis and Psoriatic Arthritis Pathogenesis. Clin Rev Allergy Immunol. 2018, 55(3):379-390).

For psoriatic arthritis (PsA): IL-17A is mechanistically relevant to PsA through NFκB activation that triggers transcription of several PsA related genes including the receptor activator of nuclear factor κB ligand (RANKL). RANKL triggers the differentiation of osteoclast precursor cells into activated osteoclasts, resulting in bone resorption and subsequently joint deformity in PsA (Adamopoulos I. and Mellins E. Nature reviews Rheumatology 2015. 11:189-94). PsA joint is enriched for IL-17+CD8+ T cells, and the levels of this T cell subset are correlated with disease activity (Menon B. et al., Arthritis & Rheumatology 2014; 66: 1272-81). Synovial fibroblasts isolated from PsA patients also contain elevated IL-17R expression and secrete increased IL-6, CXCL8 and MMP3 ex vivo compared to osteoarthritis patients. Both secukinumab and ixekizumab are FDA approval drugs for PsA. In matching-adjusted indirect comparison analysis, secukinumab was associated with higher ACR 20/50/70 response rates in patients with active PsA than anti-TNFα antibodies (Mease P. et al., Eur. J. Rheumatol. 2019 Jul. 1; 6(3):113-121; Strand V. et al., J. Comp. Eff. Res. 2019, 8(7):497-510; Nash P. et al., Rheumatol. Ther. 2018, 5(1):99-122). In a recent head-to-head study, ixekizumab was superior to adalimumab in achieving simultaneous improvement of joint and skin disease (ACR50 and PASI100) in patients with PsA and inadequate response to conventional synthetic disease-modifying antirheumatic drug (Mease, P. et al. Ann Rheum Diss 2020; 79:123-131). By hitting the same target, IL-17A small molecule inhibitor compounds may exert similar or better efficacy than biologics considering that small molecules generally have better tissue penetration.

For rheumatoid arthritis (RA): IL-17A has been recognized as critical to the progression of rheumatoid arthritis. “The recognition of IL-17 as a pro-inflammatory T cell derived cytokine, and its abundance within rheumatoid joints, provides the strongest candidate mechanism to date through which T cells can capture and localize macrophage effector functions in rheumatoid arthritis” Stamp, L. et al., Immunol. Cell Biol. 2004, 82(1): 1-9. Moreover, in rheumatoid arthritis IL-17A acts locally on synoviocytes and osteoblasts contributing to synovitis and joint destruction. Robert and Miossec have proposed the use of synovial biopsies and/or biomarkers to precisely identify patients that would respond to IL-17A inhibition. Their work concludes that IL-17 inhibitors should now be considered in the development of precision medicine in RA. (Robert M. and Miossec P., Front. Med., 2019, 5:364).

For Ankylosing Spondylitis (AS): Various studies have reported elevated IL-17A and Th17 and other cells producing IL-17 in AS blood samples (Wendling D. et al., Joint Bone Spine. 2007; 74:304-305; Shen H. et al., Arthritis Rheum. 2009; 60(6):1647-56; Zhang L. et al., PLoS One. 2012; 7(4):e31000; Jansen D. et al., Rheumatology (Oxford). 2015 April; 54(4): 728-735). In situ analysis of AS spine has revealed increased IL-17A-producing cells in bone of facet (zygapophyseal) joints (Appel H. et al., Arthritis Res. Ther. 2011; 13(3):R⁹⁵). Two advanced IL-17A neutralizing antibodies, secukinumab, approved by FDA for AS, and ixekizumab, have demonstrated efficacy over placebo even in anti-TNF inadequate responders. In contrast, anti-IL-23 p40 and p19 biologics failed to demonstrate beneficial effect (Deodhar A. et al., Arthritis Rheumatol. 2019, 71(2):258-270; Baeten D. et al., Ann. Rheum. Dis. 2018, 77(9):1295-1302), indicating the differential underling mechanism along IL-23/IL-17 pathway in AS and providing strong evidence to support continuing developing IL-17A inhibitors.

For hidradenitis suppurativa (HS): Increased IL-17 and IL-17-producing T helper cells in the skin lesions of HS patients were reported and molecular proteomics and gene expression data indicate that the IL-23/Th17 pathway is upregulated in HS lesions (Schlapbach C. et al., J. Am. Acad. Dermatol. 2011; 65(4):790; Kelly G. et al., British J. Dermatol. 2015 December; 173(6):1431-9; Moran B. et al., J. Invest. Dermatol. 2017; 137(11):2389; Thomi R. et al., JAMA Dermatol. 2018; 154(5):592). Seven of nine (78%) patients with moderate-to-severe HS achieved HiSCR in an open-label pilot-trial with Secukinumab (Prussick L. et al., British J. Dermatol. 2019 September; 181(3):609-611), and more clinical trials with anti-IL-17 mAbs in HS are on-going.

For bullous pemphigoid (BP): IL-17 is elevated in the blister fluid and perilesional skin of BP patients. (Le Jan S. et al., J. Invest. Dermatol. 2014; 134 (12):2908-2917; Chakievska L. J Autoimmun. 2019, 96:104-112). Exome sequencing of BP patients revealed mutations in twelve IL-17-related genes in one third of patients, providing the genetic link between IL-17 pathway and BP (Chakievska L. J Autoimmun. 2019, 96:104-112). In experimental murine BP, IL-17A−/− mice are protected, and anti-IL-17A treatment significantly reduced skin lesions in wild type (Chakievska L. J Autoimmun. 2019, 96:104-112). Ixekizumab Phase 2 of treatment naive and refractory BP patients is on-going (NCT03099538).

For atopic dermatitis (AD): IL-17 was found to be elevated in peripheral blood and lesions in AD patients and Th17 cells infiltrated more markedly in acute than chronic lesions, suggesting its role in acute phase of AD (Koga C. et al., J. Invest. Dermatol. 2008, 128, 2625-2630). Molecular profile analysis from ustekinumab Phase II suggest likely contribution of IL-23/Th17/IL-17 pathway in AD (Khattri S. et al., Exp. Dermatol. 2017 January; 26(1):28-35).

For vitiligo: Many studies in vitiligo patients have demonstrated an increased frequency of Th17 cells and higher levels of IL-17 in both circulation and lesions that positively correlates with disease duration, extent, and activity (Singh R et al., Autoimmun. Rev 2016, April; 15(4):397-404). Mouse studies demonstrated that depigmentation correlates with greater IL-17 expression/secretion, which modulates vitiligo development (Eby J. et al., Pigment Cell & Melanoma Res. 2014, November; 27(6):1075-85).

For multiple sclerosis (MS): IL-17 expression is increased in PBMCs, cerebrospinal fluid (CSF) as well as in brain lesions and cells from MS patients (Lock, C. et al., Nat. Med. 2002, 8: 500-508; Matusevicius, D. et al., Mult. Scler. 1999, 5: 101-104; Tzartos, J. et al., Am. J. Pathol. 2008, 172: 146-155). IL-17-producing T cells are enriched in active MS lesions (Tzartos, J. et al., Am. J. Pathol. 2008, 172: 146-155; Willing A. et al., J. Immunol. 2018, 200(3):974-982). IL-17A levels were elevated in the CSF of relapsing-remitting MS (RRMS) patients and correlated with the CSF/serum albumin quotient, a measure of blood-brain barrier (BBB) dysfunction, together with in vitro data that IL-17A in combination with IL-6 reduced the expression of tight junction-associated genes and disrupted monolayer integrity in a BBB cell line, highlighting the potential importance of targeting IL-17A in preserving BBB integrity in RRMS (Setiadi A F et al., J Neuroimmunol. 2019, 332:147-154). Secukinumab yielded promising first results in a proof-of-concept study in MS patients (Havrdová, E. et al., J. Neurol. 2016, 263: 1287-1295).

For Asthma: IL-17 expression is increased in the lung, sputum, bronchoalveolar lavage fluid, and sera in patients with asthma, and the severity of airway hyperresponsiveness is positively correlated with IL-17 expression levels. (Chakir J. et al., J. Allergy Clin. Immunol. 2003,111(6):1293-8). IL-17 was reported to be increased in asthmatic airways and induce human bronchial fibroblasts to produce cytokines (Molet S. et al., J. Allergy Clin. Immunol. 2001, 108(3):430-8). Anti-IL-17 antibody modulates airway responsiveness, inflammation, tissue remodeling, and oxidative stress in chronic mouse asthma models (Camargo LdN. et al., Front Immunol. 2018; 8:1835; dos Santos T. et al., Front. Physiol. 2018, 9:1183).

For Chronic Obstructive Pulmonary Disease (COPD): An increase in Th17 cells was observed in patients with COPD compared with current smokers without COPD and healthy subjects, and inverse correlations were found between Th17 cells with lung function (Vargas-Rojas M. et al., Respir. Med. 2011 November; 105(11):1648-54). In three recent human COPD studies, gene expression profile in bronchial epithelia showed that higher IL-17 signature expression is associated with a lack of response to inhaled corticosteroid, suggesting that there is a COPD subgroup that may benefit from IL-17 inhibitor therapy (Christenson S. et al., J. Clin. Invest. 2019; 129(1):169-181).

For Uveitis: IL-17 promotes the release of inflammatory mediators from retinal pigment epithelium cell line, disrupting the retinal pigment epithelium barrier function (Chen Y. et al., PLoS One. 2011; 6:e18139). IL-17 levels were elevated in the serum or aqueous humor of uveitis patients (El-Asrar A. et al., Clin. Immunol. 2011; 139(2):177-84; Jawad S. et al., Ocul. Immunol. Inflamm. 2013; 21(6):434-9; Kuiper J. et al., Am. J. Ophthalmol. 2011; 152(2):177-182). Anti-IL-17 antibody delayed the onset of ocular inflammation and markedly inhibited the development of experimental autoimmune uveitis in rats (Zhang R. et al., Curr. Eye Res. 2009 April; 34(4):297-303). The analysis of secondary efficacy data from subcutaneous (sc) secukinumab phase 3 trials in uveitis suggested a beneficial effect of secukinumab in reducing the use of concomitant immunosuppressive medication (Dick A. et al., Ophthalmology 2013; 120(4):777-87). Later study of intravenous secukinumab in uveitis demonstrated greater efficacy than sc dosing, suggesting requiring optimal exposure for efficacy and confirming the therapeutic potential of IL-17A inhibition (Letko E. et al., Ophthalmology 2015, 122(5), 939-948). Ustekinumab that blocks IL-23/IL-17 pathway was also reported to successfully treat a noninfectious uveitis patient who had severe concomitant psoriasis and PsA and failed to respond to conventional immune suppressants (Mugheddu C. et al., Dermatol. Ther. 2017 September; 30(5); e12527).

For multiple myeloma (MM): IL-17A serum levels were significantly higher in MM patients and also in patients with advanced stage compared with healthy subjects (Lemancewicz D. et al., Med. Sci. Monit. 2012; 18(1): BR54-BR59). Administration of secukinumab in the SCIDhu model of human myeloma weekly for 4 weeks after the first detection of tumor in mice led to a significant inhibition of tumor growth and reduced bone damage compared to isotype control mice (Prabhala R. et al., Leukemia. 2016 February; 30(2): 379-389).

For systemic lupus erythematosus (SLE): Increased serum or plasma levels of IL-17, expansion of IL-17-producing T cells in the peripheral blood, and infiltration of Th17 cells in target organs like the kidneys was observed in SLE patients (Wong C. et al., Lupus. 2000; 9(8):589-593; Wong C. et al., Clinical Immunology. 2008; 127(3):385-393; Zhao X-F. et al., Mol. Biol. Rep. 2010 January; 37(1):81-5; Chen X. et al., J. Clin. Immunol. 2010 March: 30(2):221-5; Xing Q. et al., Rheumatol. Int. 2012 April; 32(4):949-58). Imbalance between Th17 cells and regulatory T (Treg) cells has been observed in SLE patients including quiescent stage (Ma J. et al., Clin. Rheumatol. 2010; 29(11):1251-1258; DolffS. et al., Clin. Immunol. 2011, 141(2):197-204). Overexpression of IL-17A using adenovirus enhanced the severity of lupus nephritis, while blockade of IL-17A using neutralizing antibody resulted in decreased severity of lupus nephritis (Wen, Z. et al., PLoS One. 2013, 8: e58161). In a phase 2 study, ustekinumab, an anti-IL-12/23 p40 monoclonal antibody blocking IL-23/IL-17 pathway, has demonstrated efficacy in SLE patients (van Vollenhoven R et al., Lancet 2018; 392: 1330-39). Human expression studies, animal models, and clinical trials indicate that IL-17 blockade may become a promising therapeutic strategy for SLE (Koga T. et al., Expert Rev. Clin. Immunol. 2019, 15 (6) 629-637).

Accordingly, the present application is also directed to a method for treating and/or ameliorating an IL-17 mediated inflammatory syndrome, disorder or disease comprising administering to a subject in need thereof an effective amount of a compound of Formula I, or pharmaceutically acceptable salt thereof, composition thereof, or medicament thereof.

In some embodiments, disclosed herein is a method for treating or ameliorating an IL-17A mediated inflammatory syndrome, disorder, or disease comprising administering to a subject in need thereof a therapeutically effective amount of a compound of Formula I, or a pharmaceutically acceptable salt thereof.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is psoriasis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is psoriatic arthritis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is rheumatoid arthritis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is ankylosing spondylitis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is hidradenitis suppurativa.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is bullous pemphigoid.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is atopic dermatitis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is vitiligo.

In some embodiments, disclosed herein is a method for treating or ameliorating and/an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is multiple sclerosis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is systemic lupus erythematosus.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is asthma.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is uveitis.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is chronic obstructive pulmonary disorder.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is multiple myeloma.

In some embodiments, disclosed herein is a method for treating and/or ameliorating an IL-17A mediated inflammatory syndrome, disorder, or disease comprising administering to a subject in need thereof a therapeutically effective amount of a compound of Formula I, or a pharmaceutically acceptable salt thereof, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus, wherein the compound of Formula I or the pharmaceutically acceptable salt thereof is administered orally (e.g., as a tablet or capsule).

In some embodiments, disclosed herein is the use of a therapeutically effective amount of compound of Formula I, or a pharmaceutically acceptable salt thereof, for treating and/or ameliorating an IL-17A mediated inflammatory syndrome, disorder, or disease selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus.

In some embodiments, disclosed herein is the use of a compound of Formula I, or pharmaceutically acceptable salt thereof, in the manufacture of a medicament for treating and/or ameliorating an IL-17A mediated inflammatory syndrome, disorder, or disease selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus.

In some embodiments, disclosed herein is a method for treating and/or ameliorating an IL-17 mediated inflammatory syndrome, disorder or disease comprising administering to a subject in need thereof a therapeutically effective amount of a compound of Formula I, or pharmaceutically acceptable salt thereof, a composition thereof, or a medicament thereof.

In some embodiments, disclosed herein is a method of treating and/or ameliorating an IL-17 mediated inflammatory syndrome, disorder or disease, wherein the syndrome, disorder or disease is selected from the group consisting of: psoriasis, psoriatic arthritis, rheumatoid arthritis, ankylosing spondylitis, hidradenitis suppurativa, atopic dermatitis, vitiligo, multiple sclerosis, asthma, allergic asthma, steroid resistant asthma, neutrophilic asthma, chronic obstructive pulmonary disease, uveitis, multiple myeloma, and systemic lupus erythematosus, comprising administering to a subject in need thereof a therapeutically effective amount of a compound of Formula I, or pharmaceutically acceptable salt thereof, a composition thereof, or a medicament thereof.

In some embodiments, disclosed herein is a method of treating or ameliorating an IL-17 mediated inflammatory syndrome, disorder or disease, wherein the syndrome, disorder or disease is selected from the group consisting of: psoriasis, psoriatic arthritis, and ankylosing spondylitis, comprising administering to a subject in need thereof a therapeutically effective amount of a compound of Formula I, or pharmaceutically acceptable salt thereof, a composition thereof, or a medicament thereof.

In some embodiments, disclosed herein are methods of modulating IL-17 activity in a mammal by administration of a therapeutically effective amount of at least one compound of Formula I, or pharmaceutically acceptable salt thereof.

Also disclosed herein is a method of inhibiting production of interleukin-17, comprising administering to a subject in need thereof a therapeutically effective amount of a compound of Formula I, or pharmaceutically acceptable salt thereof.

### Combination Therapy

A compound of Formula I, or pharmaceutically acceptable salt thereof, a composition thereof, or a medicament thereof may also be used in combination with one or more additional therapeutic agents.

In some embodiments, the one or more additional therapeutic agents is selected from the group consisting of anti-inflammatory agents, immunomodulatory agents, and immunosuppressive agents.

In some embodiments, disclosed herein is a method of treating and/or ameliorating an IL-17 mediated inflammatory syndrome, disorder or disease, in a subject in need thereof comprising administering to the subject a therapeutically effective amount of the compound of Formula I, or pharmaceutically acceptable salt thereof, composition thereof, or medicament thereof in a combination therapy with one or more additional therapeutic agents, such as anti-inflammatory agents, or immunosuppressive agents, wherein said syndrome, disorder or disease is psoriasis, psoriatic arthritis, ankylosing spondylitis. In some embodiments, the IL-17 mediated inflammatory syndrome, disorder or disease is psoriasis. In some embodiments, the IL-17 mediated inflammatory syndrome, disorder or disease is psoriatic arthritis. In some embodiments, the IL-17 mediated inflammatory syndrome, disorder or disease is ankylosing spondylitis.

### Dosage Regimen

The dosage administered will be affected by factors such as the route of administration, the health, weight and age of the recipient, the frequency of the treatment and the presence of concurrent and unrelated treatments.

It is also apparent to one skilled in the art that the therapeutically effective dose for compounds of the present disclosure or a pharmaceutical composition thereof will vary according to the desired effect. Therefore, optimal dosages to be administered may be readily determined by one skilled in the art and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this disclosure.

### Pharmaceutical Compositions

The compounds of Formula I, or pharmaceutically acceptable salt thereof, may be formulated into pharmaceutical compositions comprising any known pharmaceutically acceptable carriers. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

The pharmaceutical compositions of the present disclosure may be administered by any means that accomplish their intended purpose. Examples include administration by parenteral, subcutaneous, intravenous, intramuscular, intraperitoneal, transdermal, topical, buccal or ocular routes. Alternatively or concurrently, administration may be by the oral route.

Also disclosed herein is a method of making a pharmaceutical composition comprising mixing a pharmaceutically acceptable carrier with any of the compounds of Formula I, or pharmaceutically acceptable salt thereof. Additionally, the present application includes pharmaceutical compositions made by mixing a pharmaceutically acceptable carrier with any of the compounds of the present disclosure.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

The present disclosure also provides the following non-limiting embodiments:
1. A compound of Formula I

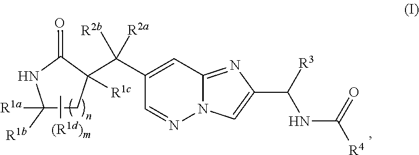

or a pharmaceutically acceptable salt thereof,
wherein:
   R^{1a} and R^{1b} are each independently H, —C₍₁₋₃₎alkyl, —C₍₃₋₅₎cycloalkyl, —C₍₁₋₃₎alkyl-O—C₍₁₋₃₎alkyl or —C₍₁₋₃₎alkyl-N(C₍₁₋₃₎alkyl)₂, wherein the —C₍₁₋₃₎alkyl is unsubstituted or substituted with one to six R^{1aa} groups; or R^{1a} and R^{1b} together with the carbon atom to which they are attached form a spiro C₍₃₋₅₎cycloalkyl or a spiro heterocyclyl selected from

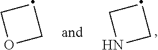

wherein · indicates the carbon atom to which R^{1a} and R^{1b} are attached; or R^{1a} and R^{1c} are taken together to form a C₍₁₋₂₎alkylenyl bridge;
R^{1c} is H, halo, —OH, —C₍₁₋₃₎alkyl, —C₍₁₋₃₎alkyl-CN, —C₍₃₋₅₎cycloalkyl, —C₍₁₋₃₎alkyl-O—C₍₁₋₃₎ alkyl, or —C₍₁₋₃₎alkyl-N(C₍₁₋₃₎alkyl)₂, wherein the —C₍₁₋₃₎alkyl is unsubstituted or substituted with one to six R^{1aa} groups;
R^{1d} is independently for each occurrence —OH, halo, —C₍₁₋₃₎alkyl, —C₍₃₋₅₎cycloalkyl, —C₍₁₋₃₎alkyl-O—C₍₁₋₃₎alkyl or —C₍₁₋₃₎alkyl-N—(C₍₁₋₃₎alkyl)₂ wherein the —C₍₁₋₃₎alkyl is unsubstituted or substituted with one to six R^{1aa} groups;
R^{1aa} independently for each occurrence is halo;
R^{2a} is H, —OH, —C₍₁₋₃₎alkyl, —C₍₃₋₅₎cycloalkyl, —C₍₁₋₃₎alkyl-O—C₍₁₋₃₎alkyl, —C₍₁₋₃₎alkyl-O—C₍₃₋₅₎ cycloalkyl, or 4- to 6-membered heterocyclyl, wherein the —C₍₁₋₃₎alkyl, —C₍₃₋₅₎cycloalkyl, —C₍₁₋₃₎ alkyl-O—C₍₁₋₃₎alkyl, and —C₍₁₋₃₎alkyl-O—C₍₃₋₅₎cycloalkyl groups are unsubstituted or substituted with one to six R^{2aa} groups; or R^{2a} and R^{1c} are taken together with the carbon atoms to which they are attached to form a C₍₃₋₅₎cycloalkyl or a 4- to 6-membered heterocyclyl;
R^{2aa} independently for each occurrence is halo, —OH, or —CN;
R^{2b} is H or —C₍₁₋₃₎alkyl; or R^{2a} and R^{2b} are taken together with the carbon atom to which they are attached to form a C═O;
R³ is —C₍₁₋₁₀₎alkyl, —C₍₁₋₆₎alkyl-O—C₍₁₋₆₎alkyl, —C₍₁₋₆₎alkyl-O—C₍₃₋₅₎cycloalkyl, —C₍₁₋₆₎alkyl-O—C₍₃₋₅₎cycloalkyl-C₍₁₋₃₎alkyl, —C₍₃₋₅₎cycloalkyl, or —C₍₁₋₃₎alkyl-(C₍₃₋₅₎cycloalkyl)₁₋₂, each of which is unsubstituted or substituted with one to six halo atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, —C₍₁₋₆₎alkyl-O—C₍₁₋₆₎alkyl, and —C₍₀₋₂₎alkyl-C₍₃₋₆₎ cycloalkyl are unsubstituted or substituted with one to six substituents independently selected from halo, —CH₃, —CH₂F, —CHF₂, and —CF₃;
n is 1, 2 or 3; and
m is 0, 1, 2, 3, or 4.
2. The compound of embodiment 1, or a pharmaceutically acceptable salt thereof, wherein:
   R^{1a} and R^{1b} are each independently H, —C₍₁₋₃₎alkyl, or —C₍₃₋₅₎cycloalkyl, wherein the —C₍₁₋₃₎ alkyl is unsubstituted or substituted with one to six R^{1aa} groups; or R^{1a} and R^{1b} together with the carbon atom to which they are attached form a spiro C₍₃₋₅₎cycloalkyl or a spiro heterocyclyl selected from

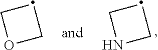

wherein · indicates the carbon atom to which R^{1a} and R^{1b} are attached; or R^{1a} and R^{1c} are taken together to form a C₍₁₋₂₎alkylenyl bridge;
R^{1c} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups;
R^{1d} is independently for each occurrence halo, —C₍₁₋₃₎alkyl, or —C₍₃₋₅₎cycloalkyl, wherein the C₍₁₋₃₎alkyl is unsubstituted or substituted with one to six R^{1aa} groups;
R^{1aa} independently for each occurrence is fluorine;
R^{2a} is H, —OH, or —C₍₁₋₃₎alkyl, wherein the —C₍₁₋₃₎alkyl is unsubstituted or substituted with one to six R^{2aa} groups; or R^{2a} and R^{1c} are taken together with the carbon atoms to which they are attached to form a C₍₃₋₅₎cycloalkyl or a 4- to 6-membered heterocycle;
R^{2aa} independently for each occurrence is fluorine, —OH, or —CN;
R^{2b} is H or —C₍₁₋₃₎alkyl; or R^{2a} and R^{2b} are taken together with the carbon atom to which they are attached to form a C═O;
R³ is —C₍₁₋₁₀₎alkyl, —C₍₁₋₁₀₎alkyl-O—C₍₁₋₆₎alkyl, —C₍₁₋₆₎alkyl-O—C₍₃₋₅₎cycloalkyl, —C(−)alkyl-O—C₍₃₋₅₎cycloalkyl-C₍₁₋₃₎alkyl, —C₍₃₋₅₎cycloalkyl, or —C₍₁₋₃₎alkyl-(C₍₃₋₅₎cycloalkyl)₁₋₂, each of which is unsubstituted or substituted with one to six fluorine atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, —C₍₁₋₆₎alkyl-O—C₍₁₋₆₎alkyl, and —C₍₀₋₂₎alkyl-C₍₃₋₆₎cycloalkyl are unsubstituted or substituted with one to six substituents independently selected from fluorine, —CH₃, —CH₂F, —CHF₂, and —CF₃;
n is 1, 2 or 3; and
m is 0, 1, 2, 3, or 4.
3. The compound of embodiment 1 or embodiment 2, or a pharmaceutically acceptable salt thereof, wherein:
   R^{1a} is H, —C₍₁₋₃₎alkyl, or —C₍₃₋₅₎cycloalkyl, wherein the —C₍₁₋₃₎alkyl is unsubstituted or substituted with one to six R^{1aa} groups; or R^{1a} and R^{1c} are taken together to form a C₍₁₋₂₎alkylenyl bridge;
   R^{1b} is H or —C₍₁₋₃₎alkyl, wherein the —C₍₁₋₃₎alkyl is unsubstituted or substituted with one to six R^{1aa} groups; or R^{1a} and R^{1b} together with the carbon atom to which they are attached form a spiro C₍₃₋₅₎cycloalkyl or a spiro heterocyclyl selected from

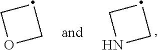

wherein · indicates the carbon atom to which R^{1a} and R^{1b} are attached;
R^{1c} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups;
R^{1d} is independently for each occurrence halo, —C₍₁₋₃₎alkyl, or —C₍₃₋₅₎cycloalkyl, wherein the —C₍₁₋₃₎alkyl is unsubstituted or substituted with one to six R^{1aa} groups;
R^{1aa} independently for each occurrence is fluorine;
R^{2a} is H, —OH, or —C₍₁₋₃₎alkyl, wherein the —C₍₁₋₃₎alkyl is unsubstituted or substituted with one to six R^{2aa} groups; or R^{2a} and R^{1c} are taken together with the carbon atoms to which they are attached to form a C₍₃₋₅₎cycloalkyl or a 4- to 6-membered heterocycle;
R^{2aa} independently for each occurrence is fluorine or —OH;
R^{2b} is H or —C₍₁₋₃₎alkyl; or R^{2a} and R^{2b} are taken together with the carbon atom to which they are attached to form a C═O;
R³ is —C₍₁₋₁₀₎alkyl, —C₍₁₋₆₎alkyl-O—C₍₁₋₆₎alkyl, —C₍₁₋₆₎alkyl-O—C₍₃₋₅₎cycloalkyl, or —C₍₃₋₈₎ cycloalkyl, each of which is unsubstituted or substituted with one to six fluorine atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, —C₍₁₋₆₎alkyl-O—C₍₁₋₆₎alkyl, and —C₍₀₋₂₎alkyl-C₍₃₋₆₎cycloalkyl are unsubstituted or substituted with one to six substituents independently selected from fluorine, —CH₃, —CH₂F, —CHF₂, and —CF₃;
n is 1, 2 or 3; and
m is 0, 1, 2, 3, or 4.
4. The compound of any one of embodiments 1-3, or a pharmaceutically acceptable salt thereof, wherein:
   R^{1a} is H, —C₍₁₋₃₎alkyl, or —C₍₃₋₅₎cycloalkyl, wherein the —C₍₁₋₃₎alkyl is unsubstituted or substituted with one to six R^{1aa} groups; or R^{1a} and R^{1c} are taken together to form a C₍₁₋₂₎alkylenyl bridge;
   R^{1b} is H or —C₍₁₋₃₎alkyl, wherein the —C₍₁₋₃₎alkyl is unsubstituted or substituted with one to six R^{1aa} groups; or R^{1a} and R^{1b} together with the carbon atom to which they are attached form a spiro C₍₃₋₅₎cycloalkyl or a spiro heterocyclyl selected from

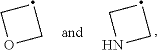

wherein · indicates the carbon atom to which R^{1a} and R^{1b} are attached;
R^{1c} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups;
R^{1d} is independently for each occurrence halo or —C₍₁₋₃₎alkyl, wherein the —C₍₁₋₃₎alkyl is unsubstituted or substituted with one to six R^{1aa} groups;
R^{1aa} independently for each occurrence is fluorine;
R^{2a} is H, —OH, or —C₍₁₋₃₎alkyl, wherein the —C₍₁₋₃₎alkyl is unsubstituted or substituted with one to six R^{2aa} groups; or R^{2a} and R^{1c} are taken together with the carbon atoms to which they are attached to form a C₍₃₋₅₎cycloalkyl or a 4- to 6-membered heterocycle;
R^{2aa} independently for each occurrence is fluorine or —OH;
R^{2b} is H or —C₍₁₋₃₎alkyl; or R^{2a} and R^{2b} are taken together with the carbon atom to which they are attached to form a C═O;
R³ is —C₍₁₋₁₀₎alkyl or —C₍₃₋₈₎cycloalkyl, each of which is unsubstituted or substituted with one to six fluorine atoms;
R⁴ is a 5-membered heteroaryl that is unsubstituted or substituted with one to two R^{4a} groups;
R^{4a} is —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎cycloalkyl;
n is 1, 2 or 3; and
m is 0, 1, or 2.
5. The compound of any one of embodiments 1-4, or a pharmaceutically acceptable salt thereof, wherein:
   R^{1a} is H, —C₍₁₋₃₎alkyl, or —C₍₃₋₅₎cycloalkyl, wherein the —C₍₁₋₃₎alkyl is unsubstituted or substituted with one to six R^{1aa} groups;
   R^{1b} is H or —C₍₁₋₃₎alkyl, wherein the —C₍₁₋₃₎alkyl is unsubstituted or substituted with one to six R^{1aa} groups;
   R^{1c} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups;
   R^{1d} is independently for each occurrence halo or —C₍₁₋₃₎alkyl, wherein the —C₍₁₋₃₎alkyl is unsubstituted or substituted with one to six R^{1aa} groups;
   R^{1aa} independently for each occurrence is fluorine;
   R^{2a} is H, —OH, or —C₍₁₋₃₎alkyl, wherein the —C₍₁₋₃₎alkyl is unsubstituted or substituted with one to six R^{2aa} groups;
   R^{2aa} independently for each occurrence is fluorine;
   R^{2b} is H or —C₍₁₋₃₎alkyl; or R^{2a} and R^{2b} are taken together with the carbon atom to which they are attached to form a C═O;
   R³ is —C₍₄₋₁₀₎alkyl or cyclohexyl, each of which is unsubstituted or substituted with one to six fluorine atoms;
   R⁴ is a 5-membered heteroaryl that is unsubstituted or substituted with one to two R^{4a} groups;
   R^{4a} is —C₍₁₋₆₎alkyl, —O—C₍₁₋₆₎alkyl, —C₍₁₋₆₎alkyl-O—C₍₁₋₆₎alkyl, or —C₍₃₋₆₎cycloalkyl;
   n is 1, 2 or 3; and
   m is 0, 1, or 2.
6. The compound of any one of embodiments 1-5, or a pharmaceutically acceptable salt thereof, wherein:
   R^{1a} is H or —C₍₁₋₃₎alkyl, wherein the —C₍₁₋₃₎alkyl is unsubstituted or substituted with one to six R^{1aa} groups;
   R^{1b} is H or —C₍₁₋₃₎alkyl, wherein the —C₍₁₋₃₎alkyl is unsubstituted or substituted with one to six R^{1aa} groups;
   R^{1c} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂;
   R^{1d} is independently for each occurrence halo or —C₍₁₋₃₎alkyl, wherein the —C₍₁₋₃₎alkyl is unsubstituted or substituted with one to six R^{1aa} groups;
   R^{1a} independently for each occurrence is fluorine;
   R^{2a} is H, —OH, or —C₍₁₋₃₎alkyl;
   R^{2b} is H or —C₍₁₋₃₎alkyl;
   R³ is —C₍₄₋₁₀₎alkyl or cyclohexyl, each of which is substituted with one to six fluorine atoms;
   R⁴ is a 5-membered heteroaryl that is unsubstituted or substituted with one to two R^{4a} groups;
   R^{4a} is —C₍₁₋₆₎alkyl, —O—C₍₁₋₆₎alkyl, —C₍₁₋₆₎alkyl-O—C₍₁₋₆₎alkyl, or —C₍₃₋₆₎cycloalkyl;
   n is 1 or 2; and
   m is 0, 1, or 2.
7. The compound of any one of embodiments 1-6, or a pharmaceutically acceptable salt thereof, wherein:
   R^{1a} is —C₍₁₋₃₎alkyl, wherein the —C₍₁₋₃₎alkyl is unsubstituted or substituted with one to three R^{1aa} groups;
   R^{1b} is H or —C₍₁₋₃₎alkyl, wherein the —C₍₁₋₃₎alkyl is unsubstituted or substituted with one to three R^{1aa} groups;
   R^{1c} is H, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂;
   R^{1aa} independently for each occurrence is fluorine;
   R^{2a} is H or —OH;
   R^{2b} is H;
   R³ is —C₍₄₋₈₎alkyl substituted with one to three fluorine atoms or

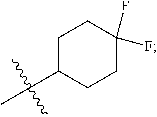 [SMILES: *C1CCC(F)(F)CC1]

R⁴ is

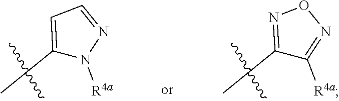

R^{4a} is —C₍₁₋₆₎alkyl, —O—C₍₁₋₆₎alkyl, or —C₍₃₋₆₎cycloalkyl;
n is 1; and
m is 0.
8. The compound of any one of embodiments 1-6, or a pharmaceutically acceptable salt thereof, having a structure selected from the group consisting of;

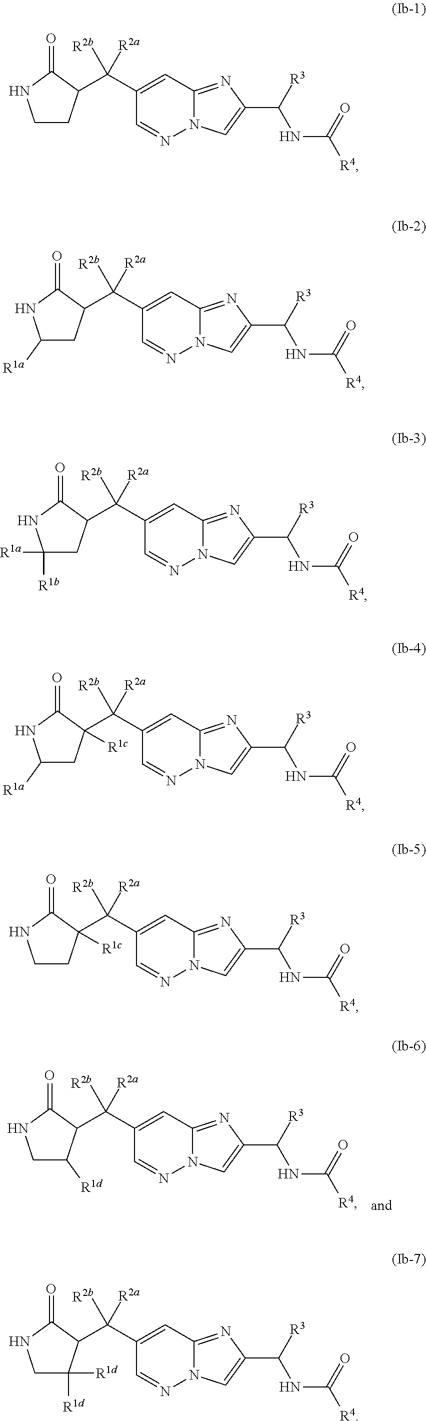

9. The compound of any one of embodiments 1-6, or a pharmaceutically acceptable salt thereof, having a structure selected from the group consisting of:

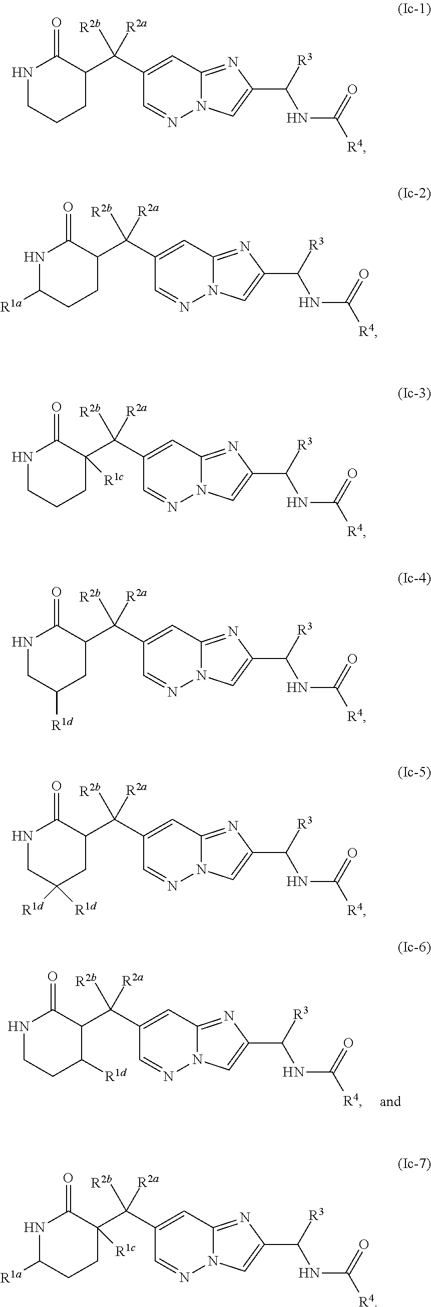

10. The compound of any one of embodiments 1-5, or a pharmaceutically acceptable salt thereof, having a structure selected from the group consisting of:

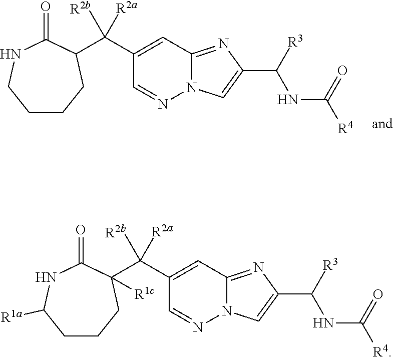

11. The compound of any one of embodiments 1-4 or 8-10, or a pharmaceutically acceptable salt thereof, wherein:
   R^{1a} is H, —C₍₁₋₃₎alkyl, or —C₍₃₋₅₎cycloalkyl, wherein the —C₍₁₋₃₎alkyl is unsubstituted or substituted with one to six fluorine atoms; and
   R^{1b} is H or —C₍₁₋₃₎alkyl, wherein the —C₍₁₋₃₎alkyl is unsubstituted or substituted with one to six fluorine atoms.
12. The compound of any one of embodiments 1-5 or 8-11, or a pharmaceutically acceptable salt thereof, wherein:
   R^{1a} is H, —CH₃, —CH₂CH₃, —CH(CH₃)₂, —CF₃, or cyclopropyl; and
   R^{1b} is H, —CH, or —CF₃.
13. The compound of any one of embodiments 1-6 or 8-11, or a pharmaceutically acceptable salt thereof, wherein R^{1a} is H or —C₍₁₋₃₎alkyl, wherein the —C₍₁₋₃₎alkyl is unsubstituted or substituted with one to three fluorine atoms.
14. The compound of any one of embodiments 1-6 or 8-13, or a pharmaceutically acceptable salt thereof, wherein R^{1a} is H, —CH₃, or —CF₃.
15. The compound of any one of embodiments 1-7 or 8-14, or a pharmaceutically acceptable salt thereof, wherein R^{1b} is H or —CH₃.
16. The compound of any one of embodiments 1-7 or 8-15, or a pharmaceutically acceptable salt thereof, wherein R^{1c} is halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₍₁₋₃₎alkyl)₂.
17. The compound of any one of embodiments 1-7 or 8-15, or a pharmaceutically acceptable salt thereof, wherein R^{1c} is H, —C₍₁₋₃₎alkyl-CN, or —C₍₁₋₃₎alkyl-O—C₍₁₋₃₎alkyl.
18. The compound of any one of embodiments 1-7 or 8-15, or a pharmaceutically acceptable salt thereof, wherein R^{1c} is H, fluorine, —OH, —CH₃, —CH₂CN, —CH₂OCH₃, or —CH₂N(CH₃)₂.
19. The compound of any one of embodiments 1-6 or 8-18, or a pharmaceutically acceptable salt thereof, wherein R^{1d} is independently for each occurrence halo or —C₍₁₋₃₎alkyl, wherein the —C₍₁₋₃₎alkyl is unsubstituted or substituted with one to three fluorine atoms.
20. The compound of any one of embodiments 1-6 or 8-19, or a pharmaceutically acceptable salt thereof, wherein R^{1d} is independently for each occurrence F, —CH₃, or —CF₃.
21. The compound of any one of embodiments 1-5, 8, 9, or 11-20, or a pharmaceutically acceptable salt thereof, wherein n is 1 or 2.
22. The compound of any one of embodiments 1-6, 8, or 11-21, or a pharmaceutically acceptable salt thereof, wherein n is 1.
23. The compound of any one of embodiments 1-6 or 8-22, or a pharmaceutically acceptable salt thereof, wherein m is 0, 1, or 2.
24. The compound of any one of embodiments 1-4, having a structure according to Formula Ie:

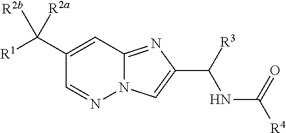

or a pharmaceutically acceptable salt thereof,
   wherein R¹ is selected from the group consisting of:

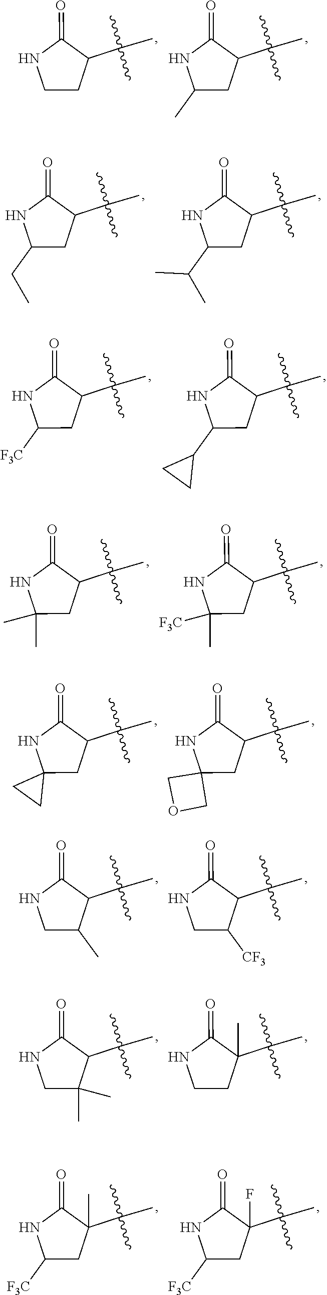

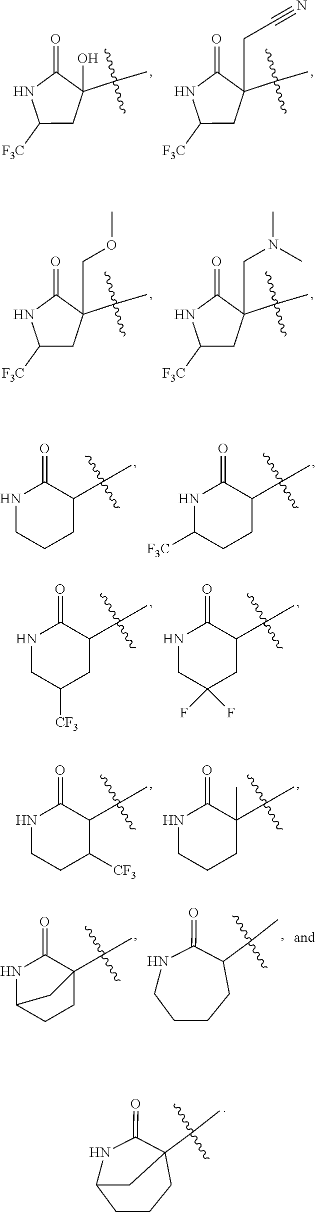

25. The compound of embodiment 24, or a pharmaceutically acceptable salt thereof, wherein R¹ is selected from the group consisting of:

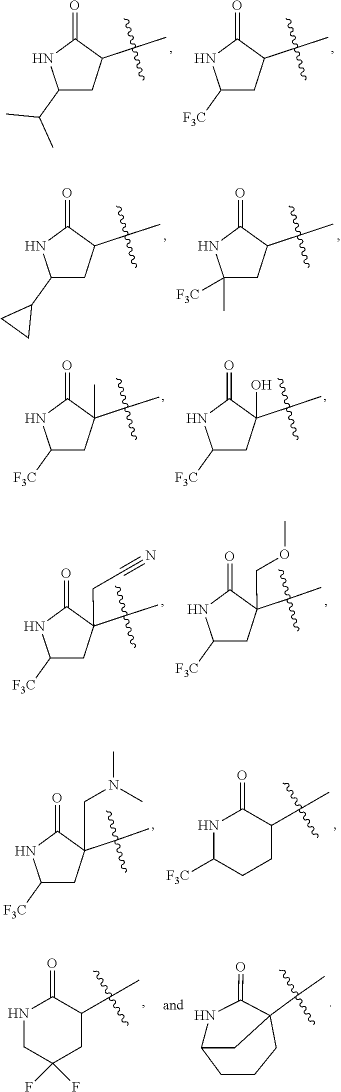

26. The compound of embodiment 24 or embodiment 25, or a pharmaceutically acceptable salt thereof, wherein R¹ is selected from the group consisting of:

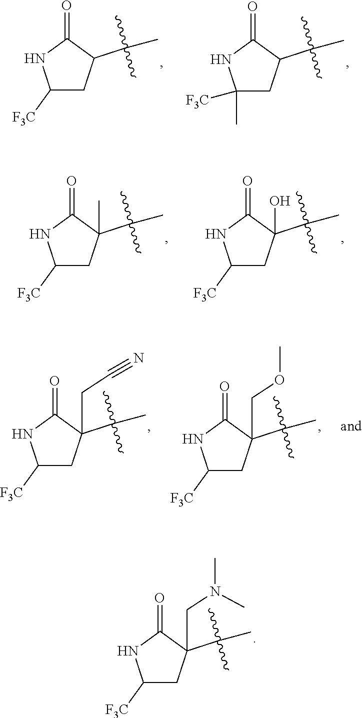

27. The compound of any one of embodiments 1-5 or 8-26, or a pharmaceutically acceptable salt thereof, wherein:
   R^{2a} is H, —OH, or —C₍₁₋₃₎alkyl; and
   R^{2b} is H or —C₍₁₋₃₎alkyl; or
   R^{2a} and R^{2b} are taken together with the carbon atom to which they are attached to form a C═O.
28. The compound of any one of embodiments 1-5 or 8-27, or a pharmaceutically acceptable salt thereof, wherein:
   R^{2a} is H, and R^{2b} is H;
   R^{2a} is —OH, and R^{2b} is H,
   R^{2a} is —CH₃, and R^{2b} is H;
   R^{2a} is —CH₂CH₃, and R^{2b} is H;
   R^{2a} is —OH, and R^{2b} is —CH₃; or
   R^{2a} and R^{2b} are taken together with the carbon atom to which they are attached to form a C═O.
29. The compound of any one of embodiments 1-6 or 8-28, or a pharmaceutically acceptable salt thereof, wherein R^{2a} is H or —OH, and R^{2b} is H.
30. The compound of any one of embodiments 1-3 or 8-29, or a pharmaceutically acceptable salt thereof, wherein R³ is —C₍₁₋₁₀₎alkyl or —C₍₃₋₈₎cycloalkyl, each of which is unsubstituted or substituted with one to six fluorine atoms.
31. The compound of any one of embodiments 1-6 or 8-30, or a pharmaceutically acceptable salt thereof, wherein R³ is —C₍₄₋₈₎alkyl or cyclohexyl, each of which is substituted with one to six fluorine atoms.
32. The compound of any one of embodiments 1-31, or a pharmaceutically acceptable salt thereof, wherein R³ is

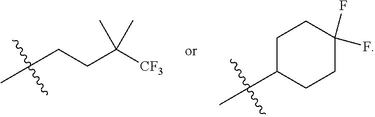

33. The compound of any one of embodiments 1-32, or a pharmaceutically acceptable salt thereof, wherein R³ is

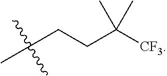 [SMILES: *CCC(C)(C)C(F)(F)F]

34. The compound of any one of embodiments 1-33, or a pharmaceutically acceptable salt thereof, wherein R³ is

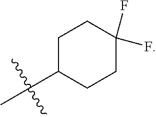 [SMILES: *C1CCC(F)(F)CC1]

35. The compound of any one of embodiments 1-6 or 8-34, or a pharmaceutically acceptable salt thereof, wherein R⁴ is a 5-membered heteroaryl comprising one to three heteroatoms selected from O and N, wherein the heteroaryl is unsubstituted or substituted with one to two R^{4a} groups.
36. The compound of any one of embodiments 1-6 or 8-35, or a pharmaceutically acceptable salt thereof, wherein R⁴ is a 5-membered heteroaryl comprising one to three heteroatoms selected from O and N, wherein the heteroaryl is substituted with one R^{4a} group.
37. The compound of any one of embodiments 1-6 or 8-36, or a pharmaceutically acceptable salt thereof, wherein R⁴ is pyrazolyl or oxadiazolyl that is substituted with one R^{4a} group.
38. The compound of any one of embodiments 1-6 or 8-37, or a pharmaceutically acceptable salt thereof, wherein R⁴ is

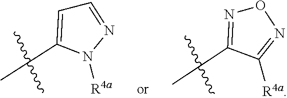

39. The compound of any one of embodiments 1-6 or 8-38, or a pharmaceutically acceptable salt thereof, wherein R^{4a} is —C₍₁₋₆₎alkyl, —O—C₍₁₋₆₎alkyl, or —C₍₃₋₆₎cycloalkyl.
40. The compound of any one of embodiments 1-39, or a pharmaceutically acceptable salt thereof, wherein R⁴ is —CH₃, —CH(CH₃)₂, —OCH₃, or cyclopropyl.
41. The compound of any one of embodiments 1-40, or a pharmaceutically acceptable salt thereof, wherein R⁴ is selected from the group consisting of;

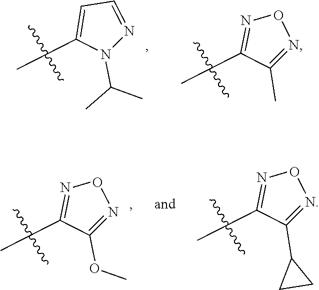

42. The compound of any one of embodiments 1-41, or a pharmaceutically acceptable salt thereof, wherein R⁴ is

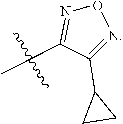 [SMILES: *c1nonc1C1CC1]

43. The compound of any one of embodiments 1-7, having the following structure

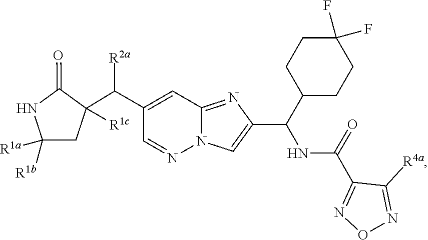

or a pharmaceutically acceptable salt thereof.
44. The compound of any one of embodiments 1-7, having the following structure

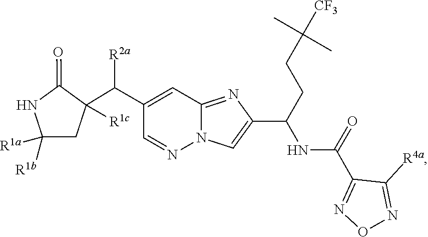

or a pharmaceutically acceptable salt thereof.
45. The compound of any one of embodiment 1, selected from the group consisting of the compounds in Table 1, or a pharmaceutically acceptable salt thereof.
46. The compound of any one of embodiment 45, selected from the group consisting of;

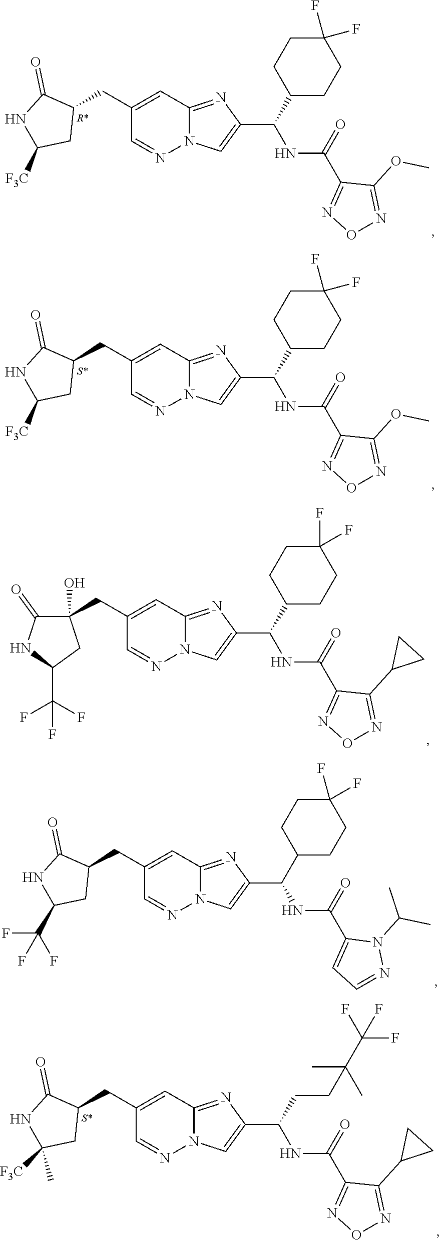

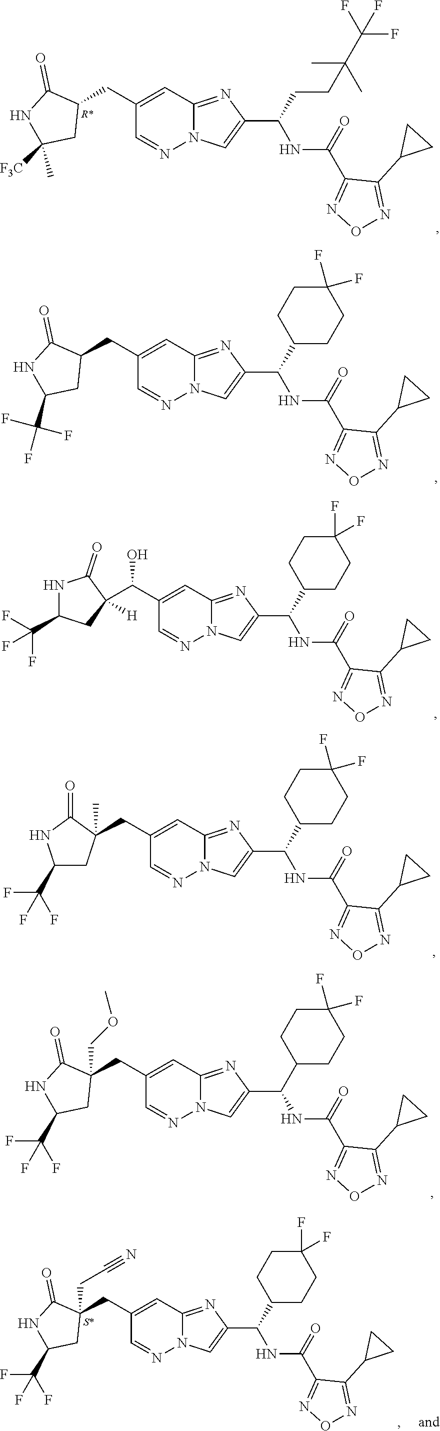

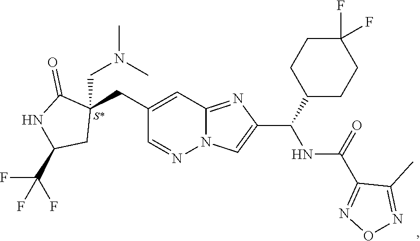 [SMILES: Cc1nonc1C(=O)N[C@H](c1cn2ncc(C[C@@]3(CN(C)C)C[C@@H](C(F)(F)F)NC3=O)cc2n1)C1CCC(F)(F)CC1]

or a pharmaceutically acceptable salt thereof.

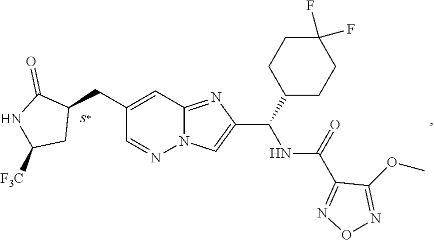 [SMILES: COc1nonc1C(=O)N[C@H](c1cn2ncc(C[C@H]3C[C@@H](C(F)(F)F)NC3=O)cc2n1)C1CCC(F)(F)CC1]

or a pharmaceutically acceptable salt thereof.
47. The compound of embodiment 46, having the following structure:

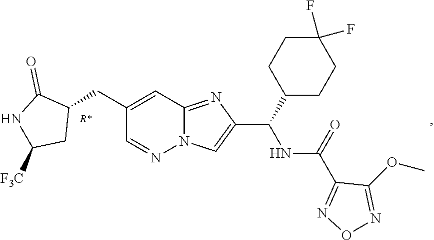 [SMILES: COc1nonc1C(=O)N[C@H](c1cn2ncc(C[C@@H]3C[C@@H](C(F)(F)F)NC3=O)cc2n1)C1CCC(F)(F)CC1]

or a pharmaceutically acceptable salt thereof.
48. The compound of embodiment 46, having the following structure:

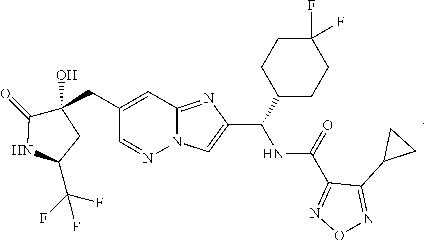 [SMILES: O=C(N[C@H](c1cn2ncc(C[C@@]3(O)C[C@@H](C(F)(F)F)NC3=O)cc2n1)C1CCC(F)(F)CC1)c1nonc1C1CC1]

or a pharmaceutically acceptable salt thereof.
50. The compound of embodiment 46, having the following structure:

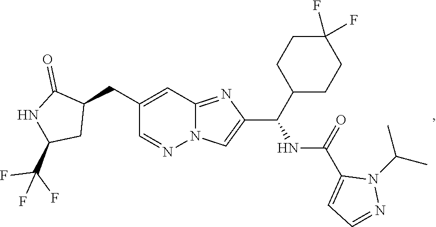 [SMILES: CC(C)n1nccc1C(=O)N[C@H](c1cn2ncc(C[C@H]3C[C@@H](C(F)(F)F)NC3=O)cc2n1)C1CCC(F)(F)CC1]

or a pharmaceutically acceptable salt thereof.
51. The compound of embodiment 46, having the following structure:

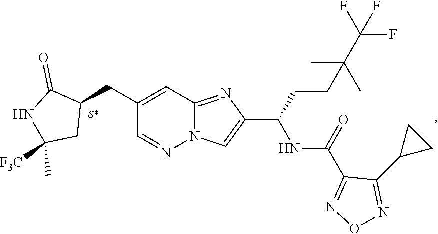 [SMILES: CC(C)(CC[C@H](NC(=O)c1nonc1C1CC1)c1cn2ncc(C[C@H]3C[C@@](C)(C(F)(F)F)NC3=O)cc2n1)C(F)(F)F]

or a pharmaceutically acceptable salt thereof.
52. The compound of embodiment 46, having the following structure:

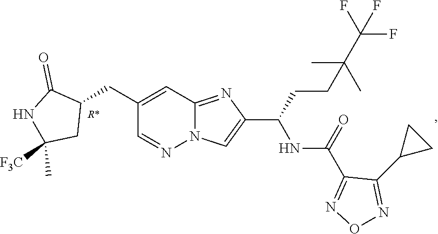 [SMILES: CC(C)(CC[C@H](NC(=O)c1nonc1C1CC1)c1cn2ncc(C[C@@H]3C[C@@](C)(C(F)(F)F)NC3=O)cc2n1)C(F)(F)F]

or a pharmaceutically acceptable salt thereof.
53. The compound of embodiment 46, having the following structure:

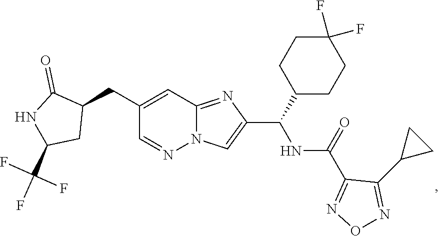 [SMILES: O=C(N[C@H](c1cn2ncc(C[C@H]3C[C@@H](C(F)(F)F)NC3=O)cc2n1)C1CCC(F)(F)CC1)c1nonc1C1CC1]

or a pharmaceutically acceptable salt thereof.
54. The compound of embodiment 46, having the following structure:

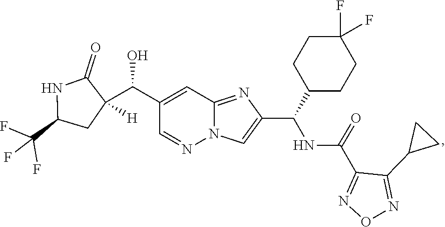 [SMILES: O=C(N[C@H](c1cn2ncc([C@@H](O)[C@H]3C[C@@H](C(F)(F)F)NC3=O)cc2n1)C1CCC(F)(F)CC1)c1nonc1C1CC1]

or a pharmaceutically acceptable salt thereof.
55. The compound of embodiment 46, having the following structure:

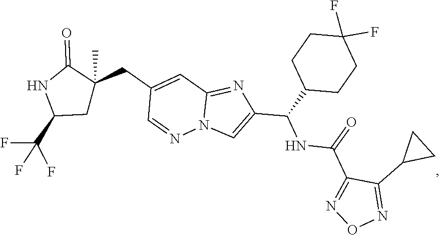 [SMILES: C[C@]1(Cc2cnn3cc([C@@H](NC(=O)c4nonc4C4CC4)C4CCC(F)(F)CC4)nc3c2)C[C@@H](C(F)(F)F)NC1=O]

or a pharmaceutically acceptable salt thereof.
56. The compound of embodiment 46, having the following structure:

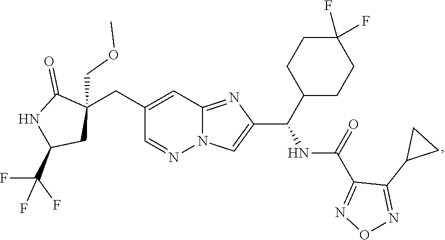 [SMILES: COC[C@@]1(Cc2cnn3cc([C@@H](NC(=O)c4nonc4C4CC4)C4CCC(F)(F)CC4)nc3c2)C[C@@H](C(F)(F)F)NC1=O]

or a pharmaceutically acceptable salt thereof.
57. The compound of embodiment 46, having the following structure:

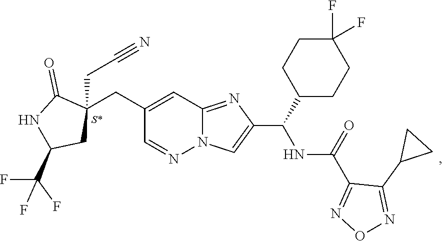 [SMILES: N#CC[C@@]1(Cc2cnn3cc([C@@H](NC(=O)c4nonc4C4CC4)C4CCC(F)(F)CC4)nc3c2)C[C@@H](C(F)(F)F)NC1=O]

or a pharmaceutically acceptable salt thereof.
58. The compound of embodiment 46, having the following structure:

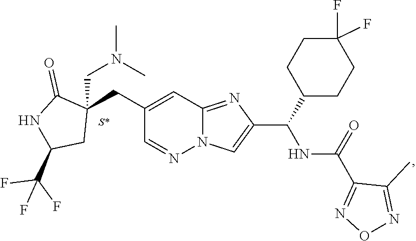 [SMILES: Cc1nonc1C(=O)N[C@H](c1cn2ncc(C[C@@]3(CN(C)C)C[C@@H](C(F)(F)F)NC3=O)cc2n1)C1CCC(F)(F)CC1]

or a pharmaceutically acceptable salt thereof.
59. A pharmaceutical composition, comprising a compound of any one of the preceding embodiments, or a pharmaceutically acceptable salt thereof, and a pharmaceutically acceptable carrier.
60. A pharmaceutical composition made by combining a compound of any one of embodiments 1 to 58, or a pharmaceutically acceptable salt thereof, and a pharmaceutically acceptable carrier.
61. The pharmaceutical composition of embodiment 59 or embodiment 60, or a pharmaceutically acceptable salt thereof, which is administered orally.
62. The pharmaceutical composition of embodiment 61, or a pharmaceutically acceptable salt thereof, which is administered as a tablet or a capsule.
63. A process for making a pharmaceutical composition comprising combining a compound of any one of embodiments 1 to 58, or a pharmaceutically acceptable salt thereof, and a pharmaceutically acceptable carrier.
64. A method for treating and/or ameliorating an IL-17A mediated inflammatory syndrome, disorder, or disease comprising administering to a subject in need thereof a therapeutically effective amount of a compound of any one of embodiments 1 to 58, or a pharmaceutically acceptable salt thereof.
65. The method of embodiment 64, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus.
66. The method of embodiment 65, wherein the IL-17A mediated inflammatory syndrome, disorder, or disease is psoriasis.
67. The method of embodiment 65, wherein the IL-17A mediated inflammatory syndrome, disorder, or disease is psoriatic arthritis.
68. The method of embodiment 65, wherein the IL-17A mediated inflammatory syndrome, disorder, or disease is rheumatoid arthritis.
69. The method of embodiment 65, wherein the IL-17A mediated inflammatory syndrome, disorder, or disease is ankylosing spondylitis.
70. The method of embodiment 65, wherein the IL-17A mediated inflammatory syndrome, disorder, or disease is hidradenitis suppurativa.
71. The method of embodiment 65, wherein the IL-17A mediated inflammatory syndrome, disorder, or disease is bullous pemphigoid.
72. The method of embodiment 65, wherein the IL-17A mediated inflammatory syndrome, disorder, or disease is atopic dermatitis.
73. The method of embodiment 65, wherein the IL-17A mediated inflammatory syndrome, disorder, or disease is vitiligo.
74. The method of embodiment 65, wherein the IL-17A mediated inflammatory syndrome, disorder, or disease is multiple sclerosis.
75. The method of embodiment 65, wherein the IL-17A mediated inflammatory syndrome, disorder, or disease is systemic lupus erythematosus.
76. The method of embodiment 65, wherein the IL-17A mediated inflammatory syndrome, disorder, or disease is asthma.
77. The method of embodiment 65, wherein the IL-17A mediated inflammatory syndrome, disorder, or disease is uveitis.
78. The method of embodiment 65, wherein the IL-17A mediated inflammatory syndrome, disorder, or disease is chronic obstructive pulmonary disorder.
79. The method of embodiment 65, wherein the IL-17A mediated inflammatory syndrome, disorder, or disease is multiple myeloma.
80. The method of any of embodiments 64-79, wherein the compound of any one of embodiments 1-58, or a pharmaceutically acceptable salt thereof, is administered orally.
81. The method of any of embodiments 64-80, wherein the compound of any one of embodiments 1-58, or a pharmaceutically acceptable salt thereof, is administered as a tablet or a capsule.
82. A compound as described herein.
83. A method as described herein.

### EXAMPLES

### Abbreviations

Herein and throughout the application, the following abbreviations may be used.
Å angstrom
Ac acetyl
ACN acetonitrile
AIBN 2,2′-azobis(2-methylpropionitrile)
atm atmosphere
Boc tert-butyloxycarbonyl
br broad
Bu butyl
CDI 1,1′-carbonyldiimidazole
δ NMR chemical shift in parts per million downfield from a standard
d doublet
d day(s)
dba dibenzylideneacetone
DBU 1,8-diazabicyclo[5.4.0]undec-7-ene
DCM dichloromethane
DEA diethylamine
DEAD diethyl azodicarboxylate
DIPEA N,N-diisopropylethylamine (Hünig's base)
DMAP 4-(dimethylamino)pyridine
DMF N,N-dimethylformamide
DMP Dess-Martin Periodinane
DMSO dimethyl sulfoxide
dr diastereomeric ratio
EDCI 1-ethyl-3-(3-dimethylaminopropyl) carbodiimide hydrochloride
Eschenmoser's salt N,N-dimethylmethyleneiminium iodide
ESI electrospray ionization
Et ethyl
EtOAc ethyl acetate
g gram(s)
h hour(s)
HOBt 1-hydroxybenzotriazole
HPLC high pressure liquid chromatography
Hz Hertz
I iso
IPA isopropanol
J coupling constant (NMR spectroscopy)
L liter(s)
LC liquid chromatography
LDA lithium diisopropylamide
LiHMDS lithium bis(trimethylsilyl)amide
m milli or multiplet
m/z mass-to-charge ratio
M⁺ parent molecular ion
M molar (moles/liter)
Me methyl
min minute(s)
μ micro
MOM methoxymethyl
MS mass spectrometry
n normal
N normal (equivalent concentration)
NMR nuclear magnetic resonance
Nos 2-nitrobenzenesulfonyl
P para
Ph phenyl
psi pounds per square inch
q quartet
rt room temperature
RuPhos Pd G3 (2-dicyclohexylphosphino-2′,6′-diisopropoxy-1,1′-biphenyl)[2-(2′-amino-1,1′-biphenyl)]palladium(II) methanesulfonate
(S,S)-(salen)Co(IIIXOAc) (S,S)—(+)-N,N′-Bis(3,5-di-tert-butylsalicylidene)-1,2-cyclohexanediaminocobalt(III) acetate
s singlet
SFC supercritical fluid chromatography
t triplet or tert
T₃P 1-propanephosphonic anhydride
TEA triethylamine
Tf trifluoromethanesulfonate
TFA trifluoroacetic acid
THF tetrahydrofuran
TLC thin layer chromatography
Ts p-toluenesulfonyl
w/v weight by volume

In some embodiments, provided herein are processes and intermediates disclosed herein that are useful for preparing a compound of the disclosure or pharmaceutically acceptable salts thereof. In the schemes below, the term PG is intended to mean “protecting group”, such as those described in T. W. Greene and P. G. M. Wuts, “Protective Groups in Organic Synthesis,” 3 ed., John Wiley & Sons, 1999. For example, a group PG attached to a nitrogen atom indicates a nitrogen protecting group, such as tert-butyloxycarbonyl (Boc), toluenesulfonyl (tosyl, Ts), or nitrobenzenesulfonyl (nosyl, Ns).

NMR spectra were recorded on a Bruker 600 MHz instrument equipped with a 5 mm BBO probe or a 500 MHz instrument with a 5 mm BBFO probe at 300K in DMSO-d₆. For ¹H NMR signal assignments and relative stereochemistry determination, spectra of 1D 1H, 1D 13C, 2D correlation spectroscopy (COSY), ¹H-¹³C heteronuclear single quantum correlation (HSQC), heteronuclear multiple bond correlation (HMBC), nuclear Overhauser effect spectroscopy (NOESY) and ¹H-¹⁹F heteronuclear Overhauser effect spectroscopy (HOESY) were acquired using the standard pulse sequences available in TopSpin 4.1 (Bruker Inc.).

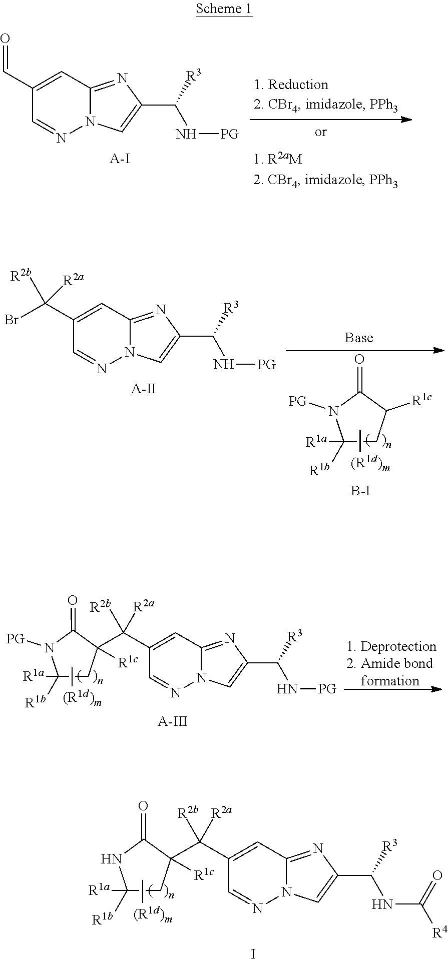

Compounds of Formula I can be prepared according to Scheme 1. Compounds A-I can be converted to compounds A-II through a two-step sequence of i) reduction of the aldehyde with a reagent such as sodium borohydride in solvents such as DCM and MeOH to provide the corresponding alcohol (structure not shown), followed by ii) bromination of the alcohol with reagents such as CBr₄ and PPh₃ with additives such as imidazole in a solvent such as DCM. Alternatively, aldehyde A-I can be treated with an agent such as R^{2a}M, for example, MeMgBr, in a solvent such as THF, followed by bromination with reagents such as CBr₄ with additives such as imidazole and PPh₃, in a solvent such as DCM to provide compounds A-II. Compounds A-Ill may be prepared by treating bromides A-II with lactams B-I in the presence of a base, such as LiHMDS, in a solvent such as THF. Compounds A-III can then be converted to compounds of Formula I by deprotection of compounds A-III and then amide bond formation at the primary amine. For example when the protecting group (PG) is a Boc group, compounds A-III can be treated with an acid such as TFA in a solvent such as DCM to afford the corresponding amines (structure not shown). Amide bond formation at the primary amine and carboxylic acids (R⁴CO₂H) can be achieved through the use of a coupling agent, such as HATU, EDCI or 2-chloro-1-methylpyridinium iodide in the presence of a base, such as DIPEA or TEA, in a solvent, such as DMF, MeCN, or DCM, with or without an additive, such as HOBt, to yield compounds of Formula I. Alternatively, amide bond formation at the primary amine can be achieved by treatment with N-hydroxysuccinate esters in the presence of reagents such as DIPEA in a solvent such as acetonitrile to provide compounds of Formula I.

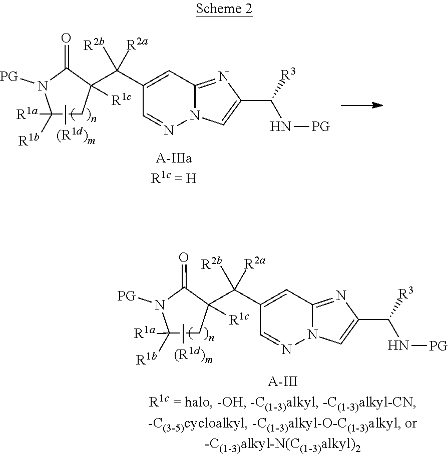

Substituted lactams A-III can also be prepared as shown in Scheme 2. Compounds A-ma wherein R^{1c} is H may be treated with agents such as methyl iodide, chloromethyl methyl ether, bromoacetonitrile, N-fluorobenzenesulfonimide or 3-phenyl-2-(phenylsulfonyl)-1,2-oxaziridine in the presence of a base, such as LiHMDS or K₂CO₃, and a solvent such as THF or DMF to afford substituted lactams A-III.

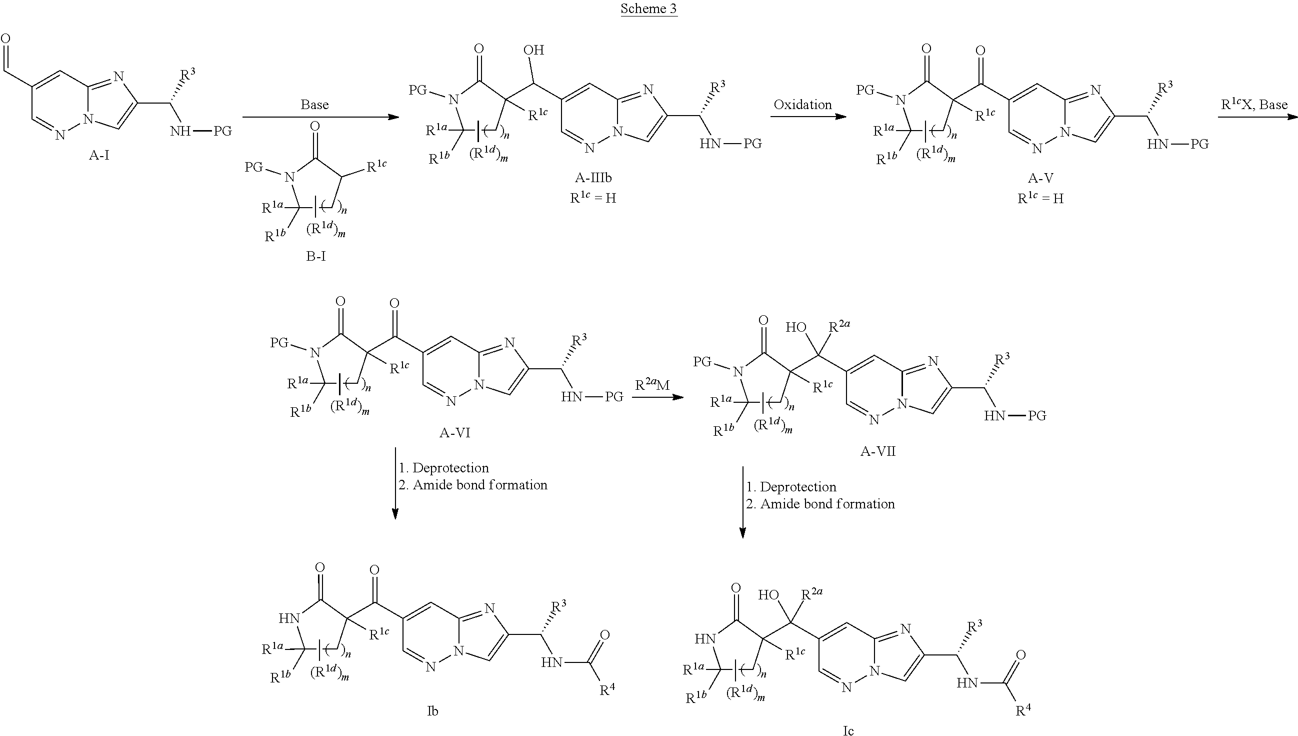

Compounds of Formula Ib and Ic may be prepared as shown in Scheme 3. Lactams A-IIIb may be prepared by treating aldehydes A-1 with lactams B-I in the presence of a base, such as LiHMDS, in a solvent such as THF. Compounds A-IIIb are then converted to ketones A-V by treatment with reagents such as DMP in solvents such as DCM. Treatment of ketones A-V with alkylating reagents such as methyl iodide in the presence of a base, such as K₂CO₃ and a solvent such as DMF affords lactams A-VT. Addition of suitable nucleophiles, such as R^{2a}M where M is Li, MgCl, or MgBr to lactams A-VT then affords the corresponding alcohols, A-VII. Compounds A-VI and A-VII can be converted to compounds of Formula I using the methods described in Scheme 1 for the conversion of compounds A-III to compounds of Formula I.

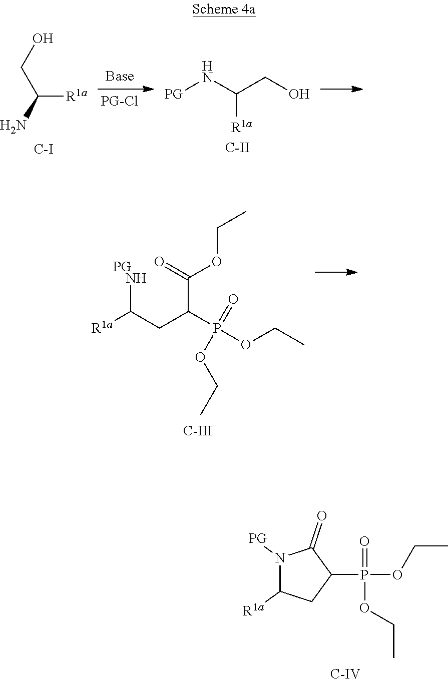

Compounds C-II can be prepared from compounds C-I by protection of the free amine with a protecting group (PG). For example, compounds C-I can be treated with a reagent such as TsCl or 4-nitrobenzenesulfonyl chloride, in the presence of a base, such as pyridine or DIPEA, and a solvent such as DCM. Phosphonates C-IV may be prepared by several methods, two of such methods are described in Scheme 4a.
Method 1: In a two-step procedure, alcohols C-II can be cyclized by treatment with a reagent, such as DIAD or DEAD, additives such as PPh₃ and solvents such as THF to form an aziridine (structure not shown) followed by formation of the phosphonate ester via reaction with triethyl phosphonoacetate, a base such as KOtBu and a solvent such as THF to afford compounds C-III. Phosphonates C-IV can be prepared by treating compounds C-III with reagents such as AlMe₃ in solvents such as IPA, MeOH and toluene.
Method 2: Phosphonate esters C-III can be prepared by treating alcohols C-I with NosCl in the presence of a base such as DIPEA in a solvent such as DCM, followed by treatment with ethyl 2-(diethoxyphosphoryl)acetate, a base such as KOtBu and a solvent such as THF. Hydrolysis of the ester in compounds C-III by reaction with a base, such as aqueous LiOH in a solvent such as THF affords the corresponding carboxylic acid (structure not shown), which can then be converted to lactams C-IV by treatment with reagents such as 2-chloro-1-methylpyridin-1-ium iodide, in the presence of a base such as DIPEA and a solvent such as DCM.

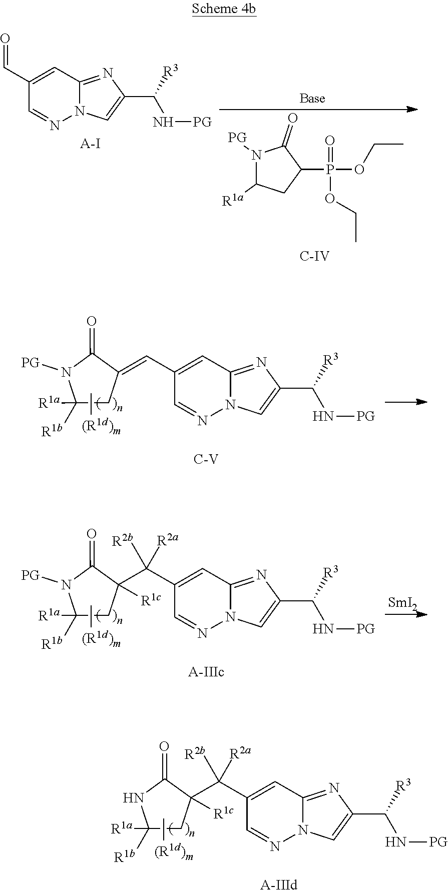

Compounds A-IIId can be prepared as shown in Scheme 4b. Olefins C-V can be prepared by treating aldehydes A-I with phosphonates C-IV in the presence of a base such as NaH, and a solvent such as THF. Hydrogenation of the olefin in compounds C-V by treatment with hydrogen, a reagent such as Pd/C and a solvent such as MeOH or EtOH affords lactams A-Ic. Alternatively, reaction of olefins C-V with suitable nucleophiles, such as R^{2a}M where M is Li, MgCl, or MgBr, in the presence of additives such as BF₃·OEt₂, catalysts such as CuBr·S(CH₃)₂, and a solvent such as DCM affords lactams A-Ic. Compounds A-IIId can be prepared by treating lactams A-IIIc with reagents such as samarium(II) iodide in solvents such as THF. Compounds A-IIId can be converted to compounds of Formula I using the methods described in Scheme I for the conversion of compounds A-III to compounds of Formula I.

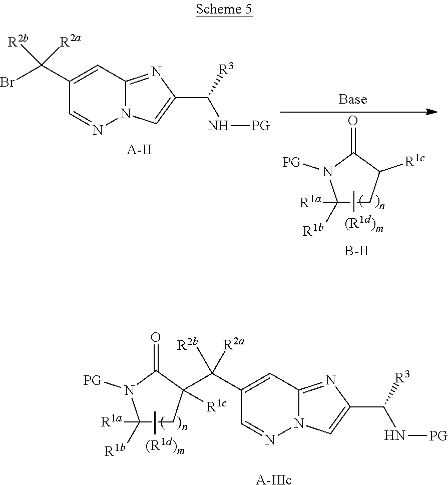

Compounds A-IIIc may also be prepared as shown in Scheme 5. Treatment of bromides A-II with protected lactams B-II (utilizing a protecting group such as a toluenesulfonyl group) in the presence of a base, such as LIHMDS and a solvent such as THF affords compounds A-IIIc.

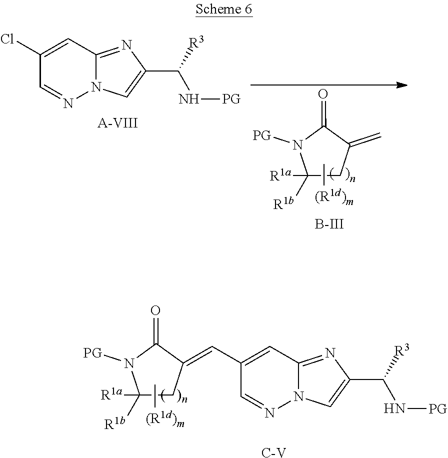

Olefins C-V may also be prepared as shown in Scheme 6. Chlorides A-VIII can be treated with lactams B-III in the presence of a base such as N-cyclohexyl-N-methylcyclohexanamine, a catalyst such as Pd₂(dba)₃ and a solvent, such as 1,4-dioxane, to afford olefins C-V. Lactams B-III may be made in a manner similar to procedures described in J. Med. Chem. 2016, 59, 3, 914-924.

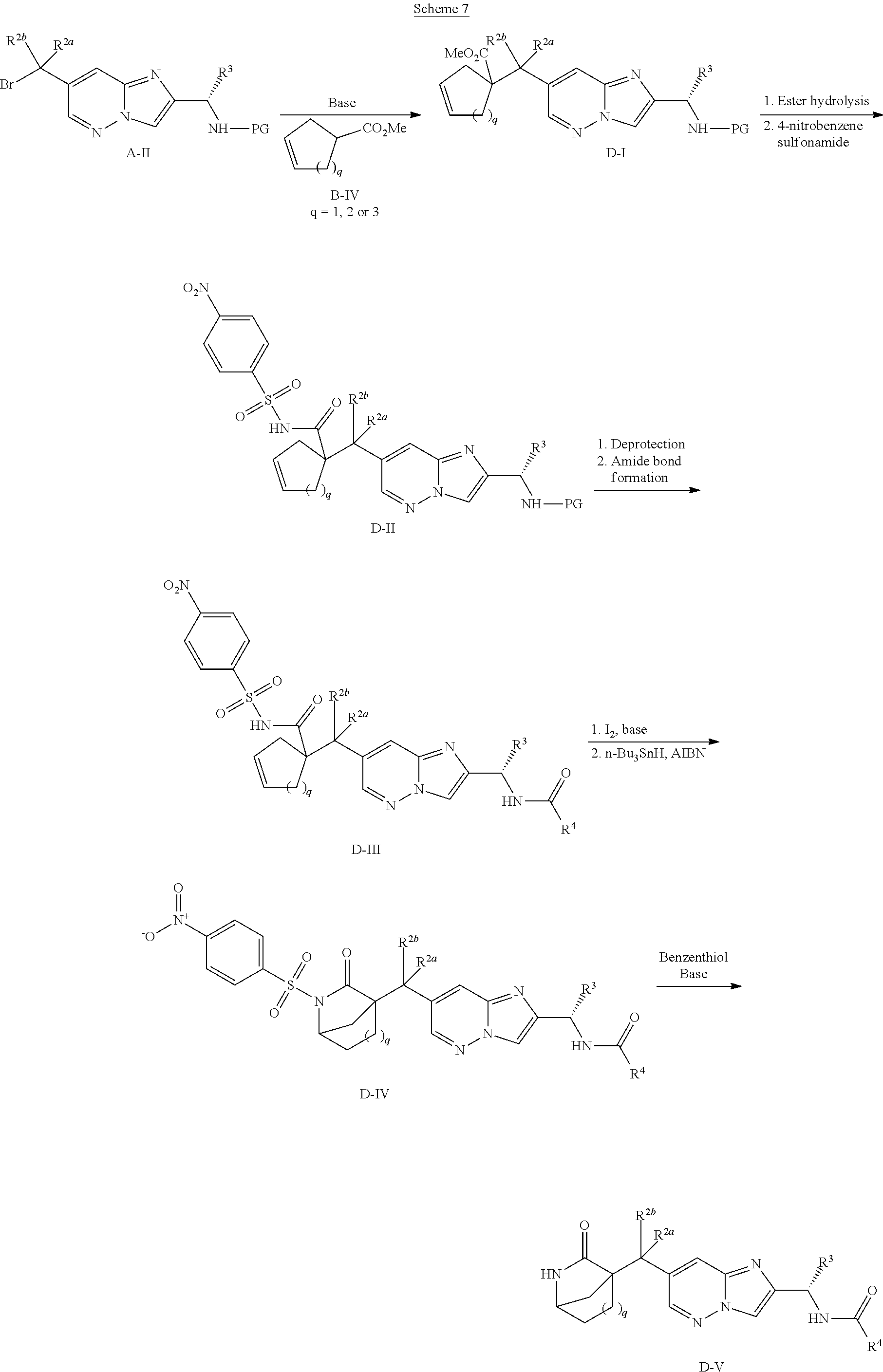

Lactams D-V may be prepared as shown in Scheme 7. Treatment of bromides A-II with esters B-IV in the presence of a base, such as LDA, and a solvent such as THF provides compounds D-I. Esters D-I can then undergo ester hydrolysis by treatment with an aqueous base, such as LiOH, in a solvent such as MeOH to provide the corresponding carboxylic acids (structure not shown). Subsequent reaction of the carboxylic acids with 4-nitrobenzenesulfonamide using coupling conditions with a coupling agent, such as HATU or EDCI in the presence of a base, such as DIPEA or TEA, in a solvent, such as DMF, MeCN, or DCM, with or without an additive, such as HOBt, to yield affords compounds D-II. Compounds D-II can be converted to amides D-III using the methods described in Scheme 1 for the conversion of compounds A-III to compounds of Formula I. Treatment of amides D-III with iodine and a base such as K₂CO₃ in a solvent such as ACN provides the corresponding bicyclic lactams (structure not shown). Subsequent reaction of the bicyclic lactams with tributyltin hydride and AIBN in a solvent such as toluene affords lactams D-IV. Treatment of lactams D-IV with benzenethiol, a base such as K₂CO₃, an additive such as DMSO and solvent such as ACN provides lactams D-V.

### INTERMEDIATES

### Intermediate 1: tert-Butyl (S)-((7-(bromomethyl)imidazo[1,2-b]pyridazin-2-yl)(4,4-difluorocyclohexyl)methyl)carbamate

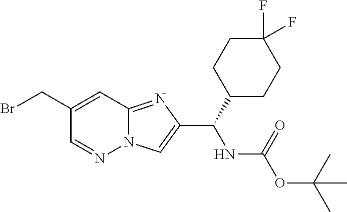 [SMILES: CC(C)(C)OC(=O)N[C@H](c1cn2ncc(CBr)cc2n1)C1CCC(F)(F)CC1]

Step A: tert-Butyl (S)-((4,4-difluorocyclohexyl)(7-(hydroxymethyl)imidazo[1,2-b]pyridazin-2-yl)methyl)carbamate. A solution of tert-butyl (S)-((4,4-difluorocyclohexyl)(7-formylimidazo[1,2-b]pyridazin-2-yl)methyl)carbamate (2.17 g, 5.50 mmol) in DCM/MeOH (5:1, 60 mL) was cooled to 0° C. Then, sodium borohydride (265 mg, 6.90 mmol) was added and the resulting mixture was stirred under an air atmosphere while warming to rt over 2.5 h. The mixture was cooled to 0° C., quenched by the dropwise addition of saturated aqueous NH₄Cl (0.2 mL) and stirred for 30 min while warming to rt. The reaction mixture was partitioned between DCM (100 mL) and water (200 mL), and the aqueous layer was further extracted with DCM (3×30 mL). The organic layers were combined, dried over anhydrous Na₂SO₄, filtered through diatomaceous earth and concentrated to dryness. The residue was purified by silica gel chromatography (0-25% MeOH/DCM) to provide the title compound as a white solid (82% yield).

Step B: tert-Butyl (S)-((7-(bromomethyl)imidazo[1,2-b]pyridazin-2-yl)(4,4-difluorocyclohexyl)methyl)carbamate. To a solution of tert-butyl (S)-((4,4-difluorocyclohexyl(7-(hydroxymethyl)imidazo[1,2-b]pyridazin-2-yl)methyl)carbamate (132 mg, 0.330 mmol, Step A), PPh₃ (105 mg, 0.400 mmol) and imidazole (46 mg, 0.67 mmol) in dry DCM (10 mL) was added a mixture of CBr₄ (221 mg, 0.670 mmol) in dry DCM (3.3 mL) dropwise at rt. The resulting mixture was stirred at rt for 1 h, then additional imidazole (11.5 mg, 0.168 mmol), PPh₃ (4.4 mg, 0.017 mmol) and CBr₄ (5.5 mg, 0.017 mmol) were added and the mixture was stirred at rt for 30 min. The reaction mixture was concentrated to dryness and the residue purified by silica gel chromatography (0-100% EtOAc/hexanes) to provide the title compound as a white foam (79% yield).

### Intermediate 2: 2,5-Dioxopyrrolidin-1-yl 4-cyclopropyl-1,2,5-oxadiazole-3-carboxylate

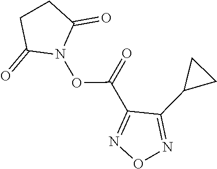 [SMILES: O=C(ON1C(=O)CCC1=O)c1nonc1C1CC1]

Step A: 4-Cyclopropyl-1,2,5-oxadiazole-3-carbonyl chloride. A flask was charged with 4-cyclopropyl-1,2,5-oxadiazole-3-carboxylic acid (200 mg, 1.30 mmol) and DCM (2.6 mL). The solution was cooled to 0° C. and then oxalyl chloride (0.224 mL, 2.60 mmol) was added dropwise followed by 1 drop of DMF. The mixture was stirred for 3 h as it warmed to rt. The reaction mixture was concentrated under reduced pressure into a yellow oil and dissolved in DCM to result in a 2 M solution of 4-cyclopropyl-1,2,5-oxadiazole-3-carbonyl chloride that was used without further purification.

Step B: 2,5-Dioxopyrrolidin-1-yl 4-cyclopropyl-1,2,5-oxadiazole-3-carboxylate. A flask was charged with N-hydroxysuccinimide (231 mg, 1.95 mmol), DCM (3.25 mL) and DIPEA (0.336 mL, 1.95 mmol). The reaction mixture was cooled to 0° C. and 4-cyclopropyl-1,2,5-oxadiazole-3-carbonyl chloride (2 M in DCM, 0.649 mL, 1.30 mmol, Step A) was then added dropwise. The reaction mixture was stirred for 1 h as it warmed to rt. The reaction mixture was then washed with water and brine sequentially, dried over anhydrous MgSO₄, filtered and concentrated to dryness. The material was purified by silica gel chromatography (0-100% (EtOAc with 10% MeOH)/hexanes) to afford the title compound as a clear oil (39% yield).

### Intermediate 3: tert-Butyl 3-methyl-2-oxopiperidine-1-carboxylate

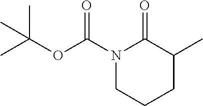

To a solution of 3-methylpiperidin-2-one (1.00 g, 8.84 mmol) and DMAP (108 mg, 0.880 mmol) in CH₂Cl₂ (18 mL) was added TEA (1.47 mL, 10.6 mmol). Next, di-tert-butyl dicarbonate (2.44 g, 11.2 mmol) was added and the reaction mixture was stirred at rt for 26 h. This mixture was then diluted with CH₂Cl₂ and washed sequentially with 10% aqueous citric acid, 10% aqueous NaHCO₃ and brine. The organic layer was dried over anhydrous Na₂SO₄, filtered, and concentrated to dryness. The residue was purified by silica gel chromatography (0-100% EtOAc/hexanes) to afford the title compound that was used without further purification (86% yield).

### Intermediate 4: tert-Butyl 2-oxoazepane-1-carboxylate

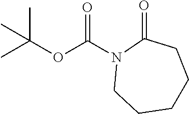

The title compound was prepared as described for the synthesis of Intermediate 3 using epsilon-caprolactam in place of 3-methylpiperidin-2-one and THF in place of CH₂Cl₂. The residue was purified by silica gel chromatography (0-100% EtOAc (with 10% MeOH)/hexanes) to provide the title compound (97% yield).

### Intermediate 5: tert-Butyl 2-oxo-6-(trifluoromethyl)piperidine-1-carboxylate

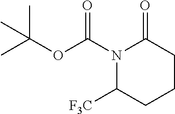 [SMILES: CC(C)(C)OC(=O)N1C(=O)CCCC1C(F)(F)F]

The title compound was prepared as described for the synthesis of Intermediate 3 using 6-(trifluoromethyl)piperidin-2-one in place of 3-methylpiperidin-2-one and THF in place of CH₂Cl₂. The residue was purified by silica gel chromatography (0-100% EtOAc/hexanes) to provide the title compound (98% yield) as a clear solid.

### Intermediate 6: tert-Butyl 2-oxo-4-(trifluoromethyl)piperidine-1-carboxylate

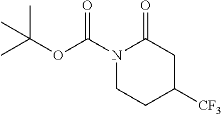 [SMILES: CC(C)(C)OC(=O)N1CCC(C(F)(F)F)CC1=O]

The title compound was prepared as described for the synthesis of Intermediate 3 using 4-(trifluoromethyl)piperidin-2-one in place of 3-methylpiperidin-2-one and THF in place of CH₂Cl₂. The residue was purified by silica gel chromatography (0-100% EtOAc (with 10% MeOH)/hexanes) to provide the title compound (99% yield).

### Intermediate 7: tert-Butyl 2-oxo-5-(trifluoromethyl)piperidine-1-carboxylate

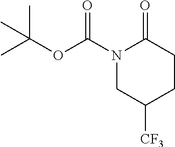 [SMILES: CC(C)(C)OC(=O)N1CC(C(F)(F)F)CCC1=O]

The title compound (89% yield) was prepared as described for the synthesis of Intermediate 3 using 5-(trifluoromethyl)piperidin-2-one in place of 3-methylpiperidin-2-one.

### Intermediate 8: 3-((2-((S)-Amino(4,4-difluorocyclohexyl)methyl)imidazo[1,2-b]pyridazin-7-yl)methyl)pyrrolidin-2-one

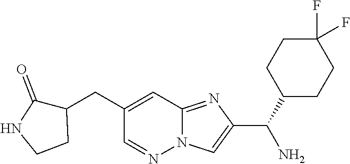 [SMILES: N[C@H](c1cn2ncc(CC3CCNC3=O)cc2n1)C1CCC(F)(F)CC1]

Step A: tert-Butyl 3-((2-((S)-((tert-butoxycarbonyl)amino(4,4-difluorocyclohexyl)methyl)imidazo[1,2-b]pyridazin-7-yl)methyl)-2-oxopyrrolidine-1-carboxylate. To a solution of tert-butyl 2-oxopyrrolidine-1-carboxylate (176 mg, 0.950 mmol) in THF (0.95 mL) cooled to −78° C. was added LiHMDS (1.0 mL, 1.0 mmol, 1 M in THF) dropwise and the resulting solution that was stirred at −78° C. for 1 h. Then, a solution of tert-butyl (S)-((7-(bromomethyl)imidazo[1,2-b]pyridazin-2-yl)4,4-difluorocyclohexyl)methyl)carbamate (300 mg, 0.65 mmol, Intermediate 1) in THF (1.5 mL) was added dropwise over 5 min. The resulting solution was stirred for 30 min at −78° C. then allowed to warm to rt over 30 min and was quenched by the addition of saturated aqueous NH₄Cl. This mixture was then diluted with EtOAc and the layers were separated. The aqueous layer was extracted with EtOAc (3×), and the combined organic layers were washed with brine, dried over anhydrous Na₂SO₄, filtered, and concentrated to dryness. The residue was purified by silica gel chromatography (0-100% EtOAc (with 10% MeOH)/hexanes) to afford the title compound (26% yield).

Step B: 3-((2-((S)-Amino(4,4-difluorocyclohexyl)methyl)imidazo[1,2-b]pyridazin-7-yl)methyl)pyrrolidin-2-one. To a solution of tert-butyl 3-((2-((S)-((tert-butoxycarbonyl)amino)(4,4-difluorocyclohexyl)methyl)imidazo[1,2-b]pyridazin-7-yl)methyl)-2-oxopyrrolidine-1-carboxylate (93 mg, 0.16 mmol, Step A) in CH₂Cl₂ (1.0 mL) was added TFA (1.0 mL) and the reaction was stirred at rt for 1 h. After this time, the pH of the mixture was adjusted to pH>10 by the careful addition of 0.5 M aqueous NaOH, then the mixture was extracted with CH₂Cl₂. The combined organic extracts were dried over anhydrous Na₂SO₄, filtered, and concentrated to dryness. The residue was purified by silica gel chromatography (0-10% (MeOH with 2.0 M ammonia)/DCM) to afford the title compound (80% yield).

### Intermediate 9: 3-((2-((S)-Amino(4,4-difluorocyclohexyl)methyl)imidazo[1,2-b]pyridazin-7-yl)methyl)piperidin-2-one

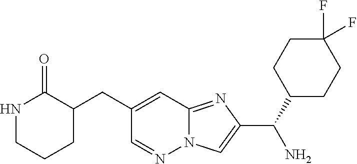 [SMILES: N[C@H](c1cn2ncc(CC3CCCNC3=O)cc2n1)C1CCC(F)(F)CC1]

Step A: tert-Butyl 3-((2-((S)-((tert-butoxycarbonyl)amino(4,4-difluorocyclohexyl)methyl)imidazo[1,2-b]pyridazin-7-yl)methyl)-2-oxopiperidine-1-carboxylate. The title compound (24% yield) was prepared as described for the synthesis of Intermediate 8 Step A using tert-butyl 2-oxopiperidine-1-carboxylate in place of tert-butyl 2-oxopyrrolidine-1-carboxylate, and cooling the reaction mixture to −45° C. instead of −78° C.

Step B: 3-((2-((S)-Amino(4,4-difluorocyclohexyl)methyl)imidazo[1,2-b]pyridazin-7-yl)methyl)piperidin-2-one. The title compound (66% yield) was prepared as described for the synthesis of Intermediate 8 Step B using tert-butyl 3-((2-((S)-((tert-butoxycarbonyl)amino)4,4-difluorocyclohexyl)methyl)imidazo[1,2-b]pyridazin-7-yl)methyl)-2-oxopiperidine-1-carboxylate (Step A) in place of tert-butyl 3-((2-((S)-((tert-butoxycarbonyl)amino)(4,4-difluorocyclohexyl)methyl)imidazo[1,2-b]pyridazin-7-yl)methyl)-2-oxopyrrolidine-1-carboxylate.

### Intermediate 10: 3-((2-((S)-Amino(4,4-difluorocyclohexyl)methyl)imidazo[1,2-b]pyridazin-7-yl)methyl)azepan-2-one

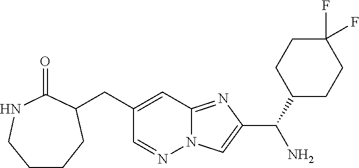 [SMILES: N[C@H](c1cn2ncc(CC3CCCCNC3=O)cc2n1)C1CCC(F)(F)CC1]

Step A: tert-Butyl 3-((2-((S)-((tert-butoxycarbonyl)amino)(4,4-difluorocyclohexyl)methyl)imidazo[1,2-b]pyridazin-7-yl)methyl)-2-oxoazepane-1-carboxylate. The title compound (16% yield) was prepared as described for the synthesis of Intermediate 8 Step A using tert-butyl 2-oxoazepane-1-carboxylate (Intermediate 4) in place of tert-butyl 2-oxopyrrolidine-1-carboxylate, and cooling the reaction mixture to −45° C. instead of −78° C.

Step B: 3-((2-((S)-Amino(4,4-difluorocyclohexyl)methyl)imidazo[1,2-b]pyridazin-7-yl)methyl)piperidin-2-one. The title compound (80% yield) was prepared as described for the synthesis of Intermediate 8 Step B using tert-butyl 3-((2-((S)-((tert-butoxycarbonyl)amino(4,4-difluorocyclohexyl)methyl)imidazo[1,2-b]pyridazin-7-yl)methyl)-2-oxoazepane-1-carboxylate (Step A) in place of tert-butyl 3-((2-((S)-((tert-butoxycarbonyl)amino)(4,4-difluorocyclohexyl)methyl)imidazo[1,2-b]pyridazin-7-yl)methyl)-2-oxopyrrolidine-1-carboxylate.

### Intermediate 11: 3-((2-((S)-Amino(4,4-difluorocyclohexyl)methyl)imidazo[1,2-b]pyridazine-7-yl)methyl)-3-methylpiperidin-2-one

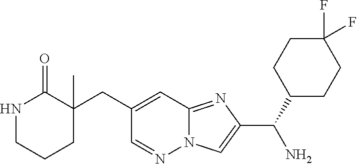 [SMILES: CC1(Cc2cnn3cc([C@@H](N)C4CCC(F)(F)CC4)nc3c2)CCCNC1=O]

Step A: tert-Butyl 3-((2-((S)-((tert-butoxycarbonyl)amino)(4,4-difluorocyclohexyl)methyl)imidazo[1,2-b]pyridazine-7-yl)methyl)-3-methyl-2-oxopiperidine-1-carboxylate. The title compound (30% yield) was prepared as described for the synthesis of Intermediate 8 Step A using tert-butyl 3-methyl-2-oxopiperidine-1-carboxylate (Intermediate 3) in place of tert-butyl 2-oxopyrrolidine-1-carboxylate.

Step B: 3-((2-((S)-Amino(4,4-difluorocyclohexyl)methyl)imidazo[1,2-b]pyridazin-7-yl)methyl)-3-methylpiperidin-2-one. The title compound (100% yield) was prepared as described for the synthesis of Intermediate 8 Step B using tert-butyl 3-((2-((S)-((tert-butoxycarbonyl)amino)(4,4-difluorocyclohexyl)methyl)imidazo[1,2-b]pyridazin-7-yl)methyl)-3-methyl-2-oxopiperidine-1-carboxylate (Step A) in place of tert-butyl 3-((2-((S)-((tert-butoxycarbonyl)amino)(4,4-difluorocyclohexyl)methyl)imidazo[1,2-b]pyridazin-7-yl)methyl)-2-oxopyrrolidine-1-carboxylate.

### Intermediate 12: 3-((2-((S)-Amino(4,4-difluorocyclohexyl)methyl)imidazo[1,2-b]pyridazin-7-yl)methyl)-6-(trifluoromethyl)piperidin-2-one

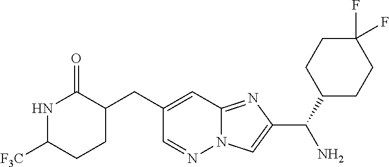 [SMILES: N[C@H](c1cn2ncc(CC3CCC(C(F)(F)F)NC3=O)cc2n1)C1CCC(F)(F)CC1]

Step A: tert-Butyl 3-((2-((S)-((tert-butoxycarbonyl)amino(4,4-difluorocyclohexyl)methyl)imidazo[1,2-b]pyridazin-7-yl)methyl)-2-oxo-6-(trifluoromethyl)piperidine-1-carboxylate. The title compound was prepared as described for the synthesis of Intermediate 8 Step A using tert-butyl 2-oxo-6-(trifluoromethyl)piperidine-1-carboxylate (Intermediate 5) in place of tert-butyl 2-oxopyrrolidine-1-carboxylate and cooling the reaction mixture to −45° C. instead of −78° C. The residue was purified by silica gel chromatography (0-100% acetone/DCM) to provide the title compound (77% yield).

Step B: 3-((2-((S)-Amino(4,4-difluorocyclohexyl)methyl)imidazo[1,2-b]pyridazin-7-yl)methyl)-6-(trifluoromethyl)piperidin-2-one. The title compound (82% yield) was prepared as described for the synthesis of Intermediate 8 Step B using ten-butyl 3-((2-((S)-((tert-butoxycarbonyl)amino)(4,4-difluorocyclohexyl)methyl)imidazo[1,2-b]pyridazin-7-yl)methyl)-2-oxo-6-(trifluoromethyl)piperidine-1-carboxylate (Step A) in place of tert-butyl 3-((2-((S)-((tert-butoxycarbonyl)amino)(4,4-difluorocyclohexyl)methyl)imidazo[1,2-b]pyridazin-7-yl)methyl)-2-oxopyrrolidine-1-carboxylate.

Intermediate 13: (3R*)-3-((2-((S)-Amino(4,4-difluorocyclohexyl)methyl)imidazo[1,2-b]pyridazin-7-yl)methyl)-5-(trifluoromethyl)piperidin-2-one

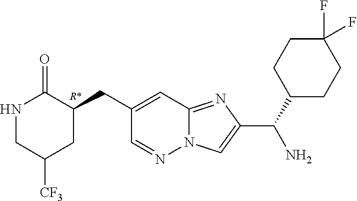 [SMILES: N[C@H](c1cn2ncc(C[C@H]3CC(C(F)(F)F)CNC3=O)cc2n1)C1CCC(F)(F)CC1]

### Intermediate 14: (3S*)-3-((2-((S)-Amino(4,4-difluorocyclohexyl)methyl)imidazo[1,2-b]pyridazin-7-yl)methyl)-5-(trifluoromethyl)piperidin-2-one

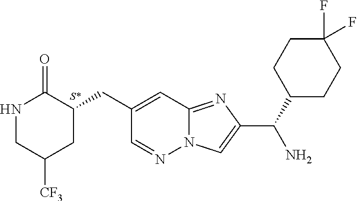 [SMILES: N[C@H](c1cn2ncc(C[C@@H]3CC(C(F)(F)F)CNC3=O)cc2n1)C1CCC(F)(F)CC1]

Step A: tert-Butyl (3R*)-3-((2-((S)-((tert-butoxycarbonyl)amino(4,4-difluorocyclohexyl)methyl)imidazo[1,2-b]pyridazin-7-yl)methyl)-2-oxo-5-(trifluoromethyl)piperidine-1-carboxylate and tert-Butyl (3S*)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)piperidine-1-carboxylate. The title compounds were prepared as described for the synthesis of Intermediate 8 Step A using tert-butyl 2-oxo-5-(trifluoromethyl)piperidine-1-carboxylate (Intermediate 7) in place of tert-butyl 2-oxopyrrolidine-1-carboxylate and cooling the reaction mixture to −45° C. instead of −78° C. to provide the R* isomer of the title compound (28% yield) as the first-eluting isomer and the S* isomer of the title compound (14% yield) as the second-eluting isomer.

Step B1: (3R*)-3-((2-((S)-Amino(4,4-difluorocyclohexyl)methyl)imidazo[1,2-b]pyridazin-7-yl)methyl)-5-(trifluoromethyl)piperidin-2-one (Intermediate 13). The title compound (73% yield) was prepared as described for the synthesis of Intermediate 8 Step B using tert-butyl (3R*)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)piperidine-1-carboxylate (Step A, first-eluting isomer) in place of tert-butyl 3-((2-((S)-((tert-butoxycarbonyl)amino)(4,4-difluorocyclohexyl)methyl)imidazo[1,2-b]pyridazin-7-yl)methyl)-2-oxopyrrolidine-1-carboxylate.

Step B2: (3S*)-3-((2-((S)-Amino(4,4-difluorocyclohexyl)methyl)imidazo[1,2-b]pyridazin-7-yl)methyl)-5-(trifluoromethyl)piperidin-2-one (Intermediate 14). The title compound (34% yield) was prepared as described for the synthesis of Intermediate 8 Step B using tert-butyl (3S*)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)piperidine-1-carboxylate (Step A, second-eluting isomer) in place of tert-butyl 3-((2-((S)-((tert-butoxycarbonyl)amino)(4,4-difluorocyclohexyl)methyl)imidazo[1,2-b]pyridazin-7-yl)methyl)-2-oxopyrrolidine-1-carboxylate.

### Intermediate 15: Diethyl ((5S)-2-oxo-1-tosyl-5-(trifluoromethyl)pyrrolidin-3-yl)phosphonate

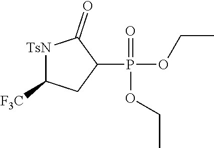 [SMILES: CCOP(=O)(OCC)C1C[C@@H](C(F)(F)F)N(S(=O)(=O)c2ccc(C)cc2)C1=O]

Step A: (R)-2-(Trifluoromethyl)oxirane. A mixture of 2-(trifluoromethyl)oxirane (150 g, 1.34 mol), (S,S)-(salen)Co(III)(OAc) (4.44 g, 6.69 mmol) and water (13.3 mL, 0.736 mol) under an inert atmosphere was stirred for 48 h at rt. The mixture was then distilled at atmospheric pressure (fraction collected at 32° C.) to afford the title compound in 35% yield as a colorless oil.

Step B: (R)-4-Methyl-N-(3,3,3-trifluoro-2-hydroxypropyl)benzenesulfonamide. A mixture of (R)-2-(trifluoromethyl)oxirane (52.2 g, 184 mmol, Step A), CHCl₃ (522 mL), 4-toluenesulfonamide (79.8 g, 466 mmol), and Ti(Oi-Pr)₄ (132 g, 466 mmol) was stirred overnight at rt. The reaction was then quenched by the addition of water (I L) and the biphasic mixture was extracted with DCM (3×800 mL). The combined organic layers were washed with brine (3×1 L), dried over anhydrous Na₂SO₄, filtered, and concentrated to dryness. The residue was purified by silica gel chromatography (20-33% EtOAc/petroleum ether) to afford the title compound in 50% yield as a white solid.

Step C: (S)-1-Tosyl-2-(trifluoromethyl)aziridine. A mixture of (R)-4-methyl-N-(3,3,3-trifluoro-2-hydroxypropyl)benzenesulfonamide (66 g, 230 mmol, Step B) and PPh₃ (67 g, 250 mmol) was dissolved in THF (660 mL) and the solution was cooled to 0° C. Then, DEAD (41 g, 230 mmol) was added over 30 min and the resulting solution was stirred overnight at rt. The reaction was quenched by the addition of water (1 L) and the biphasic solution was extracted with DCM (3×1 L). The combined organic layers were washed with brine (3×2 L), dried over anhydrous Na₂SO₄, filtered, and concentrated to dryness. The residue was purified by silica gel chromatography (5-10% EtOAc/petroleum ether) to afford the title compound in 81% yield as a white solid.

Step D: Ethyl (4S)-2-(diethoxyphosphoryl)-5,5,5-trifluoro-4-((4-methylphenyl)sulfonamido)pentanoate. A mixture of KOt-Bu (4.23 g, 37.7 mmol) and ethyl 2-(diethoxyphosphoryl)acetate (7.48 mL, 37.7 mmol) in THF (47 mL) was heated at 40° C. for 20 min. (S)-1-Tosyl-2-(trifluoromethyl)aziridine (5.00 g, 18.9 mmol, Step C) was then added and the reaction mixture was heated at 40° C. for 16 h. After this time, the reaction mixture was cooled to rt, concentrated to dryness, and purified by silica gel chromatography (0-100% EtOAc/hexanes) to afford the title compound in 69% yield as a mixture of diastereomers.

Step E: Diethyl ((5S)-2-oxo-1-tosyl-5-(trifluoromethyl)pyrrolidin-3-yl)phosphonate. Ethyl (4S)-2-(diethoxyphosphoryl)-5,5,5-trifluoro-4-((4-methylphenyl)sulfonamido)pentanoate (6.38 g, 13.0 mmol, Step D) was dissolved in toluene (32.6 mL) and the mixture was cooled to 0° C. Then, a 2 M solution of AlMe₃ (19.6 mL, 39.1 mmol) in toluene was added dropwise via an addition funnel at a rate such that the reaction temperature did not exceed 6° C. Once the addition was complete, the ice bath was removed and the reaction mixture was warmed to rt and then heated at 110° C. overnight. The reaction mixture was cooled to 0° C. and quenched slowly by the sequential dropwise addition of IPA (2.0 mL) and MeOH (1.1 mL). The resulting mixture was then diluted with saturated aqueous NH₄Cl (10 mL) and 0.1 M aqueous HCl (5 mL), filtered through a pad of diatomaceous earth and the filter cake was washed with EtOAc (2×25 mL). The layers were separated and the aqueous layer was further extracted with EtOAc (3×100 mL). The combined organic layers were washed with brine, dried over anhydrous MgSO₄, filtered, and concentrated to dryness. The residue was purified by silica gel chromatography (0-100% EtOAc/hexanes) to afford the title compound (62% yield) as a colorless oil.

### Intermediate 16: ter-Butyl (S)-(1-(dimethyl(oxo)-λ⁶-sulfaneylidene)-7,7,7-trifluoro-6,6-dimethyl-2-oxoheptan-3-yl)carbamate

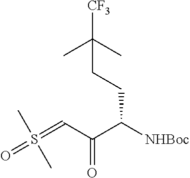 [SMILES: CC(C)(C)OC(=O)N[C@@H](CCC(C)(C)C(F)(F)F)C(=O)C=S(C)(C)=O]

Step A: 1,1,1-Trifluoro-4-iodo-2,2-dimethylbutane. To a stirring solution of 4,4,4-trifluoro-3,3-dimethylbutan-1-ol (9.75 g, 62.5 mmol) in DCM (125 mL) was added triphenylphosphine (24.6 g, 93.7 mmol) and imidazole (5.53 g, 81.2 mmol), and the resulting mixture was cooled to 0° C. Iodine (23.8 g, 93.7 mmol) was then added portion-wise and the mixture was stirred for 24 h at which time it was quenched with saturated aqueous NaHCO₃ and stirred for 15 min. The organic layer was removed and washed sequentially with half-saturated aqueous NaHCO₃ and brine, dried over anhydrous MgSO₄, filtered, and concentrated to a residue. To the residue was added chilled hexanes (50 mL), and the mixture was stirred vigorously for 10 min at which time a white precipitated formed. The white solid was removed by filtration through a pad of silica and the solids were washed with a second portion of chilled hexanes. The filtrates were concentrated to afford the title compound as a clear, colorless oil (66% yield) that was used without further purification.

Step B: (2R,5S)-2-Isopropyl-3,6-dimethoxy-5-(4,4,4-trifluoro-3,3-dimethylbutyl)-2,5-dihydropyrazine. To a stirred solution of (R)-2-isopropyl-3,6-dimethoxy-2,5-dihydropyrazine (0.66 g, 3.59 mmol) in THF (7 mL) at −78° C. was added t-BuLi (2.33 mL, 3.95 mmol, 1.7 M in pentane), and the reaction was allowed to stir at −78° C. After 1 h, a solution of 1,1,1-trifluoro-4-iodo-2,2-dimethylbutane (1.05 g, 3.95 mmol, Step A) in THF (6 mL) was added dropwise, and the reaction was stirred for 24 h at rt. The reaction was quenched by the addition of aqueous phosphate buffer (50 mL, 0.1 M, pH 7) and was diluted with diethyl ether. The organic layer was removed, and the aqueous layer was extracted with diethyl ether (2×50 mL). The combined organic layers were dried over anhydrous MgSO₄, filtered and concentrated to give an oil. LC/MS of this oil revealed a mixture of diastereomers in ~1.5:1 ratio. This mixture was separated by silica gel chromatography (0-30% ethyl acetate in hexanes) to afford the first-eluting isomer (50% yield), which was designated as the R,S diastereomer, as a yellow oil. The second-eluting isomer, designated as the R,R diastereomer, (2R,5R)-2-isopropyl-3,6-dimethoxy-5-(4,4,4-trifluoro-3,3-dimethylbutyl)-2,5-dihydropyrazine, was isolated in 28% yield.

Step C: Methyl (S)-2-amino-6,6,6-trifluoro-5,5-dimethylhexanoate. To a stirred solution of (2R,5S)-2-isopropyl-3,6-dimethoxy-5-(4,4,4-trifluoro-3,3-dimethylbutyl)-2,5-dihydropyrazine (1.15 g, 3.57 mmol, Step B first-eluting isomer) in ACN (14.3 mL) at rt was added aqueous HCl (14.3 mL, 1 M). After stirring for 2 h, the reaction mixture was poured onto saturated aqueous NaHCO₃ at 0° C. and was then extracted with EtOAc (3×150 mL). The combined organic layers were washed with brine, dried over anhydrous MgSO₄, filtered, and concentrated to give an oil that was used without further purification.

Step D: Methyl (S)-2-((tert-butoxycarbonyl)amino)-6,6,6-trifluoro-5,5-dimethylhexanoate. To a stirred solution of methyl (S)-2-amino-6,6,6-trifluoro-5,5-dimethylhexanoate (811 mg, 3.57 mmol, Step C) in DCM (15.1 mL) was added Boc₂O (1.56 g, 7.14 mmol). After 24 h, the reaction was quenched with aqueous HCl (10 mL, 1 M), and the layers were separated. The organic layer was washed sequentially with aqueous HCl (10 mL, 1 M), water and brine. The organic layer was dried over anhydrous MgSO₄, filtered, and concentrated to give an oil that was used in the next step without further purification.

Step E: (S)-2-((tert-Butoxycarbonyl)amino)-6,6,6-trifluoro-5,5-dimethylhexanoic acid. To a stirred solution of methyl (S)-2-((tert-butoxycarbonyl)amino)-6,6,6-trifluoro-5,5-dimethylhexanoate (925 mg, 2.83 mmol, Step D) in THF (22.4 mL) at 0° C. was added aqueous LiOH (8.50 mL, 1 M) and the resulting mixture was stirred at 0° C. for 2 h. The pH of the reaction mixture was then adjusted to pH 2 using aqueous HCl (1 M). The mixture was extracted with EtOAc (3×60 mL). The combined organic layers were washed with brine, dried over anhydrous MgSO₄, filtered, and concentrated to give a clear oil that was used in the next step without further purification.

Step F: tert-Butyl (S)-(1-(dimethyl(oxo)-λ⁶-sulfaneylidene)-7,7,7-trifluoro-6,6-dimethyl-2-oxoheptan-3-yl)carbamate. To a suspension of trimethylsulfoxonium chloride (0.581 g, 4.52 mmol) in THF (7 mL) was added a solution of t-BuOK (4.24 mL, 4.24 mmol, 1 M in THF) and the resulting solution was stirred at rt for 2 h. Separately, to a solution of (S)-2-((tert-butoxycarbonyl)amino)-6,6,6-trifluoro-5,5-dimethylhexanoic acid (885 mg, 2.83 mmol, Step E) in THE (7 mL) cooled to 0° C. was added CDI (550 mg, 3.39 mmol) and the resulting mixture was stirred for 2 h at 0° C. This mixture was then added via cannula to the trimethylsulfoxonium-derived suspension, and the resulting mixture was stirred at rt. After 2 h, the reaction mixture was filtered through diatomaceous earth, concentrated, and purified via silica gel chromatography (0-100% ethyl acetate in hexanes) to afford the title compound (56% yield) as a white foam.

### Intermediate 17: tert-Butyl (S)-(5,5,5-trifluoro-1-(7-formylimidazo[1,2-b]pyridazin-2-yl)-4,4-dimethylpentyl)carbamate

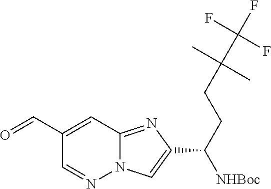 [SMILES: CC(C)(C)OC(=O)N[C@@H](CCC(C)(C)C(F)(F)F)c1cn2ncc(C=O)cc2n1]

Step A: tert-Butyl (S)-(1-(7-chloroimidazo[1,2-b]pyridazin-2-yl)-5,5,5-trifluoro-4,4-dimethylpentyl)carbamate. 5-Chloropyridazin-3-amine (1.00 g, 7.72 mmol) and tert-butyl (S)-(1-(dimethyl(oxo)-λ⁶-sulfaneylidene)-7,7,7-trifluoro-6,6-dimethyl-2-oxoheptan-3-yl)carbamate (3.29 g, 8.49 mmol, Intermediate 16) were dissolved in toluene (21 mL). 4 Å Molecular sieves (2.00 g), NaOTf (66.4 mg, 0.386 mmol), and chloro(cyclopentadienyl)bis(triphenylphosphine)ruthenium(II) (141 mg, 0.193 mmol) were added sequentially, and the resulting mixture was heated at 85° C. for 16 h. The reaction mixture was concentrated onto diatomaceous earth and purified by silica gel chromatography (0-100% EtOAc/hexanes) to afford the title compound in 30% yield.

Step B: tert-Butyl (S)-(5,5,5-trifluoro-4,4-dimethyl-1-(7-vinylimidazo[1,2-b]pyridazin-2-yl)pentyl)carbamate. tert-Butyl (S)-(1-(7-chloroimidazo[1,2-b]pyridazin-2-yl)-5,5,5-trifluoro-4,4-dimethylpentyl)carbamate (0.960 mg, 2.28 mmol, Step A), potassium trifluoro(vinyl)borate (0.460 g, 3.43 mmol), and K: P04 (1.46 g, 6.90 mmol) were added under a positive pressure of nitrogen gas to a reactor containing 1,4-dioxane and water (24 mL, 5:1 v/v, sparged with N₂ before use). The mixture was heated at reflux temperature (85° C.), and then RuPhos Pd G3 (49.7 mg, 60.0 mmol) was added. The resulting mixture was stirred at 85° C. for 6 h. After this time, the mixture was allowed to cool to rt and then concentrated to remove most of the 1,4-dioxane. The residue was then diluted with EtOAc and water, the layers were separated, and the aqueous layer was extracted twice with EtOAc. The organic layers were combined, washed with brine, dried over anhydrous MgSO₄, filtered, and concentrated to dryness to afford the title compound as a brown foam, which was used without further purification.

Step C: tert-Butyl (S)-(5,5,5-trifluoro-1-(7-formylimidazo[1,2-b]pyridazin-2-yl)-4,4-dimethylpentyl)carbamate. A solution of NaIO₄ (2.43 g, 11.4 mmol) in water (55 mL) was added to a solution of tert-butyl (S)-(5,5,5-trifluoro-4,4-dimethyl-1-(7-vinylimidazo[1,2-b]pyridazin-2-yl)pentyl)carbamate (8.81 g, 22.5 mmol, Step B) in 1,4-dioxane (55 mL). The resulting mixture was then cooled with an ice bath to just above freezing and K₂OsO₄·2H₂O (42.0 mg, 0.114 mmol) was added. The reaction mixture was then removed from the ice bath and stirred for 1 h. The resulting thick suspension was diluted with water and the solution was extracted with EtOAc (4×). The combined organic layers were washed with brine, dried over anhydrous Na₂SO₄, filtered, concentrated onto diatomaceous earth and purified by silica gel chromatography (0-50% acetone/hexanes) to afford the title compound in 64% yield.

### Intermediate 18: (5S)-3-((2-((S)-1-Amino-5,5,5-trifluoro-4,4-dimethylpentyl)imidazo[1,2-b]pyridazin-7-yl)methyl)-5-(trifluoromethyl)pyrrolidin-2-one

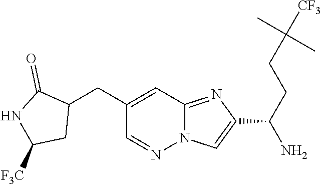 [SMILES: CC(C)(CC[C@H](N)c1cn2ncc(CC3C[C@@H](C(F)(F)F)NC3=O)cc2n1)C(F)(F)F]

Step A: tert-Butyl ((S)-5,5,5-trifluoro-4,4-dimethyl-1-(7-((S)-2-oxo-1-tosyl-5-(trifluoromethyl)pyrrolidin-3-ylidene)methyl)imidazo[1,2-b]pyridazin-2-yl)pentyl)carbamate. To a suspension of NaH (62.4 mg, 1.56 mmol) in THF (4 mL) was added a solution of diethyl ((5S)-2-oxo-1-tosyl-5-(trifluoromethyl)pyrrolidin-3-yl)phosphonate (692 mg, 1.56 mmol, Intermediate 15) in THF (4 mL) and the resulting mixture was cooled to −78° C. A solution of tert-butyl (S)-(5,5,5-trifluoro-1-(7-formylimidazo[1,2-b]pyridazin-2-yl)-4,4-dimethylpentyl)carbamate (500 mg, 1.20 mmol, Intermediate 17) in THF (4 mL) was then added dropwise to the reaction mixture and the resulting mixture was placed into an ice-water bath. The reaction stirred overnight while slowly warming to rt. After this time, the reaction was cooled to 0° C. and quenched with water. The biphasic mixture was extracted with EtOAc (3×) and the combined organic layers were washed with brine, dried over anhydrous MgSO₄, filtered, and concentrated to dryness. The residue was purified by silica gel chromatography (0-100% acetone/hexanes) to afford the title compound as a mixture of olefin isomers in 58% yield.

Step B: tert-Butyl ((1S)-5,5,5-trifluoro-4,4-dimethyl-1-(7-(((5S)-2-oxo-1-tosyl-5-(trifluoromethyl)pyrrolidin-3-yl)methyl)imidazo[1,2-b]pyridazin-2-yl)pentyl)carbamate. tert-Butyl ((S)-5,5,5-trifluoro-4,4-dimethyl-1-(7-((S)-2-oxo-1-tosyl-5-(trifluoromethyl)pyrrolidin-3-ylidene)methyl)imidazo[1,2-b]pyridazin-2-yl)pentyl)carbamate (487 mg, 0.692 mmol, Step A) was dissolved in EtOH (28 mL) and the solution was sparged with N₂. 10% wt Pd/C (286 mg, 0.234 mmol) was then added and the reaction was evacuated and refilled with H₂ (3×) with a balloon. The reaction mixture was stirred for at rt for 3 h, after which the reaction was filtered through diatomaceous earth/silica gel mixture and concentrated to dryness to afford the title compound that was used without further purification.

Step C: tert-Butyl ((1S)-5,5,5-trifluoro-4,4-dimethyl-1-(7-(((5S)-2-oxo-5-(trifluoromethyl)pyrrolidin-3-yl)methyl)imidazo[1,2-b]pyridazin-2-yl)pentyl)carbamate. SmI₂ (24 mL, 2.4 mmol, 0.1 M in THF) was added to a solution of tert-butyl ((1S)-5,5,5-trifluoro-4,4-dimethyl-1-(7-(((5S)-2-oxo-1-tosyl-5-(trifluoromethyl)pyrrolidin-3-yl)methyl)imidazo[1,2-b]pyridazin-2-yl)pentyl)carbamate (315 mg, 0.446 mmol. Step B) in THF (6.4 mL) at 0° C. The reaction mixture was warmed to rt and stirred for 1.5 h. After this time additional SmI₂ (4.5 mL, 0.45 mmol, 0.1 M in THF) was added and the mixture was stirred at rt for 30 min. The reaction was then quenched by the addition of 1 M aqueous HCl. The biphasic mixture was extracted with EtOAc (3×) and the combined organic layers were washed with brine, dried over anhydrous MgSO₄, filtered, and concentrated to dryness. The residue was purified by silica gel chromatography (0-100% EtOAc (10% MeOH)/hexanes) to afford the title compound in >99/yield.

Step D: (5S)-3-((2-((S)-1-Amino-5,5,5-trifluoro-4,4-dimethylpentyl)imidazo[1,2-b]pyridazin-7-yl)methyl)-5-(trifluoromethyl)pyrrolidin-2-one. tert-Butyl ((1S)-5,5,5-trifluoro-4,4-dimethyl-1-(7-(((5S)-2-oxo-5-(trifluoromethyl)pyrrolidin-3-yl)methyl)imidazo[1,2-b]pyridazin-2-yl)pentyl)carbamate (300 mg, 0.544 mmol, Step C) was dissolved in DCM (6.97 mL) and then TFA (1.04 mL, 13.6 mmol) was added dropwise. The reaction was stirred at rt for 45 min and was subsequently concentrated to dryness and purified by silica gel chromatography (0-20% MeOH/DCM) to afford the title compound in >99% yield.

### Intermediate 19: tert-Butyl (S)-3-methylene-2-oxo-5-(trifluoromethyl)pyrrolidine-1-carboxylate

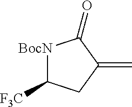 [SMILES: C=C1C[C@@H](C(F)(F)F)N(C(=O)OC(C)(C)C)C1=O]

Step A: tert-Butyl (S)-2-oxo-5-(trifluoromethyl)pyrrolidine-1-carboxylate. S-5-(Trifluoromethyl)pyrrolidin2-one (10.0 g, 65.3 mmol) and DMAP (107 mg, 0.877 mmol) were dissolved in DCM (218 mL). TEA (10.9 mL, 78.4 mmol) and di-tert-butyl dicarbonate (17.1 g, 78.4 mmol) were sequentially added and the reaction was stirred at rt overnight. The reaction was then washed sequentially with 10% aqueous citric acid, water, and brine, dried over anhydrous MgSO₄, filtered, and concentrated to dryness. The residue was purified by silica gel chromatography (0-50% EtOAc/hexanes) to afford the title compound in 70% yield.

Step B: tert-Butyl (5S)-3-((dimethylamino)methyl)-2-oxo-5-(trifluoromethyl)pyrrolidine-1-carboxylate. A solution of LiHMDS (2.0 mL, 2.0 mmol, 1 M in THF) was cooled to −78° C. Then, a solution of tert-butyl (S)-2-oxo-5-(trifluoromethyl)pyrrolidine-1-carboxylate (250 mg, 1.0 mmol, Step A) in THF (9.9 mL) was added dropwise to the LiHMDS solution and the resulting solution was stirred at −78° C. for 30 min. After this time, N,N-dimethylmethyleneiminium iodide (370 mg, 2.0 mmol) was added portion-wise via addition funnel and the resulting mixture was stirred at −78° C. for an additional 30 min before being allowed to slowly warm to rt and stir at rt for 30 min. The reaction was quenched by the addition of water and the biphasic mixture was extracted with EtOAc (3×). The combined organic layers were washed with brine, dried over anhydrous MgSO₄, filtered, and concentrated to dryness to afford the title compound that was used without further purification.

Step C: tert-Butyl (S)-3-methylene-2-oxo-5-(trifluoromethyl)pyrrolidine-1-carboxylate. tert-Butyl (5S)-3-((dimethylamino)methyl)-2-oxo-5-(trifluoromethyl)pyrrolidine-1-carboxylate (3.06 g, 9.87 mmol, Step B) was dissolved in MeOH (9.9 mL) and then Mel (1.54 mL, 24.7 mmol) was added dropwise. The reaction mixture was stirred at rt for 1 h followed by the addition of MeOH (25 mL) and the resulting mixture was stirred at rt for an additional 20 min. The reaction was then concentrated to dryness and dissolved in DCM (49 mL) and saturated aqueous NaHCO₃ (49 mL). The biphasic mixture was stirred vigorously overnight at rt. The layers were separated and the aqueous layer was extracted with DCM (3×). The combined organic layers were washed with brine, dried over anhydrous MgSO₄, filtered, and concentrated to dryness. The residue was purified by silica gel chromatography (0-60% EtOAc/hexanes) to afford the title compound in 59% yield.

### Intermediate 20: tert-Butyl (6S)-1-(2-((S)-((tert-butoxycarbonyl)amino)(4,4-difluorocyclohexyl)methyl)imidazo[1,2-b]pyridazin-7-yl)-4-oxo-6-(trifluoromethyl)-5-azaspiro[2.4]heptane-5-carboxylate

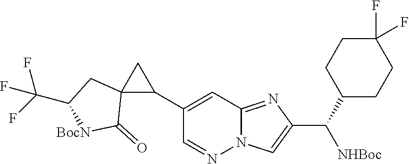

### Intermediate 21: (1R*,2R*)-2-(2-((S)-((tert-Butoxycarbonyl)amino)(4,4-difluorocyclohexyl)methyl)imidazo[1,2-b]pyridazin-7-yl)-1-((S)-2-((tert-butoxycarbonyl)amino)-3,3,3-trifluoropropyl)cyclopropane-1-carboxylic acid

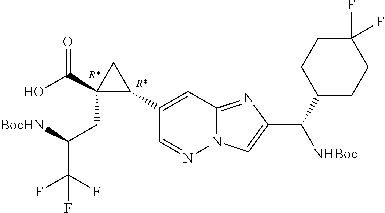 [SMILES: CC(C)(C)OC(=O)N[C@H](c1cn2ncc([C@H]3C[C@]3(C[C@H](NC(=O)OC(C)(C)C)C(F)(F)F)C(=O)O)cc2n1)C1CCC(F)(F)CC1]

Step A: tert-Butyl (S,E)-((4,4-difluorocyclohexyl)(7-((2-tosylhydrazineylidene)methyl)imidazo[1,2-b]pyridazin-2-yl)methyl)carbamate. tert-Butyl (S)-((4,4-difluorocyclohexyl)₇-formylimidazo[1,2-b]pyridazin-2-yl)methyl)carbamate (952 mg, 2.41 mmol) and 4-methylbenzenesulfonohydrazide (494 mg, 2.65 mmol) were dissolved in 1,4-dioxane (13 mL) and the reaction was heated at 85° C. for 1 h. The reaction was then concentrated to dryness to afford the title compound (>99% yield) that was used without further purification.

Step B: tert-Butyl (6S)-1-(2-((S)-((tert-butoxycarbonyl)amino(4,4-difluorocyclohexyl)methyl)imidazo[1,2-b]pyridazin-7-yl)-4-oxo-6-(trifluoromethyl)-5-azaspiro[2.4]heptane-5-carboxylate and (1R*,2R*)-2-(2-((S)-((tert-butoxycarbonyl)amino)(4,4-difluorocyclohexyl)methyl)imidazo[1,2-b]pyridazin-7-yl)-1-((S)-2-((tert-butoxycarbonyl)amino)-3,3,3-trifluoropropyl)cyclopropane-1-carboxylic acid. To a microwave vial was added tert-butyl (S,E)-((4,4-difluorocyclohexyl)(7-((2-tosylhydrazineylidene)methyl)imidazo[1,2-b]pyridazin-2-yl)methyl)carbamate (250 mg, 0.44 mmol, Step A) and 1,4-dioxane (2.2 mL). Then, tert-butyl (S)-3-methylene-2-oxo-5-(trifluoromethyl)pyrrolidine-1-carboxylate (240 mg, 0.89 mmol, Intermediate 19) and K₂CO₃ (92 mg, 0.67 mmol) were sequentially added. The reaction was sealed under a N₂ atmosphere and heated at 110° C. for 4 h. The reaction was cooled to rt and poured into water. The biphasic solution was extracted with EtOAc (3×) and the combined organic layers were washed with brine, dried over anhydrous MgSO₄, filtered, and concentrated to dryness. The residue was dissolved in DMF and was purified by basic preparative HPLC (XBridge C18, 5 μm, 50×100 mm 10-100% MeCN/H₂O (20 mM NH₄OH)) affording Intermediate 20 in 21% yield as the first-eluting fraction and Intermediate 21 in 14% yield as the second-eluting fraction.

### Intermediate 22: Diethyl ((5S)-5-cyclopropyl-2-oxo-1-tosylpyrrolidin-3-yl)phosphonate

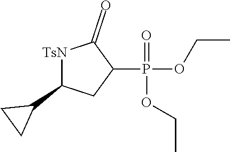

Step A: (R)—N-(1-Cyclopropyl-2-hydroxyethyl)-4-methylbenzenesulfonamide. To a solution of (R)-2-amino-2-cyclopropylethan-1-ol hydrochloride (4.0 g, 29 mmol) in DCM (100 mL) were added DIPEA (12 mL, 70 mmol) and 4-methylbenzenesulfonyl chloride (11 g, 58 mmol) sequentially. The reaction mixture was stirred for 12 h at rt. The reaction was then diluted with water and the biphasic mixture was extracted with DCM (3×50 mL). The combined organic layers were washed with brine, dried over anhydrous Na₂SO₄, filtered, and concentrated to dryness. The residue was purified by silica gel chromatography (0-100% EtOAc/petroleum ether) to afford the title compound in 81% yield.

Step B: (R)-2-Cyclopropyl-1-tosylaziridine. The title compound (32% yield) was synthesized in a manner analogous to Intermediate 15 Step C using (R)—N-(1-cyclopropyl-2-hydroxyethyl)-4-methylbenzenesulfonamide (Step A) in place of (R)-4-methyl-N-(3,3,3-trifluoro-2-hydroxypropyl)benzenesulfonamide.

Step C: Ethyl (4R)-4-cyclopropyl-2-(diethoxyphosphoryl)-4-((4-methylphenyl)sulfonamido)butanoate. The title compound (93% yield) was synthesized in a manner analogous to Intermediate 15 Step D using (R)-2-cyclopropyl-1-tosylaziridine (Step B) in place of (S)-1-tosyl-2-(trifluoromethyl)aziridine.

Step D: (4S)-4-Cyclopropyl-2-(diethoxyphosphoryl)-4-((4-methylphenyl)sulfonamido)butanoic acid. To a solution of ethyl (4R)-4-cyclopropyl-2-(diethoxyphosphoryl)-4-((4-methylphenyl)sulfonamido)butanoate (4.5 g, 9.7 mmol, Step C) in THF (120 mL) was added an aqueous solution of LiOH·H₂O (1.6 g, 39 mmol, 24 mL). The reaction was stirred at rt for 3 h. The reaction was then concentrated to remove the organic layer and the aqueous layer was washed with DCM (3×50 mL). The pH of the aqueous layer was then adjusted to pH 4-5 by the addition of aqueous 1 M HCl. The aqueous layer was extracted with EtOAc (4×100 mL) and the combined organic layers were washed with brine, dried over anhydrous Na₂SO₄, filtered, and concentrated to dryness to afford the title compound that was used without further purification (88% yield).

Step E: Diethyl ((5S)-5-cyclopropyl-2-oxo-1-tosylpyrrolidin-3-yl)phosphonate. To a solution of (4S)-4-cyclopropyl-2-(diethoxyphosphoryl)-4-((4-methylphenyl)sulfonamido)butanoic acid (3.7 g, 8.5 mmol, Step D) in DCM (300 mL) were added DIPEA (4.5 mL, 26 mmol) and 2-chloro-1-methylpyridinium iodide (3.3 g, 13 mmol) sequentially. The resulting mixture was stirred at rt for 16 h. The reaction mixture was then washed with water (3×100 mL) and the organic phase was dried over anhydrous Na₂SO₄, filtered, and concentrated to dryness. The residue was purified by silica gel chromatography (0-100% EtOAc/petroleum ether) to afford the title compound in 61% yield.

### Intermediate 23: (1S)-(7-(((5S)-5-Cyclopropyl-2-oxopyrrolidin-3-yl)methyl)imidazo[1,2-b]pyridazin-2-yl)4,4-difluorocyclohexyl)methanaminium trifluoroacetate

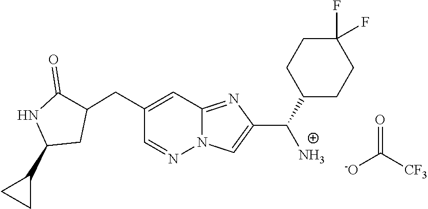

Step A: tert-Butyl ((S)-(7-((S)-5-cyclopropyl-2-oxo-1-tosylpyrrolidin-3-ylidene)methyl)imidazo[1,2-h]pyridazin-2-yl)4,4-difluorocyclohexyl)methyl)carbamate. The title compound (69% yield) was synthesized in a manner analogous to Intermediate 18 Step A using diethyl ((5S)-5-cyclopropyl-2-oxo-1-tosylpyrrolidin-3-yl)phosphonate (Intermediate 22) in place of diethyl ((5S)-2-oxo-1-tosyl-5-(trifluoromethyl)pyrrolidin-3-yl)phosphonate.

Step B: tert-Butyl ((1S)-(7-(((5S)-5-cyclopropyl-2-oxo-1-tosylpyrrolidin-3-yl)methyl)imidazo[1,2-b]pyridazin-2-yl)(4,4-difluorocyclohexyl)methyl)carbamate. The title compound was synthesized in a manner analogous to Intermediate 18 Step B using tert-butyl ((S)-(7-((S)-5-cyclopropyl-2-oxo-1-tosylpyrrolidin-3-ylidene)methyl)imidazo[1,2-b]pyridazin-2-yl)(4,4-difluorocyclohexyl)methyl)carbamate (Step A) in place of tert-butyl ((S)-5,5,5-trifluoro-4,4-dimethyl-1-(7-((S)-2-oxo-1-tosyl-5-(trifluoromethyl)pyrrolidin-3-ylidene)methyl)imidazo[1,2-b]pyridazin-2-yl)pentyl)carbamate, and was used without further purification.

Step C: tert-Butyl ((1S)-(7-(((5S)-5-cyclopropyl-2-oxopyrrolidin-3-yl)methyl)imidazo[1,2-b]pyridazin-2-yl)(4,4-difluorocyclohexyl)methyl)carbamate. The title compound (49% yield) was synthesized in a manner analogous to Intermediate 18 Step C using tert-butyl ((1S)-(7-(((5S)-5-cyclopropyl-2-oxo-1-tosylpyrrolidin-3-yl)methyl)imidazo[1,2-b]pyridazin-2-yl)(4,4-difluorocyclohexyl)methyl)carbamate (Step B) in place of tert-butyl ((1S)-5,5,5-trifluoro-4,4-dimethyl-1-(7-(((5S)-2-oxo-5-(trifluoromethyl)pyrrolidin-3-yl)methyl)imidazo[1,2-b]pyridazin-2-yl)pentyl)carbamate.

Step D: (1S)-(7-(((5S)-5-Cyclopropyl-2-oxopyrrolidin-3-yl)methyl)imidazo[1,2-b]pyridazin-2-yl)4,4-difluorocyclohexyl)methanaminium trifluoroacetate. The title compound (92% yield) was synthesized in a manner analogous to Intermediate 18 Step D using tert-butyl ((1S)-(7-(((5S)-5-cyclopropyl-2-oxopyrrolidin-3-yl)methyl)imidazo[1,2-b]pyridazin-2-yl)(4,4-difluorocyclohexyl)methyl)carbamate (Step C) in place of tert-butyl ((1S)-5,5,5-trifluoro-4,4-dimethyl-1-(7-(((5S)-2-oxo-5-(trifluoromethyl)pyrrolidin-3-yl)methyl)imidazo[1,2-b]pyridazin-2-yl)pentyl)carbamate, and the residue was used without further purification.

### Intermediate 24: 3-((2-((S)-Amino(4,4-difluorocyclohexyl)methyl)imidazo[1,2-b]pyridazin-7-yl)methyl)-4-(trifluoromethyl)piperidin-2-one

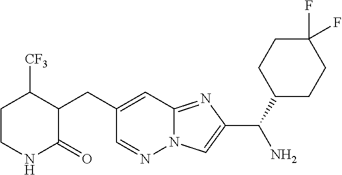 [SMILES: N[C@H](c1cn2ncc(CC3C(=O)NCCC3C(F)(F)F)cc2n1)C1CCC(F)(F)CC1]

The title compound was synthesized in a manner analogous to Intermediate 18 Step D using tert-butyl 3-((2-((S)-((tert-butoxycarbonyl)amino)4,4-difluorocyclohexyl)methyl)imidazo[1,2-b]pyridazin-7-yl)methyl)-2-oxo-4-(trifluoromethyl)piperidine-1-carboxylate (Example 4 Step A) in place of tert-butyl ((1S)-5,5,5-trifluoro-4,4-dimethyl-1-(7-(((5S)-2-oxo-5-(trifluoromethyl)pyrrolidin-3-yl)methyl)imidazo[1,2-b]pyridazin-2-yl)pentyl)carbamate. The residue was purified by silica gel chromatography (0-10% MeOH (with 2.0 M ammonia)/DCM) to provide the title compound in 74% yield.

### Intermediate 25: tert-Butyl 3-methylene-2-oxo-4-(trifluoromethyl)pyrrolidine-1-carboxylate

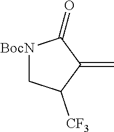 [SMILES: C=C1C(=O)N(C(=O)OC(C)(C)C)CC1C(F)(F)F]

Step A: tert-Butyl 2-oxo-4-(trifluoromethyl)pyrrolidine-1-carboxylate. The title compound (83% yield) was synthesized in a manner analogous to Intermediate 19 Step A using 4-(trifluoromethyl)pyrrolidin-2-one in place of S-5-(trifluoromethyl)pyrrolidin2-one.

Step B: tert-Butyl 3-((dimethylamino)methyl)-2-oxo-4-(trifluoromethyl)pyrrolidine-1-carboxylate. The title compound was synthesized in a manner analogous to Intermediate 19 Step B using tert-butyl 2-oxo-4-(trifluoromethyl)pyrrolidine-1-carboxylate (Step A) in place of tert-butyl (S)-2-oxo-5-(trifluoromethyl)pyrrolidine-1-carboxylate, and was used without further purification.

Step C: tert-Butyl 3-methylene-2-oxo-4-(trifluoromethyl)pyrrolidine-1-carboxylate. The title compound was synthesized in a manner analogous to Intermediate 19 Step C using tert-butyl 3-((dimethylamino)methyl)-2-oxo-4-(trifluoromethyl)pyrrolidine-1-carboxylate (Step B) in place of tert-butyl (5S)-3-((dimethylamino)methyl)-2-oxo-5-(trifluoromethyl)pyrrolidine-1-carboxylate, and was used without further purification.

### Intermediate 26: (1S)-(4,4-Difluorocyclohexyl)(7-((2-oxo-4-(trifluoromethyl)pyrrolidin-3-yl)methyl)imidazo[1,2-b]pyridazin-2-yl)methanaminium trifluoroacetate

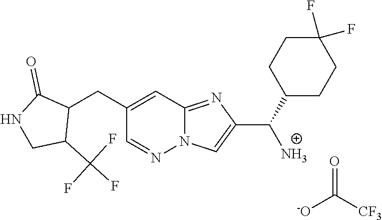

Step A: tert-Butyl 3-((2-((S)-((tert-butoxycarbonyl)amino)(4,4-difluorocyclohexyl)methyl)imidazo[1,2-b]pyridazin-7-yl)methylene)-2-oxo-4-(trifluoromethyl)pyrrolidine-1-carboxylate. tert-Butyl N—[(S)-(7-chloroimidazo[1,2-b]pyridazin-2-yl)-(4,4-difluorocyclohexyl)methyl]carbamate (100 mg, 0.249 mmol), tert-butyl 3-methylene-2-oxo-4-(trifluoromethyl)pyrrolidine-1-carboxylate (99.3 mg, 0.374 mmol, Intermediate 25), and Pd₂dba₃ (6.85 mg, 7.48 mmol) were added to a Schlenk tube and then the tube was evacuated and refilled with argon. Then, 1,4-dioxane (0.5 mL), N-cyclohexyl-N-methylcyclohexanamine (58.8 mL, 0.274 mmol), and tri-tert-butylphosphine (29.9 mL, 0.249 mmol, 1.0 M in THF) were sequentially added and the reaction was heated at 90° C. overnight. The reaction was cooled to rt, concentrated to dryness, and the residue was purified by silica gel chromatography (0-100% EtOAc/hexanes) to afford the title compound in 50% yield.

Step B: tert-Butyl 3-((2-((S)-((tert-butoxycarbonyl)amino)(4,4-difluorocyclohexyl)methyl)imidazo[1,2-b]pyridazin-7-yl)methyl)-2-oxo-4-(trifluoromethyl)pyrrolidine-1-carboxylate. The title compound (72% yield) was synthesized in a manner analogous to Intermediate 18 Step C using tert-butyl 3-((2-((S)-((tert-butoxycarbonyl)amino)(4,4-difluorocyclohexyl)methyl)imidazo[1,2-b]pyridazin-7-yl)methylene)-2-oxo-4-(trifluoromethyl)pyrrolidine-1-carboxylate (Step A) in place of tert-butyl ((1S)-5,5,5-trifluoro-4,4-dimethyl-1-(7-(((5S)-2-oxo-5-(trifluoromethyl)pyrrolidin-3-yl)methyl)imidazo[1,2-b]pyridazin-2-yl)pentyl)carbamate.

Step C: (1S)-(4,4-Difluorocyclohexyl)(7-((2-oxo-4-(trifluoromethyl)pyrrolidin-3-yl)methyl)imidazo[1,2-b]pyridazin-2-yl)methanaminium trifluoroacetate. The title compound (92% yield) was synthesized in a manner analogous to Intermediate 18 Step D using tert-butyl 3-((2-((S)-((tert-butoxycarbonyl)amino)(4,4-difluorocyclohexyl)methyl)imidazo[1,2-b]pyridazin-7-yl)methyl)-2-oxo-4-(trifluoromethyl)pyrrolidine-1-carboxylate (Step B) in place of tert-butyl ((1S)-5,5,5-trifluoro-4,4-dimethyl-1-(7-(((5S)-2-oxo-5-(trifluoromethyl)pyrrolidin-3-yl)methyl)imidazo[1,2-b]pyridazin-2-yl)pentyl)carbamate.

### Intermediate 27: 2,5-Dioxopyrrolidin-1-yl 1-isopropyl-1H-pyrazole-5-carboxylate

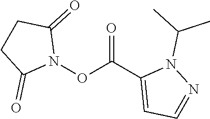 [SMILES: CC(C)n1nccc1C(=O)ON1C(=O)CCC1=O]

Step A: 1-Isopropyl-1H-pyrazole-5-carbonyl chloride. A solution of 1-isopropyl-1H-pyrazole-5-carboxylic acid (1.0 g, 6.5 mmol) in DCM (13 mL) and was cooled to 0° C. under a nitrogen atmosphere. To the solution were added oxalyl chloride (1.1 mL, 13.0 mmol) followed by DMF (0.05 mL, 0.65 mmol) dropwise. The reaction was stirred as it slowly warmed to rt. Once the gas evolution ceased the reaction was condensed into a yellow oil which was then dissolved in 12 mL of dry DCM to be stored as a 2 M solution.

Step B: 2,5-Dioxopyrrolidin-1-yl 1-isopropyl-1H-pyrazole-5-carboxylate. A solution of N-hydroxysuccinimide (1.1 g, 9.7 mmol) and DIPEA (16 mL, 6.5 mmol) in DCM (25 mL) was cooled to 0° C. under a nitrogen atmosphere. To the solution was added a solution of 1-isopropyl-1H-pyrazole-5-carbonyl chloride (13 mL, 6.5 mmol, 2 M in DCM, Step A). The resulting solution was allowed to warm to rt as it stirred for 1 h. The solution was washed with water and brine, dried over anhydrous MgSO₄, filtered, and concentrated to dryness. The residue was purified by silica gel chromatography (0-100% EtOAc (with 10% MeOH)/hexanes) to afford the title compound as an off-white solid in 61% yield.

### Intermediate 28: Diethyl ((5S)-5-methyl-1-((4-nitrophenyl)sulfonyl)-2-oxo-5-(trifluoromethyl)pyrrolidin-3-yl)phosphonate

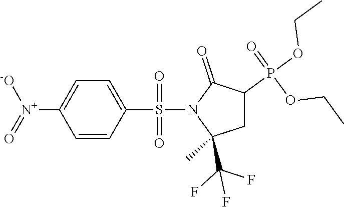

Step A: (S)-2-Methyl-1-((4-nitrophenyl)sulfonyl)-2-(trifluoromethyl)aziridine. A solution of (S)-2-amino-3,3,3-trifluoro-2-methylpropan-1-ol (7.69 g, 42.8 mmol) in anhydrous DCM (300 mL) was cooled to 0° C. Then, Nos-Cl (25.0 g, 113 mmol) was added in one portion followed by DIPEA (27.7 mL, 161 mmol) over 10 min. The resulting mixture was stirred at rt for 16 h. The reaction was quenched by the addition of 2 M aqueous HCl (200 mL) and the layers were separated. The organic layer was washed with 5% aqueous Na₂CO₃, dried over anhydrous Na₂SO₄, filtered, and concentrated to dryness. The residue was purified by silica gel chromatography (0-15% EtOAc/petroleum ether) to provide the title compound as a yellow oil (43% yield).

Step B: Ethyl (4S)-2-(diethoxyphosphoryl)-5,5,5-trifluoro-4-methyl-4-((4-nitrophenyl)sulfonamido)pentanoate. A mixture of ethyl 2-(diethoxyphosphoryl)acetate (1.3 mL, 6.4 mmol) and t-BuOK (6.4 mL, 6.4 mmol) in THF (13 mL) was heated at 40° C. for 20 min. Then, (S)-2-methyl-1-((4-nitrophenyl)sulfonyl)-2-(trifluoromethyl)aziridine (1.0 g, 3.2 mmol, Step A) was added, and the resulting mixture was heated at 40° C. for 3 h. Water (50 mL) was then added and the mixture was extracted with EtOAc (3×50 mL). The combined organic layers were washed with brine (50 mL), dried over anhydrous Na₂SO₄, filtered, and concentrated to dryness to afford a yellow oil. The oil was purified by silica gel chromatography (0-100% EtOAc/petroleum ether) to afford the title compound as a colorless oil (41% yield).

Step C: (4S)-2-(Diethoxyphosphoryl)-5,5,5-trifluoro-4-methyl-4-((4-nitrophenyl)sulfonamido)pentanoic acid. A mixture of ethyl (4S)-2-(diethoxyphosphoryl)-5,5,5-trifluoro-4-methyl-4-((4-nitrophenyl)sulfonamido)pentanoate (4.6 g, 8.61 mmol, Step B), THF (50 mL), H₂O (10 mL), and LiOH (1.81 g, 43.0 mmol) was heated at 50° C. for 16 h. Then, the reaction was diluted with H₂O (100 mL) and washed with EtOAc (100 mL). The pH of the aqueous layer was adjusted to pH 3 by the addition of 1 N aqueous HCl. The aqueous layer was then extracted with EtOAc (2×100 mL), the organic layers were combined, washed with brine and dried over anhydrous Na₂SO₄. The mixture was filtered, concentrated to dryness and used without further purification (94% yield).

Step D: Diethyl ((5S)-5-methyl-1-((4-nitrophenyl)sulfonyl)-2-oxo-5-(trifluoromethyl)pyrrolidin-3-yl)phosphonate. A mixture of (4S)-2-(diethoxyphosphoryl)-5,5,5-trifluoro-4-methyl-4-((4-nitrophenyl)sulfonamido)pentanoic acid (3.4 g, 6.7 mmol, Step C), 2-chloro-1-methylpyridin-1-ium iodide (2.57 g, 10.1 mmol) and DIPEA (3.51 mL, 20.1 mmol) in anhydrous DCM (250 mL) was stirred at rt overnight. The reaction mixture was washed sequentially with H₂O (3×100 mL) and brine (100 mL). The organic phase was dried over anhydrous Na₂SO₄, filtered, and concentrated to dryness to afford a light-yellow oil. The oil was then subjected to silica gel chromatography (0-70% EtOAc/petroleum ether) to provide the title compound as a colorless oil (29% yield).

### Intermediate 29: tert-Butyl 2-oxo-5-(trifluoromethyl)pyrrolidine-1-carboxylate

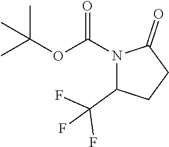 [SMILES: CC(C)(C)OC(=O)N1C(=O)CCC1C(F)(F)F]

To a solution of 5-(trifluoromethyl)pyrrolidin-2-one (5.0 g, 32 mmol) and DMAP (40 mg, 0.32 mmol) in DCM (109 mL) was added TEA (5.5 mL, 39 mmol) and then di-tert-butyl dicarbonate (8.5 g, 39 mmol) was added portion-wise and the resulting solution was stirred at rt for 18 h. The solution was partitioned between DCM and 0.5% aqueous citric acid, and the organic layer was then washed with water, dried over anhydrous Na₂SO₄, filtered, and concentrated to dryness. The residue was purified by silica gel chromatography (0-100% EtOAc/hexanes) to afford the title compound (62% yield).

### Intermediate 30: tert-Butyl (S)-2-oxo-5-(trifluoromethyl)pyrrolidine-1-carboxylate

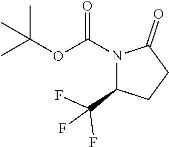 [SMILES: CC(C)(C)OC(=O)N1C(=O)CC[C@H]1C(F)(F)F]

The title compound (60% yield) was prepared as described for the synthesis of Intermediate 29 using (S)-5-(trifluoromethyl)pyrrolidin-2-one in place of 5-(trifluoromethyl)pyrrolidin-2-one.

### Intermediate 31: tert-Butyl 5-oxo-4-azaspiro[2.4]heptane-4-carboxylate

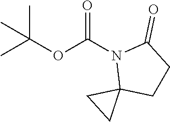

To a stirring solution of 4-azaspiro[2.4]heptan-5-one (250 mg, 2.3 mmol) in ACN (2.8 mL) was added di-tert-butyl dicarbonate (1 g, 4.6 mmol) and DMAP (274 mg, 2.3 mmol). The mixture was stirred for 3 h at rt, then diluted with water and extracted with EtOAc (3×). The organic layer was then washed with brine, dried over anhydrous Na₂SO₄, filtered, and concentrated to dryness. The residue was purified via silica gel chromatography (0-20% EtOAc/hexanes) to afford the title compound (71% yield).

### Intermediate 32: tert-Butyl 2-methyl-5-oxopyrrolidine-1-carboxylate

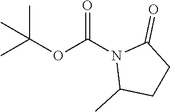

The title compound (48% yield) was prepared as described for the synthesis of Intermediate 29 using 5-methylpyrrolidin-2-one in place of 5-(trifluoromethyl)pyrrolidin-2-one.

### Intermediate 33: tert-Butyl 2-ethyl-5-oxopyrrolidine-1-carboxylate

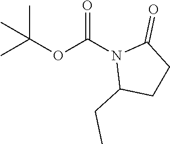

The title compound (38% yield) was prepared as described for the synthesis of Intermediate 29 using 5-ethylpyrrolidin-2-one in place of 5-(trifluoromethyl)pyrrolidin-2-one.

### Intermediate 34: tert-Butyl 2-isopropyl-5-oxopyrrolidine-1-carboxylate

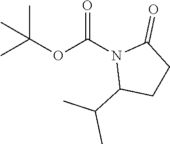 [SMILES: CC(C)C1CCC(=O)N1C(=O)OC(C)(C)C]

The title compound (38% yield) was prepared as described for the synthesis of Intermediate 29 using 5-isopropylpyrrolidin-2-one in place of 5-(trifluoromethyl)pyrrolidin-2-one.

### Intermediate 35: tert-Butyl 6-oxo-2-oxa-5-azaspiro[3.4]octane-5-carboxylate

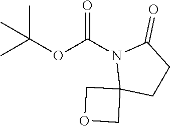 [SMILES: CC(C)(C)OC(=O)N1C(=O)CCC12COC2]

The tide compound (54% yield) was prepared as described for the synthesis of Intermediate 29 using 2-oxa-5-azaspiro[3.4]octan-6-one in place of 5-(trifluoromethyl)pyrrolidin-2-one.

### Intermediate 36: tert-Butyl 2,2-dimethyl-5-oxopyrrolidine-1-carboxylate

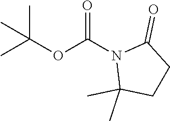

The title compound (42% yield) was prepared as described for the synthesis of Intermediate 29 using 5,5-dimethylpyrrolidin-2-one in place of 5-(trifluoromethyl)pyrrolidin-2-one.

### Intermediate 37: tert-Butyl 4,4-dimethyl-2-oxopyrrolidine-1-carboxylate

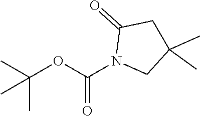

The title compound (44% yield) was prepared as described for the synthesis of Intermediate 29 using 4,4-dimethylpyrrolidin-2-one in place of 5-(trifluoromethyl)pyrrolidin-2-one.

### Intermediate 38: tert-Butyl 2-oxo-4-(trifluoromethyl)pyrrolidine-1-carboxylate

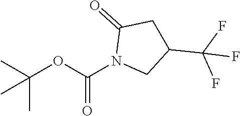 [SMILES: CC(C)(C)OC(=O)N1CC(C(F)(F)F)CC1=O]

The title compound (44% yield) was prepared as described for the synthesis of Intermediate 29 using 4-(trifluoromethyl)pyrrolidin-2-one in place of 5-(trifluoromethyl)pyrrolidin-2-one.

### Intermediate 39: tert-Butyl 4-methyl-2-oxopyrrolidine-1-carboxylate

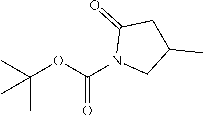

The title compound (74% yield) was prepared as described for the synthesis of Intermediate 29 using 4-methylpyrrolidin-2-one in place of 5-(trifluoromethyl)pyrrolidin-2-one.

### Intermediate 40: (5S)-3-((2-((S)-Amino(4,4-difluorocyclohexyl)methyl)imidazo[1,2-b]pyridazin-7-yl)methyl)-5-(trifluoromethyl)pyrrolidin-2-one

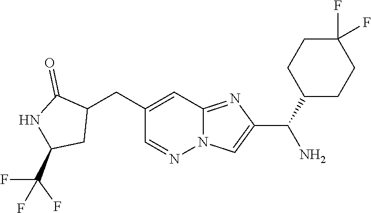 [SMILES: N[C@H](c1cn2ncc(CC3C[C@@H](C(F)(F)F)NC3=O)cc2n1)C1CCC(F)(F)CC1]

Step A: tert-Butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. A solution of tert-butyl (S)-2-oxo-5-(trifluoromethyl)pyrrolidine-1-carboxylate (Intermediate 30,455 mg, 1.80 mmol) in THF (14 mL) was cooled to −78° C. Then, a 1.5 M solution of LiHMDS in THF (1.4 mL, 2.0 mmol) was added dropwise and the resulting solution was stirred at −78° C. for 1 h. tert-Butyl (S)-((7-(bromomethyl)imidazo[1,2-b]pyridazin-2-yl)4,4-difluorocyclohexyl)methyl)carbamate (642 mg, 1.40 mmol, Intermediate 1) was dissolved in THF (2 mL) and that solution was then added into the −78° C. reaction mixture. The resulting mixture was stirred at −78° C. for 1 h. The reaction was then warmed to rt over 1 h, quenched with aqueous NH₃Cl and extracted with EtOAc (3×). The organic layers were combined, washed with brine, dried over anhydrous Na₂SO₄, filtered, and concentrated to dryness. The residue was purified by silica gel chromatography (0-100% EtOAc/hexanes) to afford the title compound (68% yield).

Step B: (5S)-3-((2-((S)-Amino(4,4-difluorocyclohexyl)methyl)imidazo[1,2-b]pyridazin-7-yl)methyl)-5-(trifluoromethyl)pyrrolidin-2-one. tert-Butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate (250 mg, 0.4 mmol, Step A) was dissolved in DCM (3.8 mL) and then TFA (0.6 mL, 8 mmol) was added. The resulting solution was stirred at rt for 1 h then concentrated to dryness. The residue was dissolved in DCM and filtered through a silica gel/K₂CO₃ plug, then concentrated to dryness to afford the title compound that was used without further purification.

### Intermediate 41: tert-Butyl (3R*,5R*)-3-((R*)-(2-((S)-((tert-butoxycarbonyl)amino(4,4-difluorocyclohexyl)methyl)imidazo[1,2-b]pyridazin-7-yl)(hydroxy)methyl)-2-oxo-5-(trifluoromethyl)pyrrolidine-1-carboxylate

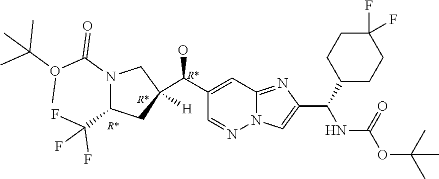

### Intermediate 42: tert-Butyl (3R*,5S*)-3-((R*)-(2-((S)-((tert-butoxycarbonyl)amino)(4,4-difluorocyclohexyl)methyl)imidazo[1,2-b]pyridazin-7-yl)(hydroxy)methyl)-2-oxo-5-(trifluoromethyl)pyrrolidine-1-carboxylate

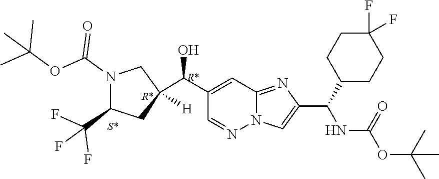 [SMILES: CC(C)(C)OC(=O)N[C@H](c1cn2ncc([C@H](O)[C@H]3C[C@@H](C(F)(F)F)N(C(=O)OC(C)(C)C)C3)cc2n1)C1CCC(F)(F)CC1]

### Intermediate 43: tert-Butyl (3S*,5R*)-3-((R*)-(2-((S)-((tert-butoxycarbonyl)amino)(4,4-difluorocyclohexyl)methyl)imidazo[1,2-b]pyridazin-7-yl)(hydroxy)methyl)-2-oxo-5-(trifluoromethyl)pyrrolidine-1-carboxylate

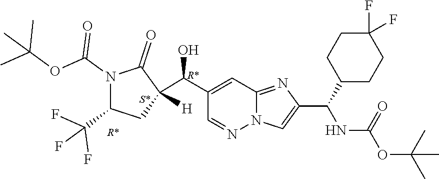 [SMILES: CC(C)(C)OC(=O)N[C@H](c1cn2ncc([C@H](O)[C@@H]3C[C@H](C(F)(F)F)N(C(=O)OC(C)(C)C)C3=O)cc2n1)C1CCC(F)(F)CC1]

### Intermediate 44: tert-Butyl (3R*,5R*)-3-((S*)-(2-((S)-((tert-butoxycarbonyl)amino)(4,4-difluorocyclohexyl)methyl)imidazo[1,2-b]pyridazin-7-yl)(hydroxy)methyl)-2-oxo-5-(trifluoromethyl)pyrrolidine-1-carboxylate

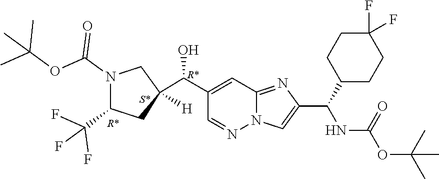 [SMILES: CC(C)(C)OC(=O)N[C@H](c1cn2ncc([C@@H](O)[C@H]3C[C@H](C(F)(F)F)N(C(=O)OC(C)(C)C)C3)cc2n1)C1CCC(F)(F)CC1]

### Intermediate 45: tert-Butyl (3R*,5S*)-3-((S*)-(2-((S)-((tert-butoxycarbonyl)amino)(4,4-difluorocyclohexyl)methyl)imidazo[1,2-b]pyridazin-7-yl)(hydroxy)methyl)-2-oxo-5-(trifluoromethyl)pyrrolidine-1-carboxylate

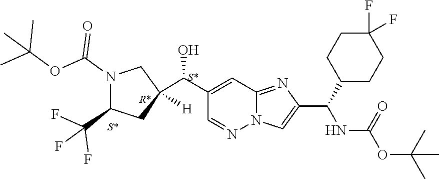 [SMILES: CC(C)(C)OC(=O)N[C@H](c1cn2ncc([C@@H](O)[C@H]3C[C@@H](C(F)(F)F)N(C(=O)OC(C)(C)C)C3)cc2n1)C1CCC(F)(F)CC1]

### Intermediate 46: tert-Butyl (3S*,5S*)-3-((R*)-(2-((S)-((tert-butoxycarbonyl)amino(4,4-difluorocyclohexyl)methyl)imidazo[1,2-b]pyridazin-7-yl)(hydroxy)methyl)-2-oxo-5-(trifluoromethyl)pyrrolidine-1-carboxylate

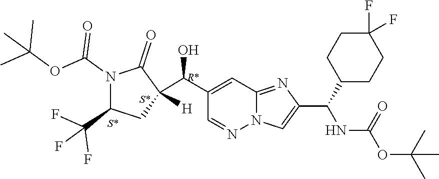 [SMILES: CC(C)(C)OC(=O)N[C@H](c1cn2ncc([C@H](O)[C@@H]3C[C@@H](C(F)(F)F)N(C(=O)OC(C)(C)C)C3=O)cc2n1)C1CCC(F)(F)CC1]

The title compounds were synthesized in a manner analogous to Intermediate 57 Step A. The diastereomers were separated by chiral SFC (Stationary phase: Whelk O1 SS, 5 μm, 250×30 mm, Mobile phase: 25% methanol:isopropanol (50:50) (with 0.2% isopropylamine), 75% CO₂). Intermediate 41 was the first isomer to elute (19% yield). Intermediate 42 was the second isomer to elute (18% yield). Intermediate 43 was the third isomer to elute (9% yield). Intermediate 44 was the fourth isomer to elute (3% yield). Intermediate 45 was the fifth isomer to elute (2% yield). Intermediate 46 was the sixth isomer to elute (5% yield).

### Intermediate 47: (1S)-5,5,5-Trifluoro-1-(7-(hydroxy((5S)-2-oxo-5-(trifluoromethyl)pyrrolidin-3-yl)methyl)imidazo[1,2-b]pyridazin-2-yl)-4,4-dimethylpentan-1-aminium 2,2,2-trifluoroacetate

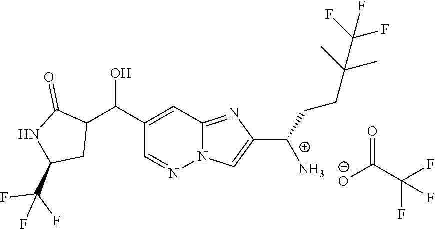 [SMILES: CC(C)(CC[C@H](N)c1cn2ncc(C(O)C3C[C@@H](C(F)(F)F)NC3=O)cc2n1)C(F)(F)F ; O=C([O-])C(F)(F)F]

Step A: tert-Butyl (5S)-3-((2-((S)-1-((tert-butoxycarbonyl)amino)-5,5,5-trifluoro-4,4-dimethylpentyl)imidazo[1,2-b]pyridazin-7-yl)(hydroxy)methyl)-2-oxo-5-(trifluoromethyl)pyrrolidine-1-carboxylate. The title compound (56% yield) was synthesized in a manner analogous to Intermediate 40 Step A using tert-butyl (S)-(5,5,5-trifluoro-1-(7-formylimidazo[1,2-b]pyridazin-2-yl)-4,4-dimethylpentyl)carbamate (Intermediate 17) in place of tert-butyl (S)-((7-(bromomethyl)imidazo[1,2-b]pyridazin-2-yl)(4,4-difluorocyclohexyl)methyl)carbamate.

Step B: (1S)-5,5,5-Trifluoro-1-(7-(hydroxy((5S)-2-oxo-5-(trifluoromethyl)pyrrolidin-3-yl)methyl)imidazo[1,2-b]pyridazin-2-yl)-4,4-dimethylpentan-1-aminium 2,2,2-trifluoroacetate. The title compound was synthesized in a manner analogous to Intermediate 57 Step B using tert-butyl (5S)-3-((2-((S)-1-((tert-butoxycarbonyl)amino)-5,5,5-trifluoro-4,4-dimethylpentyl)imidazo[1,2-b]pyridazin-7-yl)(hydroxy)methyl)-2-oxo-5-(trifluoromethyl)pyrrolidine-1-carboxylate (Step A) in place of tert-butyl 3-((2-((S)-((tert-butoxycarbonyl)amino)(4,4-difluorocyclohexyl)methyl)imidazo[1,2-b]pyridazin-7-yl)(hydroxy)methyl)-2-oxo-5-(trifluoromethyl)pyrrolidine-1-carboxylate. Purification was performed via silica gel chromatography (0-20% MeOH/DCM) to afford the title compound which was used without further purification.

### Intermediate 48: (5S)-3-(1-(2-((S)-Amino(4,4-difluorocyclohexyl)methyl)imidazo[1,2-b]pyridazin-7-yl)ethyl)-5-(trifluoromethyl)pyrrolidin-2-one

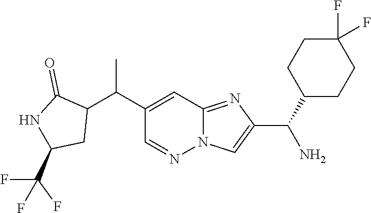 [SMILES: CC(c1cnn2cc([C@@H](N)C3CCC(F)(F)CC3)nc2c1)C1C[C@@H](C(F)(F)F)NC1=O]

Step A: tert-Butyl ((1S)-(4,4-difluorocyclohexyl)(7-(1-hydroxyethyl)imidazo[1,2-b]pyridazin-2-yl)methyl)carbamate. A solution of tert-butyl (S)-((4,4-difluorocyclohexyl)(7-formylimidazo[1,2-b]pyridazin-2-yl)methyl)carbamate (1.0 g, 2.5 mmol) in THF (32 mL) was cooled to 0° C. Then, a 3 M solution of methylmagnesium bromide in diethyl ether (2 mL, 6 mmol) was added dropwise over 5 min. The solution was stirred at 0° C. for 1 h. The reaction mixture was quenched with aqueous NH₃Cl and extracted with EtOAc (3×). The organic layers were combined, washed with brine, dried over anhydrous Na₂SO₄, filtered, and concentrated to dryness. The residue was purified via silica gel chromatography (0-100% EtOAc/hexanes) to afford the title compound (83% yield).

Step B: tert-Butyl ((1S)-(7-(1-bromoethyl)imidazo[1,2-b]pyridazin-2-yl)4,4-difluorocyclohexyl)methyl)carbamate. To a mixture of polymer-bound 100-200 mesh triphenylphosphine (500 mg, 1.5 mmol), imidazole (60 mg, 0.9 mmol) and tert-butyl ((1S)-(4,4-difluorocyclohexyl)(7-(1-hydroxyethyl)imidazo[1,2-b]pyridazin-2-yl)methyl)carbamate (300 mg, 0.731 mmol, Step A) in DCM (40 mL) was added carbon tetrabromide (291 mg, 0.877 mmol) in a single portion and the sides of the flask were rinsed with DCM (3 mL). The thick mixture was stirred overnight at rt, then filtered through a pad of diatomaceous earth that was then subsequently rinsed with DCM (2×) and the filtrate was concentrated to dryness. The residue was purified via silica gel chromatography (0-50/a EtOAc/hexanes) to afford the title compound (36% yield).

Step C: tert-Butyl (5S)-3-(1-(2-((S)-((tert-butoxycarbonyl)amino)(4,4-difluorocyclohexyl)methyl)imidazo[1,2-b]pyridazin-7-yl)ethyl)-2-oxo-5-(trifluoromethyl)pyrrolidine-1-carboxylate. The title compound (23% yield) was synthesized in a manner analogous to Intermediate 40 Step A using tert-butyl ((1S)-(7-(1-bromoethyl)imidazo[1,2-b]pyridazin-2-yl)(4,4-difluorocyclohexyl)methyl)carbamate (Step B) in place of tert-butyl (S)-((7-(bromomethyl)imidazo[1,2-b]pyridazin-2-yl)4,4-difluorocyclohexyl)methyl)carbamate.

Step D: (5S)-3-(1-(2-((S)-Amino(4,4-difluorocyclohexyl)methyl)imidazo[1,2-b]pyridazin-7-yl)ethyl)-5-(trifluoromethyl)pyrrolidin-2-one. The title compound (99% yield) was synthesized in a manner analogous to Intermediate 40 Step B using tert-butyl (5S)-3-(1-(2-((S)-((tert-butoxycarbonyl)amino)(4,4-difluorocyclohexyl)methyl)imidazo[1,2-b]pyridazin-7-yl)ethyl)-2-oxo-5-(trifluoromethyl)pyrrolidine-1-carboxylate (Step C) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate.

### Intermediate 49: (1S)-(4,4-Difluorocyclohexyl(7-((5-methyl-2-oxopyrrolidin-3-yl)methyl)imidazo[1,2-b]pyridazin-2-yl)methanaminium 2,2,2-trifluoracetate

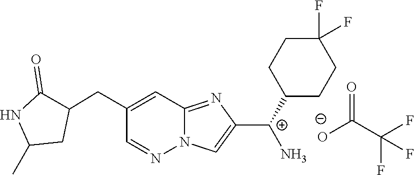

Step A: tert-Butyl 3-((2-((S)-((tert-butoxycarbonyl)amino(4,4-difluorocyclohexyl)methyl)imidazo[1,2-b]pyridazin-7-yl)methyl)-5-methyl-2-oxopyrrolidine-1-carboxylate. The title compound was synthesized in a manner analogous to Intermediate 40 Step A using tert-butyl 2-methyl-5-oxopyrrolidine-1-carboxylate (Intermediate 32) in place of tert-butyl (S)-2-oxo-5-(trifluoromethyl)pyrrolidine-1-carboxylate. The residue was purified via silica gel chromatography (0-50% EtOAc/hexanes) to afford the title compound (83% yield).

Step B: (1S)-(4,4-Difluorocyclohexyl)(7-((5-methyl-2-oxopyrrolidin-3-yl)methyl)imidazo[1,2-b]pyridazin-2-yl)methanaminium 2,2,2-trifluoracetate. The title compound was synthesized in a manner analogous to Intermediate 40 Step B using tert-butyl 3-((2-((S)-((tert-butoxycarbonyl)amino)(4,4-difluorocyclohexyl)methyl)imidazo[1,2-b]pyridazin-7-yl)methyl)-5-methyl-2-oxopyrrolidine-1-carboxylate (Step A) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The reaction mixture was concentrated to dryness and was used as the TFA salt without further purification.

### Intermediate 50: (1S)-(4,4-Difluorocyclohexyl)(7-((4-methyl-2-oxopyrrolidin-3-yl)methyl)imidazo[1,2-b]pyridazin-2-yl)methanaminium 2,2,2-trifluoroacetate

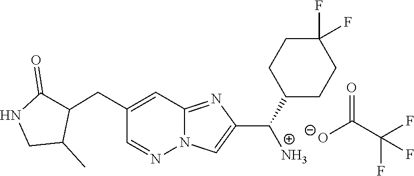 [SMILES: CC1CNC(=O)C1Cc1cnn2cc([C@@H]([NH3+])C3CCC(F)(F)CC3)nc2c1 ; O=C([O-])C(F)(F)F]

Step A: tert-Butyl 3-((2-((S)-((tert-butoxycarbonyl)amino)(4,4-difluorocyclohexyl)methyl)imidazo[1,2-b]pyridazin-7-yl)methyl)-4-methyl-2-oxopyrrolidine-1-carboxylate. The title compound was synthesized in a manner analogous to Intermediate 40 Step A using tert-butyl 4-methyl-2-oxopyrrolidine-1-carboxylate (Intermediate 39) in place of tert-butyl (S)-2-oxo-5-(trifluoromethyl)pyrrolidine-1-carboxylate. The residue was purified via silica gel chromatography (0-100% EtOAc/hexanes) to afford the title compound (50% yield).

Step B: (1S)-(4,4-Difluorocyclohexyl(7-((4-methyl-2-oxopyrrolidin-3-yl)methyl)imidazo[1,2-b]pyridazin-2-yl)methanaminium 2,2,2-trifluoroacetate. The title compound was synthesized in a manner analogous to Intermediate 40 Step B using tert-butyl 3-((2-((S)-((tert-butoxycarbonyl)amino)(4,4-difluorocyclohexyl)methyl)imidazo[1,2-b]pyridazin-7-yl)methyl)-4-methyl-2-oxopyrrolidine-1-carboxylate (Step A) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The reaction mixture was concentrated to dryness, and was used as the TFA salt without further purification.

### Intermediate 51: (1S)-(4,4-Difluorocyclohexyl)(7-((5-ethyl-2-oxopyrrolidin-3-yl)methyl)imidazo[1,2-b]pyridazin-2-yl)methanaminium 2,2,2-trifluoroacetate

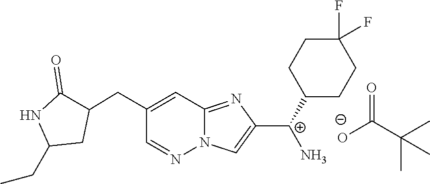

Step A: tert-Butyl 3-((2-((S)-((tert-butoxycarbonyl)amino)(4,4-difluorocyclohexyl)methyl)imidazo[1,2-b]pyridazin-7-yl)methyl)-5-ethyl-2-oxopyrrolidine-1-carboxylate. The title compound was synthesized in a manner analogous to Intermediate 40 Step A using tert-butyl 2-ethyl-5-oxopyrrolidine-1-carboxylate (Intermediate 33) in place of tert-butyl (S)-2-oxo-5-(trifluoromethyl)pyrrolidine-1-carboxylate. The residue was purified via silica gel chromatography (0-100% EtOAc/hexanes) to afford the title compound (34% yield).

Step B: tert-Butyl 3-((2-((S)-((tert-butoxycarbonyl)amino)(4,4-difluorocyclohexyl)methyl)imidazo[1,2-b]pyridazin-7-yl)methyl)-5-ethyl-2-oxopyrrolidine-1-carboxylate. The title compound was synthesized in a manner analogous to Intermediate 40 Step B using tert-butyl 3-((2-((S)-((tert-butoxycarbonyl)amino)(4,4-difluorocyclohexyl)methyl)imidazo[1,2-b]pyridazin-7-yl)methyl)-5-ethyl-2-oxopyrrolidine-1-carboxylate (Step A) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The reaction mixture was concentrated to dryness and was used as the TFA salt without further purification.

### Intermediate 52: (1S)-(4,4-Difluorocyclohexyl)(7-((5-isopropyl-2-oxopyrrolidin-3-yl)methyl)imidazo[1,2-b]pyridazin-2-yl)methanaminium 2,2,2-trifluoroacetate

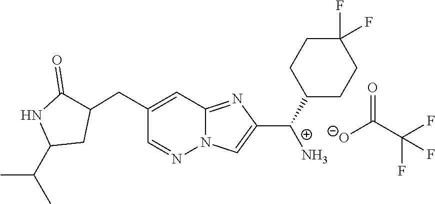 [SMILES: CC(C)C1CC(Cc2cnn3cc([C@@H]([NH3+])C4CCC(F)(F)CC4)nc3c2)C(=O)N1 ; O=C([O-])C(F)(F)F]

Step A: tert-Butyl 3-((2-((S)-((tert-butoxycarbonyl)amino)(4,4-difluorocyclohexyl)methyl)imidazo[1,2-b]pyridazin-7-yl)methyl)-5-isopropyl-2-oxopyrrolidine-1-carboxylate. The title compound was synthesized in a manner analogous to Intermediate 40 Step A using tert-butyl 2-isopropyl-5-oxopyrrolidine-1-carboxylate (Intermediate 34) in place of tert-butyl (S)-2-oxo-5-(trifluoromethyl)pyrrolidine-1-carboxylate. The residue was purified via silica gel chromatography (0-100% EtOAc/hexanes) to afford the title compound (55% yield).

Step B: (1S)-(4,4-Difluorocyclohexyl)(7-((5-isopropyl-2-oxopyrrolidin-3-yl)methyl)imidazo[1,2-b]pyridazin-2-yl)methanaminium 2,2,2-trifluoroacetate. The title compound was synthesized in a manner analogous to Intermediate 40 Step B using tert-butyl 3-((2-((S)-((tert-butoxycarbonyl)amino)(4,4-difluorocyclohexyl)methyl)imidazo[1,2-b]pyridazin-7-yl)methyl)-5-isopropyl-2-oxopyrrolidine-1-carboxylate (Step A) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The reaction mixture was concentrated to dryness and was used as the TFA salt without further purification.

### Intermediate 53: (1S)-(4,4-Difluorocyclohexyl)(7-(((5S)-3-methyl-2-oxo-5-(trifluoromethyl)pyrrolidin-3-yl)methyl)imidazo[1,2-b]pyridazin-2-yl)methanaminium 2,2,2-trifluoroacetate

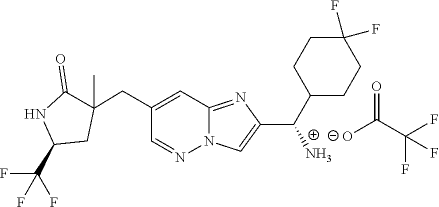 [SMILES: CC1(Cc2cnn3cc([C@@H]([NH3+])C4CCC(F)(F)CC4)nc3c2)C[C@@H](C(F)(F)F)NC1=O ; O=C([O-])C(F)(F)F]

Step A: tert-Butyl (5S)-3-((2-((S)-((tert-butoxycarbonyl)amino)(4,4-difluorocyclohexyl)methyl)imidazo[1,2-b]pyridazin-7-yl)methyl)-3-methyl-2-oxo-5-(trifluoromethyl)pyrrolidine-1-carboxylate. A solution of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate (Intermediate 40 Step A, 210 mg, 0.33 mmol) in THF (3 mL) was cooled to −78° C. Then, a solution of 1 M LiHMDS in THF (0.7 mL, 0.7 mmol) was added dropwise and the resulting solution was stirred at −78° C. for 45 min. A solution of iodomethane (22.8 μL, 0.366 mmol) in THF (0.5 mL) was added dropwise and the resulting mixture was stirred for 30 min at −78° C. Another solution of iodomethane (35 μL, 0.4316 mmol) in THF (0.4 mL) was then added dropwise. The mixture was warmed to rt over 5 min and then additional iodomethane (62 μL, 0.99 mmol) was added. The resulting solution was stirred at rt for 30 min then quenched with aqueous NH₃Cl. The mixture was extracted with EtOAc (3×), then the organic layers were combined, washed with brine, dried over anhydrous Na₂SO₄, filtered, and concentrated to dryness. Purification was performed via silica gel chromatography (0-100% EtOAc/hexanes) to afford the title compound, which was used without further purification.

Step B: (1S)-(4,4-Difluorocyclohexyl)(7-(((5S)-3-methyl-2-oxo-5-(trifluoromethyl)pyrrolidin-3-yl)methyl)imidazo[1,2-b]pyridazin-2-yl)methanaminium 2,2,2-trifluoroacetate. The title compound was synthesized in a manner analogous to Intermediate 40 Step B using tert-butyl (5S)-3-((2-((S)-((tert-butoxycarbonyl)amino(4,4-difluorocyclohexyl)methyl)imidazo[1,2-b]pyridazin-7-yl)methyl)-3-methyl-2-oxo-5-(trifluoromethyl)pyrrolidine-1-carboxylate (Step A) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The reaction mixture was concentrated to dryness and was used as the TFA salt without further purification.

### Intermediate 54: (1S)-(4,4-Difluorocyclohexyl)(7-((6-oxo-2-oxa-5-azaspiro[3.4]octan-7-yl)methyl)imidazo[1,2-b]pyridazin-2-yl)methanaminium 2,2,2-trifluoroacetate

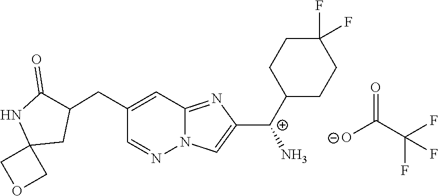

Step A: tert-Butyl 7-((2-((S)-((tert-butoxycarbonyl)amino)(4,4-difluorocyclohexyl)methyl)imidazo[1,2-b]pyridazin-7-yl)methyl)-6-oxo-2-oxa-5-azaspiro[3.4]octane-5-carboxylate. The title compound was synthesized in a manner analogous to Intermediate 40 Step A using tert-butyl 6-oxo-2-oxa-5-azaspiro[3.4]octane-5-carboxylate (Intermediate 35) in place of tert-butyl (S)-2-oxo-5-(trifluoromethyl)pyrrolidine-1-carboxylate. The residue was purified via silica gel chromatography (0-100% acetone/hexanes (0.1% TEA)) to afford the title compound (53% yield).

Step B: (1S)-(4,4-Difluorocyclohexyl)(7-((6-oxo-2-oxa-5-azaspiro[3.4]octan-7-yl)methyl)imidazo[1,2-b]pyridazin-2-yl)methanaminium 2,2,2-trifluoroacetate. The title compound was synthesized in a manner analogous to Intermediate 40 Step B using tert-butyl 7-((2-((S)-((tert-butoxycarbonyl)amino)(4,4-difluorocyclohexyl)methyl)imidazo[1,2-b]pyridazin-7-yl)methyl)-6-oxo-2-oxa-5-azaspiro[3.4]octane-5-carboxylate (Step A) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The reaction mixture was concentrated to dryness and was used as the TFA salt without further purification.

### Intermediate 55: (1S)-(4,4-Difluorocyclohexyl)(7-(((5S)-3-(methoxymethyl)-2-oxo-5-(trifluoromethyl)pyrrolidin-3-yl)methyl)imidazo[1,2-b]pyridazin-2-yl)methanaminium 2,2,2-trifluoroacetate

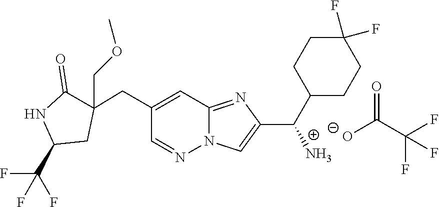 [SMILES: COCC1(Cc2cnn3cc([C@@H]([NH3+])C4CCC(F)(F)CC4)nc3c2)C[C@@H](C(F)(F)F)NC1=O ; O=C([O-])C(F)(F)F]

Step A: tert-Butyl (5S)-3-((2-((S)-((tert-butoxycarbonyl)amino)(4,4-difluorocyclohexyl)methyl)imidazo[1,2-b]pyridazin-7-yl)methyl)-3-(methoxymethyl)-2-oxo-5-(trifluoromethyl)pyrrolidine-1-carboxylate. The title compound was synthesized in a manner analogous to Intermediate 53 Step A using MOMCl in place of iodomethane. Purification was performed via silica gel chromatography (0-100% EtOAc/hexanes) to afford the title compound, which was used without further purification.

Step B: (1S)-(4,4-Difluorocyclohexyl)(7-(((5S)-3-(methoxymethyl)-2-oxo-5-(trifluoromethyl)pyrrolidin-3-yl)methyl)imidazo[1,2-b]pyridazin-2-yl)methanaminium 2,2,2-trifluoroacetate. The title compound was synthesized in a manner analogous to Intermediate 40 Step B using tert-butyl (5S)-3-((2-((S)-((tert-butoxycarbonyl)amino)(4,4-difluorocyclohexyl)methyl)imidazo[1,2-b]pyridazin-7-yl)methyl)-3-(methoxymethyl)-2-oxo-5-(trifluoromethyl)pyrrolidine-1-carboxylate (Step A) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The reaction mixture was concentrated to dryness and was used as the TFA salt without further purification.

### Intermediate 56: 3-((2-((S)-Amino(4,4-difluorocyclohexyl)methyl)imidazo[1,2-b]pyridazin-7-yl)methyl)-4-(trifluoromethyl)pyrrolidin-2-one

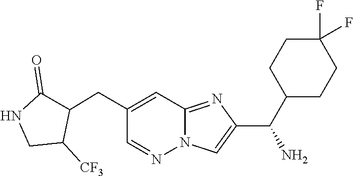 [SMILES: N[C@H](c1cn2ncc(CC3C(=O)NCC3C(F)(F)F)cc2n1)C1CCC(F)(F)CC1]

Step A: tert-Butyl 3-((2-((S)-((tert-butoxycarbonyl)amino)(4,4-difluorocyclohexyl)methyl)imidazo[1,2-b]pyridazin-7-yl)methyl)-2-oxo-4-(trifluoromethyl)pyrrolidine-1-carboxylate. The title compound was synthesized in a manner analogous to Intermediate 40 Step A using tert-butyl 2-oxo-4-(trifluoromethyl)pyrrolidine-1-carboxylate (Intermediate 38) in place of tert-butyl (S)-2-oxo-5-(trifluoromethyl)pyrrolidine-1-carboxylate. The residue was purified via silica gel chromatography (0-100% EtOAc/hexanes) to afford the title compound (30% yield).

Step B: 3-((2-((S)-Amino(4,4-difluorocyclohexyl)methyl)imidazo[1,2-b]pyridazin-7-yl)methyl)-4-(trifluoromethyl)pyrrolidin-2-one. The title compound was synthesized in a manner analogous to Intermediate 40 Step B using tert-butyl 3-((2-((S)-((tert-butoxycarbonyl)amino)4,4-difluorocyclohexyl)methyl)imidazo[1,2-b]pyridazin-7-yl)methyl)-2-oxo-4-(trifluoromethyl)pyrrolidine-1-carboxylate (Step A) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The reaction mixture was partitioned between saturated aqueous Na₂CO₃ and 20% IPA/DCM. The aqueous layer was further extracted with 20% IPA/DCM (3×). Then, the combined organic layers were dried over anhydrous Na₂SO₄, filtered, concentrated to dryness and used without any further purification.

### Intermediate 57: (1S)-(4,4-Difluorocyclohexyl)(7-(hydroxy(2-oxo-5-(trifluoromethyl)pyrrolidin-3-yl)methyl)imidazo[1,2-b]pyridazin-2-yl)methanaminium 2,2,2-trifluoroacetate

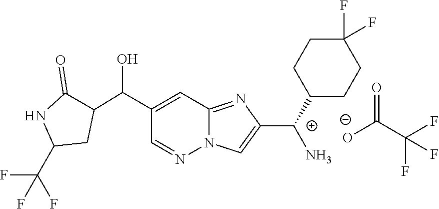 [SMILES: N[C@H](c1cn2ncc(C(O)C3CC(C(F)(F)F)NC3=O)cc2n1)C1CCC(F)(F)CC1 ; O=C([O-])C(F)(F)F]

Step A: tert-Butyl 3-((2-((S)-((tert-butoxycarbonyl)amino)(4,4-difluorocyclohexyl)methyl)imidazo[1,2-b]pyridazin-7-yl)(hydroxy)methyl)-2-oxo-5-(trifluoromethyl)pyrrolidine-1-carboxylate. The title compound (31% yield) was synthesized in a manner analogous to Intermediate 40 Step A using tert-butyl 2-oxo-5-(trifluoromethyl)pyrrolidine-1-carboxylate (Intermediate 29) in place of tert-butyl (S)-2-oxo-5-(trifluoromethyl)pyrrolidine-1-carboxylate and tert-butyl (S)-((4,4-difluorocyclohexyl)₇-formylimidazo[1,2-b]pyridazin-2-yl)methyl)carbamate in place of tert-butyl (S)-((7-(bromomethyl)imidazo[1,2-b]pyridazin-2-yl)4,4-difluorocyclohexyl)methyl)carbamate.

Step B: (1S)-(4,4-Difluorocyclohexyl)(7-(hydroxy(2-oxo-5-(trifluoromethyl)pyrrolidin-3-yl)methyl)imidazo[1,2-b]pyridazin-2-yl)methanaminium 2,2,2-trifluoroacetate. To a solution of tert-butyl 3-((2-((S)-((tert-butoxycarbonyl)amino)(4,4-difluorocyclohexyl)methyl)imidazo[1,2-b]pyridazin-7-yl)(hydroxy)methyl)-2-oxo-5-(trifluoromethyl)pyrrolidine-1-carboxylate (100 mg, 0.15 mmol, Step A) in DCM (0.5 mL) was added TFA (0.5 mL). The resulting solution was stirred at rt for 1 h, and then was concentrated to dryness. The residue was used without further purification.

### Intermediate 58: (1S)-(4,4-Difluorocyclohexyl)(7-((5-oxo-4-azaspiro[2.4]heptan-6-yl)methyl)imidazo[1,2-b]pyridazin-2-yl)methanaminium 2,2,2-trifluoroacetate

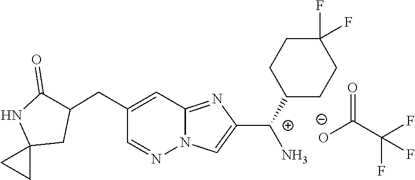 [SMILES: N[C@H](c1cn2ncc(CC3CC4(CC4)NC3=O)cc2n1)C1CCC(F)(F)CC1 ; O=C([O-])C(F)(F)F]

Step A: tert-Butyl 6-((2-((S)-((tert-butoxycarbonyl)amino)(4,4-difluorocyclohexyl)methyl)imidazo[1,2-b]pyridazin-7-yl)methyl)-5-oxo-4-azaspiro[2.4]heptane-4-carboxylate. The title compound was synthesized in a manner analogous to Intermediate 40 Step A using tert-butyl 5-oxo-4-azaspiro[2.4]heptane-4-carboxylate (Intermediate 31) in place of tert-butyl (S)-2-oxo-5-(trifluoromethyl)pyrrolidine-1-carboxylate. The residue was used without further purification.

Step B: (1S)-(4,4-Difluorocyclohexyl)(7-((5-oxo-4-azaspiro[2.4]heptan-6-yl)methyl)imidazo[1,2-b]pyridazin-2-yl)methanaminium 2,2,2-trifluoroacetate. The title compound was synthesized in a manner analogous to Intermediate 57 Step B using tert-butyl 6-((2-((S)-((tert-butoxycarbonyl)amino)(4,4-difluorocyclohexyl)methyl)imidazo[1,2-b]pyridazin-7-yl)methyl)-5-oxo-4-azaspiro[2.4]heptane-4-carboxylate (Step A) in place of tert-butyl 3-((2-((S)-((tert-butoxycarbonyl)amino)(4,4-difluorocyclohexyl)methyl)imidazo[1,2-b]pyridazin-7-yl)(hydroxy)methyl)-2-oxo-5-(trifluoromethyl)pyrrolidine-1-carboxylate. The residue was purified by acidic reverse phase preparative HPLC (C18 column, 5 μm, 30×250 mm, 0-100% ACN (with 0.05% TFA) in H₂O (with 0.05% TFA)) to afford the title compound (13% yield over two steps).

### Intermediate 59: (3R*,5R*)-3-((R*)-(2-((S)-Amino(4,4-difluorocyclohexyl)methyl)imidazo[1,2-b]pyridazin-7-yl)(hydroxy)methyl)-5-(trifluoromethyl)pyrrolidin-2-one

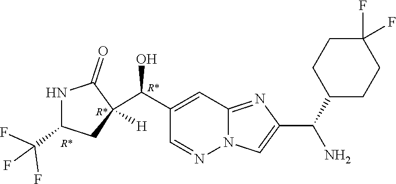 [SMILES: N[C@H](c1cn2ncc([C@H](O)[C@H]3C[C@H](C(F)(F)F)NC3=O)cc2n1)C1CCC(F)(F)CC1]

The title compound was synthesized in a manner analogous to Intermediate 40 Step B using tert-butyl (3R*,5R*)-3-((R*)-(2-((S)-((tert-butoxycarbonyl)amino)(4,4-difluorocyclohexyl)methyl)imidazo[1,2-b]pyridazin-7-yl)(hydroxy)methyl)-2-oxo-5-(trifluoromethyl)pyrrolidine-1-carboxylate (Intermediate 41) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate, and was used without further purification.

### Intermediate 60: tert-Butyl (5S)-3-(2-((S)-((tert-butoxycarbonyl)amino)(4,4-difluorocyclohexyl)methyl)imidazo[1,2-b]pyridazine-7-carbonyl)-2-oxo-5-(trifluoromethyl)pyrrolidine-1-carboxylate

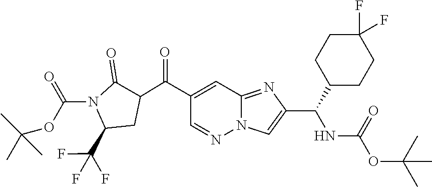 [SMILES: CC(C)(C)OC(=O)N[C@H](c1cn2ncc(C(=O)C3C[C@@H](C(F)(F)F)N(C(=O)OC(C)(C)C)C3=O)cc2n1)C1CCC(F)(F)CC1]

A solution of tert-butyl 3-((2-((S)-((tert-butoxycarbonyl)amino(4,4-difluorocyclohexyl)methyl)imidazo[1,2-b]pyridazin-7-yl)(hydroxy)methyl)-2-oxo-5-(trifluoromethyl)pyrrolidine-1-carboxylate (Intermediate 57 Step A, 500 mg, 0.77 mmol) in DCM (7.7 mL) was cooled to 0° C. and then DMP (491 mg, 1.2 mmol) was added portion-wise. The solution was stirred at rt for 15 min, then it was quenched by the addition of saturated aqueous NaS₂O₃. The layers were separated and the organic layer was washed sequentially with saturated aqueous NaHCO₃ and brine, dried over anhydrous Na₂SO₄, filtered, and concentrated to dryness. The residue was purified via silica gel chromatography (0-100% EtOAc (10% MeOH)/hexanes) to afford the title compound (74% yield).

### Intermediate 61: (3R,5S)-3-(2-((S)-Amino(4,4-difluorocyclohexyl)methyl)imidazo[1,2-b]pyridazine-7-carbonyl)-3-methyl-5-(trifluoromethyl)pyrrolidin-2-one

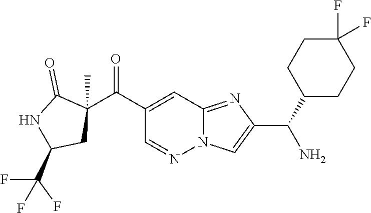 [SMILES: C[C@@]1(C(=O)c2cnn3cc([C@@H](N)C4CCC(F)(F)CC4)nc3c2)C[C@@H](C(F)(F)F)NC1=O]

Step A: tert-Butyl (3R,5S)-3-(2-((S)-((tert-butoxycarbonyl)amino)(4,4-difluorocyclohexyl)methyl)imidazo[1,2-b]pyridazine-7-carbonyl)-3-methyl-2-oxo-5-(trifluoromethyl)pyrrolidine-1-carboxylate. tert-Butyl (5S)-3-(2-((S)-((tert-butoxycarbonyl)amino)(4,4-difluorocyclohexyl)methyl)imidazo[1,2-b]pyridazine-7-carbonyl)-2-oxo-5-(trifluoromethyl)pyrrolidine-1-carboxylate (334 mg, 0.52 mmol, Intermediate 60) and K₂CO₃ (170 mg, 1.2 mmol) were dissolved in DMF (2 mL) and the mixture was stirred at rt for 5 min. Then, Mel (0.32 mL, 5.2 mmol) was added dropwise. The reaction mixture was stirred at rt for 1 h, diluted with water and extracted with EtOAc (3×). The organic layers were combined, washed with brine and dried over anhydrous Na₂SO₄. The organic layer was filtered, concentrated to dryness and purified via silica gel chromatography (0-100% EtOAc/hexanes) to afford a 3:1 mixture of the title compound and its diastereomer (tert-butyl (3S,5S)-3-(2-((S)-((tert-butoxycarbonyl)amino)(4,4-difluorocyclohexyl)methyl)imidazo[1,2-b]pyridazine-7-carbonyl)-3-methyl-2-oxo-5-(trifluoromethyl)pyrrolidine-1-carboxylate). The title compound was obtained in 53% yield and its absolute stereochemistry was determined by 2D NMR.

Step B: (3R,5S)-3-(2-((S)-Amino(4,4-difluorocyclohexyl)methyl)imidazo[1,2-b]pyridazine-7-carbonyl)-3-methyl-5-(trifluoromethyl)pyrrolidin-2-one. To a solution of tert-butyl (3R,5S)-3-(2-((S)-((tert-butoxycarbonyl)amino)(4,4-difluorocyclohexyl)methyl)imidazo[1,2-b]pyridazine-7-carbonyl)-3-methyl-2-oxo-5-(trifluoromethyl)pyrrolidine-1-carboxylate (65 mg, 0.1 mmol, Step A) in DCM (1.6 mL) was added TFA (0.4 mL, 5 mmol). The resulting mixture was stirred at rt for 1 h then concentrated to dryness. The residue was purified via silica gel chromatography (0-10% MeOH (2 M NH₃)/DCM) to afford the title compound (63% yield).

### Intermediate 62: (1S)-(7-(((5S)-3-(Cyanomethyl)-2-oxo-5-(trifluoromethyl)pyrrolidin-3-yl)methyl)imidazo[1,2-b]pyridazin-2-yl)(4,4-difluorocyclohexyl)methanaminium 2,2,2-trifluoroacetate

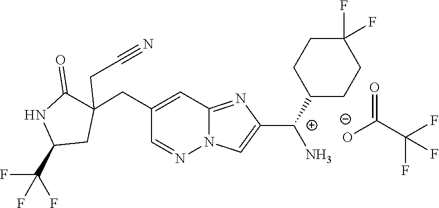 [SMILES: N#CCC1(Cc2cnn3cc([C@@H](N)C4CCC(F)(F)CC4)nc3c2)C[C@@H](C(F)(F)F)NC1=O ; O=C([O-])C(F)(F)F]

Step A: tert-Butyl (5S)-3-((2-((S)-((tert-butoxycarbonyl)amino)(4,4-difluorocyclohexyl)methyl)imidazo[1,2-b]pyridazin-7-yl)methyl)-3-(cyanomethyl)-2-oxo-5-(trifluoromethyl)pyrrolidine-1-carboxylate. The title compound was synthesized in a manner analogous to Intermediate 53 Step A using bromoacetonitrile in place of iodomethane. Purification was performed via silica gel chromatography (0-100% EtOAc/hexanes) to afford the title compound (56% yield).

Step B: (1S)-(7-(((5S)-3-(Cyanomethyl)-2-oxo-5-(trifluoromethyl)pyrrolidin-3-yl)methyl)imidazo[1,2-b]pyridazin-2-yl)(4,4-difluorocyclohexyl)methanaminium 2,2,2-trifluoroacetate. The title compound was synthesized in a manner analogous to Intermediate 57 Step B using tert-butyl (5S)-3-((2-((S)-((tert-butoxycarbonyl)amino)(4,4-difluorocyclohexyl)methyl)imidazo[1,2-b]pyridazin-7-yl)methyl)-3-(cyanomethyl)-2-oxo-5-(trifluoromethyl)pyrrolidine-1-carboxylate (Step A) in place of tert-butyl 3-((2-((S)-((tert-butoxycarbonyl)amino)(4,4-difluorocyclohexyl)methyl)imidazo[1,2-b]pyridazin-7-yl)(hydroxy)methyl)-2-oxo-5-(trifluoromethyl)pyrrolidine-1-carboxylate. Purification was performed via silica gel chromatography (0-30% MeOH/DCM) to afford the title compound (79% yield).

### Intermediate 63: 3-((2-((S)-Amino(4,4-difluorocyclohexyl)methyl)imidazo[1,2-b]pyridazin-7-yl)methyl)-5-(trifluoromethyl)pyrrolidin-2-one

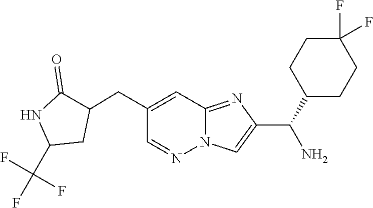 [SMILES: N[C@H](c1cn2ncc(CC3CC(C(F)(F)F)NC3=O)cc2n1)C1CCC(F)(F)CC1]

Step A: tert-Butyl 3-((2-((S)-((tert-butoxycarbonyl)amino(4,4 difluorocyclohexyl)methyl)imidazo[1,2-b]pyridazin-7-yl)methyl)-2-oxo-5 (trifluoromethyl)pyrrolidine-1-carboxylate. The title compound was synthesized in a manner analogous to Intermediate 40 using tert-butyl 2-oxo-5-(trifluoromethyl)pyrrolidine-1-carboxylate (Intermediate 29) in place of tert-butyl (S) 2-oxo-5-(trifluoromethyl)pyrrolidine-1-carboxylate (Intermediate 30), and was used without further purification.

Step B: 3-((2-((S)-Amino(4,4-difluorocyclohexyl)methyl)imidazo[1,2-b]pyridazin-7-yl)methyl)-5-(trifluoromethyl)pyrrolidin-2-one. The title compound was synthesized in a manner analogous to Intermediate 40 Step B using tert-butyl 3-((2-((S)-((tert-butoxycarbonyl)amino)(4,4 difluorocyclohexyl)methyl)imidazo[1,2-b]pyridazin-7-yl)methyl)-2-oxo-5 (trifluoromethyl)pyrrolidine-1-carboxylate (Step A) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate, and was used without further purification.

### Example 1: 4-Cyclopropyl-N—((S)-(7-(((R*)-5,5-difluoro-2-oxopiperidin-3-yl)methyl)imidazo[1,2-b]pyridazin-2-yl)(4,4-difluorocyclohexyl)methyl)-1,2,5-oxadiazole-3-carboxamide

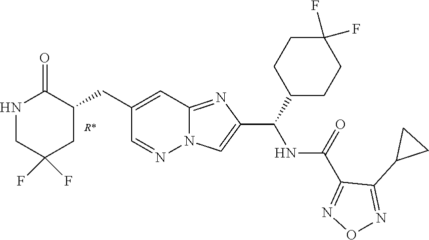 [SMILES: O=C(N[C@H](c1cn2ncc(C[C@@H]3CC(F)(F)CNC3=O)cc2n1)C1CCC(F)(F)CC1)c1nonc1C1CC1]

### Example 2: 4-Cyclopropyl-N—((S)-(7-(((S*)-5,5-difluoro-2-oxopiperidin-3-yl)methyl)imidazo[1,2-b]pyridazin-2-yl)(4,4-difluorocyclohexyl)methyl)-1,2,5-oxadiazole-3-carboxamide

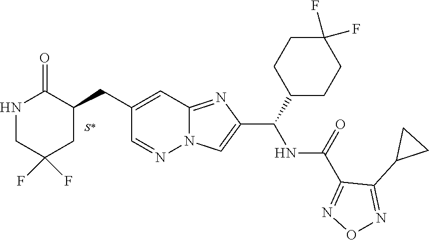 [SMILES: O=C(N[C@H](c1cn2ncc(C[C@H]3CC(F)(F)CNC3=O)cc2n1)C1CCC(F)(F)CC1)c1nonc1C1CC1]

Step A: tert-Butyl 3-((2-((S)-((tert-butoxycarbonyl)amino)(4,4-difluorocyclohexyl)methyl)imidazo[1,2-b]pyridazin-7-yl)methyl)-5,5-difluoro-2-oxopiperidine-1-carboxylate. A solution of 1,1-dimethylethyl 5,5-difluoro-2-oxo-1-piperidinecarboxylate (467 mg, 1.99 mmol) in THF (16.4 mL) was cooled to −78° C. and then LiHMDS (2.13 mL, 2.13 mmol, 1 M in THF) was added. The resulting mixture was stirred at −78° C. for 1 h. Then, tert-butyl (S)-((7-(bromomethyl)imidazo[1,2-b]pyridazin-2-yl)(4,4-difluorocyclohexyl)methyl)carbamate (754 mg, 1.64 mmol, Intermediate 1) was dissolved in THF (3.3 mL) and the resulting solution was added to the reaction mixture dropwise over 10 min. The resulting mixture was stirred at −78° C. for 20 min and was then allowed to warm to rt over 1 h. The mixture was extracted with EtOAc (3×), then the organic layers were combined, washed with brine, dried over anhydrous Na₂SO₄, filtered, and concentrated to dryness. The residue was purified by silica gel chromatography (0-50% EtOAc/hexanes) to provide the title compound (20% yield).

Step B: 3-((2-((S)-Amino(4,4-difluorocyclohexyl)methyl)imidazo[1,2-b]pyridazin-7-yl)methyl)-5,5-difluoropiperidin-2-one. To a mixture of tert-butyl 3-((2-((S)-((tert-butoxycarbonyl)amino)(4,4-difluorocyclohexyl)methyl)imidazo[1,2-b]pyridazin-7-yl)methyl)-5,5-difluoro-2-oxopiperidine-1-carboxylate (230 mg, 0.37 mmol, Step A) in DCM (4.8 mL) was added TFA (0.72 mL, 9.4 mmol) and the resulting mixture was stirred at rt for 2 h. The mixture was concentrated to remove TFA, then filtered through a plug of silica gel and K₂CO₃ and concentrated to dryness to provide the title compound (100% yield).

Step C: 4-Cyclopropyl-N—((S)-(7-(((R*)-5,5-difluoro-2-oxopiperidin-3-yl)methyl)imidazo[1,2-b]pyridazin-2-yl)(4,4-difluorocyclohexyl)methyl)-1,2,5-oxadiazole-3-carboxamide and 4-cyclopropyl-N—((S)-(7-(((S*)-5,5-difluoro-2-oxopiperidin-3-yl)methyl)imidazo[1,2-b]pyridazin-2-yl)(4,4-difluorocyclohexyl)methyl)-1,2,5-oxadiazole-3-carboxamide. To a solution of 3-((2-((S)-amino(4,4-difluorocyclohexyl)methyl)imidazo[1,2-b]pyridazin-7-yl)methyl)-5,5-difluoropiperidin-2-one (154 mg, 0.37 mmol, Step B) in ACN (3 mL) was added DIPEA (0.26 mL, 1.5 mmol) and the resulting solution was stirred under N₂ at rt for 5 min. Then, a solution of 2,5-dioxopyrrolidin-1-yl 4-cyclopropyl-1,2,5-oxadiazole-3-carboxylate (143 mg, 0.57 mmol, Intermediate 2) in ACN (1 mL) was added to the reaction mixture. The resulting mixture was stirred at rt for 2 h under N₂. The reaction was concentrated to dryness, dissolved in DCM and purified by silica gel chromatography (0-100/acetone/hexanes (with 0.1% TEA)) to provide a mixture of diastereomers. The diastereomers were separated by chiral SFC (Chiralpak IB N, 5 μm, 250×21 mm, 30% methanol, 70% CO₂). The first-eluting isomer (19% yield) was designated as the (R*) isomer (Example 1) and the second-eluting isomer (19% yield) was designated as the (S*) isomer (Example 2). Example 1: ¹H NMR (500 MHz, DMSO-d₆) δ 9.47 (d, J=9.0 Hz, 1H), 8.44 (d, J=2.0 Hz, 1H), 8.19 (s, 1H), 7.91 (d, J=1.5 Hz, 1H), 7.89-7.86 (m, 1H), 5.18 (t, J=8.5 Hz, 1H), 3.58-3.44 (m, 2H), 3.24-3.06 (m, 1H), 2.97-2.88 (m, 1H), 2.82 (dd, J=8.0, 14.0 Hz, 1H), 2.32-2.23 (m, 2H), 2.21-2.13 (m, 2H), 2.13-1.95 (m, 3H), 1.92-1.87 (m, 1H), 1.84-1.71 (m, 2H), 1.66-1.59 (m, 1H), 1.44-1.24 (m, 2H), 1.18-1.06 (m, 2H), 1.01-0.91 (m, 2H). MS (ESI) m/z: [M+H]⁺ Found 550.3. Example 2: ¹H NMR (400 MHz, DMSO-d₆) δ 9.47 (d, J=9.0 Hz, 1H), 8.44 (d, J=2.0 Hz, 1H), 8.19 (s, 1H), 7.91 (s, 1H), 7.90-7.86 (m, 1H), 5.19 (t, J=8.5 Hz, 1H), 3.58-3.47 (m, 2H), 3.19-3.13 (m, 1H), 2.99-2.89 (m, 1H), 2.87-2.79 (m, 1H), 2.31-2.23 (m, 2H), 2.21-1.96 (m, 4H), 1.95-1.71 (m, 3H), 1.63 (br d, J=13.6 Hz, 1H), 1.45-1.23 (m, 2H), 1.18-1.08 (m, 2H), 1.02-0.93 (m, 2H). MS (ESI) m/z: [M+H]⁺ Found 550.3.

### Example 3: 4-Cyclopropyl-N—[(S)-(4,4-difluorocyclohexyl)-[7-[[(3S*,4R*)-2-oxo-4-(trifluoromethyl)-3-piperidyl]methyl]imidazo[1,2-b]pyridazin-2-yl]methyl]-1,2,5-oxadiazole-3-carboxamide

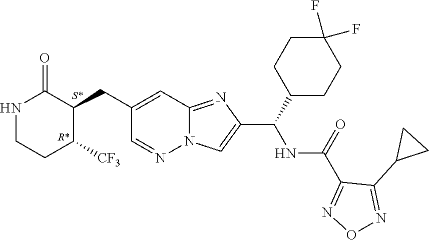 [SMILES: O=C(N[C@H](c1cn2ncc(C[C@@H]3C(=O)NCC[C@H]3C(F)(F)F)cc2n1)C1CCC(F)(F)CC1)c1nonc1C1CC1]

### Example 4: 4-Cyclopropyl-N—[(S)-(4,4-difluorocyclohexyl)-[7-[[(3R*,4S*)-2-oxo-4-(trifluoromethyl)-3-piperidyl]methyl]imidazo[1,2-b]pyridazin-2-yl]methyl]-1,2,5-oxadiazole-3-carboxamide

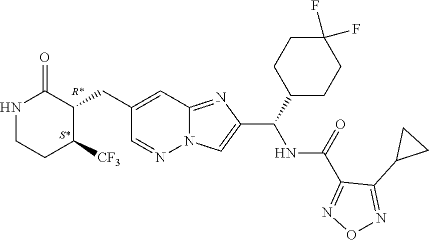 [SMILES: O=C(N[C@H](c1cn2ncc(C[C@H]3C(=O)NCC[C@@H]3C(F)(F)F)cc2n1)C1CCC(F)(F)CC1)c1nonc1C1CC1]

Step A: tert-Butyl 3-((2-((S)-((tert-butoxycarbonyl)amino(4,4-difluorocyclohexyl)methyl)imidazo[1,2-b]pyridazin-7-yl)methyl)-2-oxo-4-(trifluoromethyl)piperidine-1-carboxylate. The title compound was prepared as described for the synthesis of Example 1 and Example 2 Step A using tert-butyl 2-oxo-4-(trifluoromethyl)piperidine-1-carboxylate (Intermediate 6) in place of 1,1-dimethylethyl 5,5-difluoro-2-oxo-1-piperidinecarboxylate and cooling the reaction to −45° C. instead of −78° C. The residue was purified by silica gel chromatography (0-100% (EtOAc with 10% MeOH)/hexanes) to provide the title compound in 55% yield.

Step B: 3-((2-((S)-Amino(4,4-difluorocyclohexyl)methyl)imidazo[1,2-b]pyridazin-7-yl)methyl)-4-(trifluoromethyl)piperidin-2-one. To a mixture of tert-butyl 3-((2-((S)-((tert-butoxycarbonyl)amino)(4,4-difluorocyclohexyl)methyl)imidazo[1,2-b]pyridazin-7-yl)methyl)-2-oxo-4-(trifluoromethyl)piperidine-1-carboxylate (390 mg, 0.600 mmol, Step A) in DCM (2 mL) was added TFA (2.0 mL, 26 mmol) and the resulting mixture was stirred at rt for 1 h. The mixture was concentrated, then dissolved in DCM and 0.5 M aqueous NaOH was added slowly. The mixture was separated and the aqueous further extracted with DCM. The organic layers were combined, washed with brine, dried over anhydrous Na₂SO₄, filtered and concentrated to dryness. The residue was purified by silica gel chromatography (0-10% (MeOH with 2 M NH₃)/DCM) to provide the title compound (94% yield).

Step C: 4-Cyclopropyl-N—[(S)-(4,4-difluorocyclohexyl)-[7-[[(3S*,4R*)-2-oxo-4-(trifluoromethyl)-3-piperidyl]methyl]imidazo[1,2-b]pyridazin-2-yl]methyl]-1,2,5-oxadiazole-3-carboxamide and 4-cyclopropyl-N—[(S)-(4,4-difluorocyclohexyl)-[7-[[(3R*,4S*)-2-oxo-4-(trifluoromethyl)-3-piperidyl]methyl]imidazo[1,2-b]pyridazin-2-yl]methyl]-1,2,5-oxadiazole-3-carboxamide. To a solution of 4-cyclopropyl-1,2,5-oxadiazole-3-carboxylic acid (108 mg, 0.7 mmol) and T₃P (0.41 mL, 0.69 mmol, 50% in EtOAc) in EtOAc (2.5 mL) was added DIPEA (0.19 mL, 1.1 mmol) and the resulting mixture was stirred at rt for 2 min. Then, a solution of 3-((2-((S)-amino(4,4-difluorocyclohexyl)methyl)imidazo[1,2-b]pyridazin-7-yl)methyl)-4-(trifluoromethyl)piperidin-2-one (154 mg, 0.350 mmol, Step B) in DCM (2.5 mL) was added to the reaction mixture and the resulting mixture was stirred at rt for 18 h. A solution of 4-cyclopropyl-1,2,5-oxadiazole-3-carboxylic acid (85 mg, 0.55 mmol), T₃P (0.33 mL, 0.55 mmol, 50% in EtOAc) and DIPEA (0.19 mL, 1.1 mmol) in EtOAc (0.8 mL) was stirred at rt for 10 min and then added to the reaction mixture and the resulting mixture was stirred at rt for 4 h. Then, the reaction was partitioned between water and EtOAc. The aqueous layer was further extracted with EtOAc (1×). The organic layers were combined, washed with brine, dried over anhydrous Na₂SO₄, filtered, and concentrated to dryness. The residue was purified by silica gel chromatography (0-100% (EtOAc with 10% MeOH)/hexanes) to provide a mixture of diastereomers. The diastereomers were separated by chiral SFC (Whelk O1 SS, 5 μm, 250×21 mm, 25% methanol, 75% CO₂). The first-eluting isomer (32% yield) was designated as the (3S*,4R*) isomer (Example 3) and the second-eluting isomer (29% yield) was designated as the (3R*,4S*) isomer (Example 4). Example 3: ¹H NMR (500 MHz, DMSO-d₆) δ 9.48 (d, J=9.1 Hz, 1H), 8.38 (d, J=2.0 Hz, 1H), 8.20 (s, 1H), 7.92 (br s, 1H), 7.85 (d, J=1.6 Hz, 1H), 5.19 (t, J=8.6 Hz, 1H), 3.25-3.03 (m, 4H), 2.80-2.70 (m, 2H), 2.27 (tt, J=5.0, 8.4 Hz, 1H), 2.21-2.14 (m, 1H), 2.09-1.97 (m, 3H), 1.93-1.87 (m, 1H), 1.87-1.71 (m, 3H), 1.66-1.59 (m, 1H), 1.44-1.23 (m, 2H), 1.16-1.06 (m, 2H), 1.01-0.91 (m, 2H). MS (ESI) m/z: [M+H]⁺ Found 582.2. Example 4: ¹H NMR (500 MHz, DMSO-d₆) δ 9.47 (d, J=9.1 Hz, 1H), 8.39-8.37 (m, 1H), 8.21-8.19 (m, 1H), 7.93-7.91 (m, 1H), 7.87-7.85 (m, 1H), 5.19 (t, J=8.5 Hz, 1H), 3.24-3.19 (m, 1H), 3.17-3.11 (m, 2H), 3.09-3.03 (m, 1H), 2.81-2.70 (m, 2H), 2.28 (tt, J=5.0, 8.4 Hz, 1H), 2.22-2.13 (m, 1H), 2.09-1.97 (m, 3H), 1.92-1.87 (m, 1H), 1.86-1.71 (m, 3H), 1.66-1.60 (m, 1H), 1.44-1.23 (m, 2H), 1.17-1.06 (m, 2H), 1.01-0.93 (m, 2H). MS (ESI) m/z: [M+H]⁺ Found 582.2.

### Example 5: 4-Methoxy-N-[(1S)-5,5,5-trifluoro-4,4-dimethyl-1-[7-[[(3R*,5S)-2-oxo-5-(trifluoromethyl)pyrrolidin-3-yl]methyl]imidazo[1,2-b]pyridazin-2-yl]pentyl]-1,2,5-oxadiazole-3-carboxamide

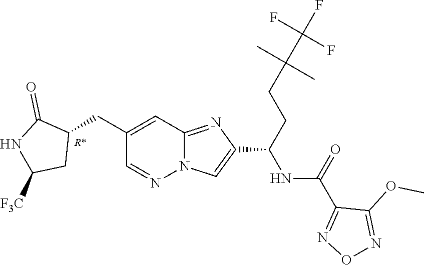 [SMILES: COc1nonc1C(=O)N[C@@H](CCC(C)(C)C(F)(F)F)c1cn2ncc(C[C@@H]3C[C@@H](C(F)(F)F)NC3=O)cc2n1]

### Example 6: 4-Methoxy-N-[(1S)-5,5,5-trifluoro-4,4-dimethyl-1-[7-[[(3S*,5S)-2-oxo-5-(trifluoromethyl)pyrrolidin-3-yl]methyl]imidazo[1,2-b]pyridazin-2-yl]pentyl]-1,2,5-oxadiazole-3-carboxamide

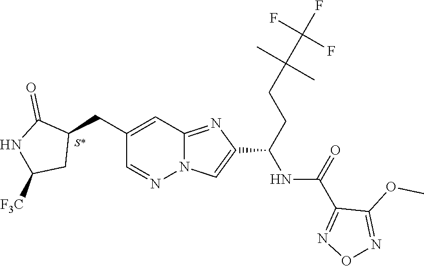 [SMILES: COc1nonc1C(=O)N[C@@H](CCC(C)(C)C(F)(F)F)c1cn2ncc(C[C@H]3C[C@@H](C(F)(F)F)NC3=O)cc2n1]

A solution of (5S)-3-((2-((S)-1-amino-5,5,5-trifluoro-4,4-dimethylpentyl)imidazo[1,2-b]pyridazin-7-yl)methyl)-5-(trifluoromethyl)pyrrolidin-2-one (138 mg, 0.31 mmol, Intermediate 18), 4-methoxy-1,2,5-oxadiazole-3-carboxylic acid (66 mg, 0.46 mmol) and DIPEA (0.26 mL, 1.53 mmol) in DCM (3.1 mL) was stirred at rt for 10 min, then T₃P (0.36 mL, 0.61 mmol, 50% in EtOAc) was added and the resulting mixture was stirred at rt for 60 min. The reaction mixture was concentrated to dryness and purified by preparative basic HPLC (X-Bridge Prep C18 5 μm column, 50×250 mm, 5-100% acetonitrile/water (with 20 mM NH₄OH)) to provide a mixture of diastereomers. The diastereomers were separated by chiral SFC (Chiralpak IF, 5 μm, 250×21 mm, 20% ACN/methanol (1/1), 80% CO₂). The first-eluting isomer (2% yield) was designated as the (R*) isomer (Example 5) and the second-eluting isomer was designated as the (S*) isomer (Example 6). The second-eluting isomer was repurified by silica gel chromatography (0-100% acetone/hexanes) to provide Example 6 in 6% yield. Example 5: ¹H NMR (500 MHz, DMSO-d₆) δ 9.39-9.35 (m, 1H), 8.71-8.68 (m, 1H), 8.48-8.45 (m, 1H), 8.13-8.11 (m, 1H), 7.97-7.95 (m, 1H), 5.20-5.15 (m, 1H), 4.25-4.20 (m, 1H), 4.11-4.09 (m, 3H), 3.12-3.06 (m, 1H), 2.95-2.87 (m, 1H), 2.76-2.69 (m, 1H), 2.19-2.13 (m, 1H), 2.12-2.04 (m, 2H), 1.99-1.90 (m, 1H), 1.68-1.60 (m, 1H), 1.55-1.48 (m, 1H), 1.29-1.24 (m, 1H), 1.10-1.08 (m, 6H). MS (ESI) m/z: [M+H]⁺ Found 578.2. Example 6: ¹H NMR (500 MHz, DMSO-do) δ 9.36 (d, J=8.6 Hz, 1H), 8.65 (s, 1H), 8.45 (d, J=2.0 Hz, 1H), 8.12 (s, 1H), 7.96-7.94 (m, 1H), 5.18 (dt, J=5.3, 8.6 Hz, 1H), 4.36-4.18 (m, 1H), 4.10 (s, 3H), 3.10 (dd, J=4.9, 14.3 Hz, 1H), 2.99-2.83 (m, 1H), 2.75 (dd, J=9.2, 14.3 Hz, 1H), 2.46-2.39 (m, 1H), 2.16-2.04 (m, 1H), 1.95 (td, J=8.6, 12.8 Hz, 1H), 1.71-1.59 (m, 2H), 1.55-1.48 (m, 1H), 1.08 (s, 6H). MS (ESI) m/z: [M+H]⁺ Found 578.2.

### Example 7: N—[(S)-(4,4-Difluorocyclohexyl)-[7-[[(3R*,5S)-2-oxo-5-(trifluoromethyl)pyrrolidin-3-yl]methyl]imidazo[1,2-b]pyridazin-2-yl]methyl]-4-methoxy-1,2,5-oxadiazole-3-carboxamide

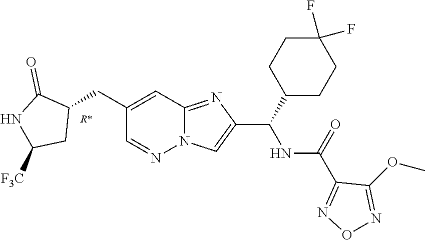 [SMILES: COc1nonc1C(=O)N[C@H](c1cn2ncc(C[C@@H]3C[C@@H](C(F)(F)F)NC3=O)cc2n1)C1CCC(F)(F)CC1]

### Example 8: N—((S)-(4,4-Difluorocyclohexyl)(7-(((3S*,5S)-2-oxo-5-(trifluoromethyl)pyrrolidin-3-yl)methyl)imidazo[1,2-b]pyridazin-2-yl)methyl)-4-methoxy-1,2,5-oxadiazole-3-carboxamide

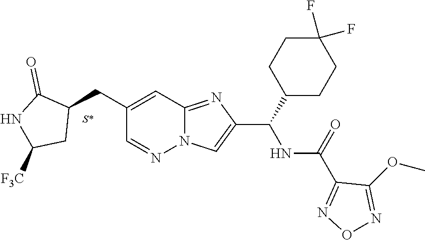 [SMILES: COc1nonc1C(=O)N[C@H](c1cn2ncc(C[C@H]3C[C@@H](C(F)(F)F)NC3=O)cc2n1)C1CCC(F)(F)CC1]

To a solution of 4-methoxy-1,2,5-oxadiazole-3-carboxylic acid (63 mg, 0.44 mmol) and T₃P (0.30 mL, 0.51 mmol, 50% in EtOAc) in EtOAc (0.77 mL) was added DIPEA (0.12 mL, 0.70 mmol) and the resulting mixture was stirred at rt for 10 min. Then, a solution of (5S)-3-((2-((S)-amino(4,4-difluorocyclohexyl)methyl)imidazo[1,2-b]pyridazin-7-yl)methyl)-5-(trifluoromethyl)pyrrolidin-2-one (100 mg, 0.230 mmol, Intermediate 40) in DCM (2.3 mL) was added to the reaction mixture and the resulting mixture was stirred at rt for 30 min. The reaction was concentrated to dryness, dissolved in DMF and purified by preparative basic HPLC to provide a mixture of diastereomers. The diastereomers were separated by chiral SFC (Chiralpak IB N3, 5 μm, 250×21 mm, 30% methanol/isopropanol (1/1), 70% CO₂). The first-eluting isomer (21% yield) was designated as the (R*) isomer (Example 7) and the second-eluting isomer (20% yield) was designated as the (S*) isomer (Example 8). Example 7: ¹H NMR (500 MHz, DMSO-d₆) δ 9.28 (d, J=9.1 Hz, 1H), 8.65 (s, 1H), 8.45 (d, J=2.0 Hz, 1H), 8.16 (s, 1H), 7.95-7.93 (m, 1H), 5.15 (s, 1H), 4.29-4.22 (m, 1H), 4.08 (s, 3H), 3.10 (dd, J=4.9, 14.4 Hz, 1H), 2.94-2.86 (m, 1H), 2.73 (dd, J=9.4, 14.4 Hz, 1H), 2.49-2.37 (m, 1H), 2.15 (br d, J=8.4 Hz, 1H), 2.11-1.94 (m, 2H), 1.88-1.79 (m, 2H), 1.78-1.71 (m, 1H), 1.71-1.64 (m, 1H), 1.63-1.56 (m, 1H), 1.41-1.33 (m, 1H), 1.31-1.23 (m, 1H). MS (ESI) m/z: [M+H]⁺ Found 558.0. Example 8: ¹H NMR (500 MHz, DMSO-d₆) δ 9.32-9.28 (m, 1H), 8.70-8.68 (m, 1H), 8.47-8.45 (m, 1H), 8.17-8.16 (m, 1H), 7.96-7.94 (m, 1H), 5.18-5.13 (m, 1H), 4.25-4.20 (m, 1H), 4.10-4.09 (m, 3H), 3.11-3.06 (m, 1H), 2.94-2.87 (m, 1H), 2.75-2.69 (m, 1H), 2.21-2.10 (m, 3H), 2.07-1.97 (m, 2H), 1.89-1.80 (m, 2H), 1.80-1.72 (m, 1H), 1.65-1.59 (m, 1H), 1.43-1.34 (m, 1H), 1.31-1.24 (m, 1H). MS (ESI) m/z: [M+H]⁺ Found 558.2.

### Example 9: N—[(S)-(4,4-Difluorocyclohexyl)-[7-[[(3S*,5S)-2-oxo-5-(trifluoromethyl)pyrrolidin-3-yl]methyl]imidazo[1,2-b]pyridazin-2-yl]methyl]-4-methyl-1,2,5-oxadiazole-3-carboxamide

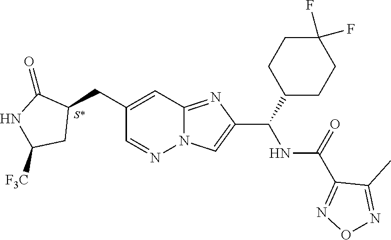 [SMILES: Cc1nonc1C(=O)N[C@H](c1cn2ncc(C[C@H]3C[C@@H](C(F)(F)F)NC3=O)cc2n1)C1CCC(F)(F)CC1]

### Example 10: N—[(S)-(4,4-Difluorocyclohexyl)-[7-[[(3R*,5S)-2-oxo-5-(trifluoromethyl)pyrrolidin-3-yl]methyl]imidazo[1,2-b]pyridazin-2-yl]methyl]-4-methyl-1,2,5-oxadiazole-3-carboxamide

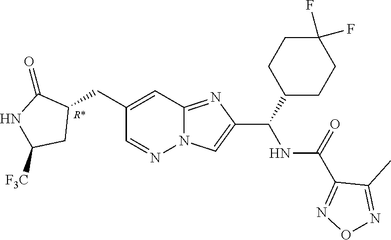 [SMILES: Cc1nonc1C(=O)N[C@H](c1cn2ncc(C[C@@H]3C[C@@H](C(F)(F)F)NC3=O)cc2n1)C1CCC(F)(F)CC1]

To a solution of (5S)-3-((2-((S)-amino(4,4-difluorocyclohexyl)methyl)imidazo[1,2-b]pyridazin-7-yl)methyl)-5-(trifluoromethyl)pyrrolidin-2-one (100 mg, 0.230 mmol, Intermediate 40) in ACN (6.2 mL) were added DIPEA (0.20 mL, 1.2 mmol) and 2,5-dioxopyrrolidin-1-yl 4-methyl-1,2,5-oxadiazole-3-carboxylate (97 mg, 0.43 mmol) sequentially. The resulting mixture was stirred at rt for 10 min, then concentrated to dryness. The residue was purified by preparative basic HPLC (X-Bridge Prep C18 5 μm column, 50×250 mm, 5-100% acetonitrile/water (with 20 mM NH₄OH)) to provide a mixture of diastereomers. The diastereomers were separated by chiral SFC (Chiralpak IB N3, 5 μm, 250×21 mm, 25% methanol, 75% CO₂). The first-eluting isomer (16% yield) was designated as the (S*) isomer (Example 9) and the second-eluting isomer (15% yield) was designated as the (R*) isomer (Example 10). Example 9: ¹H NMR (500 MHz, DMSO-d₆) δ 9.45-9.39 (m, 1H), 8.65 (s, 1H), 8.45 (d, J=2.0 Hz, 1H), 8.20 (s, 1H), 7.96-7.93 (m, 1H), 5.19-5.15 (m, 1H), 4.31-4.24 (m, 1H), 3.13-3.08 (m, 1H), 2.97-2.89 (m, 1H), 2.77-2.71 (m, 1H), 2.48-2.47 (m, 3H), 2.45-2.36 (m, 1H), 2.23-2.15 (m, 1H), 2.11-1.95 (m, 2H), 1.95-1.88 (m, 1H), 1.86-1.60 (m, 4H), 1.44-1.35 (m, 1H), 1.34-1.23 (m, 1H). MS (ESI) m/z: [M+H]⁺ Found 542.2. Example 10: ¹H NMR (500 MHz, DMSO-d₆) δ 9.33 (d, J=9.0 Hz, 1H), 8.62 (s, 1H), 8.38 (d, J=2.0 Hz, 1H), 8.13 (s, 1H), 7.87 (d, J=1.5 Hz, 1H), 5.09 (s, 1H), 4.16 (br s, 1H), 3.04-2.98 (m, 1H), 2.88-2.79 (m, 1H), 2.68-2.61 (m, 1H), 2.41-2.39 (m, 3H), 2.15-1.91 (m, 5H), 1.86-1.79 (m, 1H), 1.79-1.63 (m, 2H), 1.59-1.51 (m, 1H), 1.37-1.27 (m, 1H), 1.26-1.16 (m, 1H). MS (ESI) m/z: [M+H]⁺ Found 542.2.

### Example 11: 4-Cyclopropyl-N—[(S)-(4,4-difluorocyclohexyl)-[7-[[(1R*,5R*)-7-oxo-6-azabicyclo[3.2.1]octan-1-yl]methyl]imidazo[1,2-b]pyridazin-2-yl]methyl]-1,2,5-oxadiazole-3-carboxamide

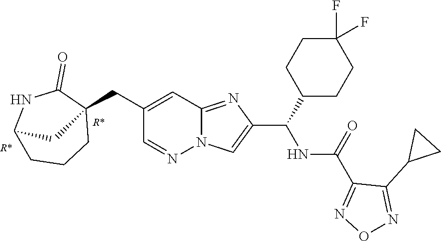 [SMILES: O=C(N[C@H](c1cn2ncc(C[C@@]34CCC[C@H](C3)NC4=O)cc2n1)C1CCC(F)(F)CC1)c1nonc1C1CC1]

### Example 12: 4-Cyclopropyl-N—[(S)-(4,4-difluorocyclohexyl)-[7-[[(1S*5S*)-7-oxo-6-azabicyclo[3.2.1]octan-1-yl]methyl]imidazo[1,2-b]pyridazin-2-yl]methyl]-1,2,5-oxadiazole-3-carboxamide

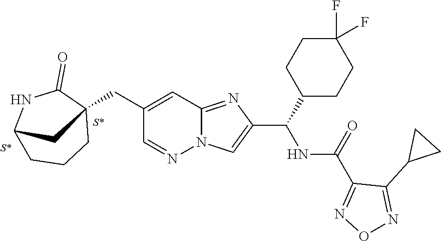 [SMILES: O=C(N[C@H](c1cn2ncc(C[C@]34CCC[C@@H](C3)NC4=O)cc2n1)C1CCC(F)(F)CC1)c1nonc1C1CC1]

Step A: Methyl 1-((2-((S)-((tert-butoxycarbonyl)amino)(4,4-difluorocyclohexyl)methyl)imidazo[1,2-b]pyridazin-7-yl)methyl)cyclohex-3-ene-1-carboxylate. To a mixture of methyl cyclohex-3-ene-1-carboxylate (0.51 mL, 3.6 mmol) in THF (30 mL) at −70° C. was added LDA (9.41 mL, 9.41 mmol, 1 M in THF) dropwise over 10 min, and the resulting mixture was stirred at −70° C. for 30 min. Then, a solution of tert-butyl (S)-((7-(bromomethyl)imidazo[1,2-b]pyridazin-2-yl)(4,4-difluorocyclohexyl)methyl)carbamate (1.5 g, 3.26 mmol, Intermediate 1) in THF (8 mL) was added to the reaction mixture over 5 min. The resulting mixture was stirred at −70° C. for 2 h. The reaction was quenched with a solution of HOAc (0.5 mL) in THF (25 mL) and the mixture was stirred at −70° C. for 5 min. Then, the cooling bath was removed and the reaction was allowed to warm to rt. The mixture was partitioned between water (40 mL) and EtOAc (50 mL), and the aqueous layer further extracted with EtOAc (3×50 mL). The organic layers were combined, washed with brine (2×50 mL), dried over anhydrous MgSO₄, filtered, and concentrated to dryness. The residue was purified by silica gel chromatography (0-30% EtOAc/petroleum ether) to provide the title compound as a white solid (53% yield).

Step B: 1-((2-((S)-((tert-Butoxycarbonyl)amino)(4,4-difluorocyclohexyl)methyl)imidazo[1,2-b]pyridazin-7-yl)methyl)cyclohex-3-ene-1-carboxylic acid. A mixture of methyl 1-((2-((S)-((tert-butoxycarbonyl)amino)(4,4-difluorocyclohexyl)methyl)imidazo[1,2-b]pyridazin-7-yl)methyl)cyclohex-3-ene-1-carboxylate (1.03 g, 1.99 mmol, Step A) and LiOH·H₂O (833 mg, 19.9 mmol) in H₂O (10 mL) and MeOH (30 mL) was heated at 35° C. overnight. The reaction mixture was concentrated to remove MeOH, diluted with water (10 mL) and extracted with DCM (20 mL). The pH of the aqueous layer was adjusted to pH 3 by the addition of 1 N aqueous HCl and then the aqueous was extracted with EtOAc (2×20 mL). The organic layers were combined, washed with brine (20 mL), dried over anhydrous Na₂SO₄, filtered, and concentrated to dryness to provide the title compound as a yellow oil (100% yield), which was used without further purification.

Step C: tert-Butyl ((1S)-(4,4-difluorocyclohexyl)(7-((1-(((4-nitrophenyl)sulfonyl)carbamoyl)cyclohex-3-en-1-yl)methyl)imidazo[1,2-b]pyridazin-2-yl)methyl)carbamate. A mixture of 1-((2-((S)-((tert-butoxycarbonyl)amino(4,4-difluorocyclohexyl)methyl)imidazo[1,2-b]pyridazin-7-yl)methyl)cyclohex-3-ene-1-carboxylic acid (1.00 g, 1.98 mmol, Step B), EDCI (379 mg, 1.98 mmol), HOBt (267 mg, 1.98 mmol) and DIPEA (0.86 mL, 4.9 mmol) in DCM (30 mL) was stirred at rt for 30 min, then 4-nitrobenzenesulfonamide (400 mg, 1.98 mmol) was added in one portion and the resulting mixture was stirred at rt for 2 h. The mixture was diluted with water (30 mL) and extracted with DCM (3×100 mL). The organic layers were combined, washed with brine (150 mL), dried over anhydrous Na₂SO₄, filtered, and concentrated to dryness. The residue was purified by silica gel chromatography (0-10% MeOH/DCM) to provide the title compound as a yellow oil (47% yield).

Step D: 1-((2-((S)-Amino(4,4-difluorocyclohexyl)methyl)imidazo[1,2-b]pyridazin-7-yl)methyl)-N-((4-nitrophenyl)sulfonyl)cyclohex-3-ene-1-carboxamide. A mixture of tert-butyl ((1S)-(4,4-difluorocyclohexyl)(7-((1-(((4-nitrophenyl)sulfonyl)carbamoyl)cyclohex-3-en-1-yl)methyl)imidazo[1,2-b]pyridazin-2-yl)methyl)carbamate (437 mg, 0.63 mmol, Step C), 4 M HCl in 1,4-dioxane (4 mL, 16 mmol) and 1,4-dioxane (4 mL) was stirred at rt for 2 h. The reaction mixture was diluted with water (15 mL), the pH of the solution was adjusted to pH 8 by the addition of saturated aqueous NaHCO₃ and the mixture was extracted with EtOAc (4×15 mL). The organic layers were combined, washed with brine (100 mL), dried over anhydrous Na₂SO₄, filtered, and concentrated to dryness to provide the title compound as a white solid (100% yield), which was used without further purification.

Step E: 4-Cyclopropyl-N-((1S)-(4,4-difluorocyclohexyl)(7-((1-(((4-nitrophenyl)sulfonyl)carbamoyl)cyclohex-3-en-1-yl)methyl)imidazo[1,2-b]pyridazin-2-yl)methyl)-1,2,5-oxadiazole-3-carboxamide. A mixture of 4-cyclopropyl-1,2,5-oxadiazole-3-carboxylic acid (195 mg, 1.27 mmol), 1-hydroxypyrrolidine-2,5-dione (146 mg, 1.27 mmol) and EDCI (304 mg, 1.58 mmol) in DCM (10 mL) was stirred at rt for 30 min. Then, 1-((2-((S)-amino(4,4-difluorocyclohexyl)methyl)imidazo[1,2-b]pyridazin-7-yl)methyl)-N-((4-nitrophenyl)sulfonyl)cyclohex-3-ene-1-carboxamide (396 mg, 0.63 mmol, Step D) and DIPEA (0.55 mL, 3.2 mmol) were added and the resulting mixture was stirred at rt for 30 min. The mixture was subjected directly to silica gel chromatography (0-10% MeOH/DCM) to provide the title compound as an off-white solid (69% yield).

Step F: 4-Cyclopropyl-N-((1S)-(4,4-difluorocyclohexyl)(7-((4-iodo-6-((4-nitrophenyl)sulfonyl)-7-oxo-6-azabicyclo[3.2.]octan-1-yl)methyl)imidazo[1,2-b]pyridazin-2-yl)methyl)-1,2,5-oxadiazole-3-carboxamide. A mixture of 4-cyclopropyl-N-((1S)-(4,4-difluorocyclohexyl)(7-((1-(((4-nitrophenyl)sulfonyl)carbamoyl)cyclohex-3-en-1-yl)methyl)imidazo[1,2-b]pyridazin-2-yl)methyl)-1,2,5-oxadiazole-3-carboxamide (370 mg, 0.51 mmol, Step E) and K₂CO₃ (430 mg, 3.06 mmol) in ACN (5 mL) was cooled to 0° C. Then, 12 (518 mg, 2.04 mmol) was added and the resulting mixture was stirred at rt for 2 h. The mixture was diluted with water (15 mL) and extracted with EtOAc (3×15 mL). The organic layers were combined, washed with brine (20 mL), dried over anhydrous Na₂SO₄, filtered, and concentrated to dryness. The residue was purified by silica gel chromatography (0-10% MeOH/DCM) to provide the title compound as an oil (65% yield).

Step G: 4-Cyclopropyl-N-((1S)-(4,4-difluorocyclohexyl)(7-((6-((4-nitrophenyl)sulfonyl)-7-oxo-6-azabicyclo[3.2.1]octan-1-yl)methyl)imidazo[1,2-b]pyridazin-2-yl)methyl)-1,2,5-oxadiazole-3-carboxamide. A mixture of 4-cyclopropyl-N-((1S)-(4,4-difluorocyclohexyl)(7-((4-iodo-6-((4-nitrophenyl)sulfonyl)-7-oxo-6-azabicyclo[3.2.1]octan-1-yl)methyl)imidazo[1,2-b]pyridazin-2-yl)methyl)-1,2,5-oxadiazole-3-carboxamide (160 mg, 0.19 mmol, Step F), n-Bu₃SnH (59 μL, 0.23 mmol) and AIBN (10 mg, 0.056 mmol) was added to a flask which was then evacuated and filled with N₂ three times. The resulting mixture was heated at 110° C. for 2 h. The reaction was cooled to rt, poured into 1 M aqueous KF solution (5 mL) and the mixture was stirred at rt for 30 min. The mixture was extracted with EtOAc (3×15 mL) and the organic layers were combined. The organic extracts were washed with brine (15 mL), dried over anhydrous MgSO₄, filtered, and concentrated to dryness. The residue was purified by silica gel chromatography (0-10% MeOH/DCM) to provide the title compound as a yellow oil (37% yield).

Step H: 4-Cyclopropyl-N—[(S)-(4,4-difluorocyclohexyl)-[7-[[(1R*,5R*)-7-oxo-6-azabicyclo[3.2.1]octan-1-yl]methyl]imidazo[1,2-b]pyridazin-2-yl]methyl]-1,2,5-oxadiazole-3-carboxamide and 4-cyclopropyl-N—[(S)-(4,4-difluorocyclohexyl)-[7-[[(1S*,5S*)-7-oxo-6-azabicyclo[3.2.1]octan-1-yl]methyl]imidazo[1,2-b]pyridazin-2-yl]methyl]-1,2,5-oxadiazole-3-carboxamide. To a mixture of benzenethiol (10 mg, 0.09 mmol) and K₂CO (38 mg, 0.28 mmol) in ACN (0.5 mL) was added a solution of 4-cyclopropyl-N-((1S)-(4,4-difluorocyclohexyl)(7-((6-((4-nitrophenyl)sulfonyl)-7-oxo-6-azabicyclo[3.2.1]octan-1-yl)methyl)imidazo[1,2-b]pyridazin-2-yl)methyl)-1,2,5-oxadiazole-3-carboxamide (50 mg, 0.069 mmol, Step G) in ACN (2.5 mL) dropwise over 2 min. Then, DMSO (0.1 mL) was added and the resulting mixture was stirred at rt for 1.5 h. The reaction was quenched by the addition of 1 N aqueous NaOH (10 mL) and the mixture extracted with EtOAc (2×150 mL). The organic layers were combined, washed with brine (15 mL) and concentrated to dryness. The residue was purified by silica gel chromatography (0-100% EtOAc/petroleum ether) to provide a mixture of diastereomers. The diastereomers were separated by chiral SFC (Phenomenex-Cellulose-2, 10 μm, 250×30 mm, 50% methanol (with 0.1% NH₄OH), 50% CO₂). The first-eluting isomer (30% yield) was designated as the (1R*,5R*) isomer (Example 11) and the second-eluting isomer (47% yield) was designated as the (1S*,5S*) isomer (Example 12). Example 11: ¹H NMR (400 MHz, DMSO-d₆) δ 9.49 (d, J=9.2 Hz, 1H), 8.35 (d, J=2.0 Hz, 1H), 8.19 (s, 1H), 7.84-7.82 (m, 1H), 7.68 (s, 1H), 5.21-5.13 (m, 1H), 3.55-3.50 (m, 1H), 2.86-2.79 (m, 1H), 2.75-2.68 (m, 1H), 2.31-2.23 (m, 1H), 2.23-2.14 (m, 1H), 2.10-1.94 (m, 2H), 1.93-1.86 (m, 2H), 1.84-1.68 (m, 2H), 1.67-1.57 (m, 2H), 1.55-1.44 (m, 3H), 1.42-1.25 (m, 5H), 1.15-1.09 (m, 2H), 0.98-0.93 (m, 2H). MS (ESI) m/z: [M+H]⁺ Found 540.3. Example 12: ¹H NMR (400 MHz, DMSO-d₆) δ 9.49 (d, 0.1=9.2 Hz, 1H), 8.35 (d, J=2.0 Hz, 1H), 8.18 (s, 1H), 7.83 (d, J=1.6 Hz, 1H), 7.67 (s, 1H), 5.17 (t, J=8.4 Hz, 1H), 3.52 (s, 1H), 2.86-2.79 (m, 1H), 2.74-2.68 (m, 1H), 2.31-2.23 (m, 1H), 2.22-2.11 (m, 1H), 2.10-1.94 (m, 2H), 1.92-1.86 (m, 2H), 1.84-1.68 (m, 2H), 1.67-1.58 (m, 2H), 1.57-1.44 (m, 3H), 1.41-1.22 (m, 5H), 1.17-1.06 (m, 2H), 1.01-0.91 (m, 2H). MS (ESI) m/z: [M+H]⁺ Found 540.3.

### Example 13: 4-Cyclopropyl-N—[(S)-(4,4-difluorocyclohexyl)-[7-[[(3R*)-5,5-dimethyl-2-oxo-pyrrolidin-3-yl]methyl]imidazo[1,2-b]pyridazin-2-yl]methyl]-1,2,5-oxadiazole-3-carboxamide

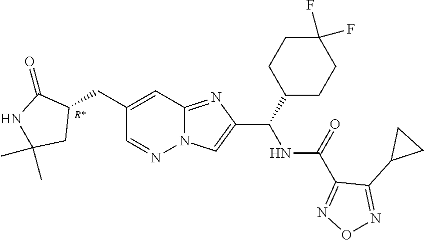 [SMILES: CC1(C)C[C@@H](Cc2cnn3cc([C@@H](NC(=O)c4nonc4C4CC4)C4CCC(F)(F)CC4)nc3c2)C(=O)N1]

### Example 14: 4-Cyclopropyl-N—[(S)-(4,4-difluorocyclohexyl)-[7-[[(S)-5,5-dimethyl-2-oxo-pyrrolidin-3-yl]methyl]imidazo[1,2-b]pyridazin-2-yl]methyl]-1,2,5-oxadiazole-3-carboxamide

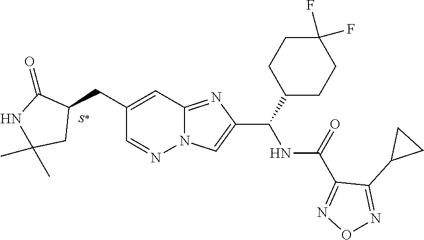 [SMILES: CC1(C)C[C@H](Cc2cnn3cc([C@@H](NC(=O)c4nonc4C4CC4)C4CCC(F)(F)CC4)nc3c2)C(=O)N1]

Step A: tert-Butyl 4-((2-((S)-((er-butoxycarbonyl)amino)(4,4-difluorocyclohexyl)methyl)imidazo[1,2-b]pyridazin-7-yl)methyl)-2,2-dimethyl-5-oxopyrrolidine-1-carboxylate. The title compound was prepared as described for the synthesis of Example 1 and Example 2 Step A using tert-butyl 2,2-dimethyl-5-oxopyrrolidine-1-carboxylate (Intermediate 36) in place of 1,1-dimethylethyl 5,5-difluoro-2-oxo-1-piperidinecarboxylate. The residue was purified by silica gel chromatography (0-100% EtOAc/hexanes) to provide the title compound in 7% yield.

Step B: 3-((2-((S)-Amino(4,4-difluorocyclohexyl)methyl)imidazo[1,2-b]pyridazin-7-yl)methyl)-5,5-dimethylpyrrolidin-2-one. To a mixture of tert-butyl 4-((2-((S)-((tert-butoxycarbonyl)amino)(4,4-difluorocyclohexyl)methyl)imidazo[1,2-b]pyridazin-7-yl)methyl)-2,2-dimethyl-5-oxopyrrolidine-1-carboxylate (33 mg, 0.056 mmol, Step A) in DCM (1 mL) was added TF A (0.2 mL, 2.6 mmol) and the resulting mixture was stirred at it for 40 min. The mixture was concentrated, then partitioned between 20% IPA/DCM (10 mL) and saturated aqueous Na₂CO₃. The aqueous layer was further extracted with 20% IPA/DCM (3×10 mL). The organic layers were combined, dried over anhydrous Na₂SO₄, filtered, and concentrated to dryness to provide the title compound that was used without further purification (100% yield).

Step C: 4-Cyclopropyl-N—[(S)-(4,4-difluorocyclohexyl)-[7-[[(3R*)-5,5-dimethyl-2-oxo-pyrrolidin-3-yl]methyl]imidazo[1,2-b]pyridazin-2-yl]methyl]-1,2,5-oxadiazole-3-carboxamide and 4-cyclopropyl-N—[(S)-(4,4-difluorocyclohexyl)-[7-[[(3S*)-5,5-dimethyl-2-oxo-pyrrolidin-3-yl]methyl]imidazo[1,2-b]pyridazin-2-yl]methyl]-1,2,5-oxadiazole-3-carboxamide. To a solution of 4-cyclopropyl-1,2,5-oxadiazole-3-carboxylic acid (20 mg, 0.13 mmol) and T₃P (0.10 mL, 0.17 mmol, 50% in EtOAc) in EtOAc (0.27 mL) was added DIPEA (28 μL, 0.16 mmol) and the resulting mixture was stirred at rt for 5 min. Then, a solution of 3-((2-((S)-amino(4,4-difluorocyclohexyl)methyl)imidazo[1,2-b]pyridazin-7-yl)methyl)-5,5-dimethylpyrrolidin-2-one (21 mg, 0.054 mmol, Step B) in DCM (0.6 mL) was added to the reaction mixture and the resulting mixture was stirred at rt for 3 h. The reaction mixture was concentrated to dryness and the residue was purified by silica gel chromatography (0-100% acetone/hexanes (with 0.1% TEA)) to provide a mixture of diastereomers. The diastereomers were separated by chiral SFC (Whelk O1 SS, 5 μm, 250×21 mm, 30% methanol, 700/c CO₂). The first-eluting isomer (19% yield) was designated as the (3R*) isomer (Example 13) and the second-eluting isomer (17% yield) was designated as the (3S*) isomer (Example 14). Example 13: ¹H NMR (500 MHz, DMSO-d₆) δ 9.48-9.45 (m, 1H), 8.45-8.43 (m, 1H), 8.19-8.17 (m, 1H), 7.93-7.91 (m, 1H), 7.83-7.80 (m, 1H), 5.21-5.16 (m, 1H), 3.12-3.07 (m, 1H), 2.99-2.90 (m, 1H), 2.66-2.60 (m, 2H), 2.31-2.24 (m, 1H), 2.23-2.14 (m, 1H), 2.10-2.01 (m, 1H), 2.01-1.95 (m, 1H), 1.93-1.88 (m, 1H), 1.84-1.74 (m, 1H), 1.66-1.59 (m, 1H), 1.56-1.49 (m, 1H), 1.44-1.34 (m, 1H), 1.33-1.21 (m, 2H), 1.17-1.14 (m, 3H), 1.14-1.10 (m, 5H), 0.98-0.94 (m, 2H). MS (ESI) m/z: [M+H]⁺ Found 528.3. Example 14: ¹H NMR (500 MHz, DMSO-d₆) δ 9.47-9.44 (m, 1H), 8.44-8.43 (m, 1H), 8.18-8.16 (m, 1H), 7.92-7.90 (m, 1H), 7.82-7.80 (m, 1H), 5.20-5.15 (m, 1H), 3.28-3.28 (m, 1H), 3.11-3.06 (m, 1H), 2.96-2.89 (m, 1H), 2.64-2.62 (m, 1H), 2.30-2.24 (m, 1H), 2.20-2.14 (m, 1H), 2.08-1.95 (m, 2H), 1.93-1.86 (m, 2H), 1.85-1.71 (m, 2H), 1.66-1.58 (m, 1H), 1.56-1.49 (m, 1H), 1.42-1.34 (m, 1H), 1.34-1.21 (m, 2H), 1.16-1.13 (m, 3H), 1.12-1.11 (m, 3H), 0.97-0.94 (m, 2H). MS (ESI) m/z: [M+H]⁺ Found 528.3.

### Example 15: 4-Cyclopropyl-N—[(S)-(4,4-difluorocyclohexyl)-[7-[[(3R*)-4,4-dimethyl-2-oxo-pyrrolidin-3-yl]methyl]imidazo[1,2-b]pyridazin-2-yl]methyl]-1,2,5-oxadiazole-3-carboxamide

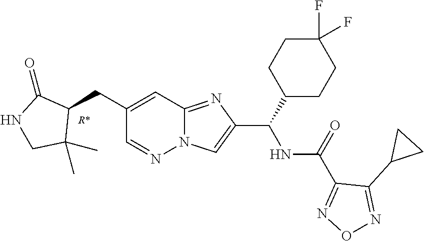 [SMILES: CC1(C)CNC(=O)[C@H]1Cc1cnn2cc([C@@H](NC(=O)c3nonc3C3CC3)C3CCC(F)(F)CC3)nc2c1]

### Example 16: 4-Cyclopropyl-N—[(S)-(4,4-difluorocyclohexyl)-[7-[[(3S*)-4,4-dimethyl-2-oxo-pyrrolidin-3-yl]methyl]imidazo[1,2-b]pyridazin-2-yl]methyl]-1,2,5-oxadiazole-3-carboxamide

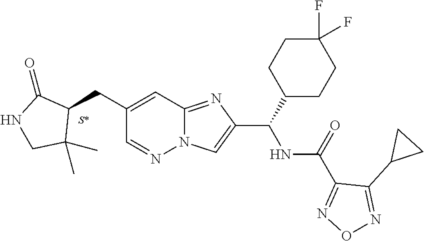 [SMILES: CC1(C)CNC(=O)[C@H]1Cc1cnn2cc([C@@H](NC(=O)c3nonc3C3CC3)C3CCC(F)(F)CC3)nc2c1]

Step A: tert-Butyl 3-((2-((S)-((tert-butoxycarbonyl)amino)(4,4-difluorocyclohexyl)methyl)imidazo[1,2-b]pyridazin-7-yl)methyl)-4,4-dimethyl-2-oxopyrrolidine-1-carboxylate. The title compound was prepared as described for the synthesis of Example 1 and Example 2 Step A using tert-butyl 4,4-dimethyl-2-oxopyrrolidine-1-carboxylate (Intermediate 37) in place of 1,1-dimethylethyl 5,5-difluoro-2-oxo-1-piperidinecarboxylate. The residue was purified by silica gel chromatography (0-100% EtOAc/hexanes) to provide the title compound in 23% yield.

Step B: 3-((2-((S)-Amino(4,4-difluorocyclohexyl)methyl)imidazo[1,2-b]pyridazin-7-yl)methyl)-4,4-dimethylpyrrolidin-2-one. The title compound (99% yield) was prepared as described for the synthesis of Example 13 and Example 14 Step B using tert-butyl 3-((2-((S)-((tert-butoxycarbonyl)amino)(4,4-difluorocyclohexyl)methyl)imidazo[1,2-b]pyridazin-7-yl)methyl)-4,4-dimethyl-2-oxopyrrolidine-1-carboxylate (Step A) in place of tert-butyl 4-((2-((S)-((tert-butoxycarbonyl)amino)(4,4-difluorocyclohexyl)methyl)imidazo[1,2-b]pyridazin-7-yl)methyl)-2,2-dimethyl-5-oxopyrrolidine-1-carboxylate

Step C: 4-Cyclopropyl-N—[(S)-(4,4-difluorocyclohexyl)-[7-[[(3R*)-4,4-dimethyl-2-oxo-pyrrolidin-3-yl]methyl]imidazo[1,2-b]pyridazin-2-yl]methyl]-1,2,5-oxadiazole-3-carboxamide and 4-cyclopropyl-N—[(S)-(4,4-difluorocyclohexyl)-[7-[[(3S*)-4,4-dimethyl-2-oxo-pyrrolidin-3-yl]methyl]imidazo[1,2-b]pyridazin-2-yl]methyl]-1,2,5-oxadiazole-3-carboxamide. To a solution of 4-cyclopropyl-1,2,5-oxadiazole-3-carboxylic acid (47 mg, 0.31 mmol) and T₃P (0.24 mL, 0.41 mmol, 50% in EtOAc) in EtOAc (1 mL) was added DIPEA (110 μL, 0.61 mmol) and the resulting mixture was stirred at rt for 5 min. Then, a solution of 3-((2-((S)-amino(4,4-difluorocyclohexyl)methyl)imidazo[1,2-b]pyridazin-7-yl)methyl)-4,4-dimethylpyrrolidin-2-one (80 mg, 0.2 mmol, Step B) in DCM (2 mL) was added to the reaction mixture and the resulting mixture was stirred at rt for 2.5 h. A solution of 4-cyclopropyl-1,2,5-oxadiazole-3-carboxylic acid (25 mg, 0.16 mmol), T₃P (0.1 mL, 0.17 mmol, 50% in EtOAc) and DIPEA (50 μL, 0.28 mmol) was sonicated at rt for 5 min and then added to the reaction mixture and the resulting mixture was stirred at rt for 16 h. Then, the reaction mixture was concentrated to dryness and purified by silica gel chromatography (0-100% acetone/hexanes (with 0.1% TEA)) to provide a mixture of diastereomers. The diastereomers were separated by chiral SFC (Whelk O1 SS, 5 μm, 250×30 mm, 35% methanol, 65% CO₂). The first-eluting isomer (9% yield) was designated as the (3R*) isomer (Example 15) and the second-eluting isomer (9% yield) was designated as the (3S*) isomer (Example 16). Example 15: ¹H NMR (500 MHz, DMSO-d₆) δ 9.50 (d, J=9.0 Hz, 1H), 8.60 (d, J=1.8 Hz, 1H), 8.24 (s, 1H), 8.03 (s, 1H), 7.65 (s, 1H), 5.20 (t, J=8.5 Hz, 2H), 2.97 (d, J=9.5 Hz, 1H), 2.91-2.84 (m, 2H), 2.76-2.70 (m, 1H), 2.59-2.54 (m, 1H), 2.31-2.26 (m, 1H), 2.19 (br s, 1H), 2.09-1.96 (m, 2H), 1.94-1.88 (m, 1H), 1.86-1.72 (m, 2H), 1.68-1.61 (m, 11H), 1.44-1.24 (m, 2H), 1.15-1.10 (m, 2H), 1.02-0.99 (m, 6H), 0.98-0.95 (m, 2H). MS (ESI) m/z: [M+H]⁺ Found 528.3. Example 16: ¹H NMR (500 MHz, DMSO-d₆) δ 9.49-9.44 (m, 1H), 8.54-8.52 (m, 11H), 8.18-8.17 (m, 1H), 7.98-7.96 (m, 11H), 7.65-7.63 (m, 1H), 5.20-5.16 (m, 11H), 2.98-2.94 (m, 1H), 2.90-2.84 (m, 211), 2.72-2.66 (m, 1H), 2.57-2.53 (m, 1H), 2.30-2.24 (m, 1H), 2.22-2.13 (m, 11H), 2.09-1.93 (m, 2H), 1.92-1.86 (m, 11H), 1.85-1.71 (m, 2H), 1.66-1.60 (m, 1H), 1.44-1.22 (m, 2H), 1.14-1.09 (m, 211), 1.00-0.99 (m, 3H), 0.98-0.97 (m, 3H), 0.96-0.94 (m, 2H). MS (ESI) m/z: [M+H]⁺ Found 528.3.

### Example 17: 4-Cyclopropyl-N—((S)-(4,4-difluorocyclohexyl)(7-(((1R*,4R*)-3-oxo-2-azabicyclo[2.2.1]heptan-4-yl)methyl)imidazo[1,2-b]pyridazin-2-yl)methyl)-1,2,5-oxadiazole-3-carboxamide

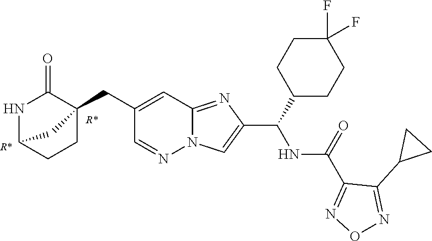 [SMILES: O=C(N[C@H](c1cn2ncc(C[C@@]34CC[C@H](C3)NC4=O)cc2n1)C1CCC(F)(F)CC1)c1nonc1C1CC1]

### Example 18: 4-Cyclopropyl-N—((S)-(4,4-difluorocyclohexyl)(7-(((1S*,4S*)-3-oxo-2-azabicyclo[2.2.1]heptan-4-yl)methyl)imidazo[1,2-b]pyridazin-2-yl)methyl)-1,2,5-oxadiazole-3-carboxamide

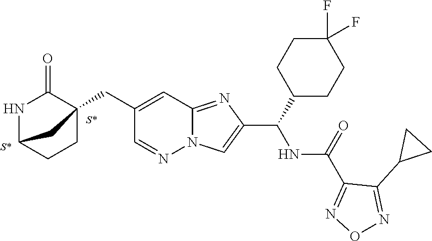 [SMILES: O=C(N[C@H](c1cn2ncc(C[C@]34CC[C@@H](C3)NC4=O)cc2n1)C1CCC(F)(F)CC1)c1nonc1C1CC1]

Step A: Methyl (S)-1-((2-(((tert-butoxycarbonyl)amino)(4,4-difluorocyclohexyl)methyl)imidazo[1,2-b]pyridazin-7-yl)methyl)cyclopent-3-ene-1-carboxylate. The title compound (55% yield) was prepared as described for the synthesis of Example 11 and Example 12 Step A using methyl cyclopent-3-ene-1-carboxylate in place of methyl cyclohex-3-ene-1-carboxylate.

Step B: (S)-1-((2-(((tert-Butoxycarbonyl)amino)(4,4-difluorocyclohexyl)methyl)imidazo[1,2-b]pyridazin-7-yl)methyl)cyclopent-3-ene-1-carboxylic acid. The title compound (92% yield) was prepared as described for the synthesis of Example 11 and Example 12 Step B using methyl (S)-1-((2-(((tert-butoxycarbonyl)amino(4,4-difluorocyclohexyl)methyl)imidazo[1,2-b]pyridazin-7-yl)methyl)cyclopent-3-ene-1-carboxylate (Step A) in place of methyl 1-((2-((S)-((tert-butoxycarbonyl)amino)(4,4-difluorocyclohexyl)methyl)imidazo[1,2-b]pyridazin-7-yl)methyl)cyclohex-3-ene-1-carboxylate.

Step C: tert-Butyl (S)-((4,4-difluorocyclohexyl)(7-((1-(((4-nitrophenyl)sulfonyl)carbamoyl)cyclopent-3-en-1-yl)methyl)imidazo[1,2-b]pyridazin-2-yl)methyl)carbamate. The title compound (73% yield) was prepared as described for the synthesis of Example 11 and Example 12 Step C using (S)-1-((2-(((tert-butoxycarbonyl)amino(4,4-difluorocyclohexyl)methyl)imidazo[1,2-b]pyridazin-7-yl)methyl)cyclopent-3-ene-1-carboxylic acid (Step B) in place of 1-((2-((S)-((tert-butoxycarbonyl)amino)(4,4-difluorocyclohexyl)methyl)imidazo[1,2-b]pyridazin-7-yl)methyl)cyclohex-3-ene-1-carboxylic acid.

Step D: tert-Butyl ((1S)-(4,4-difluorocyclohexyl)(7-((6-iodo-2-((4-nitrophenyl)sulfonyl)-3-oxo-2-azabicyclo[2.2.1]heptan-4-yl)methyl)imidazo[1,2-b]pyridazin-2-yl)methyl)carbamate. The title compound was prepared as described for the synthesis of Example 11 and Example 12 Step F using tert-butyl (S)-((4,4-difluorocyclohexyl)(7-((1-(((4-nitrophenyl)sulfonyl)carbamoyl)cyclopent-3-en-1-yl)methyl)imidazo[1,2-b]pyridazin-2-yl)methyl)carbamate (Step C) in place of 4-cyclopropyl-N-((1S)-(4,4-difluorocyclohexyl)(7-((1-(((4-nitrophenyl)sulfonyl)carbamoyl)cyclohex-3-en-1-yl)methyl)imidazo[1,2-b]pyridazin-2-yl)methyl)-1,2,5-oxadiazole-3-carboxamide. The reaction was stirred at rt overnight instead of 2 h, and was further purified by silica gel chromatography (0-80% EtOAc/petroleum ether) to provide the title compound (55% yield) as a yellow oil.

Step E: tert-Butyl ((1S)-(4,4-difluorocyclohexyl)(7-((6-iodo-3-oxo-2-azabicyclo[2.2.1]heptan-4-yl)methyl)imidazo[1,2-b]pyridazin-2-yl)methyl)carbamate. The title compound was prepared as described for the synthesis of Example 11 and Example 12 Step H using tert-butyl ((1S)-(4,4-difluorocyclohexyl)(7-((6-iodo-2-((4-nitrophenyl)sulfonyl)-3-oxo-2-azabicyclo[2.2.1]heptan-4-yl)methyl)imidazo[1,2-b]pyridazin-2-yl)methyl)carbamate (Step D) in place of 4-cyclopropyl-N-((1S)-(4,4-difluorocyclohexyl)(7-((6-((4-nitrophenyl)sulfonyl)-7-oxo-6-azabicyclo[3.2.1]octan-1-yl)methyl)imidazo[1,2-b]pyridazin-2-yl)methyl)-1,2,5-oxadiazole-3-carboxamide. The residue was purified by silica gel chromatography (0-10% MeOH/DCM) followed by preparative TLC (5% MeOH/DCM) to provide the title compound as a yellow oil (31% yield).

Step F: tert-Butyl ((1S)-(4,4-difluorocyclohexyl)(7-((3-oxo-2-azabicyclo[2.2.1]heptan-4-yl)methyl)imidazo[1,2-b]pyridazin-2-yl)methyl)carbamate. A flask with a mixture of tert-butyl ((1S)-(4,4-difluorocyclohexyl)(7-((6-iodo-3-oxo-2-azabicyclo[2.2.1]heptan-4-yl)methyl)imidazo[1,2-b]pyridazin-2-yl)methyl)carbamate (30 mg, 0.049 mmol, Step E), wet Pd/C (50 mg, 10% Pd, 50% water) and TEA (0.034 mL, 0.24 mmol) in MeOH (5 mL) was purged and refilled with H₂ three times. The resulting mixture was stirred under H₂ (15 psi) at rt for 5 min and then filtered. The filter cake was washed with MeOH (20 mL) and the filtrate concentrated to dryness. The residue was purified by silica gel chromatography (0-0.8% MeOH/DCM) to provide the title compound as a light-yellow solid.

Step G: 4-((2-((S)-Amino(4,4-difluorocyclohexyl)methyl)imidazo[1,2-b]pyridazin-7-yl)methyl)-2-azabicyclo[2.2.1]heptan-3-one hydrochloride. The title compound was prepared as described for the synthesis of Example 11 and Example 12 Step D using tert-butyl ((1S)-(4,4-difluorocyclohexyl)(7-((3-oxo-2-azabicyclo[2.2.1]heptan-4-yl)methyl)imidazo[1,2-b]pyridazin-2-yl)methyl)carbamate (Step F) in place of 4-cyclopropyl-N-((1S)-(4,4-difluorocyclohexyl)(7-((1-(((4-nitrophenyl)sulfonyl)carbamoyl)cyclohex-3-en-1-yl)methyl)imidazo[1,2-b]pyridazin-2-yl)methyl)-1,2,5-oxadiazole-3-carboxamide. The reaction mixture was concentrated to provide the title compound as a yellow oil (84% yield).

Step H: 4-Cyclopropyl-N—((S)-(4,4-difluorocyclohexyl)(7-(((R*,4R*)-3-oxo-2-azabicyclo[2.2.1]heptan-4-yl)methyl)imidazo[1,2-b]pyridazin-2-yl)methyl)-1,2,5-oxadiazole-3-carboxamide and 4-cyclopropyl-N—((S)-(4,4-difluorocyclohexyl)(7-(((1S*,4S*)-3-oxo-2-azabicyclo[2.2.1]heptan-4-yl)methyl)imidazo[1,2-b]pyridazin-2-yl)methyl)-1,2,5-oxadiazole-3-carboxamide. The title compounds were prepared as described for the synthesis of Example 11 and Example 12 Step E using 4-((2-((S)-amino(4,4-difluorocyclohexyl)methyl)imidazo[1,2-b]pyridazin-7-yl)methyl)-2-azabicyclo[2.2.1]heptan-3-one hydrochloride (Step G) in place of 1-((2-((S)-amino(4,4-difluorocyclohexyl)methyl)imidazo[1,2-b]pyridazin-7-yl)methyl)-N-((4-nitrophenyl)sulfonyl)cyclohex-3-ene-1-carboxamide. The residue was purified by preparative TLC (5% MeOH/DCM). The diastereomers were then separated by chiral SFC (DAICEL CHIRALCEL OD-H, 5 μm, 250×30 mm, 25% ethanol (with 0.1% NH₄OH), 75% CO₂). The first-eluting isomer (30% yield) was designated as the (1R*,4R*) isomer (Example 17) and the second-eluting isomer (25% yield) was designated as the (1S*4S) isomer (Example 18). Example 17: ¹H NMR (400 MHz, CDCl₃) δ 8.61-8.52 (m, 1H), 8.39-8.27 (m, 1H), 7.99-7.87 (m, 2H), 5.69-5.55 (m, 1H), 5.33-5.23 (m, 1H), 3.93-3.83 (m, 1H), 3.41-3.30 (m, 1H), 3.04-2.92 (m, 1H), 2.59-2.48 (m, 1H), 2.24-2.20 (m, 1H), 2.00-1.94 (m, 2H), 1.79-1.71 (m, 4H), 1.67-1.60 (m, 3H), 1.56-1.46 (m, 2H), 1.42-1.30 (m, 3H), 1.18-1.06 (m, 4H). MS (ESI) m/z: [M+H]⁺ Found 526.3. Example 18: ¹H NMR (400 MHz, CDCl₃) δ 8.56-8.43 (m, 1H), 8.18-7.97 (m, 1H), 7.92-7.79 (m, 2H), 5.62-5.49 (m, 1H), 5.32-5.22 (m, 1H), 3.93-3.82 (m, 1H), 3.42-3.28 (m, 1H), 3.03-2.89 (m, 1H), 2.61-2.47 (m, 1H), 2.34-2.25 (m, 1H), 2.10-2.04 (M, 2H), 2.00-1.91 (m, 2H), 1.68-1.61 (m, 4H), 1.55-1.52 (m, 1H), 1.49-1.42 (m, 2H), 1.41-1.30 (m, 3H), 1.20-1.08 (m, 4H). MS (ESI) m/z: [M+H]⁺ Found 526.3.

### Example 19: 4-Cyclopropyl-N—((S)-(4,4-difluorocyclohexyl)(7-((R*)-1-((3R*,5S)-2-oxo-5-(trifluoromethyl)pyrrolidin-3-yl)propyl)imidazo[1,2-b]pyridazin-2-yl)methyl)-1,2,5-oxadiazole-3-carboxamide

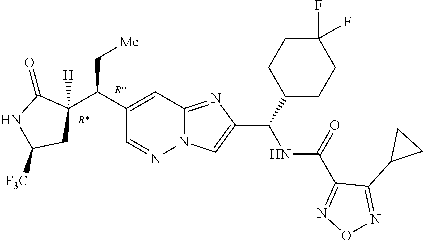 [SMILES: CC[C@@H](c1cnn2cc([C@@H](NC(=O)c3nonc3C3CC3)C3CCC(F)(F)CC3)nc2c1)[C@@H]1C[C@@H](C(F)(F)F)NC1=O]

### Example 20: 4-Cyclopropyl-N—((S)-(4,4-difluorocyclohexyl)(7-((R*)-1-((3S*,5S)-2-oxo-5-(trifluoromethyl)pyrrolidin-3-yl)propyl)imidazo[1,2-b]pyridazin-2-yl)methyl)-1,2,5-oxadiazole-3-carboxamide

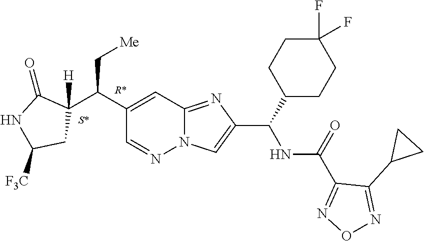

Step A: tert-Butyl ((S)-(4,4-difluorocyclohexyl)(7-((E)-((S)-2-oxo-1-tosyl-5-(trifluoromethyl)pyrrolidin-3-ylidene)methyl)imidazo[1,2-b]pyridazin-2-yl)methyl)carbamate. To a suspension of NaH (76.5 mg, 1.91 mmol, 60% in mineral oil) in THF (4 mL) was added a solution of diethyl ((5S)-2-oxo-1-tosyl-5-(trifluoromethyl)pyrrolidin-3-yl)phosphonate (848 mg, 1.91 mmol, Intermediate 15) in THF (4 mL) and the resulting mixture was cooled to −78° C. A mixture of tert-butyl (S)-((4,4-difluorocyclohexyl)(7-formylimidazo[1,2-b]pyridazin-2-yl)methyl)carbamate (580 mg, 1.47 mmol) in THF (4 mL) was then added dropwise to the phosphonate mixture. The resulting reaction mixture was allowed to warm to rt and stirred overnight. The reaction was then cooled to 0° C. and quenched with water. The biphasic mixture was extracted with EtOAc (3×) and the combined organic layers were washed with brine, dried over anhydrous MgSO₄, filtered, and concentrated to dryness. The residue was purified by silica gel chromatography (0-100% EtOAc/hexanes) to afford the title compound as a mixture of olefin isomers (75:25 E:Z) in 78% yield.

Step B: tert-Butyl ((1S)-(4,4-difluorocyclohexyl)(7-(1-((5S)-2-oxo-1-tosyl-5-(trifluoromethyl)pyrrolidin-3-yl)propyl)imidazo[1,2-b]pyridazin-2-yl)methyl)carbamate. In an oven-dried Schlenk tube copper(1) bromide-dimethyl sulfide complex (6.95 mg, 0.034 mmol) and (2R)-1-[(1R)-1-(dicyclohexylphosphino)ethyl]-2-(diphenylphosphino)ferrocene (25.97 mg, 0.041 mmol) were dissolved in dichloromethane (0.25 mL) and stirred at rt under a nitrogen atmosphere for 20 min. Then, a solution of tert-butyl ((S)-(4,4-difluorocyclohexyl)(7-((E)-((S)-2-oxo-1-tosyl-5-(trifluoromethyl)pyrrolidin-3-ylidene)methyl)imidazo[1,2-b]pyridazin-2-yl)methyl)carbamate (231 mg, 0.34 mmol, Step A) in dichloromethane (1.25 mL) was added in one portion. After stirring for 5 min at rt, the reaction mixture was cooled to −78° C., followed by the addition of boron trifluoride diethyl etherate (84 μL, 0.68 mmol). After stirring at −78° C. for 20 min, ethylmagnesium bromide solution (0.23 mL, 3.0 M in diethyl ether, 0.68 mmol) was added dropwise over 5 min. After stirring for 2 h at −78° C., the reaction was quenched with MeOH followed by the addition of saturated aqueous NH₄Cl solution and warming to rt. The reaction mixture was extracted with dichloromethane (3×10 mL), and the combined organic phases were dried over anhydrous MgSO₄, filtered, and concentrated to dryness. The resulting residue was purified by silica gel chromatography (0-100% EtOAc/hexanes) to provide the title compound as a mixture of diastereomers (55:45 dr) in 81% yield.

Step C: tert-Butyl ((1S)-(4,4-difluorocyclohexyl)(7-(1-((5S)-2-oxo-5-(trifluoromethyl)pyrrolidin-3-yl)propyl)imidazo[1,2-b]pyridazin-2-yl)methyl)carbamate. A solution of samarium(II) iodide (13.7 mL, 0.1 M in THF, 1.37 mmol) was added to a solution of tert-butyl ((1S)-(4,4-difluorocyclohexyl)(7-(1-((5S)-2-oxo-1-tosyl-5-(trifluoromethyl)pyrrolidin-3-yl)propyl)imidazo[1,2-b]pyridazin-2-yl)methyl)carbamate (196 mg, 0.28 mmol, Step B) in dry THF (2 mL) under a nitrogen atmosphere at 0° C. The reaction mixture was stirred at rt for 4 h, and then 1 M aqueous HCl (5 mL) was added and the mixture was extracted with ethyl acetate (3×10 mL). The combined organic layers were washed with brine, dried over anhydrous MgSO₄, filtered, and concentrated to dryness. The resulting residue was purified twice by silica gel chromatography (0-100% EtOAc/hexanes, then 0-40% MeOH/EtOAc) to provide the title compound as a mixture of diastereomers (55:45 dr) in 58% yield.

Step D: (5S)-3-(1-(2-((S)-Amino(4,4-difluorocyclohexyl)methyl)imidazo[1,2-b]pyridazin-7-yl)propyl)-5-(trifluoromethyl)pyrrolidin-2-one. A solution of tert-butyl ((1S)-(4,4-difluorocyclohexyl)(7-(1-((5S)-2-oxo-5-(trifluoromethyl)pyrrolidin-3-yl)propyl)imidazo[1,2-b]pyridazin-2-yl)methyl)carbamate (90 mg, 0.16 mmol, Step C) in dichloromethane (1.6 mL) was cooled to 0° C., then trifluoroacetic acid (0.7 mL, 0.22 M) was added dropwise and the resulting mixture was stirred at 0° C. for 2 h. The reaction mixture was concentrated and solvents were removed via azeotropic distillation with toluene (2×5 mL). To the resulting residue was added EtOAc (2 mL) and saturated aqueous NaHCO₃ (2 mL). The pH of the resulting mixture was adjusted to pH=10 by the addition of 3 M aqueous NaOH. The layers were separated and the aqueous layer was further extracted with EtOAc (3×10 mL). The combined organic layers were washed with brine, dried over anhydrous MgSO₄, filtered, and concentrated to dryness. The resulting residue was dried under high vacuum to provide the title compound as a mixture of diastereomers (55:45 dr) in 91% yield.

Step E: 4-Cyclopropyl-N—((S)-(4,4-difluorocyclohexyl)(7-((R*)-1-((3R*,5S)-2-oxo-5-(trifluoromethyl)pyrrolidin-3-yl)propyl)imidazo[1,2-b]pyridazin-2-yl)methyl)-1,2,5-oxadiazole-3-carboxamide and 4-cyclopropyl-N—((S)-(4,4-difluorocyclohexyl)₇-((R*)-1-((3S*,5S)-2-oxo-5-(trifluoromethyl)pyrrolidin-3-yl)propyl)imidazo[1,2-b]pyridazin-2-yl)methyl)-1,2,5-oxadiazole-3-carboxamide. To a solution of 4-cyclopropyl-1,2,5-oxadiazole-3-carboxylic acid (26 mg, 0.16 mmol) in EtOAc (1 mL) were added N,N-diisopropylethylamine (0.1 mL, 0.55 mmol) and 1-propanephosphonic anhydride (0.13 mL, 0.22 mmol, 50 wt % in EtOAc). The reaction mixture was stirred at rt for 20 min. Then, a solution of (5S)-3-(1-(2-((S)-amino(4,4-difluorocyclohexyl)methyl)imidazo[1,2-b]pyridazin-7-yl)propyl)-5-(trifluoromethyl)pyrrolidin-2-one (50 mg, 0.11 mmol, Step D) in dichloromethane (0.3 mL) was added dropwise at rt, and the resulting solution was stirred at rt for 2 h. The reaction was diluted with H₂O (5 mL), and the organic layer was extracted with EtOAc (3×10 mL). The combined organic layers were washed with brine, dried over anhydrous MgSO₄, filtered, and concentrated to dryness. The resulting residue was purified twice by silica gel chromatography (0-100% EtOAc/hexanes, then 0-20% MeOH/EtOAc) to provide the title compound as a mixture of diastereomers (55:45 dr) in 64% yield. The diastereomers were separated by chiral SFC (Stationary phase: Chiralpak IE, 5 μm, 250×30 mm, Mobile phase: 30% methanol, 70% CO₂). The first-eluting isomer (13% yield) was designated as the (R*) isomer (Example 19) and the second-eluting isomer (11% yield) was designated as the (S*) isomer (Example 20). Example 19: ¹H NMR (500 MHz, MeOH-d₄) δ 8.41 (d, J=2.1 Hz, 1H), 8.11 (s, 1H), 7.84 (d, J=2.0 Hz, 1H), 5.27 (d, J=8.6 Hz, 1H), 4.21-4.10 (m, 1H), 3.23 (dt, J=9.5, 5.6 Hz, 1H), 3.08 (td, J=9.7, 5.4 Hz, 1H), 2.52-2.42 (m, 1H), 2.40-2.35 (m, 1H), 2.29-2.19 (m, 1H), 2.13-1.94 (m, 4H), 1.94-1.85 (m, 2H), 1.81-1.70 (m, 2H), 1.65 (dd, J=13.1, 3.5 Hz, 1H), 1.52 (td, J=12.6, 3.9 Hz, 1H), 1.54-1.37 (m, 1H), 1.17-1.09 (m, 2H), 1.05-0.98 (m, 2H), 0.93 (t, J=7.3 Hz, 3H). MS (ESI) m/z: [M+H]⁺ Found 596.2. Example 20: ¹H NMR (500 MHz, MeOH-d₄) δ 8.41 (d, J=2.1 Hz, 1H), 8.10 (s, 1H), 7.81 (d, J=2.0 Hz, 1H), 5.26 (d, J=8.6 Hz, 1H), 4.15-4.08 (m, 1H), 3.02-2.96 (m, 2H), 2.44-2.31 (m, 3H), 2.29-2.18 (m, 1H), 2.19-2.07 (m, 1H), 2.05-1.99 (m, 2H), 1.98-1.91 (m, 1H), 1.89 (s, 1H), 1.87-1.60 (m, 3H), 1.20-1.09 (m, 2H), 1.54-1.38 (m, 2H), 1.06-0.97 (m, 2H), 0.84 (t, J=7.3 Hz, 3H). MS (ESI) m/z: [M+H]⁺ Found 596.2.

### Example 21: 4-Cyclopropyl-N—[(S)-(4,4-difluorocyclohexyl)-[7-[[(3R*)-2-oxopyrrolidin-3-yl]methyl]imidazo[1,2-b]pyridazin-2-yl]methyl]-1,2,5-oxadiazole-3-carboxamide

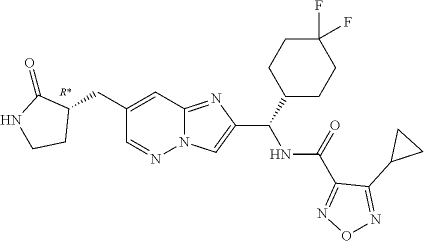 [SMILES: O=C(N[C@H](c1cn2ncc(C[C@@H]3CCNC3=O)cc2n1)C1CCC(F)(F)CC1)c1nonc1C1CC1]

### Example 22: 4-Cyclopropyl-N—[(S)-(4,4-difluorocyclohexyl)-[7-[[(3S*)-2-oxopyrrolidin-3-yl]methyl]imidazo[1,2-b]pyridazin-2-yl]methyl]-1,2,5-oxadiazole-3-carboxamide

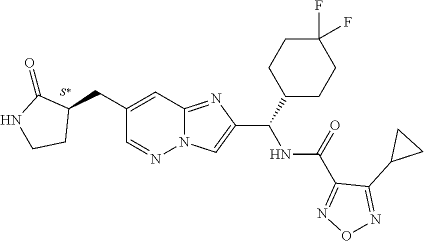 [SMILES: O=C(N[C@H](c1cn2ncc(C[C@H]3CCNC3=O)cc2n1)C1CCC(F)(F)CC1)c1nonc1C1CC1]

A mixture of 4-cyclopropyl-1,2,5-oxadiazole-3-carboxylic acid (33 mg, 0.21 mmol), T₃P (0.13 mL, 0.21 mmol, 50% w/v solution in EtOAc), and DIPEA (0.074 mL, 0.42 mmol) in EtOAc (1.0 mL) was allowed to stir at rt for 2 min. Then, a solution of 3-((2-((S)-amino(4,4-difluorocyclohexyl)methyl)imidazo[1,2-b]pyridazin-7-yl)methyl)pyrrolidin-2-one (48 mg, 0.13 mmol, Intermediate 8) in DCM (1 mL) was added to the reaction mixture and the resulting mixture was allowed to stir at rt for 18 h. Next, a solution of 4-cyclopropyl-1,2,5-oxadiazole-3-carboxylic acid (33 mg, 0.21 mmol), EtOAc (0.5 mL), T₃P (0.13 mL, 0.21 mmol, 50% w/v solution in EtOAc), and DIPEA (0.074 mL, 0.42 mmol) was allowed to stir at rt for 10 min and then the solution was added to the reaction mixture and the resulting mixture was allowed to stir at rt for 4 h. This mixture was then diluted with EtOAc and the layers were separated. The aqueous layer was extracted with EtOAc (2×), and the combined organic layers were washed with brine, dried over anhydrous Na₂SO₄, filtered, and concentrated to dryness. The residue was purified by silica gel chromatography (0-10% (MeOH with 2.0 M ammonia)/DCM) to provide a mixture of diastereomers. The diastereomers were separated by chiral SFC (Daicel CHIRALPAK® IB N3, 5 μm, 250×21 mm, 30% methanol, 70% CO₂). The first-eluting isomer (27% yield) was designated as the (3R*) isomer (Example 21) and the second-eluting isomer (28% yield) was designated as the (3S*) isomer (Example 22). Example 21: ¹H NMR (400 MHz, DMSO-d₆) δ 9.47 (d, J=9.0 Hz, 1H), 8.45 (d, J=2.0 Hz, 1H), 8.19 (s, 1H), 7.93 (d, J=2.0 Hz, 1H), 7.69 (s, 1H), 5.23-5.14 (m, 1H), 3.18-3.09 (m, 2H), 3.09-3.01 (m, 1H), 2.74-2.63 (m, 2H), 2.35-2.12 (m, 2H), 2.14-1.58 (m, 8H), 1.48-1.22 (m, 2H), 1.17-1.09 (m, 2H), 1.00-0.93 (m, 2H). MS (ESI) m/z: [M+H]⁺ Found 500.2. Example 22: ¹H NMR (400 MHz, DMSO-d₆) δ 9.47 (d, J=9.0 Hz, 1H), 8.45 (d, J=2.0 Hz, 1H), 8.19 (s, 1H), 7.93 (d, J=2.0 Hz, 1H), 7.69 (s, 1H), 5.24-5.14 (m, 1H), 3.17-3.09 (m, 2H), 3.09-3.01 (m, 1H), 2.73-2.65 (m, 2H), 2.35-2.13 (m, 2H), 2.13-1.58 (m, 8H), 1.47-1.21 (m, 2H), 1.18-1.09 (m, 2H), 1.00-0.93 (m, 2H). MS (ESI) m/z: [M+H]⁺ Found 500.2.

### Example 23: 4-Cyclopropyl-N—[(S)-(4,4-difluorocyclohexyl)-[7-[[(3R*)-2-oxo-3-piperidyl]methyl]imidazo[1,2-b]pyridazin-2-yl]methyl]-1,2,5-oxadiazole-3-carboxamide

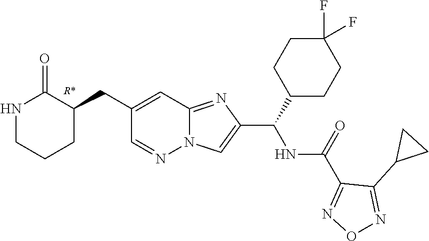 [SMILES: O=C(N[C@H](c1cn2ncc(C[C@H]3CCCNC3=O)cc2n1)C1CCC(F)(F)CC1)c1nonc1C1CC1]

### Example 24: 4-Cyclopropyl-N—[(S)-(4,4-difluorocyclohexyl)-[7-[[(3S*)-2-oxo-3-piperidyl]methyl]imidazo[1,2-b]pyridazin-2-yl]methyl]-1,2,5-oxadiazole-3-carboxamide

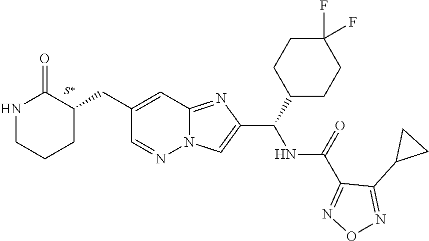 [SMILES: O=C(N[C@H](c1cn2ncc(C[C@@H]3CCCNC3=O)cc2n1)C1CCC(F)(F)CC1)c1nonc1C1CC1]

The title compounds were prepared as described for the synthesis of Example 21 and 22 using 3-((2-((S)-amino(4,4-difluorocyclohexyl)methyl)imidazo[1,2-b]pyridazin-7-yl)methyl)piperidin-2-one (Intermediate 9) in place of 3-((2-((S)-amino(4,4-difluorocyclohexyl)methyl)imidazo[1,2-b]pyridazin-7-yl)methyl)pyrrolidin-2-one. The diastereomers were separated by chiral SFC (Daicel CHIRALPAK® IB N3, 5 μm, 250×21 mm, 35% methanol, 65% CO₂). The first-eluting isomer (33% yield) was designated as the (3R*) isomer (Example 23) and the second-eluting isomer (32% yield) was designated as the (3S*) isomer (Example 24). Example 23: ¹H NMR (400 MHz, DMSO-d₆) δ 9.47 (d, J=9.0 Hz, 1H), 8.42 (d, J 2.0 Hz, 1H), 8.18 (s, 1H), 7.88 (d, J=2.0 Hz, 1H), 7.50 (s, 1H), 5.23-5.14 (m, 1H), 3.23-3.02 (m, 3H), 2.83-2.73 (m, 1H), 2.65-2.54 (m, 1H), 2.36-2.12 (m, 2H), 2.11-1.68 (m, 7H), 1.69-1.51 (m, 2H), 1.46-1.22 (m, 3H), 1.17-1.09 (m, 2H), 1.01-0.92 (m, 2H). MS (ESI) m/z: [M+H]⁺ Found 514.2. Example 24: ¹H NMR (400 MHz, DMSO-d₆) δ 9.47 (d, J=9.0 Hz, 1H), 8.42 (d, J=2.0 Hz, 1H), 8.18 (s, 1H), 7.88 (d, J=2.0 Hz, 1H), 7.49 (s, 1H), 5.24-5.13 (m, 1H), 3.21-3.03 (m, 3H), 2.83-2.74 (m, 1H), 2.64-2.54 (m, 1H), 2.35-2.12 (m, 2H), 2.10-1.68 (m, 7H), 1.68-1.51 (m, 2H), 1.46-1.22 (m, 3H), 1.17-1.11 (m, 2H), 1.00-0.92 (m, 2H). MS (ESI) m/z: [M+H]⁺ Found 514.2.

### Example 25: 4-Cyclopropyl-N—((S)-(4,4-difluorocyclohexyl(7-(((R*)-2-oxoazepan-3-yl)methyl)imidazo[1,2-b]pyridazin-2-yl)methyl)-1,2,5-oxadiazole-3-carboxamide

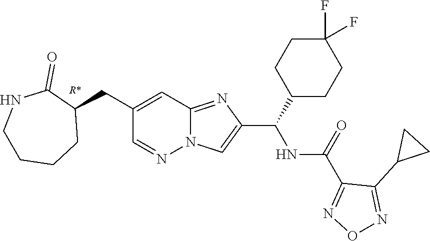 [SMILES: O=C(N[C@H](c1cn2ncc(C[C@H]3CCCCNC3=O)cc2n1)C1CCC(F)(F)CC1)c1nonc1C1CC1]

### Example 26: 4-Cyclopropyl-N—((S)-(4,4-difluorocyclohexyl)(7-(((S*)-2-oxoazepan-3-yl)methyl)imidazo[1,2-b]pyridazin-2-yl)methyl)-1,2,5-oxadiazole-3-carboxamide

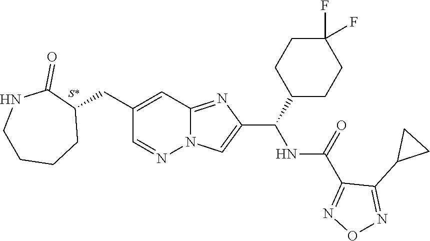 [SMILES: O=C(N[C@H](c1cn2ncc(C[C@@H]3CCCCNC3=O)cc2n1)C1CCC(F)(F)CC1)c1nonc1C1CC1]

The title compounds were prepared as described for the synthesis of Example 21 and 22 using 3-((2-((S)-amino(4,4-difluorocyclohexyl)methyl)imidazo[1,2-b]pyridazin-7-yl)methyl)azepan-2-one (Intermediate 10) in place of 3-((2-((S)-amino(4,4-difluorocyclohexyl)methyl)imidazo[1,2-b]pyridazin-7-yl)methyl)pyrrolidin-2-one. The diastereomers were separated by chiral SFC (Whelk O1 SS, 5 μm, 250×21 mm, 45% methanol, 55% CO₂). The first-eluting isomer (36% yield) was designated as the (R*) isomer (Example 25) and the second-eluting isomer (36% yield) was designated as the (S*) isomer (Example 26). Example 25: ¹H NMR (400 MHz, DMSO-d₆) δ 9.45 (d, J=9.0 Hz, 1H), 8.47 (d, J=2.0 Hz, 1H), 8.16 (s, 1H), 7.89 (d, J=2.0 Hz, 1H), 7.48 (dd, J=7.1, 5.3 Hz, 1H), 5.21-5.14 (m, 1H), 3.28-3.20 (m, 1H), 3.09-2.94 (m, 3H), 2.71-2.56 (m, 1H), 2.37-2.12 (m, 2H), 2.10-1.47 (m, 10H), 1.47-1.19 (m, 4H), 1.17-1.09 (m, 2H), 1.00-0.92 (m, 2H). MS (ESI) m/z: [M+H]⁺ Found 528.2. Example 26: ¹H NMR (400 MHz, DMSO-d₆) δ9.45 (d, J=7.0 Hz, 1H), 8.47 (d, J 2.0 Hz, 1H), 8.16 (s, 1H), 7.89 (d, J=2.0 Hz, 1H), 7.54-7.44 (m, 1H), 5.25-5.10 (m, 1H), 3.27-3.18 (m, 1H), 3.08-2.92 (m, 3H), 2.71-2.55 (m, 1H), 2.37-2.12 (m, 2H), 2.12-1.46 (m, 10H), 1.46-1.18 (m, 4H), 1.18-1.09 (m, 2H), 1.01-0.91 (m, 2H). MS (ESI) m/z: [M+H]⁺ Found 528.2.

### Example 27: 4-Cyclopropyl-N—((S)-(4,4-difluorocyclohexyl)(7-(((R*)-3-methyl-2-oxopiperidin-3-yl)methyl)imidazo[1,2-b]pyridazin-2-yl)methyl)-1,2,5-oxadiazole-3-carboxamide

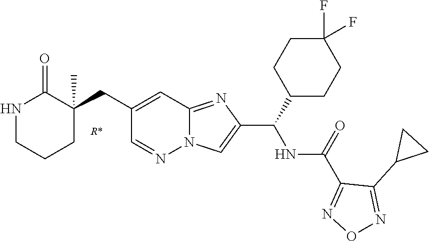 [SMILES: C[C@]1(Cc2cnn3cc([C@@H](NC(=O)c4nonc4C4CC4)C4CCC(F)(F)CC4)nc3c2)CCCNC1=O]

### Example 28: 4-Cyclopropyl-N—((S)-(4,4-difluorocyclohexyl)(7-(((S*)-3-methyl-2-oxopiperidin-3-yl)methyl)imidazo[1,2-b]pyridazin-2-yl)methyl)-1,2,5-oxadiazole-3-carboxamide

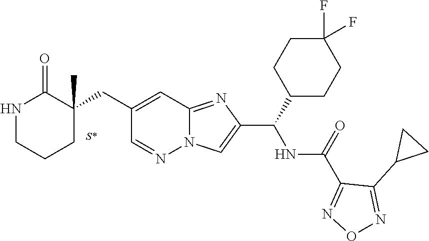 [SMILES: C[C@@]1(Cc2cnn3cc([C@@H](NC(=O)c4nonc4C4CC4)C4CCC(F)(F)CC4)nc3c2)CCCNC1=O]

The title compounds were prepared as described for the synthesis of Example 21 and 22 using 3-((2-((S)-amino(4,4-difluorocyclohexyl)methyl)imidazo[1,2-b]pyridazin-7-yl)methyl)-3-methylpiperidin-2-one (Intermediate 11) in place of 3-((2-((S)-amino(4,4-difluorocyclohexyl)methyl)imidazo[1,2-b]pyridazin-7-yl)methyl)pyrrolidin-2-one. The diastereomers were separated by chiral SFC (Daicel CHIRALPAK® IH, 5 μm, 250×21 mm, 35% methanol (with 0.2% TEA), 65% CO₂). The first-eluting isomer (9% yield) was designated as the (R*) isomer (Example 27) and the second-eluting isomer (11% yield) was designated as the (S*) isomer (Example 28). Example 27: ¹H NMR (400 MHz, DMSO-d₆) δ 9.48 (d, J 9.0 Hz, 1H), 8.32 (d, J=2.0 Hz, 1H), 8.19 (s, 1H), 7.81 (d, J=2.0 Hz, 1H), 7.44 (s, 1H), 5.24-5.15 (m, 1H), 3.13-3.02 (m, 2H), 3.01-2.91 (m, 1H), 2.77-2.64 (m, 1H), 2.37-2.25 (M, 1H), 2.25-2.12 (m, 1H), 2.09-1.60 (m, 9H), 1.57-1.22 (m, 3H), 1.21-1.11 (m, 5H), 1.00-0.92 (m, 2H). MS (ESI) m/z: [M+H]⁺ Found 528.2. Example 28: ¹H NMR (400 MHz, DMSO-d₆) δ 9.47 (d, J 9.0 Hz, 1H), 8.32 (d, J=2.0 Hz, 1H), 8.20 (s, 1H), 7.81 (d, J=2.0 Hz, 1H), 7.44 (s, 1H), 5.24-5.13 (m, 1H), 3.12-3.02 (m, 2H), 3.01-2.90 (m, 1H), 2.74-2.66 (m, 1H), 2.35-2.25 (m, 1H), 2.25-2.13 (m, 1H), 2.12-1.59 (m, 9H), 1.57-1.23 (m, 3H), 1.19-1.11 (m, 5H), 1.00-0.91 (m, 2H). MS (ESI) m/z: [M+H]⁺ Found 528.2.

### Example 29: 4-Cyclopropyl-N—((S)-(4,4-difluorocyclohexyl)(7-(((3R*,6R*)-2-oxo-6-(trifluoromethyl)piperidin-3-yl)methyl)imidazo[1,2-b]pyridazin-2-yl)methyl)-1,2,5-oxadiazole-3-carboxamide

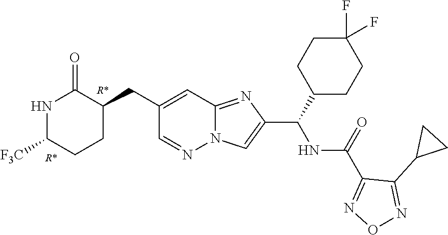 [SMILES: O=C(N[C@H](c1cn2ncc(C[C@H]3CC[C@H](C(F)(F)F)NC3=O)cc2n1)C1CCC(F)(F)CC1)c1nonc1C1CC1]

### Example 30: 4-Cyclopropyl-N—((S)-(4,4-difluorocyclohexyl)(7-(((3R*,6S*)-2-oxo-6-(trifluoromethyl)piperidin-3-yl)methyl)imidazo[1,2-b]pyridazin-2-yl)methyl)-1,2,5-oxadiazole-3-carboxamide

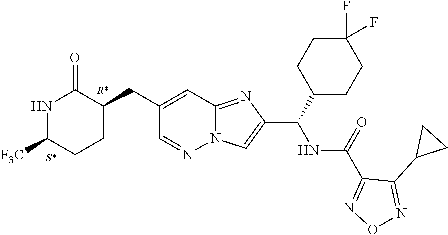 [SMILES: O=C(N[C@H](c1cn2ncc(C[C@H]3CC[C@@H](C(F)(F)F)NC3=O)cc2n1)C1CCC(F)(F)CC1)c1nonc1C1CC1]

### Example 31: 4-Cyclopropyl-N—((S)-(4,4-difluorocyclohexyl)(7-(((3S*,6R*)-2-oxo-6-(trifluoromethyl)piperidin-3-yl)methyl)imidazo[1,2-b]pyridazin-2-yl)methyl)-1,2,5-oxadiazole-3-carboxamide

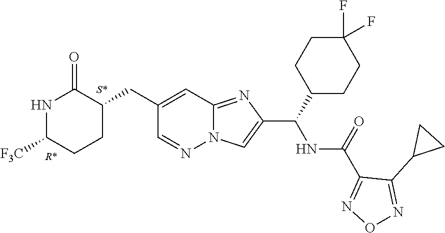 [SMILES: O=C(N[C@H](c1cn2ncc(C[C@@H]3CC[C@H](C(F)(F)F)NC3=O)cc2n1)C1CCC(F)(F)CC1)c1nonc1C1CC1]

### Example 32: 4-Cyclopropyl-N—((S)-(4,4-difluorocyclohexyl)(7-(((3S*,6S*)-2-oxo-6-(trifluoromethyl)piperidin-3-yl)methyl)imidazo[1,2-b]pyridazin-2-yl)methyl)-1,2,5-oxadiazole-3-carboxamide

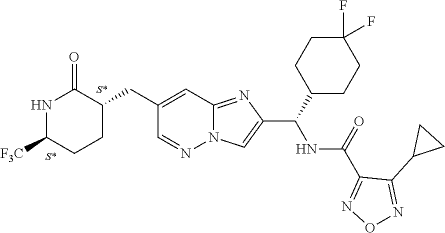 [SMILES: O=C(N[C@H](c1cn2ncc(C[C@@H]3CC[C@@H](C(F)(F)F)NC3=O)cc2n1)C1CCC(F)(F)CC1)c1nonc1C1CC1]

The title compounds were prepared as described for the synthesis of Examples 21 and 22 using 3-((2-((S)-amino(4,4-difluorocyclohexyl)methyl)imidazo[1,2-b]pyridazin-7-yl)methyl)-6-(trifluoromethyl)piperidin-2-one (Intermediate 12) in place of 3-((2-((S)-amino(4,4-difluorocyclohexyl)methyl)imidazo[1,2-b]pyridazin-7-yl)methyl)pyrrolidin-2-one. The diastereomers were separated by chiral SFC (CHIRALCEL® OX—H, 5 μm, 250×30 mm, 35% methanol, 65% CO₂). The first-eluting isomer (<1% yield) was designated as the (3R*,6R*) isomer (Example 29), the second-eluting isomer (<1% yield) was designated as the (3R*,6S*) isomer (Example 30), the third-eluting isomer (13% yield) was designated as the (3S*,6R*) isomer (Example 31), and the fourth-eluting isomer (19% yield) was designated as the (3S*,6S*) isomer (Example 32). Example 29: ¹H NMR (400 MHz, DMSO-d₆) δ 8.27-8.22 (m, 1H), 7.85 (s, 1H), 7.74 (d, J 8.8 Hz, 1H), 7.68 (s, 1H), 5.77 (s, 1H), 5.32-5.22 (m, 1H), 3.99-3.82 (m, 1H), 3.38-3.25 (m, 1H), 3.02-2.94 (m, 1H), 2.69-2.52 (m, 2H), 2.26-1.95 (m, 6H), 1.89-1.63 (m, 5H), 1.45-1.34 (m, 1H), 1.23-1.02 (m, 5H). MS (ESI) m/z: [M+H]⁺ Found 582.2. Example 30: ¹H NMR (400 MHz, DMSO-d₆) δ 8.25 (d, J=2.1 Hz, 1H), 7.85 (s, 1H), 7.75 (d, J=8.8 Hz, 1H), 7.69 (d, J=2.1 Hz, 1H), 5.78 (s, 1H), 5.32-5.23 (m, 1H), 3.96-3.86 (m, 1H), 3.37-3.28 (m, 1H), 3.02-2.92 (m, 1H), 2.67-2.50 (m, 2H), 2.24-1.94 (m, 7H), 1.86-1.64 (m, 4H), 1.45-1.34 (m, 1H), 1.23-1.03 (m, 5H). MS (ESI) m/z: [M+H]⁺ Found 582.2. Example 31: ¹H NMR (400 MHz, DMSO-d₆) δ 9.47 (d, J=9.0 Hz, 1H), 8.42 (d, J=2.0 Hz, 1H), 8.23 (d, J=3.4 Hz, 1H), 8.19 (s, 1H), 7.90 (d, J=2.0 Hz, 1H), 5.24-5.14 (m, 1H), 4.14-4.01 (m, 1H), 3.24-3.17 (m, 1H), 2.82-2.65 (m, 2H), 2.37-2.24 (m, 1H), 2.24-2.12 (m, 1H), 2.10-1.47 (m, 10H), 1.47-1.20 (m, 2H), 1.19-1.07 (m, 2H), 1.01-0.91 (m, 2H). MS (ESI) m/z: [M+H]⁺ Found 582.2. Example 32: ¹H NMR (400 MHz, DMSO-d₆) δ 9.47 (d, J=9.0 Hz, 1H), 8.42 (d, J=2.0 Hz, 1H), 8.23 (d, J=3.4 Hz, 1H), 8.19 (s, 1H), 7.90 (d, J=2.0 Hz, 1H), 5.24-5.13 (m, 1H), 4.08 (s, 1H), 3.25-3.16 (m, 1H), 2.82-2.65 (m, 2H), 2.35-2.23 (m, 1H), 2.24-2.12 (m, 1H), 2.12-1.47 (m, 10H), 1.46-1.21 (m, 2H), 1.16-1.08 (m, 2H), 1.01-0.92 (m, 2H). MS (ESI) m/z: [M+H]⁺ Found 582.2.

### Example 33: 4-Cyclopropyl-N—((S)-(4,4-difluorocyclohexyl)(7-(((3R*,5R*)-2-oxo-5-(trifluoromethyl)piperidin-3-yl)methyl)imidazo[1,2-b]pyridazin-2-yl)methyl)-1,2,5-oxadiazole-3-carboxamide

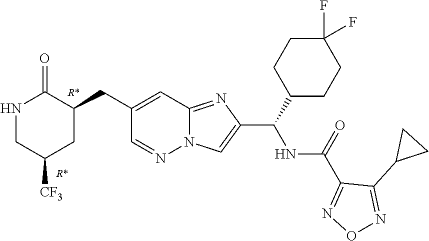 [SMILES: O=C(N[C@H](c1cn2ncc(C[C@H]3C[C@@H](C(F)(F)F)CNC3=O)cc2n1)C1CCC(F)(F)CC1)c1nonc1C1CC1]

### Example 34: 4-Cyclopropyl-N—((S)-(4,4-difluorocyclohexyl)(7-(((3R*,5S*)-2-oxo-5-(trifluoromethyl)piperidin-3-yl)methyl)imidazo[1,2-b]pyridazin-2-yl)methyl)-1,2,5-oxadiazole-3-carboxamide

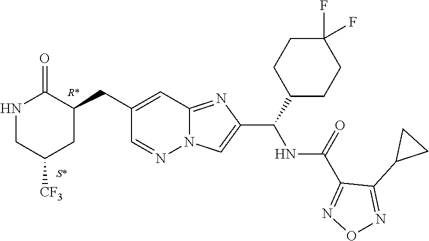 [SMILES: O=C(N[C@H](c1cn2ncc(C[C@H]3C[C@H](C(F)(F)F)CNC3=O)cc2n1)C1CCC(F)(F)CC1)c1nonc1C1CC1]

The title compounds were prepared as described for the synthesis of Examples 21 and 22 using (3R*)-3-((2-((S)-amino(4,4-difluorocyclohexyl)methyl)imidazo[1,2-b]pyridazin-7-yl)methyl)-5-(trifluoromethyl)piperidin-2-one (Intermediate 13) in place of 3-((2-((S)-amino(4,4-difluorocyclohexyl)methyl)imidazo[1,2-b]pyridazin-7-yl)methyl)pyrrolidin-2-one. The diastereomers were separated by chiral SFC (Daicel CHIRALPAK® IB N3, 3 μm, 250×21 mm, 35% methanol, 65% CO₂). The first-eluting isomer (17% yield) was designated as the (3R*,5R*) isomer (Example 33) and the second-eluting isomer (14% yield) was designated as the (3R*,5S*) isomer (Example 34). Example 33: ¹H NMR (400 MHz, DMSO-d₆) δ 9.47 (d, J=9.0 Hz, 1H), 8.44 (d, J=2.0 Hz, 1H), 8.19 (s, 1H), 7.92 (d, J=2.0 Hz, 1H), 7.73 (s, 1H), 5.26-5.13 (m, 1H), 3.27-3.10 (m, 2H), 3.05-2.89 (m, 1H), 2.84-2.72 (m, 2H), 2.36-2.24 (m, 1H), 2.24-2.12 (m, 1H), 2.13-1.59 (m, 8H), 1.57-1.22 (m, 3H), 1.17-1.08 (m, 2H), 1.03-0.93 (m, 2H). MS (ESI) m/z: [M+H]⁺ Found 582.2. Example 34: ¹H NMR (400 MHz, DMSO-d₆) δ 9.47 (d, J=9.0 Hz, 1H), 8.44 (d, J=2.0 Hz, 1H), 8.19 (s, 1H), 7.92 (d, J=2.0 Hz, 1H), 7.75-7.67 (m, 1H), 5.24-5.12 (m, 1H), 3.28-3.09 (m, 2H), 3.04-2.88 (m, 1H), 2.85-2.72 (m, 2H), 2.33-2.24 (m, 1H), 2.24-2.12 (m, 1H), 2.13-1.69 (m, 6H), 1.69-1.58 (m, 1H), 1.59-1.21 (m, 4H), 1.16-1.08 (m, 2H), 1.00-0.89 (m, 2H). MS (ESI) m/z: [M+H]⁺ Found 582.2.

### Example 35: 4-Cyclopropyl-N-((1S)-(4,4-difluorocyclohexyl)(7-(((3S*)-2-oxo-5-(trifluoromethyl)piperidin-3-yl)methyl)imidazo[1,2-b]pyridazin-2-yl)methyl)-1,2,5-oxadiazole-3-carboxamide

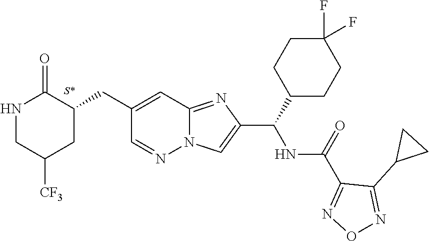 [SMILES: O=C(N[C@H](c1cn2ncc(C[C@@H]3CC(C(F)(F)F)CNC3=O)cc2n1)C1CCC(F)(F)CC1)c1nonc1C1CC1]

The title compound (34% yield) was prepared as described for the synthesis of Examples 21 and 22 using (3S*)-3-((2-((S)-amino(4,4-difluorocyclohexyl)methyl)imidazo[1,2-b]pyridazin-7-yl)methyl)-5-(trifluoromethyl)piperidin-2-one (Intermediate 14) in place of 3-((2-((S)-amino(4,4-difluorocyclohexyl)methyl)imidazo[1,2-b]pyridazin-7-yl)methyl)pyrrolidin-2-one. The diastereomers were not further separated by SFC. ¹H NMR (400 MHz, DMSO-d₆) δ 9.48 (d, J=9.0 Hz, 1H), 8.46 (d, J 2.0 Hz, 1H), 8.20 (s, 1H), 7.93 (d, J=2.0 Hz, 1H), 7.76 (d, J=3.6 Hz, 1H), 5.25-5.15 (m, 1H), 3.27-3.19 (m, 1H), 3.17-2.96 (m, 2H), 2.88-2.74 (m, 2H), 2.36-2.13 (m, 2H), 2.12-1.58 (m, 8H), 1.47-1.20 (m, 3H), 1.19-1.07 (m, 2H), 1.04-0.91 (m, 2H). MS (ESI) m/z: [M+H]⁺ Found 582.2.

### Example 36: 4-Cyclopropyl-N—((S)-(4,4-difluorocyclohexyl(7-(((3R,5S)-3-hydroxy-2-oxo-5-(trifluoromethyl)pyrrolidin-3-yl)methyl)imidazo[1,2-b]pyridazin-2-yl)methyl)-1,2,5-oxadiazole-3-carboxamide

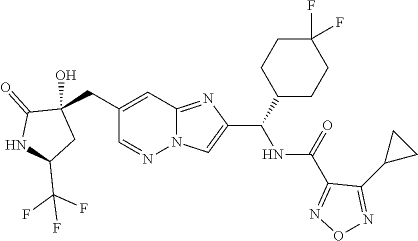 [SMILES: O=C(N[C@H](c1cn2ncc(C[C@@]3(O)C[C@@H](C(F)(F)F)NC3=O)cc2n1)C1CCC(F)(F)CC1)c1nonc1C1CC1]

Step A: tert-Butyl (3R,5S)-3-((2-((S)-((tert-butoxycarbonyl)amino(4,4-difluorocyclohexyl)methyl)imidazo[1,2-b]pyridazin-7-yl)methyl)-3-hydroxy-2-oxo-5-(trifluoromethyl)pyrrolidine-1-carboxylate. A solution of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate (Intermediate 40 Step A, 280 mg, 0.44 mmol) in THF (1.8 mL) was cooled to −78° C. Then, LiHMDS (0.98 mL, 0.98 mmol, 1.0 M in hexanes) was added dropwise and the resulting solution was stirred at −78° C. for 45 min. Then a separate solution of 3-phenyl-2-(phenylsulfonyl)-1,2-oxaziridine (170 mg, 0.67 mmol) in THF (0.5 mL) was added to the reaction mixture dropwise and the reaction was stirred at −78° C. for 30 min. After this time the reaction was quenched with a saturated aqueous solution of NH₄Cl and warmed to rt. The biphasic mixture was then extracted with EtOAc (3×) and the combined organic layers were washed with brine, dried over anhydrous MgSO₄, filtered and concentrated to dryness. The residue was purified by preparative HPLC (Xbridge C18, 5 μm, 50×100 mm, 10-100% MeCN/water (with 20 mM NH₄OH)) to afford the title compound in 7% yield.

Step B: (S)-(4,4-Difluorocyclohexyl)(7-(((3R,5S)-3-hydroxy-2-oxo-5-(trifluoromethyl)pyrrolidin-3-yl)methyl)imidazo[1,2-b]pyridazin-2-yl)methanaminium trifluoroacetate. tert-Butyl (3R,5S)-3-((2-((S)-((tert-butoxycarbonyl)amino)(4,4-difluorocyclohexyl)methyl)imidazo[1,2-b]pyridazin-7-yl)methyl)-3-hydroxy-2-oxo-5-(trifluoromethyl)pyrrolidine-1-carboxylate (23 mg, 35 mmol, Step A) was dissolved in DCM (0.35 mL) and the solution was cooled to 0° C. TFA (0.35 mL) was added and the reaction was stirred at rt for 1.5 h. and the reaction was warmed to rt and concentrated to dryness. The residue was further dried under high vacuum for at least 3 h to afford the title compound, which was used without further purification.

Step C: 4-Cyclopropyl-N—((S)-(4,4-difluorocyclohexyl(7-(((3R,5S)-3-hydroxy-2-oxo-5-(trifluoromethyl)pyrrolidin-3-yl)methyl)imidazo[1,2-b]pyridazin-2-yl)methyl)-1,2,5-oxadiazole-3-carboxamide. To a solution of (S)-(4,4-difluorocyclohexyl)(7-(((3R,5S)-3-hydroxy-2-oxo-5-(trifluoromethyl)pyrrolidin-3-yl)methyl)imidazo[1,2-b]pyridazin-2-yl)methanaminium trifluoroacetate (15 mg, 26 mmol, Step B) in MeCN (0.75 mL) were added DIPEA (68 mL, 0.40 mmol) and 2,5-dioxopyrrolidin-1-yl 4-cyclopropyl-1,2,5-oxadiazole-3-carboxylate (7.9 mg, 32 mmol, Intermediate 2) sequentially. The reaction was stirred at rt for 8 h and was subsequently concentrated to dryness and purified by preparative HPLC (XBridge C18, 5 μm, 50×100 mm, 10-100% MeCN/water (with 20 mM NH₄OH)) to afford the title compound in 58% yield. ¹H NMR (600 MHz, DMSO-d₆) δ 9.48 (d, J=9.0 Hz, 1H), 8.85 (s, 1H), 8.44 (d, J=2.0 Hz, 1H), 8.20 (s, 1H), 7.97-7.91 (m, 1H), 6.00 (s, 1H), 5.20 (t, J=8.5 Hz, 1H), 4.36-4.28 (m, 1H), 3.08 (d, J=13.9 Hz, 1H), 2.84 (d, J=13.9 Hz, 1H), 2.32-2.25 (m, 1H), 2.25-2.14 (m, 1H), 2.14-1.95 (m, 4H), 1.94-1.87 (m, 1H), 1.87-1.72 (m, 2H), 1.68-1.60 (m, 1H), 1.46-1.35 (m, 1H), 1.34-1.23 (m, 1H), 1.16-1.09 (m, 2H), 1.00-0.93 (m, 2H). MS (ESI) m/z: [M+H]⁺ Found 584.1.

### Example 37: 4-Methyl-N—((S)-5,5,5-trifluoro-4,4-dimethyl-1-(7-(((3S,5S)-2-oxo-5-(trifluoromethyl)pyrrolidin-3-yl)methyl)imidazo[1,2-b]pyridazin-2-yl)pentyl)-1,2,5-oxadiazole-3-carboxamide

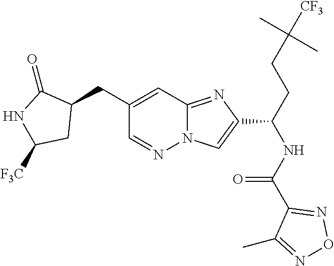 [SMILES: Cc1nonc1C(=O)N[C@@H](CCC(C)(C)C(F)(F)F)c1cn2ncc(C[C@H]3C[C@@H](C(F)(F)F)NC3=O)cc2n1]

(5S)-3-((2-((S)-1-Amino-5,5,5-trifluoro-4,4-dimethylpentyl)imidazo[1,2-b]pyridazin-7-yl)methyl)-5-(trifluoromethyl)pyrrolidin-2-one (50 mg, 0.11 mmol, Intermediate 18) was dissolved in MeCN (3.2 mL). Then DIPEA (0.29 mL, 1.7 mmol) and 2,5-dioxopyrrolidin-1-yl 4-methyl-1,2,5-oxadiazole-3-carboxylate (31 mg, 0.14 mmol) were added and the mixture was stirred at for 1 h. The mixture was concentrated to dryness and purified by silica gel chromatography (0-100% acetone/hexanes (0.1% TEA)) to afford the title compound as a mixture of diastereomers. The diastereomers were separated by chiral SFC (Chiralcel OD-H, 5 μm, 250×21 mm, 25% MeOH, 75% CO₂) to afford the title compound as the first-eluting fraction in 21% yield. ¹H NMR (500 MHz, DMSO-do) δ 9.49 (d, J=8.6 Hz, 1H), 8.64 (s, 1H), 8.44 (d, J=2.0 Hz, 1H), 8.14 (s, 1H), 7.96-7.92 (m, 1H), 5.24-5.16 (m, 1H), 4.30-4.20 (m, 1H), 3.13-3.05 (m, 1H), 2.97-2.87 (m, 1H), 2.78-2.70 (m, 1H), 2.49 (s, 3H), 2.45-2.35 (m, 1H), 2.16-2.05 (m, 1H), 2.04-1.92 (m, 1H), 1.71-1.59 (m, 2H), 1.57-1.47 (m, 1H), 1.08 (s, 6H). MS (ESI) m/z: [M+H]⁺ Found 562.2.

### Example 38: 4-Cyclopropyl-N—((S)-(4,4-difluorocyclohexyl)(7-((1S*,3R*,6S)-4-oxo-6-(trifluoromethyl)-5-azaspiro[2.4]heptan-1-yl)imidazo[1,2-b]pyridazin-2-yl)methyl)-1,2,5-oxadiazole-3-carboxamide

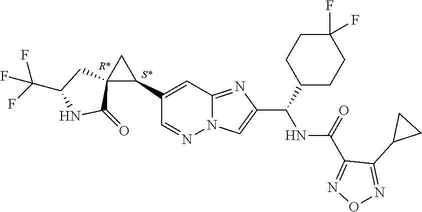 [SMILES: O=C(N[C@H](c1cn2ncc([C@@H]3C[C@@]34C[C@@H](C(F)(F)F)NC4=O)cc2n1)C1CCC(F)(F)CC1)c1nonc1C1CC1]

### Example 39: 4-Cyclopropyl-N—((S)-(4,4-difluorocyclohexyl)(7-((S*,3S*,6S)-4-oxo-6-(trifluoromethyl)-5-azaspiro[2.4]heptan-1-yl)imidazo[1,2-b]pyridazin-2-yl)methyl)-1,2,5-oxadiazole-3-carboxamide

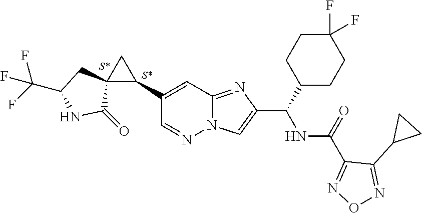 [SMILES: O=C(N[C@H](c1cn2ncc([C@@H]3C[C@]34C[C@@H](C(F)(F)F)NC4=O)cc2n1)C1CCC(F)(F)CC1)c1nonc1C1CC1]

### Example 40: 4-Cyclopropyl-N—((S)-(4,4-difluorocyclohexyl)(7-((1R,3S,6S)-4-oxo-6-(trifluoromethyl)-5-azaspiro[2.4]heptan-1-yl)imidazo[1,2-b]pyridazin-2-yl)methyl)-1,2,5-oxadiazole-3-carboxamide

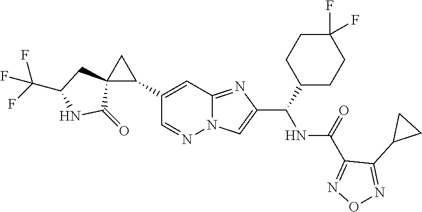 [SMILES: O=C(N[C@H](c1cn2ncc([C@H]3C[C@]34C[C@@H](C(F)(F)F)NC4=O)cc2n1)C1CCC(F)(F)CC1)c1nonc1C1CC1]

Step A: (1S)-(4,4-Difluorocyclohexyl)(7-((6S)-4-oxo-6-(trifluoromethyl)-5-azaspiro[2.4]heptan-1-yl)imidazo[1,2-b]pyridazin-2-yl)methanaminium trifluoroacetate. The title compound (79% yield) was synthesized in a manner analogous to Example 36 Step B using tert-butyl (6S)-1-(2-((S)-((tert-butoxycarbonyl)amino)(4,4-difluorocyclohexyl)methyl)imidazo[1,2-b]pyridazin-7-yl)-4-oxo-6-(trifluoromethyl)-5-azaspiro[2.4]heptane-5-carboxylate (Intermediate 20) in place of tert-butyl (3R,5S)-3-((2-((S)-((tert-butoxycarbonyl)amino)(4,4-difluorocyclohexyl)methyl)imidazo[1,2-b]pyridazin-7-yl)methyl)-3-hydroxy-2-oxo-5-(trifluoromethyl)pyrrolidine-1-carboxylate, and was used without further purification.

Step B: 4-Cyclopropyl-N—((S)-(4,4-difluorocyclohexyl)(7-((1S*,3R*,6S)-4-oxo-6-(trifluoromethyl)-5-azaspiro[2.4]heptan-1-yl)imidazo[1,2-b]pyridazin-2-yl)methyl)-1,2,5-oxadiazole-3-carboxamide, 4-cyclopropyl-N—((S)-(4,4-difluorocyclohexyl)(7-((1S*,3S*,6S)-4-oxo-6-(trifluoromethyl)-5-azaspiro[2.4]heptan-1-yl)imidazo[1,2-b]pyridazin-2-yl)methyl)-1,2,5-oxadiazole-3-carboxamide and 4-cyclopropyl-N—((S)-(4,4-difluorocyclohexyl)(7-((1R,3S,6S)-4-oxo-6-(trifluoromethyl)-5-azaspiro[2.4]heptan-1-yl)imidazo[1,2-b]pyridazin-2-yl)methyl)-1,2,5-oxadiazole-3-carboxamide. To a mixture of (1S)-(4,4-difluorocyclohexyl)(7-((6S)-4-oxo-6-(trifluoromethyl)-5-azaspiro[2.4]heptan-1-yl)imidazo[1,2-b]pyridazin-2-yl)methanaminium trifluoroacetate (28 mg, 0.062 mmol, Step A) and 4-cyclopropyl-1,2,5-oxadiazole-3-carboxylic acid (19 mg, 0.12 mmol) in MeCN (0.5 mL) were added DIPEA (0.064 mL, 0.37 mmol) and T₃P (0.092 mL, 0.16 mmol, 50% in EtOAc) sequentially. The reaction mixture was stirred at rt for 12 h then DIPEA (0.064 mL, 0.37 mmol) and T₃P (0.092 mL, 0.16 mmol, 50% in EtOAc) were added. The reaction was stirred at rt for 1 h then concentrated to dryness. Purification by preparative HPLC (XBridge C18, 5 μm, 50×100 mm 10-100% MeCN/H₂O (with 20 mM NH₄OH)) provided Example 38 as the first-eluting isomer (16% yield), Example 39 as the second-eluting isomer (16% yield), and Example 40 as the third-eluting isomer (7% yield). The absolute stereochemistry of Example 40 was determined via ¹H and 2D NMR correlation experiments. Example 38: ¹H NMR (600 MHz, DMSO-d₆) δ 9.45 (d, J=9.0 Hz, 1H), 8.79-8.73 (m, 1H), 8.43 (d, J=2.1 Hz, 1H), 8.22 (d, J=0.6 Hz, 1H), 7.86-7.82 (m, 1H), 5.20 (t, J=8.5 Hz, 1H), 4.42-4.37 (m, 1H), 2.57-2.51 (m, 2H), 2.32-2.24 (m, 1H), 2.23-2.14 (m, 1H), 2.10-1.94 (m, 2H), 1.93-1.87 (m, 1H), 1.85-1.68 (m, 3H), 1.68-1.63 (m, 1H), 1.56-1.48 (m, 2H), 1.45-1.25 (m, 2H), 1.15-1.06 (m, 2H), 0.99-0.91 (m, 2H). MS (ESI) m/z: [M+H]⁺ Found 580.2. Example 39: ¹H NMR (600 MHz, DMSO-d₆) δ 9.44 (s, 1H), 8.53 (m, 1H), 8.45 (d, J=2.1 Hz, 1H), 8.29 (d, J=2.0 Hz, 1H), 8.17 (s, 1H), 7.89-7.86 (m, 1H), 7.73 (m, 1H), 5.18 (m, 1H), 4.36 (q, J=8.3 Hz, 1H), 2.74-2.65 (m, 2H), 2.31-2.22 (m, 2H), 2.22-2.13 (m, 1H), 2.09-1.93 (m, 2H), 1.89 (d, J=13.3 Hz, 2H), 1.87-1.79 (m, 1H), 1.47 (dd, J=8.7, 5.2 Hz, 1H), 1.42-1.34 (m, 2H), 1.11 (m, 2H), 0.96 (m, 2H). MS (ESI) m/z: [M+H]⁺ Found 580.2. Example 40: ¹H NMR (600 MHz, DMSO-d₆) δ 9.45 (d, J=9.0 Hz, 1H), 8.51 (s, 1H), 8.45 (d, J=2.0 Hz, 1H), 8.19-8.15 (m, 1H), 7.90-7.87 (m, 1H), 5.19 (t, J=8.5 Hz, 1H), 4.53-4.46 (m, 1H), 2.69 (dd, J=13.8, 8.9 Hz, 1H), 2.63-2.56 (m, 1H), 2.32-2.24 (m, 1H), 2.22-2.14 (m, 1H), 2.07-1.97 (m, 2H), 1.92-1.87 (m, 1H), 1.86-1.72 (m, 3H), 1.67-1.62 (m, 1H), 1.55-1.50 (m, 1H), 1.45-1.37 (m, 1H), 1.34-1.24 (m, 2H), 1.16-1.08 (m, 2H), 0.99-0.93 (m, 2H). MS (ESI) m/z: [M+H]⁺ Found 580.2.

### Example 41: 4-Cyclopropyl-N—((S)-(4,4-difluorocyclohexyl)(7-((1R*,3R*,6S)-4-oxo-6-(trifluoromethyl)-5-azaspiro[2.4]heptan-1-yl)imidazo[1,2-b]pyridazin-2-yl)methyl)-1,2,5-oxadiazole-3-carboxamide

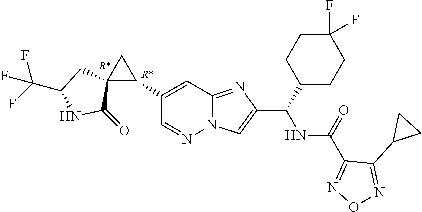 [SMILES: O=C(N[C@H](c1cn2ncc([C@H]3C[C@@]34C[C@@H](C(F)(F)F)NC4=O)cc2n1)C1CCC(F)(F)CC1)c1nonc1C1CC1]

Step A: (S)-(4,4-Difluorocyclohexyl)(7-((1R*,3R*,6S)-4-oxo-6-(trifluoromethyl)-5-azaspiro[2.4]heptan-1-yl)imidazo[1,2-b]pyridazin-2-yl)methanaminium trifluoroacetate. The title compound (81% yield) was synthesized in a manner analogous to Example 36 Step B using (1R*,2R*)-2-(2-((S)-((tert-butoxycarbonyl)amino)(4,4-difluorocyclohexyl)methyl)imidazo[1,2-b]pyridazin-7-yl)-1-((S)-2-((tert-butoxycarbonyl)amino)-3,3,3-trifluoropropyl)cyclopropane-1-carboxylic acid (Intermediate 21) in place of tert-butyl (3R,5S)-3-((2-((S)-((tert-butoxycarbonyl)amino)(4,4-difluorocyclohexyl)methyl)imidazo[1,2-b]pyridazin-7-yl)methyl)-3-hydroxy-2-oxo-5-(trifluoromethyl)pyrrolidine-1-carboxylate, and was used without further purification

Step B: 4-Cyclopropyl-N—((S)-(4,4-difluorocyclohexyl)(7-((1R*,3R*,6S)-4-oxo-6-(trifluoromethyl)-5-azaspiro[2.4]heptan-1-yl)imidazo[1,2-b]pyridazin-2-yl)methyl)-1,2,5-oxadiazole-3-carboxamide. The title compound (24% yield) was synthesized in a manner analogous to Example 38 Step B using (S)-(4,4-difluorocyclohexyl)(7-((1R*,3R*,6S)-4-oxo-6-(trifluoromethyl)-5-azaspiro[2.4]heptan-1-yl)imidazo[1,2-b]pyridazin-2-yl)methanaminium trifluoroacetate (Step A) in place of (1S)-(4,4-difluorocyclohexyl)(7-((6S)-4-oxo-6-(trifluoromethyl)-5-azaspiro[2.4]heptan-1-yl)imidazo[1,2-b]pyridazin-2-yl)methanaminium trifluoroacetate. ¹H NMR (600 MHz, DMSO-d₆) δ 9.47 (d, J=9.0 Hz, 1H), 8.73-8.70 (m, 1H), 8.49 (d, J=2.1 Hz, 1H), 8.21 (s, 1H), 7.89 (d, J=2.1 Hz, 1H), 5.19 (t, J=8.5 Hz, 11H), 4.33-4.26 (m, 1H), 2.59-2.54 (m, 1H), 2.31-2.24 (m, 1H), 2.22-2.14 (m, 1H), 2.14-1.94 (m, 4H), 1.93-1.87 (m, 1H), 1.86-1.69 (m, 3H), 1.66-1.61 (m, 1H), 1.49-1.35 (m, 2H), 1.33-1.22 (m, 1H), 1.16-1.07 (m, 2H), 1.01-0.91 (m, 2H). MS (ESI) m/z: [M+H]⁺ Found 580.6.

### Example 42: 4-Cyclopropyl-N—((S)-(7-(((3S*,5S)-5-cyclopropyl-2-oxopyrrolidin-3-yl)methyl)imidazo[1,2-b]pyridazin-2-yl)(4,4-difluorocyclohexyl)methyl)-1,2,5-oxadiazole-3-carboxamide

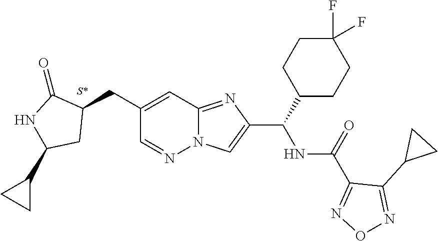 [SMILES: O=C(N[C@H](c1cn2ncc(C[C@H]3C[C@@H](C4CC4)NC3=O)cc2n1)C1CCC(F)(F)CC1)c1nonc1C1CC1]

### Example 43: 4-Cyclopropyl-N—((S)-(7-(((3R*,5S)-5-cyclopropyl-2-oxopyrrolidin-3-yl)methyl)imidazo[1,2-b]pyridazin-2-yl)(4,4-difluorocyclohexyl)methyl)-1,2,5-oxadiazole-3-carboxamide

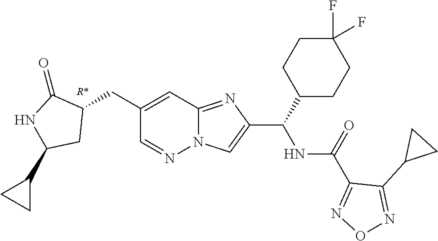 [SMILES: O=C(N[C@H](c1cn2ncc(C[C@@H]3C[C@@H](C4CC4)NC3=O)cc2n1)C1CCC(F)(F)CC1)c1nonc1C1CC1]

The title compounds were synthesized in a manner analogous to Example 36 Step C using (1S)-(7-(((5S)-5-cyclopropyl-2-oxopyrrolidin-3-yl)methyl)imidazo[1,2-b]pyridazin-2-yl)(4,4-difluorocyclohexyl)methanaminium trifluoroacetate (Intermediate 23) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The resulting material was purified by preparative HPLC (Xbridge C18, 5 μm, 50×100 mm, 10-100% MeCN/water (with 20 mM NH₄OH)) to afford a mixture of diastereomers. The diastereomers were separated by chiral SFC (Stationary phase: IB, 5 μm, 21×250 mm, Mobile phase: 20% MeOH, 80% CO₂) affording Example 42 as the first-eluting isomer (19% yield) and Example 43 as the second-eluting isomer (12% yield). Example 42: ¹H NMR (600 MHz, DMSO-d₆) δ 9.46 (d, J=9.0 Hz, 1H), 8.44 (d, J=2.0 Hz, 1H), 8.18 (s, 1H), 7.98-7.90 (m, 2H), 5.18 (t, J=8.5 Hz, 1H), 3.09 (dd, J=13.8, 4.4 Hz, 1H), 2.89-2.80 (m, 1H), 2.76-2.69 (m, 1H), 2.69-2.64 (m, 1H), 2.31-2.25 (m, 1H), 2.23-2.13 (m, 2H), 2.10-1.94 (m, 2H), 1.93-1.86 (m, 1H), 1.85-1.71 (m, 2H), 1.67-1.55 (m, 1H), 1.47-1.34 (m, 2H), 1.34-1.22 (m, 1H), 1.15-1.08 (m, 2H), 1.00-0.92 (m, 2H), 0.75-0.64 (m, 1H), 0.44-0.30 (m, 2H), 0.26-0.04 (m, 2H). MS (ESI) m/z: [M+H]⁺ Found 540.2. Example 43: ¹H NMR (600 MHz, DMSO-d₆) δ 9.46 (d, J=9.0 Hz, 1H), 8.44 (d, J=2.0 Hz, 1H), 8.18 (s, 1H), 7.98-7.83 (m, 2H), 5.18 (t, J=8.5 Hz, 1H), 3.07-2.99 (m, 1H), 2.95-2.87 (m, 1H), 2.87-2.82 (m, 1H), 2.72-2.63 (m, 1H), 2.31-2.23 (m, 1H), 2.23-2.12 (m, 1H), 2.12-1.95 (m, 2H), 1.95-1.84 (m, 3H), 1.84-1.70 (m, 2H), 1.69-1.58 (m, 1H), 1.46-1.32 (m, 1H), 1.32-1.23 (m, 1H), 1.16-1.10 (m, 2H), 1.02-0.90 (m, 2H), 0.90-0.77 (m, 1H), 0.43-0.36 (m, 1H), 0.36-0.31 (m, 1H), 0.17-0.10 (m, 2H). MS (ESI) m/z: [M+H]⁺ Found 540.2.

### Example 44: 4-Cyclopropyl-N—((S)-(4,4-difluorocyclohexyl)(7-(((3R*,4R*)-2-oxo-4-(trifluoromethyl)piperidin-3-yl)methyl)imidazo[1,2-b]pyridazin-2-yl)methyl)-1,2,5-oxadiazole-3-carboxamide

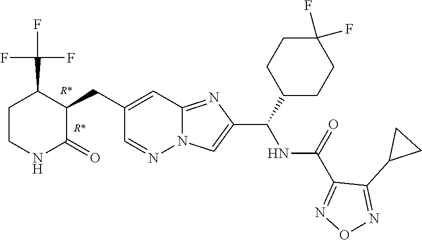 [SMILES: O=C(N[C@H](c1cn2ncc(C[C@H]3C(=O)NCC[C@H]3C(F)(F)F)cc2n1)C1CCC(F)(F)CC1)c1nonc1C1CC1]

### Example 45: 4-Cyclopropyl-N—((S)-(4,4-difluorocyclohexyl)(7-(((3S*,4S*)-2-oxo-4-(trifluoromethyl)piperidin-3-yl)methyl)imidazo[1,2-b]pyridazin-2-yl)methyl)-1,2,5-oxadiazole-3-carboxamide

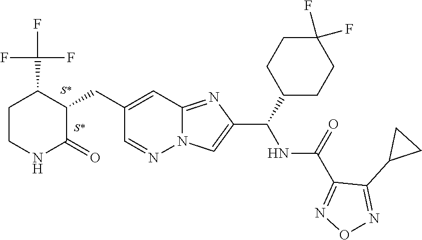 [SMILES: O=C(N[C@H](c1cn2ncc(C[C@@H]3C(=O)NCC[C@@H]3C(F)(F)F)cc2n1)C1CCC(F)(F)CC1)c1nonc1C1CC1]

The title compounds were synthesized in a manner analogous to Example 36 Step C using 3-((2-((S)-amino(4,4-difluorocyclohexyl)methyl)imidazo[1,2-b]pyridazin-7-yl)methyl)-4-(trifluoromethyl)piperidin-2-one (Intermediate 24) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The resulting material was purified by silica gel chromatography (0-10% MeOH (with 2.0 M ammonia)/DCM) to afford a mixture of diastereomers. The diastereomers were separated by chiral SFC (Stationary phase: Chiralpak IH, 5 μm, 250×21 mm, Mobile phase: 30% acetonitrile:methanol (1:1), 70% CO₂), affording Example 44 as the first-eluting isomer (20% yield) and Example 45 as the second-eluting isomer (18% yield). Example 44: ¹H NMR (600 MHz, DMSO-d₆) δ 9.48-9.44 (m, 1H), 8.45 (d, J=2.0 Hz, 1H), 8.17 (s, 1H), 7.89 (s, 1H), 7.70-7.63 (m, 1H), 5.18 (s, 1H), 3.23-3.18 (m, 2H), 3.15-3.11 (m, 2H), 2.85-2.79 (m, 1H), 2.32-2.25 (m, 1H), 2.23-2.15 (m, 1H), 2.10-2.02 (m, 2H), 2.02-1.94 (m, 2H), 1.94-1.88 (m, 2H), 1.86-1.73 (m, 2H), 1.67-1.61 (m, 1H), 1.45-1.37 (m, 1H), 1.33-1.25 (m, 1H), 1.15-1.11 (m, 2H), 0.98-0.95 (m, 2H). MS (ESI) m/z: [M+H]⁺ Found 582.8. Example 45: ¹H NMR (600 MHz, DMSO-d₆) δ 9.49-9.45 (m, 1H), 8.45 (s, 1H), 8.17 (s, 1H), 7.89 (s, 1H), 7.66-7.64 (m, 1H), 5.21-5.16 (m, 1H), 3.21-3.17 (m, 2H), 3.15-3.10 (m, 2H), 2.84-2.78 (m, 1H), 2.31-2.25 (m, 1H), 2.22-2.15 (m, 1H), 2.11-2.00 (m, 3H), 1.98-1.87 (m, 3H), 1.86-1.71 (m, 2H), 1.66-1.61 (m, 1H), 1.44-1.36 (m, 1H), 1.34-1.25 (m, 1H), 1.15-1.10 (m, 2H), 0.99-0.95 (m, 2H). MS (ESI) m/z: [M+H]⁺ Found 582.8.

### Example 46: N—((S)-(4,4-difluorocyclohexyl)(7-(((3R*,6R*)-2-oxo-6-(trifluoromethyl)piperidin-3-yl)methyl)imidazo[1,2-b]pyridazin-2-yl)methyl)-4-methyl-1,2,5-oxadiazole-3-carboxamide

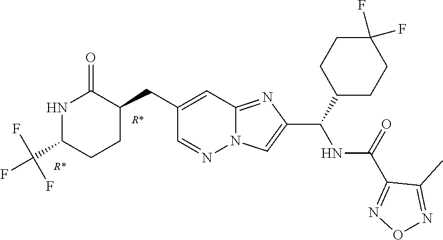 [SMILES: Cc1nonc1C(=O)N[C@H](c1cn2ncc(C[C@H]3CC[C@H](C(F)(F)F)NC3=O)cc2n1)C1CCC(F)(F)CC1]

### Example 47: N—((S)-(4,4-difluorocyclohexyl)₇-(((3R*,6S*)-2-oxo-6-(trifluoromethyl)piperidin-3-yl)methyl)imidazo[1,2-b]pyridazin-2-yl)methyl)-4-methyl-1,2,5-oxadiazole-3-carboxamide

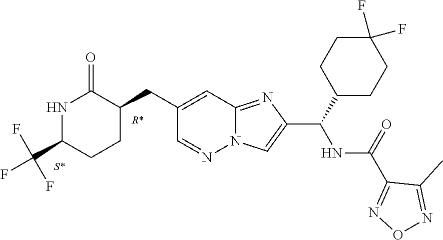 [SMILES: Cc1nonc1C(=O)N[C@H](c1cn2ncc(C[C@H]3CC[C@@H](C(F)(F)F)NC3=O)cc2n1)C1CCC(F)(F)CC1]

### Example 48: N—((S)-(4,4-difluorocyclohexyl)₇-(((3S*,6R*)-2-oxo-6-(trifluoromethyl)piperidin-3-yl)methyl)imidazo[1,2-b]pyridazin-2-yl)methyl)-4-methyl-1,2,5-oxadiazole-3-carboxamide

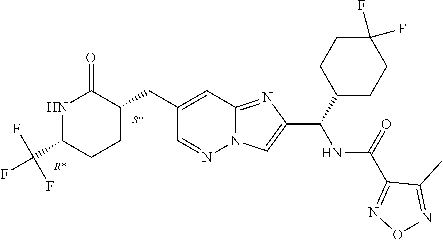 [SMILES: Cc1nonc1C(=O)N[C@H](c1cn2ncc(C[C@@H]3CC[C@H](C(F)(F)F)NC3=O)cc2n1)C1CCC(F)(F)CC1]

The title compounds were synthesized in a manner analogous to Example 36 Step C using 3-((2-((S)-amino(4,4-difluorocyclohexyl)methyl)imidazo[1,2-b]pyridazin-7-yl)methyl)-6-(trifluoromethyl)piperidin-2-one (Intermediate 12) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate and 2,5-dioxopyrrolidin-1-yl 4-methyl-1,2,5-oxadiazole-3-carboxylate in place of 2,5-dioxopyrrolidin-1-yl 4-cyclopropyl-1,2,5-oxadiazole-3-carboxylate. The residue was purified by preparative HPLC (Xbridge C18, 5 μm, 50×100 mm, 10-100% MeCN/water (with 20 mM NH₄OH)) followed by silica gel chromatography (0-10/0 MeOH (with 2.0 M ammonia)/DCM) to give a mixture of diastereomers. The diastereomers were separated by chiral SFC (Stationary phase: Chiralpak IH, 5 μm, 250×21 mm, Mobile phase: 25% methanol, 75% CO₂) to provide Example 46 as the first-eluting isomer (2% yield), Example 47 as the second-eluting isomer (22% yield), and Example 48 as the third-eluting isomer (26% yield). Example 46: ¹H NMR (600 MHz, DMSO-d₆) δ 9.39 (d, J=9.0 Hz, 1H), 8.42 (d, J=2.0 Hz, 1H), 8.18 (s, 1H), 8.11-8.09 (m, 1H), 7.90-7.89 (m, 1H), 5.18-5.14 (m, 1H), 4.16-4.08 (m, 1H), 3.21-3.17 (m, 1H), 2.81-2.74 (m, 1H), 2.47-2.46 (m, 3H), 2.22-2.14 (m, 1H), 2.08-1.95 (m, 3H), 1.93-1.87 (m, 1H), 1.83-1.71 (m, 3H), 1.66-1.59 (m, 2H), 1.48-1.36 (m, 2H), 1.32-1.23 (m, 2H). MS (ESI) m/z: [M+H]⁺ Found 556.2. Example 47: ¹H NMR (600 MHz, DMSO-d₆) δ 9.42-9.38 (m, 1H), 8.41-8.39 (m, 1H), 8.24-8.21 (m, 1H), 8.20-8.17 (m, 1H), 7.90-7.88 (m, 1H), 5.18-5.13 (m, 1H), 4.12-4.04 (m, 1H), 3.22-3.17 (m, 1H), 2.81-2.67 (m, 2H), 2.47-2.45 (m, 3H), 2.23-2.14 (m, 1H), 2.08-1.97 (m, 2H), 1.96-1.88 (m, 3H), 1.84-1.72 (m, 2H), 1.72-1.67 (m, 1H), 1.65-1.59 (m, 1H), 1.56-1.48 (m, 1H), 1.43-1.34 (m, 1H), 1.32-1.23 (m, 1H). MS (ESI) m/z: [M+H]⁺ Found 556.2. Example 48: ¹H NMR (600 MHz, DMSO-d₆) δ 9.41-9.38 (m, 1H), 8.42-8.39 (m, 1H), 8.23-8.21 (m, 1H), 8.19-8.18 (m, 1H), 7.90-7.89 (m, 1H), 5.19-5.14 (m, 1H), 4.10-4.03 (m, 1H), 3.22-3.16 (m, 1H), 2.79-2.74 (m, 1H), 2.73-2.67 (m, 1H), 2.48-2.45 (m, 3H), 2.23-2.14 (m, 1H), 2.09-1.96 (m, 2H), 1.96-1.91 (m, 2H), 1.91-1.87 (m, 1H), 1.85-1.72 (m, 2H), 1.72-1.65 (m, 1H), 1.64-1.57 (m, 1H), 1.56-1.47 (m, 1H), 1.43-1.33 (m, 1H), 1.31-1.22 (m, 1H). MS (ESI) m/z: [M+H]⁺ Found 556.2.

### Example 49: 4-Cyclopropyl-N—((S)-(4,4-difluorocyclohexyl)(7-(((3S*,4R*)-2-oxo-4-(trifluoromethyl)pyrrolidin-3-yl)methyl)imidazo[1,2-b]pyridazin-2-yl)methyl)-1,2,5-oxadiazole-3-carboxamide

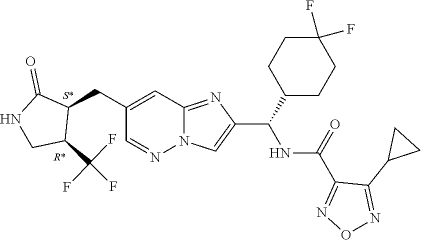 [SMILES: O=C(N[C@H](c1cn2ncc(C[C@@H]3C(=O)NC[C@@H]3C(F)(F)F)cc2n1)C1CCC(F)(F)CC1)c1nonc1C1CC1]

### Example 50: 4-Cyclopropyl-N—((S)-(4,4-difluorocyclohexyl)(7-2-oxo-4-(trifluoromethyl)pyrrolidin-3-yl)methyl)imidazo[1,2-b]pyridazin-2-yl)methyl)-1,2,5-oxadiazole-3-carboxamide

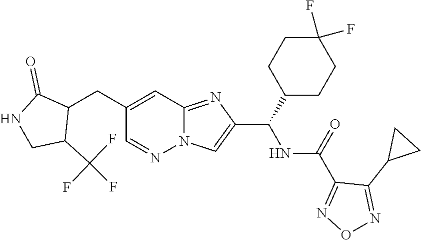 [SMILES: O=C(N[C@H](c1cn2ncc(CC3C(=O)NCC3C(F)(F)F)cc2n1)C1CCC(F)(F)CC1)c1nonc1C1CC1]

The title compounds were synthesized in a manner analogous to Example 36 Step C using (1S)-(4,4-difluorocyclohexyl)₇-((2-oxo-4-(trifluoromethyl)pyrrolidin-3-yl)methyl)imidazo[1,2-b]pyridazin-2-yl)methanaminium trifluoroacetate (Intermediate 26) in place of tert-butyl (5S)-3-((2-((S)-((tert-butoxycarbonyl)amino)(4,4-difluorocyclohexyl)methyl)imidazo[1,2-b]196yridazine-7-yl)methyl)-2-oxo-5-(trifluoromethyl)pyrrolidine-1-carboxylate. The resulting material was purified by silica gel chromatography (0-100% acetone/hexanes) to afford a mixture of isomers. The isomers were separated by chiral SFC (Chiralpak IH, 5 μm, 250×21 mm, 25% MeCN/MeOH (1/1), 75% CO₂) affording Example 101 (6% yield) as the first-eluting isomer, Example 49 (17% yield) as the second-eluting isomer, and Example 50 (22% yield) as a 2:1 mixture of the two remaining diastereomers as the third-eluting fraction.

Example 49: ¹H NMR (600 MHz, DMSO-d₆) δ 9.45 (d, J=9.0 Hz, 1H), 8.48 (d, J=2.1 Hz, 1H), 8.18 (s, 1H), 8.01-7.95 (m, 2H), 5.18 (t, J=8.5 Hz, 1H), 3.59-3.47 (m, 2H), 3.39-3.32 (m, 2H), 3.15 (dd, J=16.0, 6.6 Hz, 1H), 2.82 (dd, J=15.9, 9.1 Hz, 1H), 2.33-2.24 (m, 1H), 2.24-2.13 (m, 1H), 2.11-1.95 (m, 2H), 1.92-1.87 (m, 1H), 1.85-1.69 (m, 2H), 1.67-1.59 (m, 1H), 1.46-1.34 (m, 1H), 1.34-1.21 (m, 1H), 1.18-1.05 (m, 2H), 0.99-0.94 (m, 2H). MS (ESI) m/z: [M+H]⁺ Found 568.0. Example 50: MS (ESI) m/z: [M+H]⁺ Found 568.2.

### Example 51: N—((S)-(4,4-Difluorocyclohexyl)(7-(((3S,5S)-2-oxo-5-(trifluoromethyl)pyrrolidin-3-yl)methyl)imidazo[1,2-b]pyridazin-2-yl)methyl)-1-isopropyl-1H-pyrazole-5-carboxamide

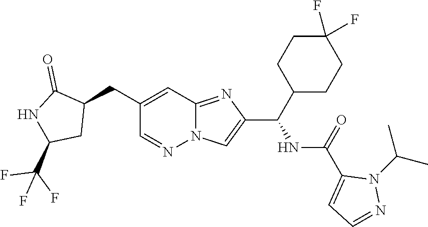 [SMILES: CC(C)n1nccc1C(=O)N[C@H](c1cn2ncc(C[C@H]3C[C@@H](C(F)(F)F)NC3=O)cc2n1)C1CCC(F)(F)CC1]

### Example 52: N—((S)-(4,4-Difluorocyclohexyl)(7-(((3R,5S)-2-oxo-5-(trifluoromethyl)pyrrolidin-3-yl)methyl)imidazo[1,2-b]pyridazin-2-yl)methyl)-1-isopropyl-1H-pyrazole-5-carboxamide

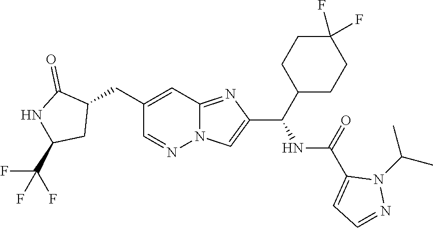 [SMILES: CC(C)n1nccc1C(=O)N[C@H](c1cn2ncc(C[C@@H]3C[C@@H](C(F)(F)F)NC3=O)cc2n1)C1CCC(F)(F)CC1]

The title compounds were synthesized in a manner analogous to Example 36 Step C using (5S)-3-((2-((S)-amino(4,4-difluorocyclohexyl)methyl)imidazo[1,2-b]pyridazin-7-yl)methyl)-5-(trifluoromethyl)pyrrolidin-2-one (Intermediate 40) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate and 2,5-dioxopyrrolidin-1-yl 1-isopropyl-1H-pyrazole-5-carboxylate (Intermediate 27) in place of 2,5-dioxopyrrolidin-1-yl 4-cyclopropyl-1,2,5-oxadiazole-3-carboxylate. The residue was purified by preparative HPLC (Xbridge C18, 5 μm, 50×100 mm, 10-100% MeCN/water (with 20 mM NH₄OH)) to afford a mixture of diastereomers. The diastereomers were separated by chiral SFC (Stationary phase: Chiralpak AS-H, 5 μm, 250×21 mm, 35% MeOH/IPA (1:1), 65% CO₂) to afford Example 51 (19% yield) as the first-eluting isomer and Example 52 (6% yield) as the second-eluting isomer. Example 51: ¹H NMR (600 MHz, DMSO-d₆) δ 8.71 (d, J=9.1 Hz, 1H), 8.65 (s, 1H), 8.44 (d, J=2.1 Hz, 1H), 8.17 (s, 1H), 7.98-7.88 (m, 1H), 7.49 (d, J=1.9 Hz, 1H), 6.91 (d, J=2.0 Hz, 1H), 5.45-5.35 (m, 1H), 5.16 (t, J=8.6 Hz, 1H), 4.31-4.21 (m, 1H), 3.10 (dd, J=14.3, 4.9 Hz, 1H), 3.02-2.86 (m, 1H), 2.73 (dd, J=14.4, 9.3 Hz, 1H), 2.45-2.34 (m, 1H), 2.23-2.14 (m, 1H), 2.11-1.94 (m, 2H), 1.90-1.84 (m, 1H), 1.84-1.71 (m, 2H), 1.71-1.65 (m, 1H), 1.65-1.59 (m, 1H), 1.43-1.28 (m, 7H), 1.31-1.19 (m, 1H). MS (ESI) m/z: [M+H]⁺ Found 568.1. Further NMR analysis confirmed the stereochemistry of Example 51 as shown here:

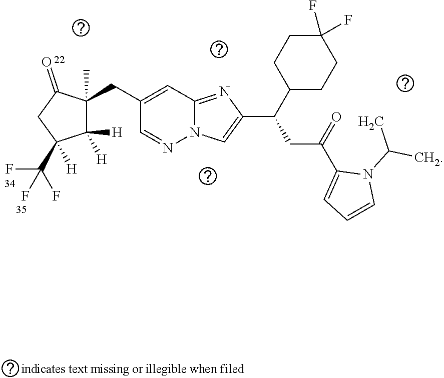

Key ROESY correlations were observed between the respective proton 13a and the proton 11a to the same methylene proton 12a on the lactam, confirming that they are all on the same side of the ring. Additional ROESY correlations were also observed between both protons in the methylene group at the C-10 position to methylene proton 12b on the lactam, showing they are on the same side of the ring. These correlations support relative cis stereochemistry between the C-10 sidechain and C-20 sidechain. Example 52: ¹H NMR (600 MHz, DMSO-d₆) δ 8.71 (d, J=9.1 Hz, 1H), 8.68 (s, 1H), 8.45 (d, J=2.1 Hz, 1H), 8.21-8.10 (m, 1H), 7.99-7.87 (m, 1H), 7.49 (d, J=2.0 Hz, 1H), 6.92 (d, J=2.0 Hz, 1H), 5.42-5.32 (m, 1H), 5.15 (t, J=8.7 Hz, 1H), 4.26-4.16 (m, 1H), 3.08 (dd, J=14.1, 4.9 Hz, 1H), 2.93-2.85 (m, 1H), 2.72 (dd, J=14.1, 9.1 Hz, 1H), 2.22-1.95 (m, 5H), 1.91-1.85 (m, 1H), 1.85-1.70 (m, 2H), 1.65-1.59 (m, 1H), 1.44-1.32 (m, 7H), 1.31-1.22 (m, 1H). MS (ESI) m/z: [M+H]⁺ Found 568.1. Further NMR analysis confirmed the stereochemistry of Example 52 as shown here:

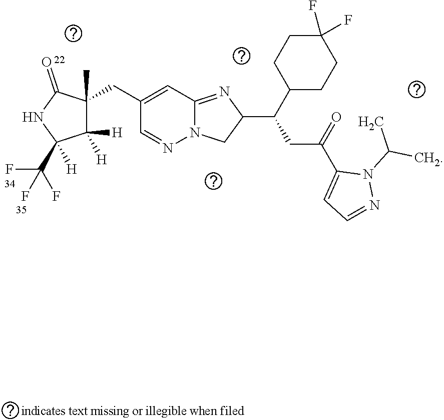

Key ROESY correlations were observed from proton 13a and both protons at the C-10 position to the same methylene proton 12a on the lactam, indicating they are all on the same side of the ring. Additional ROESY correlations were observed from proton 11a to methylene proton 12b only, indicating they are on the opposite side of the ring to proton 13a. These correlations support relative trans stereochemistry between the C-10 sidechain and the C-20 sidechain.

### Example 53: 4-Cyclopropyl-N—((S)-(4,4-difluorocyclohexyl)(7-(((R*)-3-methyl-2-oxopyrrolidin-3-yl)methyl)imidazo[1,2-b]pyridazin-2-yl)methyl)-1,2,5-oxadiazole-3-carboxamide

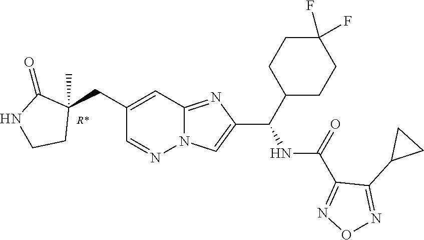 [SMILES: C[C@]1(Cc2cnn3cc([C@@H](NC(=O)c4nonc4C4CC4)C4CCC(F)(F)CC4)nc3c2)CCNC1=O]

### Example 54: 4-Cyclopropyl-N—((S)-(4,4-difluorocyclohexyl)(7-(((S*)-3-methyl-2-oxopyrrolidin-3-yl)methyl)imidazo[1,2-b]pyridazin-2-yl)methyl)-1,2,5-oxadiazole-3-carboxamide

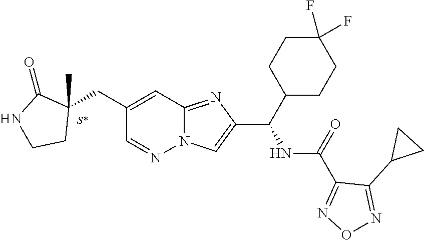 [SMILES: C[C@@]1(Cc2cnn3cc([C@@H](NC(=O)c4nonc4C4CC4)C4CCC(F)(F)CC4)nc3c2)CCNC1=O]

Step A: tert-Butyl 3-((2-((S)-((tert-butoxycarbonyl)amino)(4,4-difluorocyclohexyl)methyl)imidazo[1,2-b]pyridazin-7-yl)methyl)-3-methyl-2-oxopyrrolidine-1-carboxylate. The title compound was synthesized in a manner analogous to Intermediate 40 Step A using tert-butyl 3-methyl-2-oxopyrrolidine-1-carboxylate in place of tert-butyl (S)-2-oxo-5-(trifluoromethyl)pyrrolidine-1-carboxylate. The residue was purified by silica gel chromatography (0-100% (10% MeOH in EtOAc)/hexanes) to provide the title compound as a yellow foam (66% yield).

Step B: 3-((2-((S)-Amino(4,4-difluorocyclohexyl)methyl)imidazo[1,2-b]pyridazin-7-yl)methyl)-3-methylpyrrolidin-2-one. tert-Butyl 3-((2-((S)-((tert-butoxycarbonyl)amino(4,4-difluorocyclohexyl)methyl)imidazo[1,2-b]pyridazin-7-yl)methyl)-3-methyl-2-oxopyrrolidine-1-carboxylate (288 mg, 0.499 mmol, Step A) was dissolved in DCM (0.2 mL) and then TFA (0.4 mL) was added and the resulting mixture was stirred at rt for 4 h. The reaction was concentrated to dryness and saturated aqueous NaHCO₃ was added. The mixture was extracted with EtOAc, then the combined organic layers were dried over anhydrous MgSO₄, filtered, and concentrated to dryness to afford the title compound as a yellow foam which was used without further purification.

Step C: 4-Cyclopropyl-N—((S)-(4,4-difluorocyclohexyl)(7-(((R*)-3-methyl-2-oxopyrrolidin-3-yl)methyl)imidazo[1,2-b]pyridazin-2-yl)methyl)-1,2,5-oxadiazole-3-carboxamide and 4-cyclopropyl-N—((S)-(4,4-difluorocyclohexyl)(7-(((S*)-3-methyl-2-oxopyrrolidin-3-yl)methyl)imidazo[1,2-b]pyridazin-2-yl)methyl)-1,2,5-oxadiazole-3-carboxamide. A mixture of 4-cyclopropyl-1,2,5-oxadiazole-3-carboxylic acid (176 mg, 1.15 mmol) and DIPEA (0.46 mL) in EtOAc (0.5 mL) was stirred for 10 min at rt after which 2,4,6-tripropyl-1,3,5,2,4,6-trioxatriphosphinane 2,4,6-trioxide (0.908 mL, 1.53 mmol) was added dropwise over 3 min. After stirring this mixture for 10 min at rt, a solution of 3-((2-((S)-amino(4,4-difluorocyclohexyl)methyl)imidazo[1,2-b]pyridazin-7-yl)methyl)-3-methylpyrrolidin-2-one (288 mg, 0.763 mmol, Step B) in 0.5 mL of EtOAc and 2 mL of DCM was added to the reaction. The resulting mixture was stirred at rt for 15 min, then concentrated to dryness. The residue was purified by basic preparative HPLC (XBridge C18, 5 μm, 50×100 mm, 10-100% MeCN/H₂O (20 mM NH₄OH)) affording a mixture of diastereomers as an off-white solid. The diastereomers were separated by chiral SFC (Chiralpak IB N3, 5 μm, 250×21 mm, 30% (1/1 ACN/methanol), 70% CO₂). The first-eluting isomer (12.5% yield) was designated as the (R*) isomer (Example 53) and the second-eluting isomer (11.2% yield) was designated as the (S*) isomer (Example 54). Example 53: ¹H NMR (500 MHz, CDCl₃) δ 8.25 (d, J=2.1 Hz, 1H), 7.85 (s, 1H), 7.73 (d, J=8.8 Hz, 1H), 7.71-7.69 (m, 1H), 5.37 (s, 1H), 5.32-5.22 (m, 1H), 3.37-3.22 (m, 1H), 3.22-2.97 (m, 2H), 2.74 (d, J=13.9 Hz, 1H), 2.67-2.49 (m, 1H), 2.21-2.11 (m, 3H), 2.06 (d, J=6.0 Hz, 1H), 2.00 (d, J=12.6 Hz, 1H), 1.95-1.82 (m, 1H), 1.82-1.62 (m, 1H), 1.39 (d, J=12.5 Hz, 1H), 1.28 (s, 3H), 1.26 (s, 1H), 1.22-1.11 (m, 3H), 1.11-1.04 (m, 1H), 0.07 (s, 2H). MS (ESI) m/z: [M+H]⁺ Found 514.3. Example 54: ¹H NMR (500 MHz, CDCl₃) δ 8.25 (d, J=2.1 Hz, 1H), 7.85 (s, 1H), 7.74 (s, 1H), 7.73-7.70 (m, 2H), 5.39 (s, 1H), 5.30-5.24 (m, 1H), 3.36-3.24 (m, 1H), 3.16-3.01 (m, 2H), 2.74 (d, J=13.9 Hz, 1H), 2.63-2.51 (m, 1H), 2.25-2.11 (m, 3H), 2.11-1.96 (m, 2H), 1.96-1.84 (m, 1H), 1.71 (d, J=14.8 Hz, 2H), 1.44-1.36 (m, 1H), 1.28 (s, 3H), 1.25 (s, 1H), 1.22-1.11 (m, 3H), 1.11-1.03 (m, 1H). MS (ESI) m/z: [M+H]⁺ Found 514.3.

### Example 55: 4-Cyclopropyl-N—((S)-(4,4-difluorocyclohexyl(7-(((3S*,5S)-5-methyl-2-oxo-5-(trifluoromethyl)pyrrolidine-3-yl)methyl)imidazo[1,2-b]pyrrolidin-2-yl)methyl)-1,2,5-oxadiazole-3-carboxamide

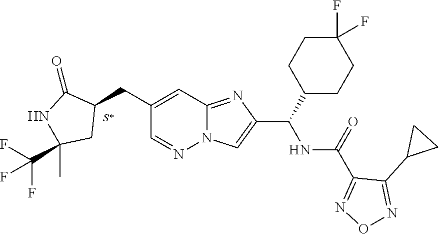 [SMILES: C[C@@]1(C(F)(F)F)C[C@H](Cc2cnn3cc([C@@H](NC(=O)c4nonc4C4CC4)C4CCC(F)(F)CC4)nc3c2)C(=O)N1]

### Example 56: 4-Cyclopropyl-N—((S)-(4,4-difluorocyclohexyl)(7-(((3R*,5S)-5-methyl-2-oxo-5-(trifluoromethyl)pyrrolidin-3-yl)methyl)imidazo[1,2-b]pyridazin-2-yl)methyl)-1,2,5-oxadiazole-3-carboxamide

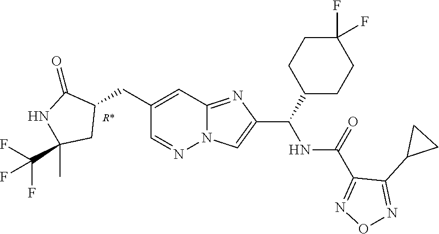 [SMILES: C[C@@]1(C(F)(F)F)C[C@@H](Cc2cnn3cc([C@@H](NC(=O)c4nonc4C4CC4)C4CCC(F)(F)CC4)nc3c2)C(=O)N1]

Step A: tert-Butyl ((S)-(4,4-difluorocyclohexyl)(7-((E)-((S)-5-methyl-1-((4-nitrophenyl)sulfonyl)-2-oxo-5-(trifluoromethyl)pyrrolidin-3-ylidene)methyl)imidazo[1,2-b]pyridazin-2-yl)methyl)carbamate. NaH (20.5 mg, 0.513 mmol, 60% dispersion in mineral oil) was suspended in dry THF (2 mL) and cooled to −78° C. under a N₂ atmosphere. Then a solution of diethyl ((5S)-5-methyl-1-((4-nitrophenyl)sulfonyl)-2-oxo-5-(trifluoromethyl)pyrrolidin-3-yl)phosphonate (244 mg, 0.500 mmol, Intermediate 28) in dry THF (2 mL) was added dropwise. This mixture was stirred at −78° C. for 30 min before a solution of tert-butyl (S)-((4,4-difluorocyclohexyl)(7-formylimidazo[1,2-b]pyridazine-2-yl)methyl)carbamate (125 mg, 0.320 mmol) in dry THF (2 mL) was added dropwise. The resulting mixture was stirred at −78° C. for 40 min before the cooling bath was removed and the reaction was allowed to warm to rt. After 20 h, the flask was cooled to 0° C. and water was added slowly, after which the mixture was extracted with EtOAc several times. The combined organic layers were dried over anhydrous MgSO₄, filtered, and concentrated to dryness. The residue was purified by silica gel chromatography (0-100% (10% MeOH in EtOAc)/hexanes) to provide the title compound as a yellow foam (79.6% yield).

Step B: tert-Butyl ((1S)-(7-(((5S)-1-((4-aminophenyl)sulfonyl)-5-methyl-2-oxo-5-(trifluoromethyl)pyrrolidin-3-yl)methyl)imidazo[1,2-b]pyridazin-2-yl)(4,4-difluorocyclohexyl)methyl)carbamate. tert-Butyl ((S)-(4,4-difluorocyclohexyl)(7-((E)-((S)-5-methyl-1-((4-nitrophenyl)sulfonyl)-2-oxo-5-(trifluoromethyl)pyrrolidin-3-ylidene)methyl)imidazo[1,2-b]pyridazin-2-yl)methyl)carbamate (258 mg, 0.354 mmol, Step A) was dissolved in MeOH (80 mL) and then 10% Pd/C (565 mg, 0.531 mmol) was added. The reaction was placed under H₂ gas (1 atm) with a balloon and the flask was evacuated and backfilled 3 times before the mixture was stirred at rt for 3 h. The mixture was filtered through a pad of diatomaceous earth and the pad was washed with MeOH. The filtrate was concentrated to dryness to afford a yellow oil. The residue was purified by basic preparative HPLC (XBridge C18, 5 μm, 50×100 mm, 10-100% MeCN/H₂O (20 mM NH₄OH)) to provide the title compound as an off-white solid (60.5% yield).

Step C: tert-Butyl ((1S)-(4,4-difluorocyclohexyl)(7-(((5S)-5-methyl-2-oxo-5-(trifluoromethyl)pyrrolidin-3-yl)methyl)imidazo[1,2-b]pyridazin-2-yl)methyl)carbamate. tert-Butyl ((1S)-(7-(((5S)-1-((4-aminophenyl)sulfonyl)-5-methyl-2-oxo-5-(trifluoromethyl)pyrrolidin-3-yl)methyl)imidazo[1,2-b]pyridazin-2-yl)(4,4-difluorocyclohexyl)methyl)carbamate (88 mg, 0.13 mmol, Step B) was dissolved in dry THE (1 mL) and the mixture was cooled to 0° C. under a nitrogen atmosphere. Then SmI₂ (12.8 mL, 1.28 mmol, 0.1 M in THF) was added dropwise and the mixture was stirred for 1 h. The mixture was then diluted with aqueous HCl (1 M) and extracted with ethyl acetate (3×50 mL). The combined organic layers were dried over anhydrous MgSO₄, filtered, and concentrated to dryness. The residue was purified by basic preparative HPLC (XBridge C18, 5 μm, 50×100 mm, 10-100% MeCN/H₂O (20 mM NH₄OH)) to provide the title compound as an off-white foam (54% yield).

Step D: (5S)-3-((2-((S)-Amino(4,4-difluorocyclohexyl)methyl)imidazo[1,2-b]pyridazin-7-yl)methyl)-5-methyl-5-(trifluoromethyl)pyrrolidin-2-one. tert-Butyl ((1S)-(4,4-difluorocyclohexyl)(7-(((5S)-5-methyl-2-oxo-5-(trifluoromethyl)pyrrolidin-3-yl)methyl)imidazo[1,2-b]pyridazin-2-yl)methyl)carbamate (58 mg, 0.11 mmol, Step C) was dissolved in DCM (0.1 mL) and then TFA (0.2 mL, 1.3 mmol) was added. The reaction was stirred at rt for 3 h. The mixture was concentrated to dryness and the residue was diluted with saturated aqueous Na₂SO₃ and extracted with EtOAc (6×3 mL). The combined organic layers were dried over anhydrous MgSO₄, filtered, and concentrated to dryness to afford the title compound as an orange foam which was used without further purification.

Step E: 4-Cyclopropyl-N—((S)-(4,4-difluorocyclohexyl)(7-(((3S*,5S)-5-methyl-2-oxo-5-(trifluoromethyl)pyrrolidine-3-yl)methyl)imidazo[1,2-b]pyrrolidin-2-yl)methyl)-1,2,5-oxadiazole-3-carboxamide and 4-cyclopropyl-N—((S)-(4,4-difluorocyclohexyl(7-(((3R*,5S)-5-methyl-2-oxo-5-(trifluoromethyl)pyrrolidin-3-yl)methyl)imidazo[1,2-b]pyridazin-2-yl)methyl)-1,2,5-oxadiazole-3-carboxamide. To a mixture of (5S)-3-((2-((S)-amino(4,4-difluorocyclohexyl)methyl)imidazo[1,2-b]pyrrolidin-7-yl)methyl)-5-methyl-5-(trifluoromethyl)pyrrolidine-2-one (61 mg, 0.14 mmol, Step D) in dry acetonitrile (0.1 mL) was added DIPEA (71 μL, 0.41 mmol). Then, a solution of 2,5-dioxopyrrolidin-1-yl 4-cyclopropyl-1,2,5-oxadiazole-3-carboxylate (86 mg, 0.34 mmol, Intermediate 2) in dry acetonitrile (0.1 mL) was added in one portion. The reaction was stirred at rt for 2 h. The mixture was concentrated to dryness and the residue was purified by basic preparative HPLC (Xbridge C18, 5 μm, 50×100 mm, 10-100% MeCN/H₂O (20 mM NH₄OH)) to provide a mixture of diastereomers as an off-white foam. The diastereomers were separated by chiral SFC (Stationary phase: Chiralpak LB N3, 5 μm, 250×21 mm, Mobile phase: 30% methanol, 70% CO₂) to afford Example 55 (10% yield) as the first-eluting isomer and Example 56 (12% yield) as the second-eluting isomer. Example 55: ¹H NMR (500 MHz, CDCl₃) δ 8.23 (d, J=2.1 Hz, 1H), 7.87 (s, 1H), 7.79 (d, J=8.8 Hz, 1H), 7.69 (dd, J=2.1, 0.9 Hz, 1H), 5.29 (dd, J=8.8, 7.6 Hz, 1H), 3.42-3.22 (m, 1H), 3.06-2.88 (m, 1H), 2.81 (dd, J=14.7, 9.7 Hz, 1H), 2.60-2.52 (m, 1H), 2.19-2.11 (m, 5H), 2.10-1.96 (m, 1H), 1.83-1.61 (m, 4H), 1.60-1.51 (m, 1H), 1.48 (s, 3H), 1.43-1.32 (m, 1H), 1.22-1.04 (m, 4H). MS (ESI) m/z: [M+H]⁺ Found 582.3. Example 56: ¹H NMR (500 MHz, CDCl₃) δ 8.23 (d, J=2.1 Hz, 1H), 7.87 (s, 1H), 7.73 (d, J=8.9 Hz, 1H), 7.70 (dd, J=2.2, 0.8 Hz, 1H), 5.73 (s, 1H), 5.28 (dd, J=8.8, 7.6 Hz, 1H), 3.32-3.26 (m, 1H), 3.07-2.97 (m, 1H), 2.78 (dd, J=14.7, 9.0 Hz, 1H), 2.68-2.50 (m, 2H), 2.21-2.11 (m, 1H), 2.11-1.96 (m, 1H), 1.84-1.63 (m, 2H), 1.44 (s, 3H), 1.29-1.23 (m, 4H), 1.20-1.04 (m, 4H), 0.91-0.80 (m, 2H). MS (ESI) m/z: [M+H]⁺ Found 582.3.

### Example 57: 4-Cyclopropyl-N—((S)-5,5,5-trifluoro-4,4-dimethyl-1-(7-(((3S*,5S)-5-methyl-2-oxo-5-(trifluoromethyl)pyrrolidin-3-yl)methyl)imidazo[1,2-b]pyridazin-2-yl)pentyl)-1,2,5-oxadiazole-3-carboxamide

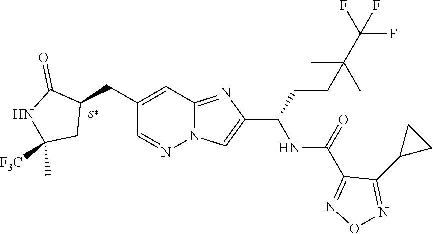 [SMILES: CC(C)(CC[C@H](NC(=O)c1nonc1C1CC1)c1cn2ncc(C[C@H]3C[C@@](C)(C(F)(F)F)NC3=O)cc2n1)C(F)(F)F]

### Example 58: 4-Cyclopropyl-N—((S)-5,5,5-trifluoro-4,4-dimethyl-1-(7-(((3R*,5S)-5-methyl-2-oxo-5-(trifluoromethyl)pyrrolidin-3-yl)methyl)imidazo[1,2-b]pyridazin-2-yl)pentyl)-1,2,5-oxadiazole-3-carboxamide

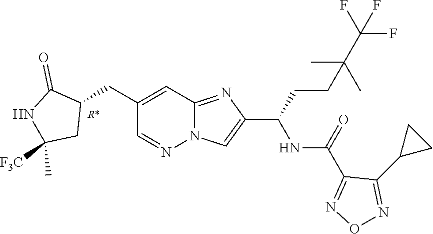 [SMILES: CC(C)(CC[C@H](NC(=O)c1nonc1C1CC1)c1cn2ncc(C[C@@H]3C[C@@](C)(C(F)(F)F)NC3=O)cc2n1)C(F)(F)F]

Step A: tert-Butyl ((S)-5,5,5-trifluoro-4,4-dimethyl-1-(7-((E)-((S)-5-methyl-1-((4-nitrophenyl)sulfonyl)-2-oxo-5-(trifluoromethyl)pyrrolidin-3-ylidene)methyl)imidazo[1,2-b]pyridazin-2-yl)pentyl)carbamate. A suspension of NaH (20 mg, 0.5 mmol, 60% dispersion in mineral oil) in dry THE (1 mL) under a N₂ atmosphere was cooled to −78° C. and then a solution of diethyl ((5S)-5-methyl-1-((4-nitrophenyl)sulfonyl)-2-oxo-5-(trifluoromethyl)pyrrolidin-3-yl)phosphonate (244 mg, 0.5 mmol, Intermediate 28) in dry THE (1.5 mL) was added dropwise. The resulting mixture was stirred for 15 min at −78° C. before a solution of tert-butyl (S)-(5,5,5-trifluoro-1-(7-formylimidazo[1,2-b]pyridazin-2-yl)-4,4-dimethylpentyl)carbamate (125 mg, 0.302 mmol, Intermediate 17) in dry THE (1.5 mL) was added dropwise at −78° C. The reaction mixture was stirred at −78° C. for 35 min before the cooling bath was removed and the reaction was allowed to warm to rt. The reaction was stirred for 20 h, then cooled to 0° C. and water was added dropwise. This mixture was extracted with EtOAc (5×15 mL), the organic layers were combined, dried over anhydrous MgSO₄, filtered, and concentrated to dryness. The residue was purified by silica gel chromatography (0-100% (10% MeOH in EtOAc)/hexanes) to provide the title compound as a yellow foam in 90% yield.

Step B: tert-Butyl ((1S)-1-(7-(((5S)-1-((4-aminophenyl)sulfonyl)-5-methyl-2-oxo-5-(trifluoromethyl)pyrrolidin-3-yl)methyl)imidazo[1,2-b]pyridazin-2-yl)-5,5,5-trifluoro-4,4-dimethylpentyl)carbamate. To a solution of tert-butyl ((S)-5,5,5-trifluoro-4,4-dimethyl-1-(7-((E)-((S)-5-methyl-1-((4-nitrophenyl)sulfonyl)-2-oxo-5-(trifluoromethyl)pyrrolidin-3-ylidene)methyl)imidazo[1,2-b]pyridazin-2-yl)pentyl)carbamate (180 mg, 0.24 mmol, Step A) in MeOH (16 mL) was added 10% Pd/C (426 mg, 0.401 mmol). The reaction flask was evacuated and backfilled three times with H₂ gas from a balloon (1 atm) and the reaction mixture was stirred under a H₂ atmosphere at rt for 2 h. Then, the mixture was filtered through a pad of diatomaceous earth and the pad was washed with MeOH. The filtrate was concentrated to dryness and the resulting yellow oil was purified by basic preparative HPLC (XBridge C18, 5 μm, 50×100 mm, 10-100% MeCN/H₂O (20 mM NH₄OH)) to provide the title compound as an off-white solid (58% yield).

Step C: tert-Butyl ((1S)-5,5,5-trifluoro-4,4-dimethyl-1-(7-(((5S)-5-methyl-2-oxo-5-(trifluoromethyl)pyrrolidin-3-yl)methyl)imidazo[1,2-b]pyridazin-2-yl)pentyl)carbamate. tert-Butyl ((1S)-1-(7-(((5S)-1-((4-aminophenyl)sulfonyl)-5-methyl-2-oxo-5-(trifluoromethyl)pyrrolidin-3-yl)methyl)imidazo[1,2-b]pyridazin-2-yl)-5,5,5-trifluoro-4,4-dimethylpentyl)carbamate (107 mg, 0.15 mmol, Step B) was dissolved in dry THF (1 mL) and the solution was cooled to 0° C. under a nitrogen atmosphere. Then, SmI₂ (0.1 M in THF, 9.7 mL, 0.97 mmol) was added dropwise. Upon completion of the addition of SmI₂, the ice bath was removed and the reaction was warmed to rt. The reaction was then diluted with aqueous HCl (1 M) and extracted with EtOAc (3×50 mL). The combined organic layers were dried over anhydrous MgSO₄, filtered, and concentrated to dryness. The residue was purified by basic preparative HPLC (XBridge C18, 5 μm, 50×100 mm, 10-100% MeCN/H₂O (20 mM NH₄OH)) affording the title compound as an off-white solid (95% yield).

Step D: (5S)-3-((2-((S)-1-Amino-5,5,5-trifluoro-4,4-dimethylpentyl)imidazo[1,2-b]pyridazin-7-yl)methyl)-5-methyl-5-(trifluoromethyl)pyrrolidin-2-one. tert-Butyl ((1S)-5,5,5-trifluoro-4,4-dimethyl-1-(7-(((5S)-5-methyl-2-oxo-5-(trifluoromethyl)pyrrolidin-3-yl)methyl)imidazo[1,2-b]pyridazin-2-yl)pentyl)carbamate (80 mg, 0.14 mmol, Step C) was dissolved in DCM (0.3 mL) and then TFA (0.4 mL, 5.2 mmol) was added. The reaction mixture was stirred at rt for 2 h. Then, the reaction mixture was concentrated to dryness, the residue was diluted with saturated aqueous Na₂CO₃ (4 mL) and the residue was extracted with EtOAc (6×3 mL). The combined organic layers were dried over anhydrous MgSO₄, filtered, and concentrated to dryness and the material was used without further purification.

Step E: 4-Cyclopropyl-N—((S)-5,5,5-trifluoro-4,4-dimethyl-1-(7-(((3S,5S)-5-methyl-2-oxo-5-(trifluoromethyl)pyrrolidin-3-yl)methyl)imidazo[1,2-b]pyridazin-2-yl)pentyl)-1,2,5-oxadiazole-3-carboxamide and 4-cyclopropyl-N—((S)-5,5,5-trifluoro-4,4-dimethyl-1-(7-(((3R,5S)-5-methyl-2-oxo-5-(trifluoromethyl)pyrrolidin-3-yl)methyl)imidazo[1,2-b]pyridazin-2-yl)pentyl)-1,2,5-oxadiazole-3-carboxamide. To a solution of (5S)-3-((2-((S)-1-amino-5,5,5-trifluoro-4,4-dimethylpentyl)imidazo[1,2-b]pyridazin-7-yl)methyl)-5-methyl-5-(trifluoromethyl)pyrrolidin-2-one (122 mg, 0.262 mmol, Step D) in acetonitrile (1.5 mL) were added sequentially DIPEA (0.113 mL, 0.655 mmol) and a solution of 2,5-dioxopyrrolidin-1-yl 4-cyclopropyl-1,2,5-oxadiazole-3-carboxylate (102 mg, 0.406 mmol, Intermediate 2) in acetonitrile (1.5 mL) in one portion. The resulting mixture was stirred at rt for 3 h then concentrated to dryness. The residue was purified by basic preparative HPLC (XBridge C18, 5 μm, 50×100 mm, 0-100% MeCN/H₂O (20 mM NH₄OH)) to afford a mixture of diastereomers as a yellow foam. The diastereomers were separated by chiral SFC (Stationary phase: Chiralpak IH, 5 μm, 250×21 mm, Mobile phase: 17% methanol:isopropanol (1:1) with 0.2% isopropylamine, 83% CO₂) to provide Example 57 as the first-eluting isomer (8.4% yield) and Example 58 as the second-eluting isomer (3.9% yield). Example 57: ¹H NMR (500 MHz, CDCl₃) δ 8.24 (d, J=2.1 Hz, 1H), 7.91 (s, 1H), 7.76 (d, J=8.4 Hz, 1H), 7.70 (dd, J=2.1, 0.9 Hz, 1H), 6.95 (s, 1H), 5.38-5.29 (m, 1H), 3.38-3.25 (m, 1H), 3.05-2.89 (m, 1H), 2.88-2.76 (m, 1H), 2.70-2.51 (m, 1H), 2.23-2.04 (m, 4H), 1.72-1.58 (m, 2H), 1.55-1.49 (m, 1H), 1.48 (s, 3H), 1.19-1.12 (m, 3H), 1.12-1.07 ((m, 6H). MS (ESI) m/z: [M+H]⁺ Found 602.1. Example 58: ¹H NMR (500 MHz, CDCl₃) δ 8.23 (d, J=2.1 Hz, 1H), 7.90 (s, 1H), 7.70 (dd, J=2.1, 0.9 Hz, 1H), 7.67 (d, J=8.4 Hz, 1H), 6.53 (s, 1H), 5.41-5.28 (m, 1H), 3.28 (ddd, J=14.7, 4.6, 0.9 Hz, 1H), 3.03 (qd, J=9.3, 4.5 Hz, 1H), 2.79 (dd, J=14.7, 9.0 Hz, 1H), 2.70-2.50 (m, 2H), 2.21-1.99 (m, 2H), 1.75-1.58 (m, 3H), 1.57-1.47 (m, 1H), 1.44 (s, 3H), 1.19-1.12 (m, 3H), 1.10 (d, J=13.5 Hz, 6H). MS (ESI) m/z: [M+H]⁺ Found 602.1.

### Example 59: 4-Cyclopropyl-N—[(S)-(4,4-difluorocyclohexyl)-[7-[[(3S,5S)-2-oxo-5-(trifluoromethyl)pyrrolidin-3-yl]methyl]imidazo[1,2-b]pyridazin-2-yl]methyl]-1,2,5-oxadiazole-3-carboxamide

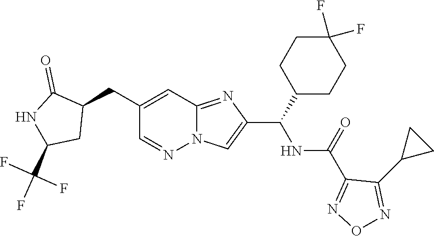 [SMILES: O=C(N[C@H](c1cn2ncc(C[C@H]3C[C@@H](C(F)(F)F)NC3=O)cc2n1)C1CCC(F)(F)CC1)c1nonc1C1CC1]

### Example 60: 4-Cyclopropyl-N—[(S)-(4,4-difluorocyclohexyl)-[7-[[(3R,5S)-2-oxo-5-(trifluoromethyl)pyrrolidin-3-yl]methyl]imidazo[1,2-b]pyridazin-2-yl]methyl]-1,2,5-oxadiazole-3-carboxamide

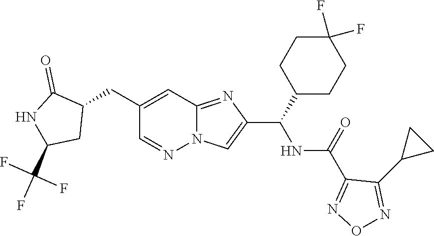 [SMILES: O=C(N[C@H](c1cn2ncc(C[C@@H]3C[C@@H](C(F)(F)F)NC3=O)cc2n1)C1CCC(F)(F)CC1)c1nonc1C1CC1]

To a solution of (5S)-3-((2-((S)-amino(4,4-difluorocyclohexyl)methyl)imidazo[1,2-b]pyridazin-7-yl)methyl)-5-(trifluoromethyl)pyrrolidin-2-one (Intermediate 40, 170 mg, 0.4 mmol) in ACN (6.6 mL) was added DIPEA (0.20 mL, 1.2 mmol) and the solution was stirred at rt for 5 min. Then, a solution of 2,5-dioxopyrrolidin-1-yl 4-cyclopropyl-1,2,5-oxadiazole-3-carboxylate (Intermediate 2, 128 mg, 0.5 mmol) in ACN (0.6 mL) was added and the resulting mixture was stirred at rt for 5 min. The solution was concentrated to dryness, dissolved in DMF and purified via preparative basic HPLC (X-Bridge Prep C18 5 μm column, 50×250 mm, 5-100% acetonitrile/water (with 20 mM NH₄OH). The diastereomers were separated by chiral SFC (Chiralpak IBN, 5 μm, 250×21 mm, 25% methanol, 75% CO₂). The first eluting isomer (4% yield, absolute stereochemistry determined by 2D NMR) was designated as the (35,55) isomer (Example 59) and the second eluting isomer (23% yield, absolute stereochemistry determined by 2D NMR) was designated as the (3R,5S) isomer (Example 60). Example 59: ¹H NMR (500 MHz, DMSO-d₆) δ 9.50-9.46 (m, 1H), 8.65 (s, 1H), 8.48-8.45 (m, 1H), 8.21-8.18 (m, 1H), 7.96-7.93 (m, 1H), 5.22-5.17 (m, 1H), 4.30-4.23 (m, 1H), 3.15-3.07 (m, 1H), 2.96-2.88 (m, 1H), 2.80-2.70 (m, 1H), 2.45-2.38 (m, 1H), 2.33-2.25 (m, 1H), 2.23-2.14 (m, 1H), 2.10-1.95 (m, 2H), 1.94-1.87 (m, 1H), 1.87-1.71 (m, 2H), 1.71-1.60 (m, 2H), 1.45-1.35 (m, 1H), 1.34-1.24 (m, 1H), 1.15-1.10 (m, 2H), 0.99-0.94 (m, 2H). MS (ESI) m/z: [M+H]⁺ Found 568.2. Further NMR analysis confirmed the stereochemistry of Example 59 as shown here:

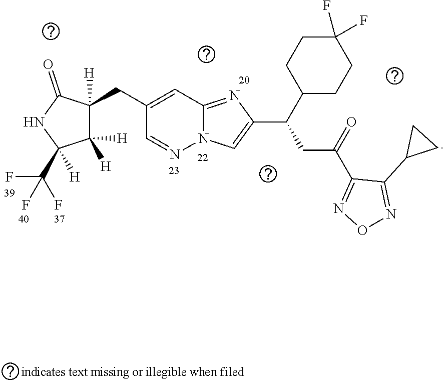

Key NOESY correlations were observed between protons 33a and 32a to proton 31a, confirming that they are all on the same side of the ring. Additionally, ¹⁹F-¹H-HOESY correlations were observed only between fluorines (37, 39, and 40) to protons 33a and 32b. These correlations support relative cis stereochemistry between the C-30 sidechain and the C-36 sidechain. Example 60: ¹H NMR (500 MHz, DMSO-d₆) δ 9.46 (d, J=9.0 Hz, 1H), 8.69-8.67 (m, 1H), 8.46-8.43 (m, 1H), 8.20-8.16 (m, 1H), 7.95-7.91 (m, 1H), 5.21-5.13 (m, 1H), 4.26-4.17 (m, 1H), 3.12-3.05 (m, 1H), 2.95-2.86 (m, 1H), 2.74-2.66 (m, 1H), 2.32-2.25 (m, 1H), 2.22-2.12 (m, 2H), 2.11-1.95 (m, 3H), 1.93-1.86 (m, 1H), 1.85-1.71 (m, 2H), 1.66-1.58 (m, 1H), 1.44-1.33 (m, 1H), 1.32-1.22 (m, 1H), 1.16-1.07 (m, 2H), 0.99-0.92 (m, 2H). MS (ESI) m/z: [M+H]⁺ Found 568.2. Further NMR analysis confirmed the stereochemistry of Example 60, as shown here:

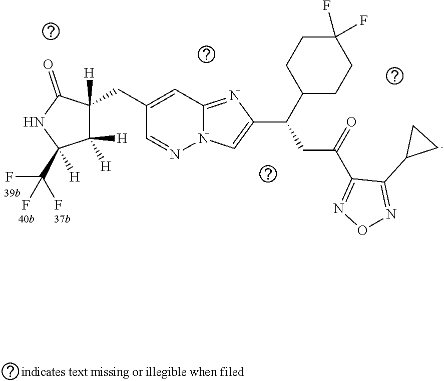

Key ¹⁹F-¹H-HOESY correlations were observed between fluorines (37b, 39b, and 40b) and the proton 32b′, confirming that the C-36b sidechain (CF₃) and proton 32b′ are both on the same side of the lactam ring. Key NOESY correlations were observed between protons 31b′ and 32b′. These correlations support relative trans stereochemistry between the C-30b sidechain and the C-36b sidechain.

### Example 61: 4-Cyclopropyl-N—[(S)-(4,4-difluorocyclohexyl)-[7-[hydroxy-[2-oxo-5-(trifluoromethyl)pyrrolidin-3-yl]methyl]imidazo[1,2-b]pyridazin-2-yl]methyl]-1,2,5-oxadiazole-3-carboxamide

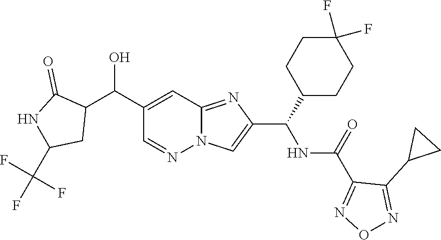 [SMILES: O=C(N[C@H](c1cn2ncc(C(O)C3CC(C(F)(F)F)NC3=O)cc2n1)C1CCC(F)(F)CC1)c1nonc1C1CC1]

The title compound was synthesized in a manner analogous to Example 59 using (13)-(4,4-difluorocyclohexyl)(7-(hydroxy(2-oxo-5-(trifluoromethyl)pyrrolidin-3-yl)methyl)imidazo[1,2-b]pyridazin-2-yl)methanaminium 2,2,2-trifluoroacetate (Intermediate 57 Step B) in place of (5S)-3-((2-((S)-amino(4,4-difluorocyclohexyl)methyl)imidazo[1,2-b]pyridazin-7-yl)methyl)-5-(trifluoromethyl)pyrrolidin-2-one. The reaction mixture was purified via preparative basic HPLC (X-Bridge Prep C18 column, 5 μm, 50×250 mm, 5-100% acetonitrile/water (with 20 mM NH₄OH)) to afford the title compound (20% yield). ¹H NMR (500 MHz, DMSO-d₆) δ 9.53-9.47 (m, 1H), 8.63 (s, 1H), 8.53-8.46 (m, 1H), 8.24 (s, 1H), 7.90 (s, 1H), 6.05-6.01 (m, 1H), 5.20 (t, J=8.5 Hz, 1H), 5.07-5.00 (m, 1H), 4.20-4.12 (m, 1H), 3.09-3.02 (m, 1H), 2.35-2.23 (m, 2H), 2.22-2.14 (m, 2H), 2.10-1.94 (m, 2H), 1.93-1.87 (m, 1H), 1.87-1.71 (m, 2H), 1.64-1.59 (m, 1H), 1.45-1.32 (m, 1H), 1.32-1.26 (m, 1H), 1.17-1.07 (m, 2H), 1.01-0.91 (m, 2H). MS (ESI) m/z: [M+H]⁺ Found 584.2.

### Example 62: 4-Cyclopropyl-N-((1S)-(4,4-difluorocyclohexyl)(7-((5-oxo-4-azaspiro[2.4]heptan-6-yl)methyl)imidazo[1,2-b]pyridazin-2-yl)methyl)-1,2,5-oxadiazole-3-carboxamide

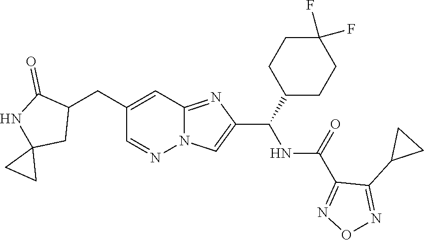 [SMILES: O=C(N[C@H](c1cn2ncc(CC3CC4(CC4)NC3=O)cc2n1)C1CCC(F)(F)CC1)c1nonc1C1CC1]

The title compound was synthesized in a manner analogous to Example 59 using (1S)-(4,4-difluorocyclohexyl)(7-((5-oxo-4-azaspiro[2.4]heptan-6-yl)methyl)imidazo[1,2-b]pyridazin-2-yl)methanaminium 2,2,2-trifluoroacetate (Intermediate 58 Step B) in place of (5S)-3-((2-((S)-amino(4,4-difluorocyclohexyl)methyl)imidazo[1,2-b]pyridazin-7-yl)methyl)-5-(trifluoromethyl)pyrrolidin-2-one. Purification via preparative acidic HPLC (C18 column, 5 μm, 30×250 mm; 0-100% ACN (with 0.05% TFA) in H₂O (with 0.05% TFA)) afforded the title compound (25% yield). ¹H NMR (400 MHz, DMSO-d₆) δ 9.51-9.46 (m, 1H), 8.51-8.46 (m, 1H), 8.24-8.20 (m, 1H), 7.97-7.94 (m, 1H), 7.79-7.76 (m, 1H), 5.23-5.17 (m, 1H), 3.16-3.10 (m, 1H), 3.02-2.93 (m, 1H), 2.81-2.73 (m, 1H), 2.35-2.25 (m, 1H), 2.23-2.13 (m, 1H), 2.11-1.98 (m, 2H), 1.97-1.92 (m, 3H), 1.87-1.70 (m, 2H), 1.68-1.60 (m, 1H), 1.46-1.23 (m, 2H), 1.17-1.10 (m, 2H), 1.00-0.94 (m, 2H), 0.70-0.63 (m, 1H), 0.61-0.53 (m, 2H), 0.51-0.45 (m, 1H). MS (ESI) m/z: [M+H]⁺ Found 526.2.

### Example 63: 4-Cyclopropyl-N—((S)-(4,4-difluorocyclohexyl)(7-((R*)-hydroxy((3R*,5R*)-2-oxo-5-(trifluoromethyl)pyrrolidin-3-yl)methyl)imidazo[1,2-b]pyridazin-2-yl)methyl)-1,2,5-oxadiazole-3-carboxamide

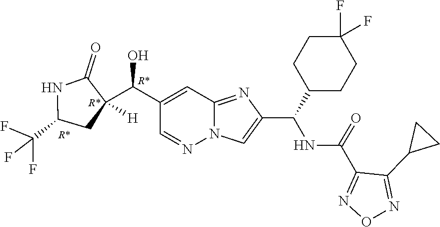 [SMILES: O=C(N[C@H](c1cn2ncc([C@H](O)[C@H]3C[C@H](C(F)(F)F)NC3=O)cc2n1)C1CCC(F)(F)CC1)c1nonc1C1CC1]

The title compound (42% yield) was synthesized in a manner analogous to Example 59 using (3R*,5R*)-3-((R*)-(2-((S)-amino(4,4-difluorocyclohexyl)methyl)imidazo[1,2-b]pyridazin-7-yl)(hydroxy)methyl)-5-(trifluoromethyl)pyrrolidin-2-one (Intermediate 59) in place of (5S)-3-((2-((S)-amino(4,4-difluorocyclohexyl)methyl)imidazo[1,2-b]pyridazin-7-yl)methyl)-5-(trifluoromethyl)pyrrolidin-2-one. ¹H NMR (500 MHz, DMSO-d₆) δ 9.49 (d, J=9.01 Hz, 1H), 8.67 (s, 1H), 8.56 (d, J=2.00 Hz, 1H), 8.22 (s, 1H), 7.94-7.92 (m, 1H), 6.02 (d, J=4.75 Hz, 1H), 5.22-5.17 (m, 1H), 4.28-4.20 (m, 1H), 2.97-2.92 (m, 1H), 2.85 (s, 1H), 2.43-2.35 (m, 1H), 2.31-2.24 (m, 1H), 2.23-2.15 (m, 1H), 2.07-1.96 (m, 2H), 1.95-1.86 (m, 1H), 1.84-1.78 (m, 2H), 1.76-1.70 (m, 1H), 1.66-1.59 (m, 1H), 1.42-1.37 (m, 1H), 1.33-1.24 (m, 1H), 1.15-1.10 (m, 2H), 0.99-0.94 (m, 2H). MS (ESI) m/z: [M+H]⁺ Found 584.2.

### Example 64: 4-Cyclopropyl-N—((S)-(4,4-difluorocyclohexyl)(7-((R*)-hydroxy((3S*,5R*)-2-oxo-5-(trifluoromethyl)pyrrolidin-3-yl)methyl)imidazo[1,2-b]pyridazin-2-yl)methyl)-1,2,5-oxadiazole-3-carboxamide

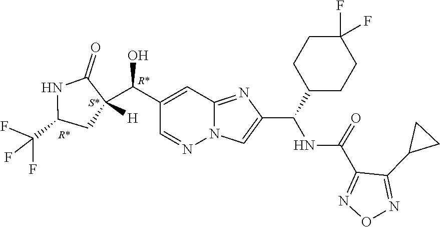 [SMILES: O=C(N[C@H](c1cn2ncc([C@H](O)[C@@H]3C[C@H](C(F)(F)F)NC3=O)cc2n1)C1CCC(F)(F)CC1)c1nonc1C1CC1]

Step A: (S)-(4,4-Difluorocyclohexyl)₇-((R*)-hydroxy((3S*,5R*)-2-oxo-5-(trifluoromethyl)pyrrolidin-3-yl)methyl)imidazo[1,2-b]pyridazin-2-yl)methanaminium 2,2,2-trifluoroacetate. The title compound was synthesized in a manner analogous to Intermediate 57 Step B using tert-butyl (3S*,5R*)-3-((R*)-(2-((S)-((tert-butoxycarbonyl)amino(4,4-difluorocyclohexyl)methyl)imidazo[1,2-b]pyridazin-7-yl)(hydroxy)methyl)-2-oxo-5-(trifluoromethyl)pyrrolidine-1-carboxylate (Intermediate 43) in place of tert-butyl 3-((2-((S)-((tert-butoxycarbonyl)amino)(4,4-difluorocyclohexyl)methyl)imidazo[1,2-b]pyridazin-7-yl)(hydroxy)methyl)-2-oxo-5-(trifluoromethyl)pyrrolidine-1-carboxylate, and was used without further purification.

Step B: 4-Cyclopropyl-N—((S)-(4,4-difluorocyclohexyl)(7-((R*)-hydroxy((3S*,5R*)-2-oxo-5-(trifluoromethyl)pyrrolidin-3-yl)methyl)imidazo[1,2-b]pyridazin-2-yl)methyl)-1,2,5-oxadiazole-3-carboxamide. The title compound (11% yield over Steps A and B) was synthesized in a manner analogous to Example 59 using (S)-(4,4-difluorocyclohexyl)(7-((R*)-hydroxy((3S*,5R*)-2-oxo-5-(trifluoromethyl)pyrrolidin-3-yl)methyl)imidazo[1,2-b]pyridazin-2-yl)methanaminium 2,2,2-trifluoroacetate (Step A) in place of (5S)-3-((2-((S)-amino(4,4 difluorocyclohexyl)methyl)imidazo[1,2-b]pyridazin-7-yl)methyl)-5-(trifluoromethyl)pyrrolidin-2-one. ¹H NMR (600 MHz, DMSO-d₆) δ 9.47 (d, J=9.3 Hz, 1H), 8.64-8.61 (m, 1H), 8.48 (d, J=2.0 Hz, 1H), 8.23-8.20 (m, 1H), 7.90-7.87 (m, 1H), 6.01 (t, J=4.5 Hz, 1H), 5.19 (t, J=8.5 Hz, 1H), 5.04-5.00 (m, 1H), 4.19-4.13 (m, 1H), 3.10-3.00 (m, 1H), 2.33-2.24 (m, 2H), 2.23-2.13 (m, 2H), 2.10-1.94 (m, 2H), 1.94-1.86 (m, 2H), 1.85-1.71 (m, 1H), 1.66-1.58 (m, 1H), 1.34-1.21 (m, 2H), 1.15-1.08 (m, 2H), 0.99-0.91 (m, 2H). MS (ESI) m/z: [M+H]⁺ Found 584.2.

### Example 65: 4-Cyclopropyl-N—((S)-5,5,5-trifluoro-1-(7-((R*)-hydroxy((3R*,5S)-2-oxo-5-(trifluoromethyl)pyrrolidin-3-yl)methyl)imidazo[1,2-b]pyridazin-2-yl)-4,4-dimethylpentyl)-1,2,5-oxadiazole-3-carboxamide

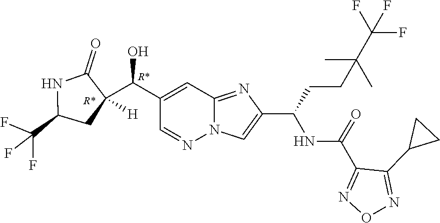 [SMILES: CC(C)(CC[C@H](NC(=O)c1nonc1C1CC1)c1cn2ncc([C@H](O)[C@H]3C[C@@H](C(F)(F)F)NC3=O)cc2n1)C(F)(F)F]

### Example 66: 4-Cyclopropyl-N—((S)-5,5,5-trifluoro-1-(7-((S*)-hydroxy((3R*,5S)-2-oxo-5-(trifluoromethyl)pyrrolidin-3-yl)methyl)imidazo[1,2-b]pyridazin-2-yl)-4,4-dimethylpentyl)-1,2,5-oxadiazole-3-carboxamide

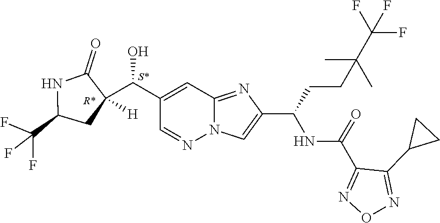 [SMILES: CC(C)(CC[C@H](NC(=O)c1nonc1C1CC1)c1cn2ncc([C@@H](O)[C@H]3C[C@@H](C(F)(F)F)NC3=O)cc2n1)C(F)(F)F]

### Example 67: 4-Cyclopropyl-N—((S)-5,5,5-trifluoro-1-(7-((S*)-hydroxy((3S*,5S)-2-oxo-5-(trifluoromethyl)pyrrolidin-3-yl)methyl)imidazo[1,2-b]pyridazin-2-yl)-4,4-dimethylpentyl)-1,2,5-oxadiazole-3-carboxamide

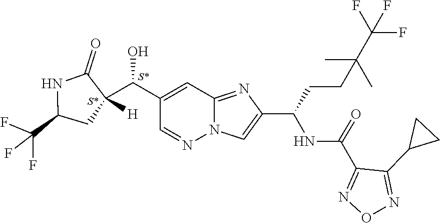 [SMILES: CC(C)(CC[C@H](NC(=O)c1nonc1C1CC1)c1cn2ncc([C@@H](O)[C@@H]3C[C@@H](C(F)(F)F)NC3=O)cc2n1)C(F)(F)F]

The title compounds were synthesized in a manner analogous to Example 59 using (1S)-5,5,5-trifluoro-1-(7-(hydroxy((5S)-2-oxo-5-(trifluoromethyl)pyrrolidin-3-yl)methyl)imidazo[1,2-b]pyridazin-2-yl)-4,4-dimethylpentan-1-aminium 2,2,2-trifluoroacetate (Intermediate 47) in place of (5S)-3-((2-((S)-amino(4,4-difluorocyclohexyl)methyl)imidazo[1,2-b]pyridazin-7-yl)methyl)-5-(trifluoromethyl)pyrrolidin-2-one. The residue was purified by silica gel chromatography (0-100% EtOAc (10% MeOH)/hexanes) to provide a mixture of diastereomers. The diastereomers were separated by chiral SFC (Chiralpak IH, 5 μm, 250×21 mm, 25% ACN/methanol (1/1), 75% CO₂). The first-eluting isomer (2% yield) was designated as the (3R*,5S) isomer (Example 65), the second-eluting isomer (7% yield) was designated as the (3R*,5S) isomer (Example 66) and the third-eluting isomer (13% yield) was designated as the (3S*,5S) isomer (Example 67). Example 65: ¹H NMR (500 MHz, DMSO-d₆) δ 9.56 (d, J=8.5 Hz, 1H), 8.68 (s, 1H), 8.56 (d, J=2.0 Hz, 1H), 8.17 (s, 1H), 7.96-7.94 (m, 1H), 6.08-6.02 (m, 1H), 5.26-5.20 (m, 1H), 5.19-5.16 (m, 1H), 4.28-4.21 (m, 1H), 2.99-2.92 (m, 1H), 2.43-2.31 (m, 2H), 2.16-2.07 (m, 1H), 2.03-1.93 (m, 1H), 1.84-1.77 (m, 1H), 1.70-1.62 (m, 1H), 1.59-1.50 (m, 1H), 1.17-1.12 (m, 2H), 1.11-1.08 (m, 6H), 1.02-0.97 (m, 2H). MS (ESI) m/z: [M+H]⁺ Found 604.2. Example 66: ¹H NMR (500 MHz, DMSO-do) δ 9.59-9.56 (m, 1H), 8.65-8.63 (m, 1H), 8.50-8.48 (m, 1H), 8.18-8.17 (m, 1H), 7.91-7.88 (m, 1H), 6.05-6.01 (m, 1H), 5.27-5.21 (m, 1H), 5.06-5.02 (m, 1H), 4.21-4.14 (m, 1H), 3.10-3.03 (m, 1H), 2.36-2.26 (m, 2H), 2.22-2.15 (m, 1H), 2.14-2.06 (m, 1H), 2.04-1.95 (m, 1H), 1.70-1.62 (m, 1H), 1.60-1.52 (m, 1H), 1.17-1.12 (m, 2H), 1.11-1.09 (m, 6H), 1.01-0.98 (m, 2H). MS (ESI) m/z: [M+H]⁺ Found 604.2. Example 67: ¹H NMR (500 MHz, DMSO-d₆) δ 9.57-9.54 (m, 1H), 8.59-8.57 (m, 1H), 8.45-8.43 (m, 1H), 8.17-8.15 (m, 1H), 7.89-7.86 (m, 1H), 5.98-5.96 (m, 1H), 5.26-5.19 (m, 11H), 5.07-5.03 (m, 11H), 4.25-4.19 (m, 1H), 3.14-3.09 (m, 11H), 2.14-2.06 (m, 1H), 2.03-1.94 (m, 1H), 1.89-1.82 (m, 1H), 1.66-1.58 (m, 1H), 1.56-1.47 (m, 1H), 1.25-1.23 (m, 1H), 1.14-1.11 (m, 3H), 1.09-1.07 (m, 6H), 0.99-0.96 (m, 2H). MS (ESI) m/z: [M+H]⁺ Found 604.2.

### Example 68: 4-Cyclopropyl-N—((S)-(4,4-difluorocyclohexyl)(7-(((3R*,5R*)-2-oxo-5-(trifluoromethyl)pyrrolidin-3-yl)methyl)imidazo[1,2-b]pyridazin-2-yl)methyl)-1,2,5-oxadiazole-3-carboxamide

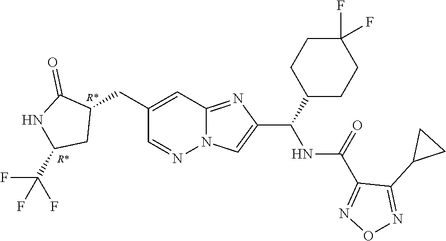 [SMILES: O=C(N[C@H](c1cn2ncc(C[C@@H]3C[C@H](C(F)(F)F)NC3=O)cc2n1)C1CCC(F)(F)CC1)c1nonc1C1CC1]

### Example 69: 4-Cyclopropyl-N—((S)-(4,4-difluorocyclohexyl)(7-(((3S*,5R*)-2-oxo-5-(trifluoromethyl)pyrrolidin-3-yl)methyl)imidazo[1,2-b]pyridazin-2-yl)methyl)-1,2,5-oxadiazole-3-carboxamide

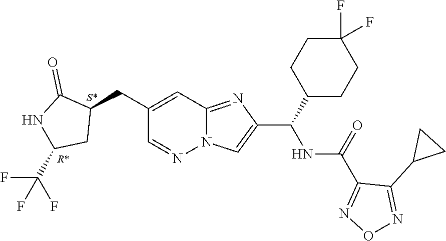 [SMILES: O=C(N[C@H](c1cn2ncc(C[C@H]3C[C@H](C(F)(F)F)NC3=O)cc2n1)C1CCC(F)(F)CC1)c1nonc1C1CC1]

### Example 70: 4-Cyclopropyl-N—((S)-(4,4-difluorocyclohexyl)(7-(((3R*,5S*)-2-oxo-5-(trifluoromethyl)pyrrolidin-3-yl)methyl)imidazo[1,2-b]pyridazin-2-yl)methyl)-1,2,5-oxadiazole-3-carboxamide

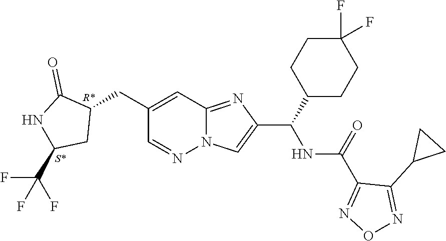 [SMILES: O=C(N[C@H](c1cn2ncc(C[C@@H]3C[C@@H](C(F)(F)F)NC3=O)cc2n1)C1CCC(F)(F)CC1)c1nonc1C1CC1]

### Example 71: 4-Cyclopropyl-N—((S)-(4,4-difluorocyclohexyl)(7-(((3S*,5S*)-2-oxo-5-(trifluoromethyl)pyrrolidin-3-yl)methyl)imidazo[1,2-b]pyridazin-2-yl)methyl)-1,2,5-oxadiazole-3-carboxamide

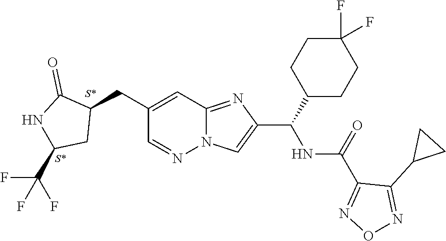 [SMILES: O=C(N[C@H](c1cn2ncc(C[C@H]3C[C@@H](C(F)(F)F)NC3=O)cc2n1)C1CCC(F)(F)CC1)c1nonc1C1CC1]

The title compounds were synthesized in a manner analogous to Example 59 using 3-((2-((S)-amino(4,4-difluorocyclohexyl)methyl)imidazo[1,2-b]pyridazin-7-yl)methyl)-5-(trifluoromethyl)pyrrolidin-2-one (Intermediate 63) in place of (5S)-3-((2-((S)-amino(4,4-difluorocyclohexyl)methyl)imidazo[1,2-b]pyridazin-7-yl)methyl)-5-(trifluoromethyl)pyrrolidin-2-one. The isomers were separated by chiral SFC (Chiralpak IB N3, 5 μm, 250×21 mm, 20% methanol, 80% CO₂). The first-eluting isomer (3% yield) was designated as the (3R*,5R*) isomer (Example 68), the second-eluting isomer (0.4% yield) was designated as the (3S*,5R*) isomer (Example 69), the third-eluting isomer ₍₁₋₆₎% yield) was designated as the (3R*,5S*) isomer (Example 70) and the fourth-eluting isomer (3% yield) was designated as the (3S*,5S′*) isomer (Example 71). Example 68: ¹H NMR (500 MHz, DMSO-d₆) δ 9.51-9.48 (m, 1H), 8.70 (s, 1H), 8.52-8.48 (m, 1H), 8.24-8.21 (m, 1H), 7.99-796 (m, 1H), 5.20 (t, J=8.50 Hz, 1H), 4.23 (br t, J=8.07 Hz, 2H), 3.15-3.07 (m, 1H), 2.95-2.89 (m, 1H), 2.78-2.71 (m, 1H), 2.32-2.26 (m, 1H), 2.20-2.03 (m, 4H), 1.95-1.89 (m, 1H), 1.86-1.73 (m, 2H), 1.67-1.61 (m, 1H), 1.45-1.36 (m, 1H), 1.33-1.26 (m, 1H), 1.15-1.11 (m, 2H), 0.98-0.96 (m, 2H). MS (ESI) m/z: [M+H]⁻ Found 568.2. Example 69: ¹H NMR (500 MHz, DMSO-d₆) δ 9.49-9.45 (m, 1H), 8.66-8.64 (m, 1H), 8.48-8.45 (m, 1H), 8.22-8.19 (m, 1H), 7.98-7.94 (m, 1H), 5.22-5.17 (m, 1H), 4.30-4.24 (m, 1H), 3.13-3.08 (m, 1H), 2.97-2.90 (m, 1H), 2.78-2.71 (m, 1H), 2.44-2.36 (m, 1H), 2.31-2.24 (m, 1H), 2.22-2.16 (m, 1H), 2.08-1.97 (m, 2H), 1.92-1.88 (m, 1H), 1.85-1.74 (m, 2H), 1.71-1.62 (m, 2H), 1.42-1.36 (m, 1H), 1.32-1.25 (m, 1H), 1.15-1.11 (m, 2H), 0.99-0.95 (m, 2H). MS (ESI) m/z: [M+H]⁺ Found 568.2. Example 70: ¹H NMR (500 MHz, DMSO-d₆) δ 9.48 (d, J=9.01 Hz, 1H), 8.65 (s, 1H), 8.46 (d, J=2.00 Hz, 1H), 8.20 (s, 1H), 7.96-7.94 (m, 1H), 5.22-5.17 (m, 1H), 4.30-4.24 (m, 1H), 3.30-3.28 (m, 1H), 3.13-3.07 (m, 1H), 2.96-2.89 (m, 1H), 2.77-2.72 (m, 1H), 2.31-2.26 (m, 1H), 2.23-2.16 (m, 1H), 2.10-1.96 (m, 2H), 1.93-1.89 (m, 1H), 1.87-1.72 (m, 2H), 1.70-1.60 (m, 2H), 1.43-1.39 (m, 1H), 1.33-1.26 (m, 1H), 1.14-1.11 (m, 2H), 0.96-0.99 (m, 2H). MS (ESI) m/z: [M+H]⁺ Found 568.2. Example 71: ¹H NMR (500 MHz, DMSO-d₆) δ 9.48 (d, J=9.01 Hz, 1H), 8.69 (s, 1H), 8.46 (d, J 2.00 Hz, 1H), 8.20 (s, 1H), 7.95 (s, 1H), 5.21-5.16 (m, 1H), 4.27-4.17 (m, 1H), 3.14-3.06 (m, 1H), 2.94-2.87 (m, 1H), 2.77-2.69 (m, 1H), 2.32-2.26 (m, 1H), 2.20-2.03 (m, 4H), 1.94-1.88 (m, 1H), 1.86-1.72 (m, 2H), 1.66-1.61 (m, 1H), 1.42-1.37 (m, 2H), 1.33-1.25 (m, 1H), 1.15-1.11 (m, 2H), 0.99-0.95 (m, 2H). MS (ESI) m/z: [M+H]⁺ Found 568.2.

### Example 72: 4-Cyclopropyl-N—((S)-(4,4-difluorocyclohexyl(7-((S*)-hydroxy((3S*,5S*)-2-oxo-5-(trifluoromethyl)pyrrolidin-3-yl)methyl)imidazo[1,2-b]pyridazin-2-yl)methyl)-1,2,5-oxadiazole-3-carboxamide

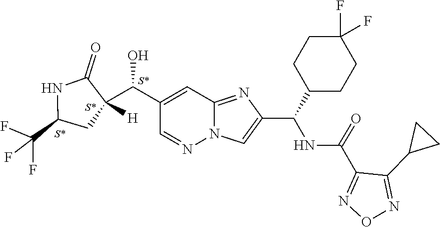 [SMILES: O=C(N[C@H](c1cn2ncc([C@@H](O)[C@@H]3C[C@@H](C(F)(F)F)NC3=O)cc2n1)C1CCC(F)(F)CC1)c1nonc1C1CC1]

### Example 73: 4-Cyclopropyl-N—((S)-(4,4-difluorocyclohexyl)(7-((S*)-2-oxo-5-(trifluoromethyl)pyrrolidin-3-yl)methyl)imidazo[1,2-b]pyridazin-2-yl)methyl)-1,2,5-oxadiazole-3-carboxamide

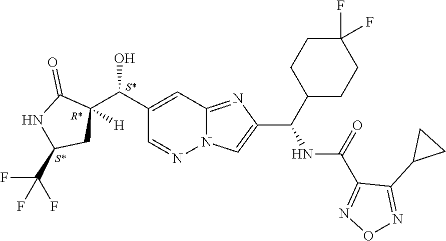 [SMILES: O=C(N[C@H](c1cn2ncc([C@@H](O)[C@H]3C[C@@H](C(F)(F)F)NC3=O)cc2n1)C1CCC(F)(F)CC1)c1nonc1C1CC1]

### Example 74: 4-Cyclopropyl-N—((S)-(4,4-difluorocyclohexyl(7-((S)-hydroxy((3R,5S)-2-oxo-5-(trifluoromethyl)pyrrolidin-3-yl)methyl)imidazo[1,2-b]pyridazin-2-yl)methyl)-1,2,5-oxadiazole-3-carboxamide

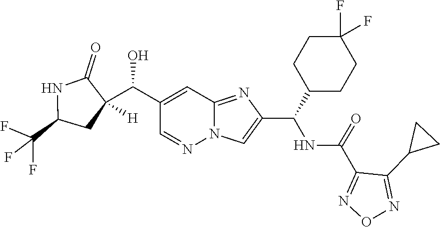 [SMILES: O=C(N[C@H](c1cn2ncc([C@@H](O)[C@H]3C[C@@H](C(F)(F)F)NC3=O)cc2n1)C1CCC(F)(F)CC1)c1nonc1C1CC1]

### Example 75: 4-Cyclopropyl-N—((S)-(4,4-difluorocyclohexyl)(7-((R*)-hydroxy((3S*,5S*)-2-oxo-5-(trifluoromethyl)pyrrolidin-3-yl)methyl)imidazo[1,2-b]pyridazin-2-yl)methyl)-1,2,5-oxadiazole-3-carboxamide

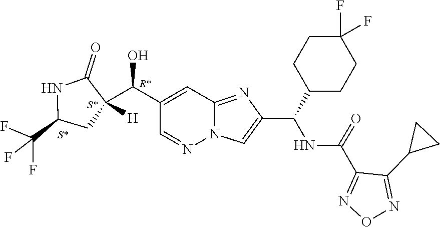 [SMILES: O=C(N[C@H](c1cn2ncc([C@H](O)[C@@H]3C[C@@H](C(F)(F)F)NC3=O)cc2n1)C1CCC(F)(F)CC1)c1nonc1C1CC1]

### Example 76: 4-Cyclopropyl-N—((S)-(4,4-difluorocyclohexyl)(7-((3S*,5R*)-2-oxo-5-(trifluoromethyl)pyrrolidin-3-yl)methyl)imidazo[1,2-b]pyridazin-2-yl)methyl)-1,2,5-oxadiazole-3-carboxamide

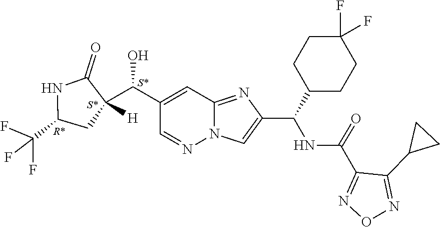 [SMILES: O=C(N[C@H](c1cn2ncc([C@@H](O)[C@@H]3C[C@H](C(F)(F)F)NC3=O)cc2n1)C1CCC(F)(F)CC1)c1nonc1C1CC1]

### Example 77: 4-Cyclopropyl-N—((S)-(4,4-difluorocyclohexyl)(7-((R*)-hydroxy((3R*,5S*)-2-oxo-5-(trifluoromethyl)pyrrolidin-3-yl)methyl)imidazo[1,2-b]pyridazin-2-yl)methyl)-1,2,5-oxadiazole-3-carboxamide

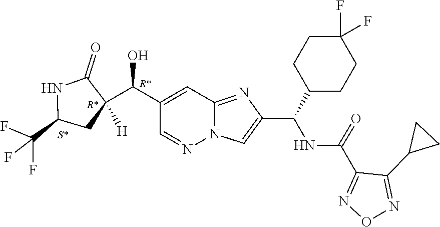 [SMILES: O=C(N[C@H](c1cn2ncc([C@H](O)[C@H]3C[C@@H](C(F)(F)F)NC3=O)cc2n1)C1CCC(F)(F)CC1)c1nonc1C1CC1]

The title compounds were synthesized in a manner analogous to Example 61. The isomers were separated by chiral SFC (Chiralpak AS-H, 5 μm, 250×21 mm, 25% ACN, 75% CO₂) to afford: Example 72 as the first-eluting isomer (1.7% yield), Example 73 as the second-eluting isomer (2% yield), Example 74 as the third-eluting isomer (0.7% yield, absolute stereochemistry was determined by 2D NMR), Example 75 as the fourth-eluting isomer (1% yield, absolute stereochemistry was determined by single-crystal x-ray crystallography), Example 76 as the fifth-eluting isomer (0.8% yield) and Example 77 as the sixth-eluting isomer (3% yield). Example 72: ¹H NMR (500 MHz, DMSO-d₆) δ 9.50-9.46 (m, 1H), 8.65-8.62 (m, 1H), 8.51-8.47 (m, 1H), 8.23-8.20 (m, 1H), 7.90-7.88 (m, 1H), 6.03-6.00 (m, 1H), 5.22-5.17 (m, 1H), 5.04-5.01 (m, 1H), 4.20-4.13 (m, 1H), 3.08-3.02 (m, 1H), 2.31-2.25 (m, 2H), 2.22-2.16 (m, 2H), 2.08-1.97 (m, 2H), 1.93-1.87 (m, 1H), 1.85-1.74 (m, 2H), 1.67-1.60 (m, 1H), 1.43-1.38 (m, 1H), 1.33-1.26 (m, 1H), 1.14-1.09 (m, 2H), 0.98-0.93 (m, 2H). MS (ESI) m/z: [M+H]⁺ Found 584.4. Example 73: ¹H NMR (500 MHz, DMSO-d₆) δ 9.51-9.48 (m, 1H), 8.69-8.66 (m, 1H), 8.58-8.56 (m, 1H), 8.24-8.22 (m, 1H), 7.96-7.93 (m, 1H), 6.04-6.02 (m, 1H), 5.22-5.17 (m, 2H), 4.28-4.21 (m, 1H), 2.98-2.93 (m, 1H), 2.44-2.36 (m, 1H), 2.31-2.26 (m, 1H), 2.22-2.16 (m, 1H), 2.10-1.98 (m, 2H), 1.94-1.88 (m, 1H), 1.86-1.72 (m, 3H), 1.66-1.60 (m, 1H), 1.46-1.37 (m, 1H), 1.34-1.24 (m, 1H), 1.14-1.10 (m, 2H), 0.98-0.95 (m, 2H). MS (ESI) m/z: [M+H]⁺ Found 584.4. Example 74: ¹H NMR (600 MHz, DMSO-d₆) δ 9.50-9.48 (m, 1H), 8.59-8.57 (m, 1H), 8.47-8.45 (m, 1H), 8.23-8.22 (m, 1H), 7.89-7.88 (m, 1H), 5.99-5.95 (m, 1H), 5.22-5.19 (m, 1H), 5.06-5.03 (m, 1H), 4.26-4.20 (m, 1H), 3.28-3.26 (m, 1H), 3.14-3.10 (m, 1H), 2.30-2.26 (m, 1H), 2.22-2.18 (m, 1H), 2.09-2.04 (m, 1H), 2.03-1.98 (m, 1H), 1.92-1.89 (m, 1H), 1.84-1.81 (m, 1H), 1.64-1.60 (m, 1H), 1.42-1.38 (m, 1H), 1.29-1.26 (m, 1H), 1.25-1.23 (m, 1H), 1.14-1.10 (m, 2H), 0.99-0.95 (m, 3H). MS (ESI) m/z: [M+H]⁺ Found 584.4. Example 75: ¹H NMR (500 MHz, DMSO-d₆) δ 9.50 (d, J=9.0 Hz, 1H), 8.64 (s, 1H), 8.50-8.46 (m, 1H), 8.23-8.21 (m, 1H), 7.89 (s, 1H), 6.04-5.97 (m, 1H), 5.20 (t, J=8.5 Hz, 1H), 5.05-5.02 (m, 1H), 4.21-4.13 (m, 1H), 3.09-3.02 (m, 1H), 2.34-2.27 (m, 2H), 2.23-2.17 (m, 2H), 2.11-1.97 (m, 2H), 1.95-1.73 (m, 3H), 1.69-1.61 (m, 1H), 1.47-1.39 (m, 1H), 1.38-1.26 (m, 1H), 1.16-1.09 (m, 2H), 1.00-0.94 (m, 2H). MS (ESI) m/z: [M+H]⁺ Found 584.4. Example 76: ¹H NMR (500 MHz, DMSO-d₆) δ 9.51-9.47 (m, 1H), 8.59-8.57 (m, 1H), 8.46-8.45 (m, 1H), 8.23-8.21 (m, 1H), 7.89-7.87 (m, 1H), 5.98-5.95 (m, 11H), 5.23-5.18 (m, 1H), 5.07-5.03 (m, 1H), 4.25-4.19 (m, 1H), 3.15-3.08 (m, 1H), 2.42-2.36 (m, 1H), 2.30-2.25 (m, 1H), 2.22-2.16 (m, 1H), 2.08-2.02 (m, 1H), 2.01-1.95 (m, 1H), 1.91-1.85 (m, 2H), 1.80-1.72 (m, 1H), 1.65-1.60 (m, 1H), 1.43-1.37 (m, 1H), 1.33-1.26 (m, 1H), 1.25-1.21 (m, 1H), 1.13-1.09 (m, 2H), 0.98-0.94 (m, 2H). MS (ESI) m/z: [M+H]⁺ Found 584.4. Example 77: ¹H NMR (500 MHz, DMSO-d₆) δ 9.51-9.47 (m, 1H), 8.69-8.66 (m, 111), 8.58-8.55 (m, 1H), 8.24-8.21 (m, 1H), 7.96-7.93 (m, 1H), 6.06-6.02 (m, 11H), 5.24-5.16 (m, 2H), 4.28-4.20 (m, 1H), 2.99-2.94 (m, 1H), 2.45-2.36 (m, 1H), 2.32-2.26 (m, 1H), 2.23-2.16 (m, 1H), 2.09-1.96 (m, 2H), 1.92-1.88 (m, 1H), 1.86-1.74 (m, 3H), 1.66-1.60 (m, 1H), 1.44-1.34 (m, 1H), 1.35-1.24 (m, 1H), 1.16-1.10 (m, 2H), 1.00-0.93 (m, 2H). MS (ESI) m/z: [M+H]⁺ Found 584.4.

### Example 78: 4-Cyclopropyl-N—((S)-(4,4-difluorocyclohexyl)(7-((R*)-1-((3R*,5S)-2-oxo-5-(trifluoromethyl)pyrrolidin-3-yl)ethyl)imidazo[1,2-b]pyridazin-2-yl)methyl)-1,2,5-oxadiazole-3-carboxamide

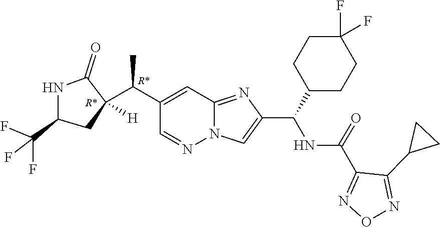 [SMILES: C[C@@H](c1cnn2cc([C@@H](NC(=O)c3nonc3C3CC3)C3CCC(F)(F)CC3)nc2c1)[C@H]1C[C@@H](C(F)(F)F)NC1=O]

### Example 79: 4-Cyclopropyl-N—((S)-(4,4-difluorocyclohexyl)(7-((S*)—1-((3R*,5S)-2-oxo-5-(trifluoromethyl)pyrrolidin-3-yl)ethyl)imidazo[1,2-b]pyridazin-2-yl)methyl)-1,2,5-oxadiazole-3-carboxamide

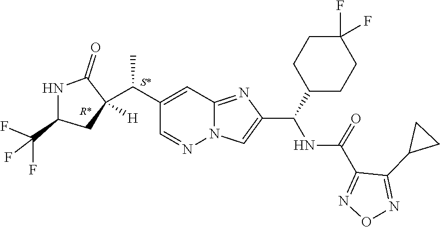 [SMILES: C[C@H](c1cnn2cc([C@@H](NC(=O)c3nonc3C3CC3)C3CCC(F)(F)CC3)nc2c1)[C@H]1C[C@@H](C(F)(F)F)NC1=O]

### Example 80: 4-Cyclopropyl-N—((S)-(4,4-difluorocyclohexyl(7-((R*)-1-((3S*,5S)-2-oxo-5-(trifluoromethyl)pyrrolidin-3-yl)ethyl)imidazo[1,2-b]pyridazin-2-yl)methyl)-1,2,5-oxadiazole-3-carboxamide

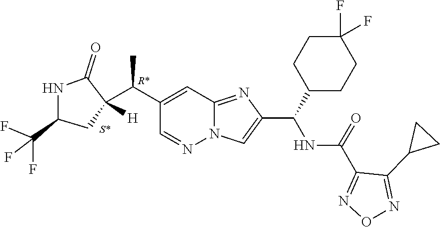 [SMILES: C[C@@H](c1cnn2cc([C@@H](NC(=O)c3nonc3C3CC3)C3CCC(F)(F)CC3)nc2c1)[C@@H]1C[C@@H](C(F)(F)F)NC1=O]

### Example 81: 4-Cyclopropyl-N—((S)-(4,4-difluorocyclohexyl)(7-((S*)-1-((3S*,5S)-2-oxo-5-(trifluoromethyl)pyrrolidin-3-yl)ethyl)imidazo[1,2-b]pyridazin-2-yl)methyl)-1,2,5-oxadiazole-3-carboxamide

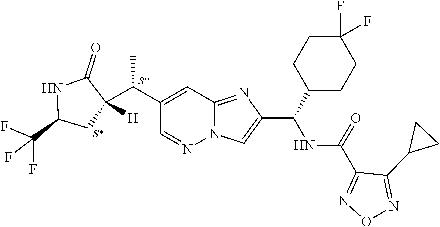 [SMILES: C[C@H](c1cnn2cc([C@@H](NC(=O)c3nonc3C3CC3)C3CCC(F)(F)CC3)nc2c1)[C@@H]1C[C@@H](C(F)(F)F)NC1=O]

The title compounds were synthesized in a manner analogous to Example 59 using (5S)-3-(1-(2-((S)-amino(4,4-difluorocyclohexyl)methyl)imidazo[1,2-b]pyridazin-7-yl)ethyl)-5-(trifluoromethyl)pyrrolidin-2-one (Intermediate 48) in place of (5S)-3-((2-((S)-amino(4,4-difluorocyclohexyl)methyl)imidazo[1,2-b]pyridazin-7-yl)methyl)-5-(trifluoromethyl)pyrrolidin-2-one to provide a mixture of diastereomers. The diastereomers were separated by chiral SFC (Chiralpak IA, 5 μm, 250×21 mm, 25% MeOH, 75% CO₂). The first-eluting isomer (2% yield) was designated as the (R*)-1-((3R*,5S)) isomer (Example 78), the second-eluting isomer (3% yield) was designated as the (S*)-1-((3R*,5S)) isomer (Example 79), the third-eluting isomer (10% yield) was designated as the (R*)-1-((3S*,5S)) isomer (Example 80) and the fourth-eluting isomer (4% yield) was designated as the (S*)-1-((3S*,5S)) isomer (Example 81). Example 78: ¹H NMR (500 MHz, DMSO-d₆) δ 9.49-9.42 (m, 1H), 8.62 (s, 1H), 8.54-8.49 (m, 1H), 8.46 (d, J=2.0 Hz, 1H), 8.21-8.16 (m, 1H), 7.95 (d, J=2.2 Hz, 1H), 5.18 (t, J=8.5 Hz, 1H), 4.27-4.18 (m, 1H), 3.31-3.21 (m, 2H), 3.00-2.85 (m, 1H), 2.32-2.23 (m, 2H), 2.19-2.15 (m, 2H), 2.12-1.94 (m, 2H), 1.94-1.69 (m, 2H), 1.68-1.58 (m, 2H), 1.45-1.40 (m, 3H), 1.16-1.07 (m, 2H), 1.01-0.92 (m, 2H). MS (ESI) m/z: [M+H]⁺ Found 582.3. Example 79: ¹H NMR (500 MHz, DMSO-d₆) δ 9.48-9.45 (m, 1H), 8.58-8.57 (m, 1H), 8.45-8.43 (m, 1H), 8.20-8.19 (m, 1H), 7.96-7.95 (m, 1H), 5.22-5.17 (m, 1H), 4.23-4.16 (m, 1H), 3.29-3.25 (m, 1H), 2.97-2.89 (m, 1H), 2.36-2.31 (m, 1H), 2.30-2.24 (m, 1H), 2.21-2.16 (m, 1H), 2.08-1.96 (m, 2H), 1.93-1.87 (m, 1H), 1.86-1.73 (m, 2H), 1.66-1.60 (m, 1H), 1.58-1.53 (m, 1H), 1.44-1.41 (m, 3H), 1.26-1.23 (m, 2H), 1.13-1.10 (m, 2H), 0.98-0.94 (m, 2H). MS (ESI) m/z: [M+H]⁺ Found 582.3.

Example 80: ¹H NMR (500 MHz, DMSO-d₆) δ 9.49-9.46 (m, 1H), 8.65-8.61 (m, 1H), 8.49-8.46 (m, 1H), 8.22-8.18 (m, 1H), 7.98-7.94 (m, 1H), 5.21-5.17 (m, 1H), 4.14-4.07 (m, 1H), 3.27-3.24 (m, 1H), 2.93-2.86 (m, 1H), 2.30-2.27 (m, 1H), 2.22-2.15 (m, 1H), 2.09-2.03 (m, 2H), 2.02-1.96 (m, 2H), 1.94-1.89 (m, 1H), 1.87-1.75 (m, 2H), 1.68-1.62 ((m, 1H), 1.46-1.42 (m, 3H), 1.34-1.28 (m, 1H), 1.27-1.23 (m, 1H), 1.15-1.11 (m, 2H), 0.99-0.95 (m, 2H). MS (ESI) m/z: [M+H]⁺ Found 582.2. Example 81: ¹H NMR (500 MHz, DMSO-d₆) δ 9.48-9.44 (m, 1H), 8.63-8.62 (m, 1H), 8.53-8.51 (m, 1H), 8.19-8.18 (m, 1H), 7.94-7.93 (m, 1H), 5.21-5.17 (m, 1H), 4.28-4.22 (m, 1H), 3.00-2.93 (m, 1H), 2.32-2.24 (m, 2H), 2.23-2.15 (m, 1H), 2.11-1.95 (m, 2H), 1.94-1.72 (m, 4H), 1.67-1.62 (m, 1H), 1.47-1.35 (m, 1H), 1.34-1.28 (m, 1H), 1.27-1.24 (m, 4H), 1.15-1.12 (m, 2H), 1.00-0.96 (m, 2H). MS (ESI) m/z: [M+H]⁺ Found 582.3.

### Example 82: 4-Cyclopropyl-N—((S)-(4,4-difluorocyclohexyl)(7-(((3R*,5S*)-5-methyl-2-oxopyrrolidin-3-yl)methyl)imidazo[1,2-b]pyridazin-2-yl)methyl)-1,2,5-oxadiazole-3-carboxamide

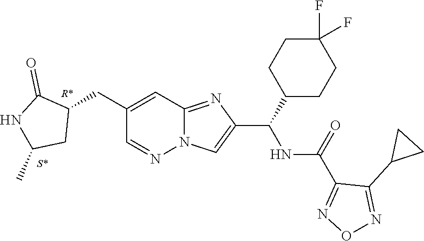 [SMILES: C[C@H]1C[C@@H](Cc2cnn3cc([C@@H](NC(=O)c4nonc4C4CC4)C4CCC(F)(F)CC4)nc3c2)C(=O)N1]

### Example 83: 4-Cyclopropyl-N—((S)-(4,4-difluorocyclohexyl)(7-(((3S*,5S*)-5-methyl-2-oxopyrrolidin-3-yl)methyl)imidazo[1,2-b]pyridazin-2-yl)methyl)-1,2,5-oxadiazole-3-carboxamide

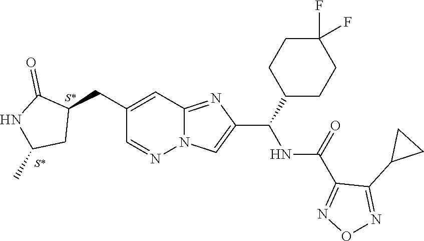 [SMILES: C[C@H]1C[C@H](Cc2cnn3cc([C@@H](NC(=O)c4nonc4C4CC4)C4CCC(F)(F)CC4)nc3c2)C(=O)N1]

### Example 84: Cyclopropyl-N—((S)-(4,4-difluorocyclohexyl)(7-(((3R*,5R*)-5-methyl-2-oxopyrrolidin-3-yl)methyl)imidazo[1,2-b]pyridazin-2-yl)methyl)-1,2,5-oxadiazole-3-carboxamide

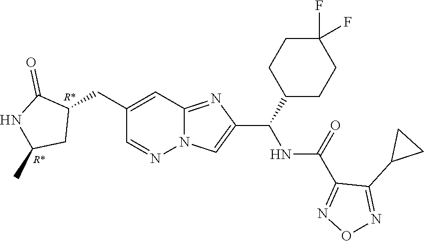 [SMILES: C[C@@H]1C[C@@H](Cc2cnn3cc([C@@H](NC(=O)c4nonc4C4CC4)C4CCC(F)(F)CC4)nc3c2)C(=O)N1]

### Example 85: 4-Cyclopropyl-N—((S)-(4,4-difluorocyclohexyl(7-(((3S*,5R*)-5-methyl-2-oxopyrrolidin-3-yl)methyl)imidazo[1,2-b]pyridazin-2-yl)methyl)-1,2,5-oxadiazole-3-carboxamide

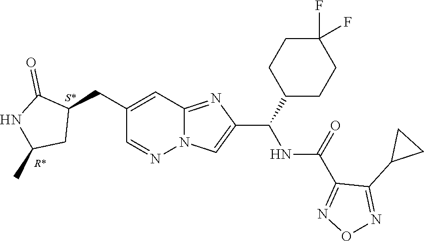 [SMILES: C[C@@H]1C[C@H](Cc2cnn3cc([C@@H](NC(=O)c4nonc4C4CC4)C4CCC(F)(F)CC4)nc3c2)C(=O)N1]

The title compounds were synthesized in a manner analogous to Example 59 using (13)-(4,4-difluorocyclohexyl)(7-((5-methyl-2-oxopyrrolidin-3-yl)methyl)imidazo[1,2-b]pyridazin-2-yl)methanaminium 2,2,2-trifluoracetate (Intermediate 49) in place of (5S)-3-((2-((S)-amino(4,4-difluorocyclohexyl)methyl)imidazo[1,2-b]pyridazin-7-yl)methyl)-5-(trifluoromethyl)pyrrolidin-2-one. The isomers were separated by chiral SFC (Stationary phase: Chiralcel OD-H, 5 μm, 250×21 mm, Mobile phase: 25% methanol with 0.2% diethylamine, 75% CO₂) to afford Example 82 as the first-eluting isomer (10% yield), Example 83 as the second-eluting isomer (2% yield), Example 84 as the third-eluting isomer (2% yield) and Example 85 as the fourth-eluting isomer (8% yield). Example 82: ¹H NMR (500 MHz, DMSO-d₆) δ 9.47 (d, J=9.0 Hz, 1H), 8.44 (d, J=2.0 Hz, 1H), 8.19 (s, 1H), 7.92 (d, J=1.6 Hz, 1H), 7.79 (s, 1H), 5.19 (t, J=8.5 Hz, 1H), 3.56-3.50 (m, 1H), 3.06-3.01 (m, 1H), 2.97-2.90 (m, 1H), 2.85-2.79 (m, 1H), 2.22-2.15 (m, 1H), 2.08-1.97 (m, 2H), 1.94-1.87 (m, 2H), 1.86-1.72 (m, 2H), 1.72-1.61 (m, 2H), 1.45-1.24 (m, 2H), 1.18-1.15 (m, 1H), 1.15-1.10 (m, 2H), 1.07-1.05 (m, 3H), 0.99-0.94 (m, 2H). MS (ESI) m/z: [M+H]⁺ Found 514.3. Example 83: ¹H NMR (500 MHz, DMSO-de) δ 9.50-9.45 (m, 1H), 8.45-8.43 (m, 1H), 8.19-8.18 (m, 1H), 7.93-7.91 (m, 1H), 7.82-7.79 (m, 1H), 5.21-5.16 (m, 1H), 3.53-3.48 (m, 1H), 3.12-3.08 (m, 1H), 2.79-2.73 (m, 1H), 2.68-2.62 (m, 1H), 2.31-2.25 (m, 1H), 2.22-2.16 (m, 2H), 2.09-1.96 (m, 2H), 1.93-1.87 (m, 1H), 1.86-1.72 (m, 2H), 1.66-1.59 (m, 1H), 1.45-1.35 (m, 1H), 1.32-1.20 (m, 2H), 1.19-1.10 (m, 3H), 1.09-1.06 (m, 2H), 0.99-0.95 (m, 2H). MS (ESI) m/z: [M+H]⁺ Found 514.3. Example 84: ¹H NMR (500 MHz, DMSO-d₆) δ 9.49-9.45 (m, 1H), 8.45-8.43 (m, 1H), 8.19-8.18 (m, 1H), 7.92-7.91 (m, 1H), 7.82-7.79 (m, 1H), 5.18 (t, J=8.5 Hz, 1H), 3.54-3.46 (m, 1H), 3.11-3.06 (m, 1H), 2.81-2.72 (m, 1H), 2.67-2.61 (m, 1H), 2.32-2.25 (m, 1H), 2.21-2.14 (m, 2H), 2.09-1.96 (m, 2H), 1.93-1.87 (m, 1H), 1.85-1.71 (m, 2H), 1.67-1.59 (m, 1H), 1.44-1.35 (m, 1H), 1.31-1.26 (m, 1H), 1.25-1.19 (m, 1H), 1.13-1.10 (m, 2H), 1.07-1.05 (m, 3H), 0.97-0.94 (m, 2H). MS (ESI) m/z: [M+H]⁺ Found 514.3. Example 85: ¹H NMR (500 MHz, DMSO-d₆) δ 9.46 (d, J=9.1 Hz, 1H), 8.43 (d, J=1.9 Hz, 1H), 8.18 (s, 1H), 7.92 (s, 1H), 7.79 (s, 1H), 5.21-5.16 (m, 1H), 3.55-3.50 (m, 1H), 3.05-3.00 (m, 1H), 2.84-2.80 (m, 1H), 2.71-2.66 (m, 1H), 2.31-2.24 (m, 1H), 2.21-2.14 (m, 1H), 2.07-1.97 (m, 2H), 1.93-1.87 (m, 2H), 1.85-1.72 (m, 2H), 1.70-1.61 (m, 2H), 1.44-1.36 (m, 1H), 1.32-1.24 (m, 1H), 1.13-1.11 (m, 2H), 1.07-1.05 (m, 3H), 0.97-0.95 (m, 2H). MS (ESI) m/z: [M+H]⁺ Found 514.3.

### Example 86: 4-Cyclopropyl-N—[(S)-(4,4-difluorocyclohexyl)-[7-[[(3S*,4R*)-4-methyl-2-oxo-pyrrolidin-3-yl]methyl]imidazo[1,2-b]pyridazin-2-yl]methyl]-1,2,5-oxadiazole-3-carboxamide

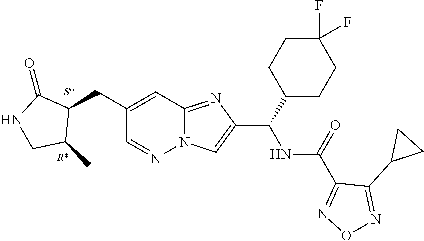 [SMILES: C[C@H]1CNC(=O)[C@H]1Cc1cnn2cc([C@@H](NC(=O)c3nonc3C3CC3)C3CCC(F)(F)CC3)nc2c1]

### Example 87: 4-Cyclopropyl-N—[(S)-(4,4-difluorocyclohexyl)-[7-[[(3R*,4R*)-4-methyl-2-oxo-pyrrolidin-3-yl]methyl]imidazo[1,2-b]pyridazin-2-yl]methyl]-1,2,5-oxadiazole-3-carboxamide

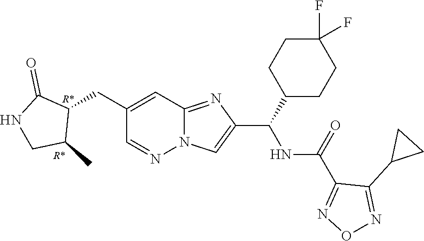 [SMILES: C[C@H]1CNC(=O)[C@@H]1Cc1cnn2cc([C@@H](NC(=O)c3nonc3C3CC3)C3CCC(F)(F)CC3)nc2c1]

### Example 88: 4-Cyclopropyl-N—[(S)-(4,4-difluorocyclohexyl)-[7-[[(3R*,4S*)-4-methyl-2-oxo-pyrrolidin-3-yl]methyl]imidazo[1,2-b]pyridazin-2-yl]methyl]-1,2,5-oxadiazole-3-carboxamide

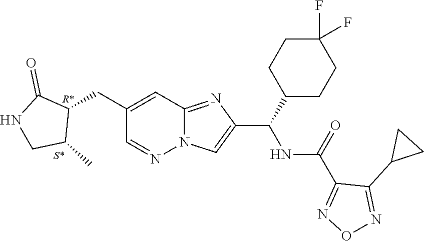 [SMILES: C[C@@H]1CNC(=O)[C@@H]1Cc1cnn2cc([C@@H](NC(=O)c3nonc3C3CC3)C3CCC(F)(F)CC3)nc2c1]

The title compounds were synthesized in a manner analogous to Example 59 using (1S)-(4,4-difluorocyclohexyl)(7-((4-methyl-2-oxopyrrolidin-3-yl)methyl)imidazo[1,2-b]pyridazin-2-yl)methanaminium 2,2,2-trifluoroacetate (Intermediate 50) in place of (5S)-3-((2-((S)-amino(4,4-difluorocyclohexyl)methyl)imidazo[1,2-b]pyridazin-7-yl)methyl)-5-(trifluoromethyl)pyrrolidin-2-one. The isomers were separated by chiral SFC (Stationary phase: Chiralcel OD-H, 5 μm, 250×30 mm, Mobile phase: 25% acetonitrile:methanol (1:1), 75% CO₂) to afford Example 86 as the first-eluting isomer (15% yield), Example 87 as the second-eluting isomer (1% yield) and Example 88 as the third-eluting isomer (11% yield). Example 86: ¹H NMR (500 MHz, DMSO-d₆) δ 9.49-9.44 (m, 1H), 8.45 (d, J=2.0 Hz, 1H), 8.18 (s, 1H), 7.92 (d, J=1.6 Hz, 1H), 7.64 (s, 1H), 5.18 (t, J=8.6 Hz, 1H), 3.27-3.20 (m, 1H), 3.01-2.95 (m, 1H), 2.93-2.89 (m, 1H), 2.74-2.70 (m, 1H), 2.35-2.25 (m, 2H), 2.23-2.16 (m, 1H), 2.10-1.96 (m, 3H), 1.92-1.72 (m, 3H), 1.67-1.60 (m, 1H), 1.45-1.26 (m, 2H), 1.15-1.06 (m, 2H), 0.97-0.93 (m, 5H). MS (ESI) m/z: [M+H]⁺ Found 514.3. Example 87: ¹H NMR (600 MHz, MeOH-d₄) δ 8.46-8.45 (m, 1H), 8.10-8.08 (m, 1H), 7.89-7.87 (m, 1H), 5.29-5.27 (m, 1H), 3.55-3.51 (m, 1H), 3.11-3.07 (m, 1H), 3.03-3.00 (m, 1H), 2.99-2.96 (m, 1H), 2.91-2.87 (m, 1H), 2.69-2.64 (m, 1H), 2.44-2.39 (m, 1H), 2.28-2.22 (m, 1H), 2.16-2.09 (m, 1H), 2.07-2.01 (m, 2H), 1.89-1.72 (m, 2H), 1.71-1.66 (m, 1H), 1.56-1.49 (m, 1H), 1.46-1.39 (m, 1H), 1.18-1.14 (m, 2H), 1.11-1.08 (m, 3H), 1.06-1.03 (m, 2H). MS (ESI) m/z: [M+H]⁺ Found 514.3. Example 88: ¹H NMR (500 MHz, DMSO-d₆) δ 9.47-9.44 (m, 1H), 8.44 (d, J=2.1 Hz, 1H), 8.18 (s, 1H), 7.91 (d, J=1.3 Hz, 1H), 7.63-7.62 (m, 1H), 5.19 (s, 1H), 3.24-3.19 (m, 1H), 3.00-2.94 (m, 1H), 2.87-2.82 (m, 1H), 2.75-2.70 (m, 1H), 2.33-2.29 (m, 1H), 2.28-2.24 (m, 1H), 2.21-2.14 (m, 1H), 2.08-2.02 (m, 2H), 2.01-1.94 (m, 1H), 1.92-1.85 (m, 1H), 1.85-1.70 (m, 2H), 1.65-1.59 (m, 1H), 1.45 (s, 2H), 1.43-1.35 (m, 1H), 1.33-1.22 (m, 1H), 1.13-1.08 (m, 2H), 0.96-0.94 (m, 2H), 0.94-0.93 (m, 1H). MS (ESI) m/z: [M+H]⁺ Found 514.3.

### Example 89: 4-Cyclopropyl-N—[(S)-(4,4-difluorocyclohexyl)-[7-[[(3S*,5R*)-5-ethyl-2-oxo-pyrrolidin-3-yl]methyl]imidazo[1,2-b]pyridazin-2-yl]methyl]-1,2,5-oxadiazole-3-carboxamide

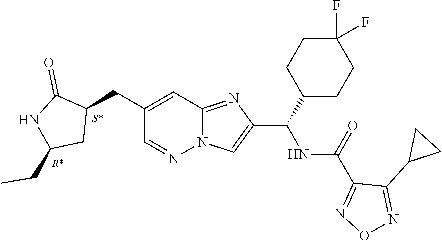 [SMILES: CC[C@@H]1C[C@H](Cc2cnn3cc([C@@H](NC(=O)c4nonc4C4CC4)C4CCC(F)(F)CC4)nc3c2)C(=O)N1]

### Example 90: 4-Cyclopropyl-N—[(S)-(4,4-difluorocyclohexyl)-[7-[[(3R*,5R*)-5-ethyl-2-oxo-pyrrolidin-3-yl]methyl]imidazo[1,2-b]pyridazin-2-yl]methyl]-1,2,5-oxadiazole-3-carboxamide

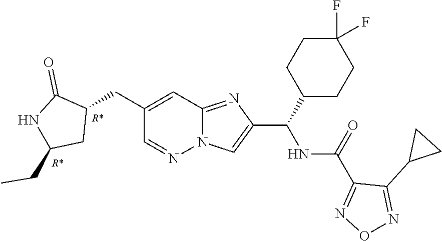 [SMILES: CC[C@@H]1C[C@@H](Cc2cnn3cc([C@@H](NC(=O)c4nonc4C4CC4)C4CCC(F)(F)CC4)nc3c2)C(=O)N1]

### Example 91: 4-Cyclopropyl-N—[(S)-(4,4-difluorocyclohexyl)-[7-[[(3S*,5S*)-5-ethyl-2-oxo-pyrrolidin-3-yl]methyl]imidazo[1,2-b]pyridazin-2-yl]methyl]-1,2,5-oxadiazole-3-carboxamide

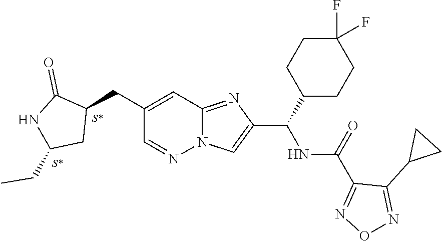 [SMILES: CC[C@H]1C[C@H](Cc2cnn3cc([C@@H](NC(=O)c4nonc4C4CC4)C4CCC(F)(F)CC4)nc3c2)C(=O)N1]

### Example 92: 4-Cyclopropyl-N—[(S)-(4,4-difluorocyclohexyl)-[7-[[(3R*,5S*)-5-ethyl-2-oxo-pyrrolidin-3-yl]methyl]imidazo[1,2-b]pyridazin-2-yl]methyl]-1,2,5-oxadiazole-3-carboxamide

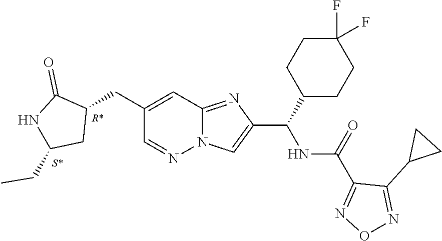 [SMILES: CC[C@H]1C[C@@H](Cc2cnn3cc([C@@H](NC(=O)c4nonc4C4CC4)C4CCC(F)(F)CC4)nc3c2)C(=O)N1]

The title compounds were synthesized in a manner analogous to Example 59 using (1S)-(4,4-difluorocyclohexyl(7-((5-ethyl-2-oxopyrrolidin-3-yl)methyl)imidazo[1,2-b]pyridazin-2-yl)methanaminium 2,2,2-trifluoroacetate (Intermediate 51) in place of (5S)-3-((2-((S)-amino(4,4-difluorocyclohexyl)methyl)imidazo[1,2-b]pyridazin-7-yl)methyl)-5-(trifluoromethyl)pyrrolidin-2-one. The isomers were separated by chiral SFC (Stationary phase: Chiralcel OD-H, 5 μm, 250×21 mm, Mobile phase: 25% methanol, 75% CO₂) to afford Example 89 as the first-eluting isomer (16% yield), Example 90 as the second-eluting isomer (3% yield), Example 91 as the third-eluting isomer (4% yield) and Example 92 as the fourth-eluting isomer (14% yield). Example 89: ¹H NMR (500 MHz, DMSO-d₆) δ 9.48-9.45 (m, 1H), 8.44 (d, J=2.0 Hz, 1H), 8.18 (s, 1H), 7.92 (s, 2H), 5.21-5.16 (m, 1H), 3.07-3.01 (m, 1H), 2.82-2.74 (m, 1H), 2.71-2.63 (m, 1H), 2.33-2.25 (m, 1H), 2.25-2.14 (m, 1H), 2.10-1.96 (m, 2H), 1.94-1.70 (m, 5H), 1.68-1.59 (m, 1H), 1.44-1.23 (m, 5H), 1.15-1.08 (m, 2H), 0.99-0.94 (m, 2H), 0.81 (t, J=7.4 Hz, 3H). MS (ESI) m/z: [M+H]⁺ Found 528.3. Example 90: ¹H NMR (500 MHz, DMSO-d₆) δ 9.50-9.45 (m, 1H), 8.47-8.44 (m, 1H), 8.21-8.18 (m, 1H), 7.94-7.89 (m, 2H), 5.23-5.17 (m, 1H), 3.13-3.07 (m, 1H), 2.81-2.73 (m, 1H), 2.69-2.61 (m, 1H), 2.31-2.25 (m, 1H), 2.23-2.13 (m, 2H), 2.10-1.94 (m, 2H), 1.95-1.87 (m, 1H), 1.87-1.72 (m, 2H), 1.67-1.60 (m, 1H), 1.49-1.35 (m, 2H), 1.36-1.20 (m, 4H), 1.17-1.10 (m, 2H), 1.01-0.94 (m, 2H), 0.86-0.79 (m, 3H). MS (ESI) m/z: [M+H]⁺ Found 528.3. Example 91: ¹H NMR (500 MHz, DMSO-d₆) δ 9.49-9.45 (m, 1H), 8.45 (d, J=2.0 Hz, 1H), 8.19 (s, 1H), 7.93-7.92 (m, 1H), 7.91-7.89 (m, 1H), 5.22-5.16 (m, 1H), 3.13-3.07 (m, 1H), 2.81-2.74 (m, 1H), 2.67-2.61 (m, 1H), 2.32-2.25 (m, 1H), 2.22-2.14 (m, 2H), 2.10-1.95 (m, 2H), 1.94-1.88 (m, 1H), 1.87-1.73 (m, 2H), 1.68-1.61 (m, 1H), 1.46-1.38 (m, 2H), 1.34-1.19 (m, 4H), 1.15-1.10 (m, 2H), 0.99-0.94 (m, 2H), 0.84-0.79 (m, 3H). MS (ESI) m/z: [M+H]⁺ Found 528.3. Example 92: ¹H NMR (500 MHz, DMSO-d₆) δ 9.46 (d, J=9.0 Hz, 1H), 8.43 (d, J=2.0 Hz, 1H), 8.17 (s, 1H), 7.91 (s, 2H), 5.17 (t, J=8.6 Hz, 1H), 3.05-3.00 (m, 1H), 2.81-2.74 (m, 1H), 2.70-2.63 (m, 1H), 2.30-2.24 (m, 1H), 2.22-2.12 (m, 1H), 2.09-1.94 (m, 2H), 1.93-1.69 (m, 5H), 1.65-1.58 (m, 1H), 1.45-1.22 (m, 5H), 1.13-1.08 (m, 2H), 0.98-0.93 (m, 2H), 0.83-0.78 (m, 3H). MS (ESI) m/z: [M+H]⁺ Found 528.3.

### Example 93: 4-Cyclopropyl-N—[(S)-(4,4-difluorocyclohexyl)-[7-[[(3S*,5R*)-5-isopropyl-2-oxo-pyrrolidin-3-yl]methyl]imidazo[1,2-b]pyridazin-2-yl]methyl]-1,2,5-oxadiazole-3-carboxamide

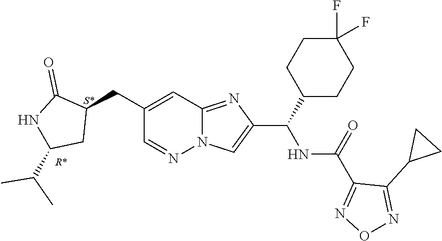 [SMILES: CC(C)[C@H]1C[C@H](Cc2cnn3cc([C@@H](NC(=O)c4nonc4C4CC4)C4CCC(F)(F)CC4)nc3c2)C(=O)N1]

### Example 94: 4-Cyclopropyl-N—[(S)-(4,4-difluorocyclohexyl)-[7-[[(3R*,5R*)-5-isopropyl-2-oxo-pyrrolidin-3-yl]methyl]imidazo[1,2-b]pyridazin-2-yl]methyl]-1,2,5-oxadiazole-3-carboxamide

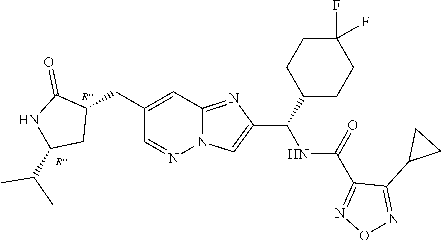 [SMILES: CC(C)[C@H]1C[C@@H](Cc2cnn3cc([C@@H](NC(=O)c4nonc4C4CC4)C4CCC(F)(F)CC4)nc3c2)C(=O)N1]

### Example 95: 4-Cyclopropyl-N—[(S)-(4,4-difluorocyclohexyl)-[7-[[(3R*,5S*)-5-isopropyl-2-oxo-pyrrolidin-3-yl]methyl]imidazo[1,2-b]pyridazin-2-yl]methyl]-1,2,5-oxadiazole-3-carboxamide

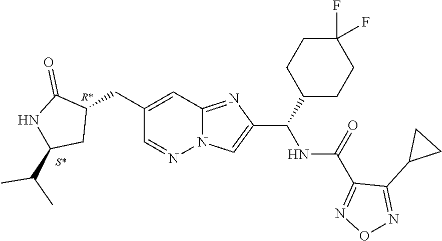 [SMILES: CC(C)[C@@H]1C[C@@H](Cc2cnn3cc([C@@H](NC(=O)c4nonc4C4CC4)C4CCC(F)(F)CC4)nc3c2)C(=O)N1]

### Example 96: 4-Cyclopropyl-N—[(S)-(4,4-difluorocyclohexyl)-[7-[[(3S*,5S*)-5-isopropyl-2-oxo-pyrrolidin-3-yl]methyl]imidazo[1,2-b]pyridazin-2-yl]methyl]-1,2,5-oxadiazole-3-carboxamide

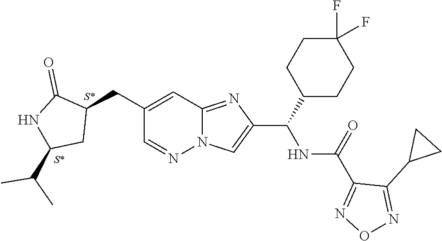 [SMILES: CC(C)[C@@H]1C[C@H](Cc2cnn3cc([C@@H](NC(=O)c4nonc4C4CC4)C4CCC(F)(F)CC4)nc3c2)C(=O)N1]

The title compounds were synthesized in a manner analogous to Example 59 using (1S)-(4,4-difluorocyclohexyl)(7-((5-isopropyl-2-oxopyrrolidin-3-yl)methyl)imidazo[1,2-b]pyridazin-2-yl)methanaminium 2,2,2-trifluoroacetate (Intermediate 52) in place of (5S)-3-((2-((S)-amino(4,4-difluorocyclohexyl)methyl)imidazo[1,2-b]pyridazin-7-yl)methyl)-5-(trifluoromethyl)pyrrolidin-2-one. The isomers were separated by chiral SFC (Stationary phase: Chiralcel OD-H, 5 μm, 250×21 mm, Mobile phase: 25% methanol, 75% CO₂) to afford Example 93 as the first-eluting isomer (4% yield), Example 94 as the second-eluting isomer (4% yield), Example 95 as the third-eluting isomer (6% yield) and Example 96 as the fourth-eluting isomer (4% yield). Example 93: ¹H NMR (600 MHz, DMSO-do) δ 9.47 (d, J=9.0 Hz, 1H), 8.46 (d, J=1.7 Hz, 1H), 8.20 (s, 1H), 7.93 (d, J=7.6 Hz, 2H), 5.18 (t, J=8.6 Hz, 1H), 3.20-3.15 (m, 2H), 3.06-3.01 (m, 2H), 2.76-2.65 (m, 2H), 2.30-2.25 (m, 1H), 2.21-2.14 (m, 1H), 2.09-1.95 (m, 2H), 1.93-1.88 (m, 1H), 1.84-1.81 (m, 2H), 1.66-1.60 (m, 1H), 1.56-1.50 (m, 1H), 1.44-1.35 (m, 1H), 1.33-1.24 (m, 1H), 1.14-1.08 (m, 2H), 0.98-0.94 (m, 2H), 0.81 (d, J=6.7 Hz, 3H), 0.80-0.77 (m, 3H). MS (ESI) m/z: [M+H]⁺ Found 542.3. Example 94: ¹H NMR (600 MHz, DMSO-d₆) δ 9.51-9.45 (m, 1H), 8.50-8.46 (m, 1H), 8.23-8.19 (m, 1H), 7.97-7.94 (m, 1H), 7.94-7.91 (m, 1H), 5.21-5.17 (m, 1H), 3.20-3.14 (m, 1H), 3.13-3.08 (m, 1H), 2.81-2.74 (m, 1H), 2.69-2.63 (m, 1H), 2.31-2.26 (m, 1H), 2.22-2.14 (m, 1H), 2.10-1.96 (m, 3H), 1.93-1.87 (m, 1H), 1.86-1.72 (m, 2H), 1.66-1.60 (m, 1H), 1.51-1.35 (m, 2H), 1.33-1.23 (m, 2H), 1.15-1.10 (m, 2H), 0.99-0.95 (m, 2H), 0.84 (d, J=6.7 Hz, 3H), 0.76 (d, J=6.7 Hz, 3H). MS (ESI) m/z: [M+H]⁺ Found 542.3. Example 95: ¹H NMR (600 MHz, DMSO-d₆) δ 9.49-9.45 (m, 1H), 8.49-8.45 (m, 1H), 8.22-8.18 (m, 1H), 7.97-7.93 (m, 1H), 7.93-7.91 (m, 1H), 5.21-5.17 (m, 1H), 3.19-3.14 (m, 1H), 3.11-3.07 (m, 1H), 2.81-2.73 (m, 1H), 2.68-2.63 (m, 1H), 2.30-2.25 (m, 1H), 2.21-2.14 (m, 1H), 2.10-1.95 (m, 3H), 1.91-1.87 (m, 1H), 1.84-1.73 (m, 2H), 1.65-1.60 (m, 1H), 1.48-1.35 (m, 2H), 1.32-1.25 (m, 2H), 1.14-1.10 (m, 2H), 0.98-0.94 (m, 2H), 0.85-0.82 (m, 3H), 0.77-0.74 (m, 3H). MS (ESI) m/z: [M+H]⁺ Found 542.3. Example 96: ¹H NMR (600 MHz, DMSO-d₆) δ 9.48-9.44 (m, 1H), 8.49-8.43 (m, 1H), 8.21-8.18 (m, 1H), 7.95-7.91 (m, 2H), 5.20-5.16 (m, 1H), 3.19-3.15 (m, 1H), 3.06-3.01 (m, 1H), 2.76-2.70 (m, 1H), 2.70-2.64 (m, 1H), 2.30-2.25 (m, 1H), 2.22-2.13 (m, 1H), 2.08-1.94 (m, 2H), 1.92-1.86 (m, 1H), 1.85-1.70 (m, 4H), 1.67-1.59 (m, IH), 1.56-1.49 (m, 1H), 1.44-1.35 (m, 1H), 1.33-1.23 (m, 1H), 1.13-1.08 (m, 2H), 0.97-0.92 (m, 2H), 0.82-0.79 (m, 3H), 0.79-0.77 (m, 3H). MS (ESI) m/z: [M+H]⁺ Found 542.3.

### Example 97: 4-Cyclopropyl-N—[(S)-(4,4-difluorocyclohexyl)-[7-[[(3S,5S)-3-methyl-2-oxo-5-(trifluoromethyl)pyrrolidin-3-yl]methyl]imidazo[1,2-b]pyridazin-2-yl]methyl]-1,2,5-oxadiazole-3-carboxamide

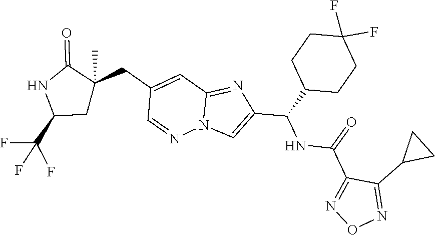 [SMILES: C[C@]1(Cc2cnn3cc([C@@H](NC(=O)c4nonc4C4CC4)C4CCC(F)(F)CC4)nc3c2)C[C@@H](C(F)(F)F)NC1=O]

The title compound was synthesized in a manner analogous to Example 21 using (1S)-(4,4-difluorocyclohexyl)(7-(((5S)-3-methyl-2-oxo-5-(trifluoromethyl)pyrrolidin-3-yl)methyl)imidazo[1,2-b]pyridazin-2-yl)methanaminium 2,2,2-trifluoroacetate (Intermediate 53 Step B) in place of 3-((2-((S)-amino(4,4-difluorocyclohexyl)methyl)imidazo[1,2-b]pyridazin-7-yl)methyl)pyrrolidin-2-one. Purification was performed via preparative basic HPLC (X-Bridge Prep C18 column, 5 μm, 50×250 mm, 5-100% acetonitrile/water (with 20 mM NH₄OH) and a chiral separation of the diastereomers was performed via SFC (Stationary phase: Chiralpak IC, 5 μm, 250×30 mm, Mobile phase: 25% methanol, 75% CO₂) to afford the title compound as the second-eluting isomer (15% yield, absolute stereochemistry was determined by 2D NMR). ¹H NMR (600 MHz, DMSO-d₆) δ 9.48 (d, J=9.2 Hz, 1H), 8.60 (s, 1H), 8.35 (d, J=2.1 Hz, 1H), 8.21 (s, 1H), 7.88 (d, J=1.5 Hz, 1H), 5.20 (t, J=8.5 Hz, 1H), 4.39-4.31 (m, 1H), 2.93-2.90 (m, 1H), 2.85-2.81 (m, 1H), 2.32-2.24 (m, 1H), 2.23-2.16 (m, 1H), 2.09-2.02 (m, 2H), 2.02-1.97 (m, 2H), 1.93-1.86 (m, 1H), 1.85-1.73 (m, 2H), 1.65-1.60 (m, 1H), 1.43-1.37 (m, 1H), 1.33-1.24 (m, 1H), 1.17-1.15 (m, 3H), 1.14-1.09 (m, 2H), 0.98-0.95 (m, 2H). MS (ESI) m/z: [M+H]⁺ Found 582.3.

### Example 98: 4-Cyclopropyl-N-((1S)-(4,4-difluorocyclohexyl)(7-((6-oxo-2-oxa-5-azaspiro[3.4]octan-7-yl)methyl)imidazo[1,2-b]pyridazin-2-yl)methyl)-1,2,5-oxadiazole-3-carboxamide

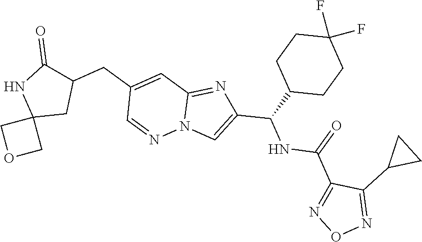 [SMILES: O=C(N[C@H](c1cn2ncc(CC3CC4(COC4)NC3=O)cc2n1)C1CCC(F)(F)CC1)c1nonc1C1CC1]

The title compound was synthesized in a manner analogous to Example 21 using (1S)-(4,4-difluorocyclohexyl)(7-((6-oxo-2-oxa-5-azaspiro[3.4]octan-7-yl)methyl)imidazo[1,2-b]pyridazin-2-yl)methanaminium 2,2,2-trifluoroacetate (Intermediate 54 Step B) in place of 3-((2-((S)-amino(4,4-difluorocyclohexyl)methyl)imidazo[1,2-b]pyridazin-7-yl)methyl)pyrrolidin-2-one. Purification was performed via preparative basic HPLC (X-Bridge Prep C18 column, 5 μm, 50×250 mm, 5-100% acetonitrile/water (with 20 mM NH₄OH)) to afford the title compound (34% yield). ¹H NMR (500 MHz, DMSO-d₆) δ 9.50-9.46 (m, 1H), 8.56 (s, 1H), 8.20 (s, 1H), 8.03 (s, 1H), 7.76 (s, 1H), 5.22-5.17 (m, 1H), 4.87-4.85 (m, 1H), 4.44-4.41 (m, 1H), 4.41-4.37 (m, 2H), 3.53-3.49 (m, 2H), 3.13-3.00 (m, 2H), 2.90-2.85 (m, 1H), 2.31-2.25 (m, 1H), 2.24-2.15 (m, 1H), 2.09-1.98 (m, 2H), 1.94-1.87 (m, 1H), 1.86-1.73 (m, 2H), 1.68-1.62 (m, 1H), 1.45-1.35 (m, 1H), 1.35-1.24 (m, 1H), 1.14-1.10 (m, 2H), 0.99-0.94 (m, 2H). MS (ESI) m/z: [M+H]⁺ Found 542.2.

### Example 99: 4-Cyclopropyl-N—((S)-(4,4-difluorocyclohexyl)(7-(((3S,5S)-3-(methoxymethyl)-2-oxo-5-(trifluoromethyl)pyrrolidin-3-yl)methyl)imidazo[1,2-b]pyridazin-2-yl)methyl)-1,2,5-oxadiazole-3-carboxamide

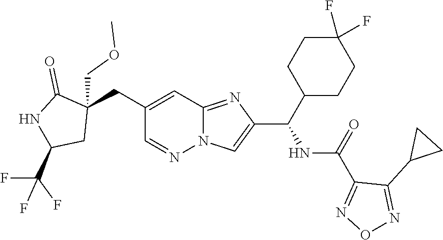 [SMILES: COC[C@]1(Cc2cnn3cc([C@@H](NC(=O)c4nonc4C4CC4)C4CCC(F)(F)CC4)nc3c2)C[C@@H](C(F)(F)F)NC1=O]

The title compound was synthesized in a manner analogous to Example 21 using (1S)-(4,4-difluorocyclohexyl)(7-(((5S)-3-(methoxymethyl)-2-oxo-5-(trifluoromethyl)pyrrolidin-3-yl)methyl)imidazo[1,2-b]pyridazin-2-yl)methanaminium 2,2,2-trifluoroacetate (Intermediate 55 Step B) in place of 3-((2-((S)-amino(4,4-difluorocyclohexyl)methyl)imidazo[1,2-b]pyridazin-7-yl)methyl)pyrrolidin-2-one. Purification was performed via preparative basic HPLC (X-Bridge Prep C18 column, 5 μm, 50×250 mm, 5-100% acetonitrile/water (with 20 mM NH₄OH)) and a chiral separation was performed via SFC (Stationary phase: Chiralpak IC, 5 μm, 250×30 mm, Mobile phase: 20% methanol, 80% CO₂) to afford the title compound as the second-eluting isomer (4% yield, absolute stereochemistry was determined by 2D NMR). ¹H NMR (500 MHz, DMSO-d₆) δ 9.49 (d, J=9.0 Hz, 1H), 8.69 (s, 1H), 8.31 (d, J=2.0 Hz, 1H), 8.22 (s, 1H), 7.87 (d, J=1.6 Hz, 1H), 5.20 (t, J=8.5 Hz, 1H), 4.19-4.10 (m, 1H), 3.49 (d, J=8.6 Hz, 1H), 3.31-3.29 (m, 1H), 3.29-3.28 (m, 3H), 2.90 (d, J=13.8 Hz, 1H), 2.84-2.78 (m, 1H), 2.39-2.33 (m, 1H), 2.31-2.24 (m, 1H), 2.23-2.16 (m, 1H), 2.10-1.71 (m, 6H), 1.65-1.58 (m, 1H), 1.45-1.35 (m, 1H), 1.34-1.23 (m, 1H), 1.15-1.09 (m, 2H), 0.99-0.94 (m, 2H). MS (ESI) m/z: [M+H]⁺ Found 612.3.

### Example 100: 4-Cyclopropyl-N—[(S)-(4,4-difluorocyclohexyl)-[7-[[(3S*,4S*)-2-oxo-4-(trifluoromethyl)pyrrolidin-3-yl]methyl]imidazo[1,2-b]pyridazin-2-yl]methyl]-1,2,5-oxadiazole-3-carboxamide

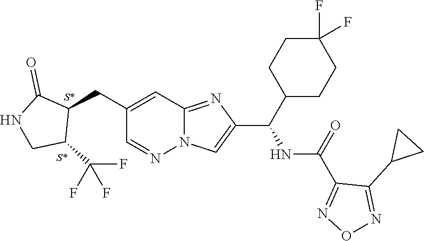 [SMILES: O=C(N[C@H](c1cn2ncc(C[C@@H]3C(=O)NC[C@H]3C(F)(F)F)cc2n1)C1CCC(F)(F)CC1)c1nonc1C1CC1]

### Example 101: 4-Cyclopropyl-N—[(S)-(4,4-difluorocyclohexyl)-[7-[[(3R*,4R*)-2-oxo-4-(trifluoromethyl)pyrrolidin-3-yl]methyl]imidazo[1,2-b]pyridazin-2-yl]methyl]-1,2,5-oxadiazole-3-carboxamide

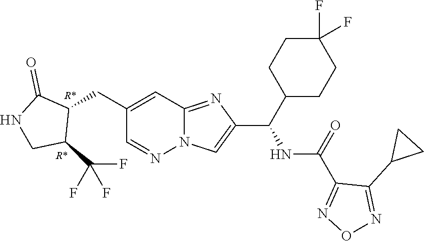 [SMILES: O=C(N[C@H](c1cn2ncc(C[C@H]3C(=O)NC[C@@H]3C(F)(F)F)cc2n1)C1CCC(F)(F)CC1)c1nonc1C1CC1]

The title compounds were synthesized in a manner analogous to Example 21 using 3-((2-((S)-amino(4,4-difluorocyclohexyl)methyl)imidazo[1,2-b]pyridazin-7-yl)methyl)-4-(trifluoromethyl)pyrrolidin-2-one (Intermediate 56 Step B) in place of 3-((2-((S)-amino(4,4-difluorocyclohexyl)methyl)imidazo[1,2-b]pyridazin-7-yl)methyl)pyrrolidin-2-one. Purification was performed via silica gel chromatography (0-100% acetone/hexanes (0.1% TEA)) to provide a mixture of diastereomers. The diastereomers were separated by chiral SFC (Stationary phase: Chiralpak AS-H, 5 μm, 250×30 mm, Mobile phase: 25% methanol, 75% CO₂) to afford Example 100 as the first-eluting isomer (14% yield) and Example 101 as the second-eluting isomer (11% yield). Example 100: ¹H NMR (400 MHz, DMSO-d₆) δ 9.48 (d, J=9.0 Hz, 1H), 8.45 (d, J=2.0 Hz, 1H), 8.21 (s, 1H), 8.05 (s, 1H), 7.94 (s, 1H), 5.19 (t, J=8.5 Hz, 1H), 3.47-3.39 (m, 1H), 3.24-3.18 (m, 2H), 3.11-3.04 (m, 1H), 3.01-2.92 (m, 2H), 2.32-2.24 (m, 1H), 2.24-2.14 (m, 1H), 2.12-1.95 (m, 2H), 1.95-1.70 (m, 3H), 1.68-1.59 (m, 1H), 1.46-1.23 (m, 2H), 1.16-1.09 (m, 2H), 1.01-0.94 (m, 2H). MS (ESI) m/z: [M+H]⁺ Found 568.2. Example 101: ¹H NMR (400 MHz, DMSO-d₆) δ 9.48 (d, J=9.0 Hz, 1H), 8.46 (d, J=1.9 Hz, 1H), 8.21 (s, 1H), 8.05 (s, 1H), 7.97-7.92 (m, 1H), 5.20 (s, 1H), 3.43 (s, 1H), 3.24-3.18 (m, 2H), 3.12-3.04 (m, 1H), 3.02-2.91 (m, 2H), 2.32-2.24 (m, 1H), 2.24-2.15 (m, 1H), 2.08-1.96 (m, 2H), 1.94-1.70 (m, 3H), 1.68-1.60 (m, 1H), 1.48-1.25 (m, 2H), 1.16-1.08 (m, 2H), 0.96 (m, 2H). MS (ESI) m/z: [M+H]⁺ Found 568.2.

### Example 102: 4-Cyclopropyl-N—[(S)-(4,4-difluorocyclohexyl)-[7-[(3RS,5S)-3-methyl-2-oxo-5-(trifluoromethyl)pyrrolidine-3-carbonyl]imidazo[1,2-b]pyridazin-2-yl]methyl]-1,2,5-oxadiazole-3-carboxamide

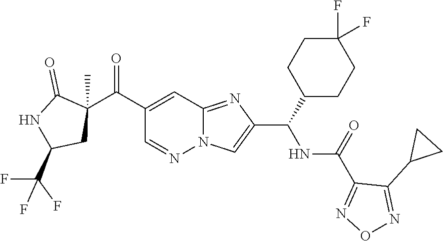 [SMILES: C[C@@]1(C(=O)c2cnn3cc([C@@H](NC(=O)c4nonc4C4CC4)C4CCC(F)(F)CC4)nc3c2)C[C@@H](C(F)(F)F)NC1=O]

The title compound was synthesized in a manner analogous to Example 21 using (3R,5S)-3-(2-((S)-amino(4,4-difluorocyclohexyl)methyl)imidazo[1,2-b]pyridazine-7-carbonyl)-3-methyl-5-(trifluoromethyl)pyrrolidin-2-one (Intermediate 61) in place of 3-((2-((S)-amino(4,4-difluorocyclohexyl)methyl)imidazo[1,2-b]pyridazin-7-yl)methyl)pyrrolidin-2-one. Purification was performed via preparative basic HPLC (X-Bridge Prep C18 column, 5 μm, 50×250 mm, 5-100% acetonitrile/water (with 20 mM NH₄OH)) to afford the title compound as a 3:1 mixture of diastereomers wherein the major product was the title compound. (34% yield, absolute stereochemistry was determined by 2D NMR). ¹H NMR (500 MHz, DMSO-d₆) δ 9.61-9.58 (m, 1H), 9.22 (s, 1H), 8.81-8.80 (m, 1N), 8.66-8.64 (m, 1H), 8.51-8.49 (m, 1H), 5.30-5.23 (m, 1H), 2.89-2.82 (m, 1H), 2.39-2.34 (m, 1H), 2.32-2.26 (m, 1H), 2.25-2.19 (m, 1H), 2.07-1.98 (m, 2H), 1.93-1.89 (m, 1H), 1.85-1.75 (m, 2H), 1.67-1.62 (m, 1H), 1.56-1.53 (m, 3H), 1.43-1.38 (m, 1H), 1.34-1.28 (m, 1H), 1.13-1.12 (m, 1H), 1.11-1.10 (m, 1H), 0.99-0.95 (m, 3H). MS (ESI) m/z: [M+H]⁺ Found 596.2.

### Example 103: N—((S)-(7-(((3S*,5S)-3-(Cyanomethyl)-2-oxo-5-(trifluoromethyl)pyrrolidin-3-yl)methyl)imidazo[1,2-b]pyridazin-2-yl)(4,4-difluorocyclohexyl)methyl)-4-methyl-1,2,5-oxadiazole-3-carboxamide

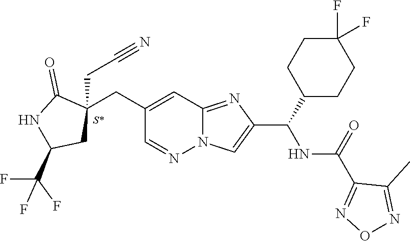 [SMILES: Cc1nonc1C(=O)N[C@H](c1cn2ncc(C[C@]3(CC#N)C[C@@H](C(F)(F)F)NC3=O)cc2n1)C1CCC(F)(F)CC1]

The title compound was synthesized in a manner analogous to Example 21 using (1S)-(7-(((5S)-3-(cyanomethyl)-2-oxo-5-(trifluoromethyl)pyrrolidin-3-yl)methyl)imidazo[1,2-b]pyridazin-2-yl)(4,4-difluorocyclohexyl)methanaminium 2,2,2-trifluoroacetate (Intermediate 62) in place of 3-((2-((S)-amino(4,4-difluorocyclohexyl)methyl)imidazo[1,2-b]pyridazin-7-yl)methyl)pyrrolidin-2-one and 2,5-dioxopyrrolidin-1-yl 4-methyl-1,2,5-oxadiazole-3-carboxylate in place of 2,5-dioxopyrrolidin-1-yl 4-cyclopropyl-1,2,5-oxadiazole-3-carboxylate. Purification was performed via preparative basic HPLC (X-Bridge C18 column, 5 μm, 50×250 mm, 5-100% acetonitrile/water (with 20 mM NH₄OH)) to provide the title compound (71% yield). ¹H NMR (500 MHz, DMSO-d₆) δ9.45-9.41 (m, 1H), 9.04 (br s, 1H), 8.33 (d, J=2.0 Hz, 1H), 8.24 (s, 1H), 7.93-7.88 (m, 1H), 5.21-5.16 (m, 1H), 4.39-4.30 (m, 1H), 3.08-2.99 (m, 1H), 2.97-2.86 (m, 3H), 2.46 (s, 3H), 2.36-2.31 (m, 1H), 2.22-2.16 (m, 1H), 2.15-2.09 (m, 1H), 2.08-1.96 (m, 2H), 1.92-1.86 (m, 1H), 1.85-1.70 (m, 2H), 1.64-1.55 (m, 1H), 1.45-1.33 (m, 1H), 1.33-1.20 (m, 1H). MS (ESI) m/z: [M+H]⁺ Found 581.3.

### Example 104: N—((S)-(7-(((3S*,5S)-3-(Cyanomethyl)-2-oxo-5-(trifluoromethyl)pyrrolidin-3-yl)methyl)imidazo[1,2-b]pyridazin-2-yl)(4,4-difluorocyclohexyl)methyl)-4-cyclopropyl-1,2,5-oxadiazole-3-carboxamide

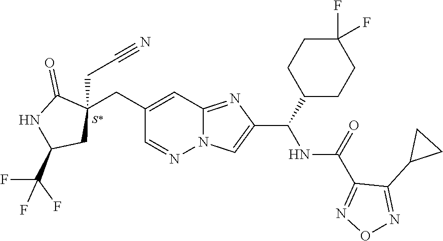 [SMILES: N#CC[C@@]1(Cc2cnn3cc([C@@H](NC(=O)c4nonc4C4CC4)C4CCC(F)(F)CC4)nc3c2)C[C@@H](C(F)(F)F)NC1=O]

The title compound was synthesized in a manner analogous to Example 21 using (1S)-(7-(((5S)-3-(cyanomethyl)-2-oxo-5-(trifluoromethyl)pyrrolidin-3-yl)methyl)imidazo[1,2-b]pyridazin-2-yl)(4,4-difluorocyclohexyl)methanaminium 2,2,2-trifluoroacetate (Intermediate 62) in place of 3-((2-((S)-amino(4,4-difluorocyclohexyl)methyl)imidazo[1,2-b]pyridazin-7-yl)methyl)pyrrolidin-2-one. Purification was performed via preparative basic HPLC (X-Bridge Prep C18 column, 5 μm, 50×250 mm, 5-100% acetonitrile/water (with 20 mM NH₄OH)). Chiral separation was performed via SFC (Stationary phase: Chiralcel OD-H, 5 μm, 250×21 mm, Mobile phase: 20% methanol:isopropanol (1:1) (with 0.2% isopropylamine), 80% CO₂) to afford the title compound (39% yield). ¹H NMR (500 MHz, DMSO-d₆) δ 9.49 (d, J=9.0 Hz, 1H), 9.03 (s, 1H), 8.34 (d, J=2.1 Hz, 1H), 8.23 (s, 1H), 7.90 (d, J=2.0 Hz, 1H), 5.22-5.19 (m, 8.0 Hz, 1H), 4.63 (d, J=3.5 Hz, 1H), 4.41-4.29 (m, 1H), 3.09-3.00 (m, 1H), 2.97-2.92 (m, 1H), 2.91 (d, J=6.7 Hz, 2H), 2.33 (m, 1H), 2.27 (m, 1H), 2.23-2.14 (m, 1H), 2.13-2.09 (m, 1H), 2.08-1.92 (m, 1H), 1.93-1.70 (m, 3H), 1.64-1.55 (m, 1H), 1.42-1.35 (m, 1H), 1.33-1.22 (m, 1H), 1.13-1.07 (m, 2H), 0.97-0.93 (m, 2H). MS (ESI) m/z: [M+H]⁺ Found 607.0.

### Example 105: N—((S)-(4,4-Difluorocyclohexyl)(7-(((3S*,5S)-3-((dimethylamino)methyl)-2-oxo-5-(trifluoromethyl)pyrrolidin-3-yl)methyl)imidazo[1,2-b]pyridazin-2-yl)methyl)-4-methyl-1,2,5-oxadiazole-3-carboxamide

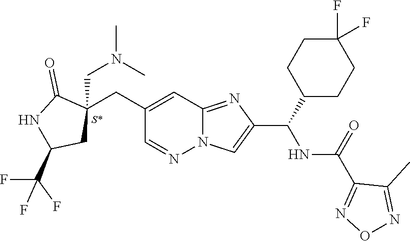 [SMILES: Cc1nonc1C(=O)N[C@H](c1cn2ncc(C[C@]3(CN(C)C)C[C@@H](C(F)(F)F)NC3=O)cc2n1)C1CCC(F)(F)CC1]

Step A: tert-Butyl (5S)-3-((2-((S)-((tert-butoxycarbonyl)amino(4,4-difluorocyclohexyl)methyl)imidazo[1,2-b]pyridazin-7-yl)methyl)-3-((dimethylamino)methyl)-2-oxo-5-(trifluoromethyl)pyrrolidine-1-carboxylate. The title compound was synthesized in a manner analogous to Intermediate 55 Step A using Eschenmoser's salt in place of MOMCl. The residue was purified twice by silica gel chromatography (0-100% EtOAc/hexanes), followed by (0-20% MeOH/EtOAc) to afford the title compound (87% yield).

Step B: (1S)-(4,4-Difluorocyclohexyl)(7-(((5S)-3-((dimethylamino)methyl)-2-oxo-5-(trifluoromethyl)pyrrolidin-3-yl)methyl)imidazo[1,2-b]pyridazin-2-yl)methanaminium 2,2,2-trifluoroacetate. The title compound was synthesized in a manner analogous to Intermediate 40 Step B using tert-butyl (5S)-3-((2-((S)-((tert-butoxycarbonyl)amino)(4,4-difluorocyclohexyl)methyl)imidazo[1,2-b]pyridazin-7-yl)methyl)-3-((dimethylamino)methyl)-2-oxo-5-(trifluoromethyl)pyrrolidine-1-carboxylate (Step A) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The reaction mixture was concentrated to dryness, (not passed through a silica/carbonate plug) and was used as the TFA salt without further purification.

Step C: N—((S)-(4,4-Difluorocyclohexyl)(7-(((3S*,5S)-3-((dimethylamino)methyl)-2-oxo-5-(trifluoromethyl)pyrrolidin-3-yl)methyl)imidazo[1,2-b]pyridazin-2-yl)methyl)-4-methyl-1,2,5-oxadiazole-3-carboxamide. The title compound was synthesized in a manner analogous to Example 21 using (1S)-(4,4-difluorocyclohexyl)(7-(((5S)-3-((dimethylamino)methyl)-2-oxo-5-(trifluoromethyl)pyrrolidin-3-yl)methyl)imidazo[1,2-b]pyridazin-2-yl)methanaminium 2,2,2-trifluoroacetate (Step B) in place of 3-((2-((S)-amino(4,4-difluorocyclohexyl)methyl)imidazo[1,2-b]pyridazin-7-yl)methyl)pyrrolidin-2-one and 2,5-dioxopyrrolidin-1-yl 4-methyl-1,2,5-oxadiazole-3-carboxylate in place of 2,5-dioxopyrrolidin-1-yl 4-cyclopropyl-1,2,5-oxadiazole-3-carboxylate. Purification was performed via preparative basic HPLC (X-Bridge Prep C18 column, 5 μm, 50×250 mm, 5-100% acetonitrile/water (with 20 mM NH₄OH)) to afford the title compound (29% yield, 20:1 dr). ¹H NMR (500 MHz, DMSO-d₆) δ 9.41 (d, J=9.0 Hz, 1H), 8.71 (s, 1H), 8.29 (d, J=2.0 Hz, 1H), 8.21 (s, 1H), 7.83 (d, J=1.6 Hz, 1H), 5.18 (t, J=8.6 Hz, 1H), 4.18 (br d, J=7.4 Hz, 1H), 2.87 (d, J=13.6 Hz, 1H), 2.69 (d, J=13.6 Hz, 1H), 2.55-2.51 (m, 1H), 2.46-2.45 (m, 3H), 2.45-2.40 (m, 2H), 2.23 (s, 6H), 2.21-2.15 (m, 1H), 2.08-1.95 (m, 2H), 1.94-1.85 (m, 2H), 1.85-1.70 (m, 2H), 1.64-1.57 (m, 1H), 1.44-1.33 (m, 1H), 1.32-1.21 (m, 1H). MS (ESI) m/z: [M+H]⁺ Found 599.2.

### Example 106: N—((S)-(4,4-Difluorocyclohexyl)(7-((R)-1-hydroxy-1-((3R,5S)-3-methyl-2-oxo-5-(trifluoromethyl)pyrrolidin-3-yl)ethyl)imidazo[1,2-b]pyridazin-2-yl)methyl)-4-methyl-1,2,5-oxadiazole-3-carboxamide

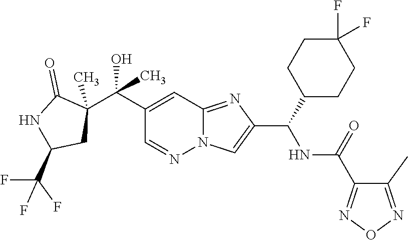 [SMILES: Cc1nonc1C(=O)N[C@H](c1cn2ncc([C@@](C)(O)[C@]3(C)C[C@@H](C(F)(F)F)NC3=O)cc2n1)C1CCC(F)(F)CC1]

Step A: tert-Butyl (3R,5S)-3-(1-(2-((S)-((tert-butoxycarbonyl)amino(4,4-difluorocyclohexyl)methyl)imidazo[1,2-b]pyridazin-7-yl)-1-hydroxyethyl)-3-methyl-2-oxo-5-(trifluoromethyl)pyrrolidine-1-carboxylate. A solution of tert-butyl (3R,5S)-3-(2-((S)-((tert-butoxycarbonyl)amino)(4,4-difluorocyclohexyl)methyl)imidazo[1,2-b]pyridazine-7-carbonyl)-3-methyl-2-oxo-5-(trifluoromethyl)pyrrolidine-1-carboxylate (50 mg, 0.07 mmol, Intermediate 61 Step A) in THF (1 mL) was cooled to 0° C. Then, methyl magnesium bromide (0.1 mL, 0.3 mmol, 3M in diethyl ether) was added dropwise. The resulting mixture was stirred at 0° C. for 15 min, quenched with saturated aqueous NH₄C1, diluted with EtOAc and the layers were separated. The aqueous layer was further extracted with EtOAc (3×). The combined organic layers were washed with brine, dried over anhydrous Na₂SO₄, filtered, concentrated to dryness and used without further purification.

Step B: (3R,5S)-3-(1-(2-((S)-Amino(4,4-difluorocyclohexyl)methyl)imidazo[1,2-b]pyridazin-7-yl)-1-hydroxyethyl)-3-methyl-5-(trifluoromethyl)pyrrolidin-2-one. The title compound, as its TFA salt, was synthesized in a manner analogous to Intermediate 40 Step B using tert-butyl (3R,5S)-3-(1-(2-((S)-((tert-butoxycarbonyl)amino)(4,4-difluorocyclohexyl)methyl)imidazo[1,2-b]pyridazin-7-yl)-1-hydroxyethyl)-3-methyl-2-oxo-5-(trifluoromethyl)pyrrolidine-1-carboxylate (Step A) in place of tert-butyl (5S)-3-((2-((S)-((tert-butoxycarbonyl)amino)(4,4-difluorocyclohexyl)methyl)imidazo[1,2-b]pyridazin-7-yl)methyl)-2-oxo-5-(trifluoromethyl)pyrrolidine-1-carboxylate. The reaction mixture was concentrated to dryness, (not passed through a silica/carbonate plug) and was used as the TFA salt without further purification.

Step C: N—((S)-(4,4-Difluorocyclohexyl)(7-((R)-1-hydroxy-1-((3R,5S)-3-methyl-2-oxo-5-(trifluoromethyl)pyrrolidin-3-yl)ethyl)imidazo[1,2-b]pyridazin-2-yl)methyl)-4-methyl-1,2,5-oxadiazole-3-carboxamide. The title compound was synthesized in a manner analogous to Example 21 using (3R,5S)-3-(1-(2-((S)-amino(4,4-difluorocyclohexyl)methyl)imidazo[1,2-b]pyridazin-7-yl)-1-hydroxyethyl)-3-methyl-5-(trifluoromethyl)pyrrolidin-2-one (Step B) in place of 3-((2-((S)-amino(4,4-difluorocyclohexyl)methyl)imidazo[1,2-b]pyridazin-7-yl)methyl)pyrrolidin-2-one and 2,5-dioxopyrrolidin-1-yl 4-methyl-1,2,5-oxadiazole-3-carboxylate. Purification was performed twice via silica gel chromatography (0-100% EtOAc/hexanes), then (0-20% MeOH/EtOAc). The diastereomers were separated by chiral SFC (Diacel CHIRALPAK IH, 20×250 mm, 20% EtOH (0.1% DEA), 80% CO₂) to afford the title compound as the first-eluting isomer (17% yield, absolute stereochemistry was determined by 2D NMR). ¹H NMR (500 MHz, DMSO-d₆) δ 9.41-9.37 (m, 1H), 8.55-8.53 (m, 1H), 8.52-8.50 (m, 1H), 8.21-8.18 (m, 1H), 7.95-7.93 (m, 1H), 5.21-5.13 (m, 1H), 2.46-2.45 (m, 3H), 2.29-2.24 (m, 1H), 2.22-2.15 (m, 1H), 2.07-2.00 (m, 1H), 1.99-1.95 (m, 1H), 1.94-1.86 (m, 2H), 1.83-1.73 (m, 2H), 1.66-1.64 (m, 3H), 1.64-1.57 (m, 2H), 1.23-1.22 (m, 3H), 1.21-1.20 (m, 3H). MS (ESI) m/z: [M+H]⁺ Found 586.0.

### In Vitro Biological Data

IL-17A(FLAG-tagged): IL-17RA(His-tagged) binding disruption Eu-HTRF assay An antibody directed against the FLAG tag of IL-17A (SEQ ID NO: 1) is labeled with the HTRF donor chromophore (Europium-cryptate). IL-17A is present as a dimer that is “locked into” this quaternary structure due to the formation of loop-spanning intramolecular disulfide bridges. The construct of IL-17RA used in the assay excludes the outer-membrane portion of the receptor and is fused to a C-terminal 10×His tag (SEQ ID NO:2). An antibody directed against the His tag of the IL-17RA chimera is labeled with the HTRF acceptor chromophore (“D2”). The fluorescence-resonance energy transfer (FRET) depends on the vicinity of the donor chromophore to the acceptor, and interruption of the binding between the IL-17A and IL-17RA causes the reduction/loss of FRET. Therefore, this assay allows to evaluate the compound effect on the binding IL-17A and IL-17RA by monitoring the fluorescence intensity of donor vs acceptor. The assay is run as described below.

40 nl of 2-fold serial diluted compound solution for total 22 dilution points is added into each well of a 1536-well, white, low-volume, non-binding plate (Greiner #782904), then 2 μl of FLAG tagged IL-17A at 2× final concentration (1 μM) in solution of PBS+0.01% Triton-X100 is added to each well. The assay plate is briefly centrifuged then incubated for 1 h at rt. A mixed solution is prepared containing 2×5 nM 10HIS×IL-17RA, 2×2.5 nM Eu-anti-FLAG (CISBIO), 2×5 nM D2-anti-HIS (CISBIO) in PBS+0.01% Triton-X100+200 mM Potassium Fluoride (Sigma 60238) and 2 μl of mix is added to each well of the assay plate. The plate is briefly centrifuged then incubated for 2 h at rt. The HTRF intensities at the wavelength of donor (615 nm) and acceptor (665 nm) are measured using BMG Pherastar. The ratio between intensities at two wavelengths is calculated and plotted against the compound concentration and the data is fitted to a one-site competition model to yield IC₅₀ of the compound.

IL-17A acts directly on keratinocytes through binding to dimeric receptor IL-17RA/RC and drives the production of a number of inflammatory mediators known to be elevated in psoriasis lesional tissue. IL-17A small molecule inhibitors that block the IL-17A to interact with IL-17R would inhibit the IL-17A signaling in its targeted cells such as keratinocytes. The compound functional activity is evaluated for its impact on IL-17A-induced G-CSF production in human normal keratinocyte (NHK).

### NHK Assay

Adult normal human keratinocytes are cultured in keratinocyte growth medium (Lonza) in a flask till reaching ~90% confluence, then cells are transferred to a 384-well plate at density of 3000-4000 cell/well. Recombinant human IL-17A (Gibco PHC9174) is pre-incubated with titrated compound or DMSO for 1 h at rt then added to the cell culture plate. The final concentration of IL-17A is 5 ng/mL and DMSO is 0.2%, in the culture containing 5% FBS. Cells are cultured/treated for 24 h at 37° C. Supernatants are collected and G-CSF production is measured through HTRF technology using Human G-CSF Kit (CisBio). G-CSF concentration was extrapolated from the standard curve and IC₅₀ is determined using GraphPad Prism. Cell viability is also evaluated using CellTiter-Glo kit (Promega) and effect of compound on cell viability is compared to DMSO control.

In cases where the compound was tested more than once, the IC₅₀ value was calculated as a simple average of the measured values. The average was then used to categorize the compound within one of the following ranges.
A: IC₅₀<0.4 μM;
B: 0.4 μM≤IC₅₀≤4 μM;
C: IC₅₀>4 μM
   Not available

TABLE 3

| Example | HTRF IC₅₀ | NHK IC₅₀ |
|---|---|---|
| 1 | A | A |
| 2 | B | C |
| 3 | — | C |
| 4 | — | B |
| 5 | C | C |
| 6 | A | A |
| 7 | A | A |
| 8 | C | C |
| 9 | A | A |
| 10 | C | C |
| 11 | A | A |
| 12 | C | B |
| 13 | A | B |
| 14 | C | C |
| 15 | C | C |
| 16 | B | C |
| 17 | B | C |
| 18 | B | B |
| 19 | A | A |
| 20 | B | B |
| 21 | B | C |
| 22 | C | C |
| 23 | B | — |
| 24 | B | — |
| 25 | B | C |
| 26 | B | C |
| 27 | B | C |
| 28 | A | B |
| 29 | — | B |
| 30 | — | C |
| 31 | — | A |
| 32 | — | C |
| 33 | — | B |
| 34 | — | C |
| 35 | — | B |
| 36 | A | A |
| 37 | — | A |
| 38 | A | B |
| 39 | C | C |
| 40 | C | C |
| 41 | C | C |
| 42 | A | A |
| 43 | B | C |
| 44 | B | C |
| 45 | B | C |
| 46 | C | C |
| 47 | A | B |
| 48 | C | C |
| 49 | B | C |
| 50 | B | C |
| 51 | A | A |
| 52 | B | B |
| 53 | B | B |
| 54 | B | C |
| 55 | — | A |
| 56 | B | B |
| 57 | — | A |
| 58 | A | B |
| 59 | A | A |
| 60 | B | C |
| 61 | B | A |
| 62 | B | C |
| 63 | B | C |
| 64 | B | A |
| 65 | A | A |
| 66 | — | B |
| 67 | — | C |
| 68 | B | B |
| 69 | B | B |
| 70 | A | A |
| 71 | B | C |
| 72 | B | B |
| 73 | B | B |
| 74 | A | A |
| 75 | A | A |
| 76 | C | B |
| 77 | C | B |
| 78 | C | C |
| 79 | A | A |
| 80 | B | B |
| 81 | A | A |
| 82 | B | C |
| 83 | A | A |
| 84 | — | C |
| 85 | B | C |
| 86 | B | C |
| 87 | — | C |
| 88 | B | C |
| 89 | A | C |
| 90 | — | A |
| 91 | B | — |
| 92 | C | — |
| 93 | B | B |
| 94 | A | A |
| 95 | B | C |
| 96 | C | C |
| 97 | A | A |
| 98 | C | C |
| 99 | — | A |
| 100 | B | C |
| 101 | B | B |
| 102 | A | A |
| 103 | A | A |
| 104 | A | A |
| 105 | A | A |
| 106 | — | A |

TABLE 4

| | cis/trans-isomer regarding | | |
|---|---|---|---|
| | the substituents on the | | |
| Example | C-3 and C-5 positions | HTRF IC₅₀ | NHK IC₅₀ |
| 51 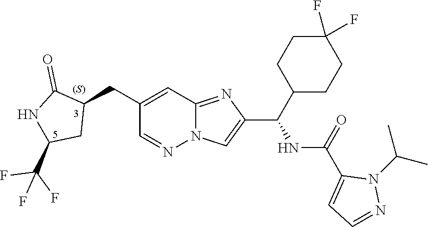 | cis-isomer | A (0.15 μM) | A (0.05 μM) |
| 52 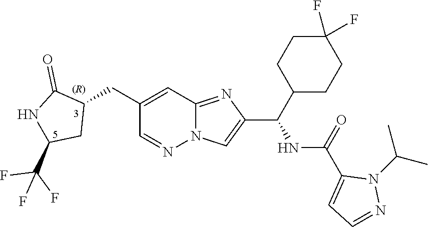 | trans-isomer | B (2.45 μM) | B (2.83 μM) |
| 59 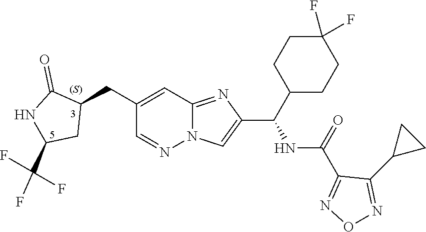 | cis-isomer | A (0.014 μM) | A (0.0078 μM) |
| 60 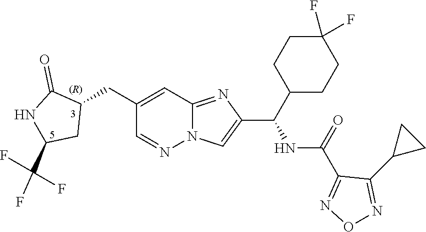 | trans-isomer | B (1.14 μM) | C (>4 μM) |

As shown in Table 4, unexpectedly and surprisingly, Example 51, with an (S)-configuration on the C-3 position, showed a more than 100-fold potency increase in both the HTRF IC₅₀ and NHK IC₅₀ when compared to its C-3 epimer, Example 52 with an (R)-configuration. The same trend was observed when comparing another pair of epimers, Example 59 and Example 60, where Example 59, the epimer with an (S)-configuration on the C-3 position showed a more than 80-fold potency increase in both the HTRF ICs and NHK IC₅₀ when compared to its C-3 epimer, Example 60, with a (R)-configuration. These unexpected and surprising results demonstrated the significantly higher potency of the compounds of Formula Ig-2:

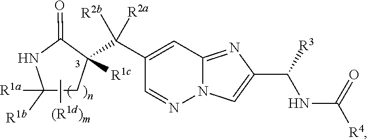

as described and provided here, with an absolute configuration as indicated herein for the carbon next to the carbonyl of the lactam ring (e.g., the respective C-3 carbon of Examples 51 and 59). Additionally, Example 51 and Example 59, the cis-isomers, showed an unexpected and surprising potency increase of more than 100 fold and more than 80 fold, respectively, when compared to the corresponding trans-isomers, Example 52 and Example 60. These unexpected and surprising results demonstrated the significantly higher potency of the compounds of Formula Ib-8:

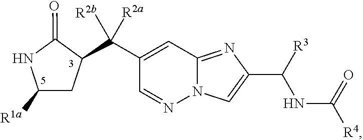

as described and provided here, containing a cis-configuration regarding the substituents on the respective C-3 and C-5 positions of the lactam ring.

While the foregoing specification teaches the principles of the present disclosure, with examples provided for the purpose of illustration, it will be understood that the practice of the compounds and methods disclosed herein encompasses all of the usual variations, adaptations and/or modifications as come within the scope of the following claims and their equivalents.

All documents cited herein are incorporated by reference.

Name: IL-17A-Flag
SEQ ID NO: 1
MATGSRTSLLLAFGLLCLPWLQEGSAGSDYKDDDDKGSGSGSLEVLFQGP
GITIPRNPGCPNSEDKNFPRTVMVNLNIHNRNTNTNPKRSSDYYNRSTSP
WNLHRNEDPERYPSVIWEAQCRHLGCINADGNVDYHMNSVPIQQEILVLR
REPPHCPNSFRLEKILVSVGCTCVTPIVHHVQ
Name: IL-17RA
SEQ ID NO: 2
MKFLVNVALVEMVVYISYIYALRLLDHRALVCSQPGLNCTVKNSTCLDDS
WIHPRNLTPSSPKDLQIQLHFAHTQOGDLFPVAHIEWTLOTDASILYLEG
AELSVLQLNTNERLCVRFEFLSKLRHHHRRWRFTFSHFVVDPDQEYEVTV
HHLPKPIPDGDPNHQSKNFLVPDCEHARMKVTTPCMSSGSLWDPNITVET
LEAHQLRVSFTLWNESTHYQILLTSFPHMENHSCFEHMHHIPAPRPEEFH
QRSNVTLTLRNLKGCCRHQVQIQPFFSSCLNDCLRHSATVSCPEMPDTPE
PIPDYMPLWGSGGHHHHHHHHHH*

1. A compound of Formula I 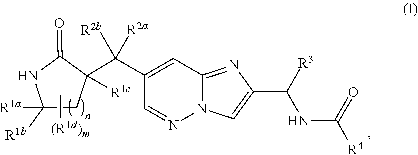
or a pharmaceutically acceptable salt thereof,
wherein:
R^{1a} and R^{1b} are each independently H, —C₍₁₋₃₎alkyl, —C₍₃₋₅₎cycloalkyl, —C₍₁₋₃₎alkyl-O—C₍₁₋₃₎alkyl or —C₍₁₋₃₎alkyl-N(C₍₁₋₃₎alkyl)₂, wherein the —C₍₁₋₃₎alkyl is unsubstituted or substituted with one to six R^{1aa} groups; or R^{1a} and R^{1b} together with the carbon atom to which the are attached form a spiro C₍₃₋₅₎cycloalkyl or a spiro heterocyclyl selected from 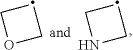
wherein · indicates the carbon atom to which R^{1a} and R^{1b} are attached; or R^{1a} and R^{1c} are taken together to form a C₍₁₋₂₎alkylenyl bridge;
R^{1c} is H, halo, —OH, —C₍₁₋₃₎alkyl, —C₍₁₋₃₎alkyl-CN, —C₍₃₋₅₎cycloalkyl, —C₍₁₋₃₎alkyl-O—C₍₁₋₃₎alkyl, or —C₍₁₋₃₎alkyl-N(C₍₁₋₃₎alkyl)₂, wherein the —C₍₁₋₃₎alkyl is unsubstituted or substituted with one to six R^{1aa} groups;
R^{1d} is independently for each occurrence —OH, halo, —C₍₁₋₃₎alkyl, —C₍₃₋₅₎cycloalkyl, —C₍₁₋₃₎alkyl-O—C₍₁₋₃₎alkyl or —C₍₁₋₃₎alkyl-N—(C₍₁₋₃₎alkyl)₂ wherein the —C₍₁₋₃₎alkyl is unsubstituted or substituted with one to six R^{1aa} groups;
R^{1aa} independently for each occurrence is halo;
R^{2a} is H, —OH, —C₍₁₋₃₎alkyl, —C₍₃₋₅₎cycloalkyl, —C₍₁₋₃₎alkyl-O—C₍₁₋₃₎alkyl, —C₍₁₋₃₎alkyl-O—C₍₃₋₅₎ cycloalkyl, or 4- to 6-membered heterocyclyl, wherein the —C₍₁₋₃₎alkyl, —C₍₃₋₅₎cycloalkyl, —C₍₁₋₃₎alkyl-O—C₍₁₋₃₎alkyl, and —C₍₁₋₃₎alkyl-O—C₍₃₋₅₎cycloalkyl groups are unsubstituted or substituted with one to six R^{2aa} groups; or R^{2a} and R^{1c} are taken together with the carbon atoms to which they are attached to form a C₍₃₋₅₎cycloalkyl or a 4- to 6-membered heterocyclyl;
R^{2aa} independently for each occurrence is halo, —OH, or —CN;
R^{2b} is H or —C₍₁₋₃₎alkyl; or R^{2a} and R^{2b} are taken together with the carbon atom to which they are attached to form a C═O;
R³ is —C₍₁₋₁₀₎alkyl, —C₍₁₋₆₎alkyl-O—C₍₁₋₆₎alkyl, —C₍₁₋₆₎alkyl-O—C₍₃₋₅₎cycloalkyl, —C₍₁₋₆₎alkyl-O—C₍₃₋₅₎cycloalkyl-C₍₁₋₃₎alkyl, each of which is unsubstituted or substituted with one to six halo atoms;
R⁴ is a 5-membered heteroaryl that is unsubstituted or substituted with one to two R^{4a} groups;
R^{4a} is halo, —C₍₁₋₆₎alkyl, —O—C₍₁₋₆₎alkyl, —C₍₁₋₆₎alkyl-O—C₍₁₋₆₎alkyl, or —C₍₀₋₂₎alkyl-C₍₃₋₆₎cycloalkyl, wherein the —C₍₁₋₆₎alkyl, —O—C₍₁₋₆₎alkyl, —C₍₁₋₆₎alkyl-O—C₍₁₋₆₎alkyl, and —C₍₀₋₂₎alkyl-C₍₃₋₆₎cycloalkyl are unsubstituted or substituted with one to six substituents independently selected from halo, —CH₃, —CH₂F, —CHF₂, and —CF₃;
n is 1, 2 or 3; and
m is 0, 1, 2, 3, or 4.

2. The compound of claim 1, or a pharmaceutically acceptable salt thereof, wherein the compound has a formula of Formula Ig-1:

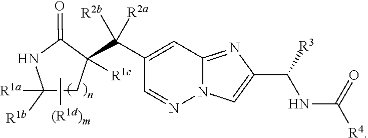

3-6. (canceled)

7. The compound of claim 1, or a pharmaceutically acceptable salt thereof, wherein:
R^{1a} is —C₍₁₋₃₎alkyl, wherein the —C₍₁₋₃₎alkyl is unsubstituted or substituted with one to three R^{1aa} groups;
R^{1b} is H or —C₍₁₋₃₎alkyl, wherein the —C₍₁₋₃₎alkyl is unsubstituted or substituted with one to three R^{1aa} groups;
R^{1c} isH, halo, —OH, —C₍₁₋₃₎alkyl, —C₍₁₋₃₎alkyl-CN, —C₍₁₋₃₎alkyl-O—C₍₁₋₃₎alkyl, or —C₍₁₋₃₎alkyl-N(C₁₋₃₎alkyl)₂;
R^{1aa} independently for each occurrence is fluorine;
R^{2a} is H, or —OH;
R^{2b} is H;
R³ is —C₍₄₋₈₎alkyl substituted with one to three fluorine atoms or 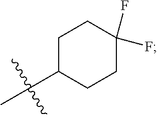 [SMILES: *C1CCC(F)(F)CC1]
R⁴ is 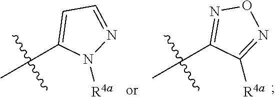
R^{4a} is —C₍₁₋₆₎alkyl, —O—C₍₁₋₆₎alkyl, or —C₍₃₋₆₎cycloalkyl;
n is 1; and
m is 0.

8. (canceled)

9. The compound of claim 1, or a pharmaceutically acceptable salt thereof, having a formula selected from the group consisting of Formulas Ib-1 to Ib-7:

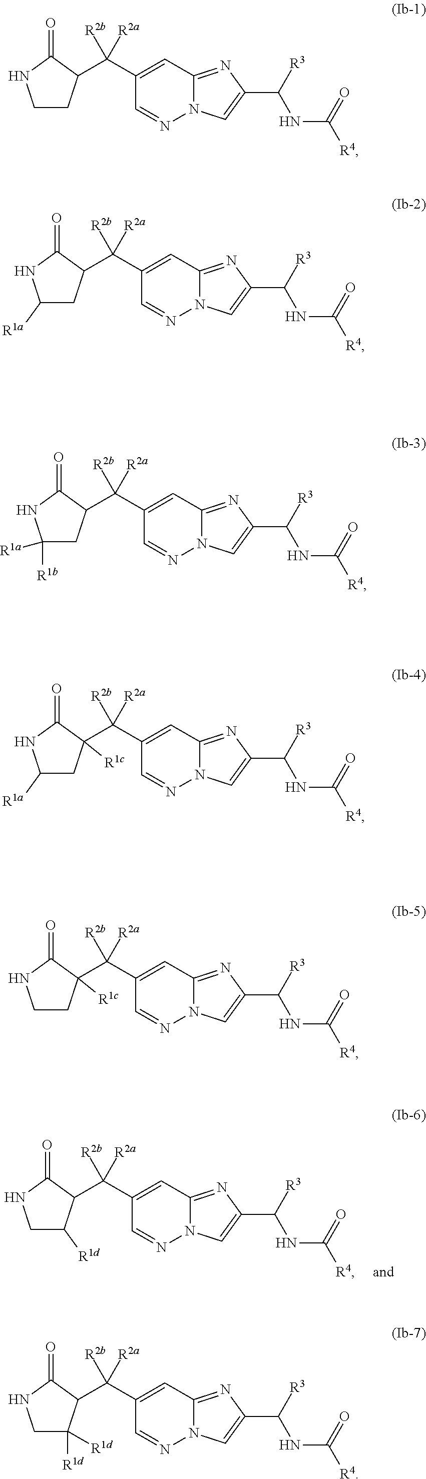

10. The compound of claim 1, or a pharmaceutically acceptable salt thereof, having a formula selected from the group consisting of Formulas Ib-1a to Ib-7a:

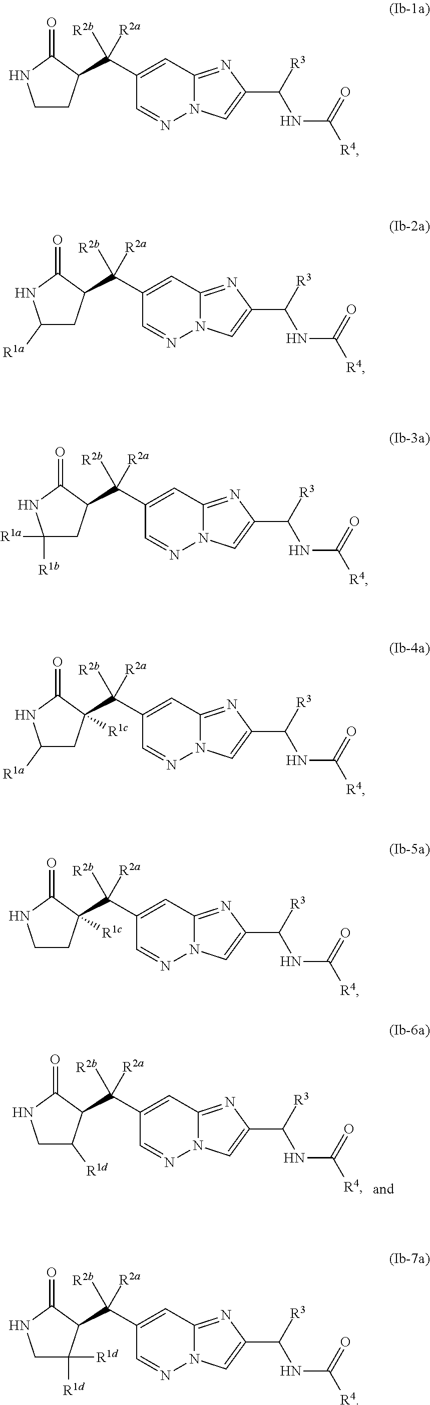

11. The compound of claim 1, or a pharmaceutically acceptable salt thereof, having a formula of Formula Ib-8:

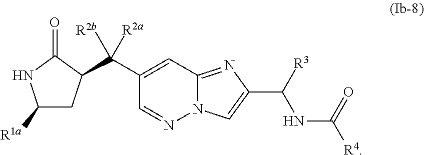

12. (canceled)

13. The compound of claim 1, or a pharmaceutically acceptable salt thereof, having a formula selected from the group consisting of Formulas Ic-1 to Ic-7:

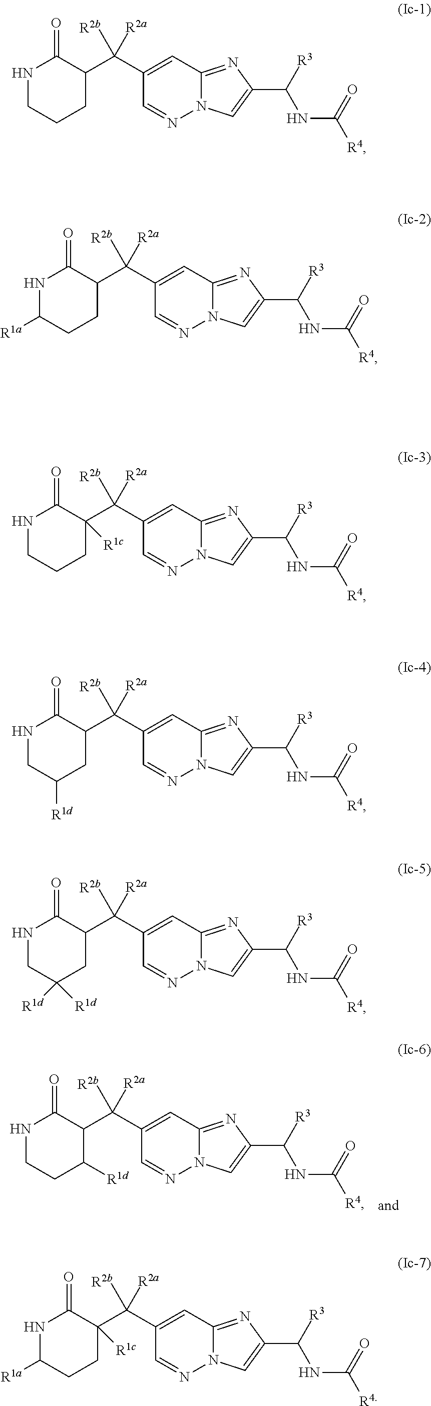

14. The compound of claim 1, or a pharmaceutically acceptable salt thereof, having a formula selected from the group consisting of Formulas Ic-1a to Ic-7a:

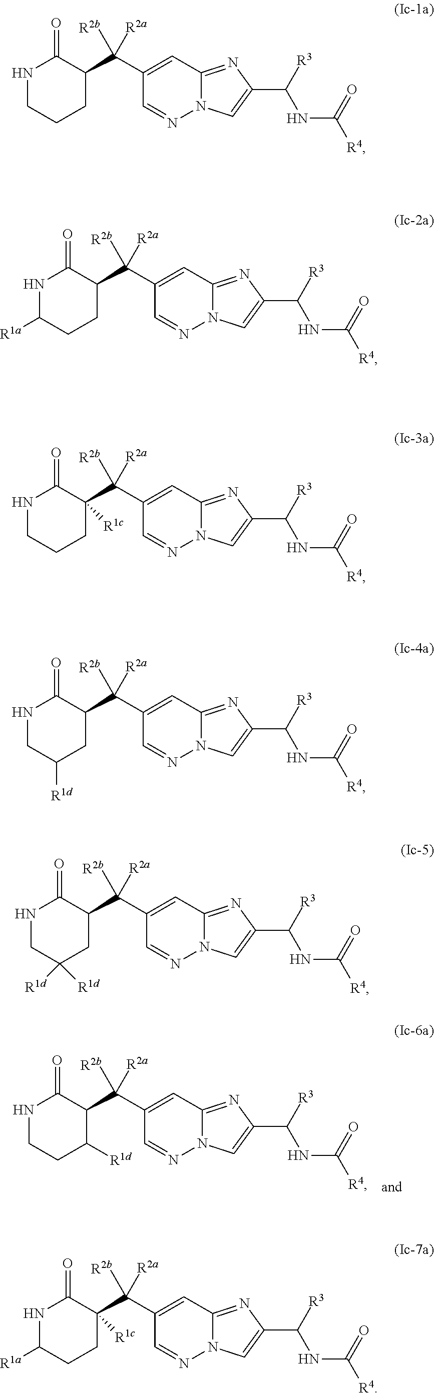

15. (canceled)

16. The compound of claim 1, or a pharmaceutically acceptable salt thereof, having a formula selected from the group consisting of Formulas Id-1 to Id-2:

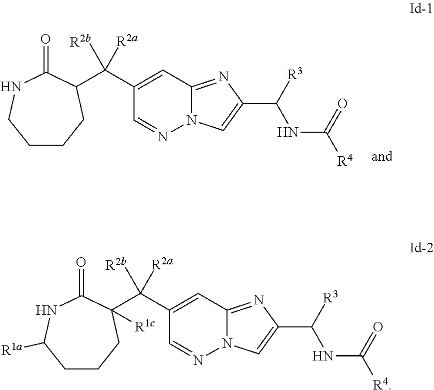

17. The compound of claim 1, or a pharmaceutically acceptable salt thereof, having a formula selected from the group consisting of Formulas Id-1a to Id-2a:

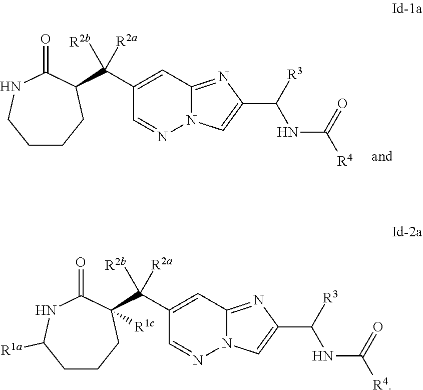

18-27. (canceled)

28. The compound of claim 1, or a pharmaceutically acceptable salt thereof, having a formula according to Formula Ie-1: 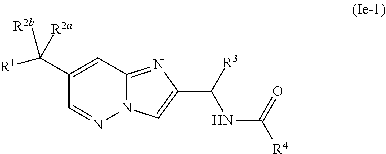
wherein R¹ is selected from the group consisting of:

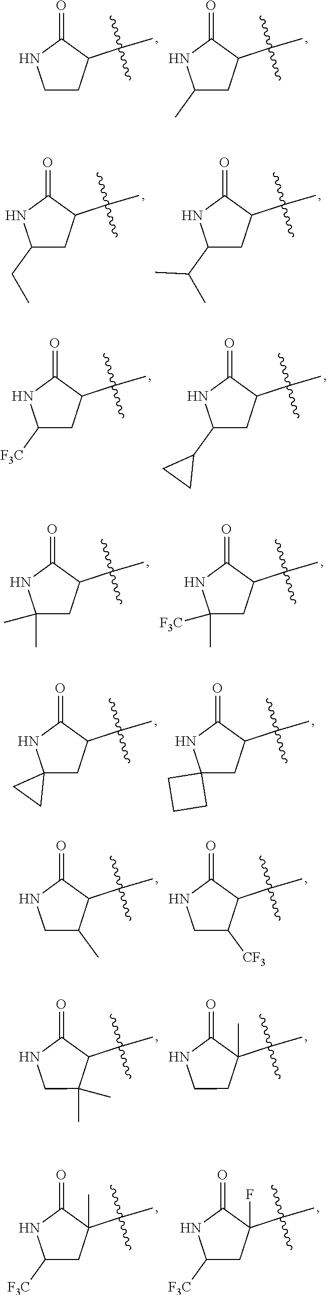

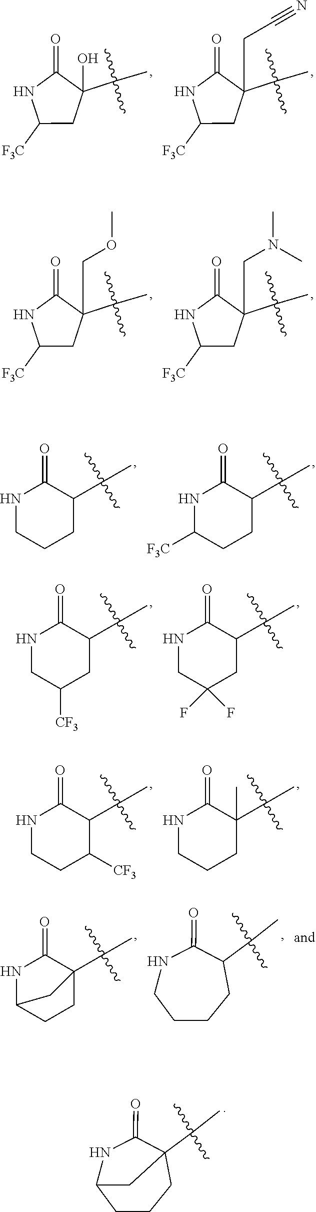

29-30. (canceled)

31. The compound of claim 1, or a pharmaceutically acceptable salt thereof, having a formula according to Formula Ie-2: 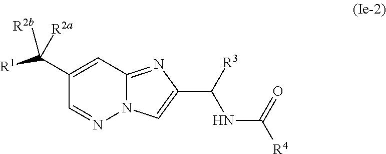
wherein R¹ is selected from the group consisting of:

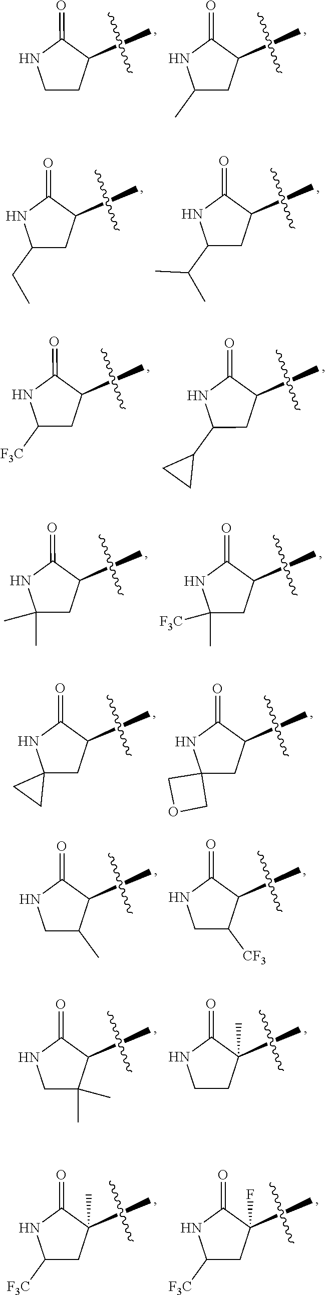

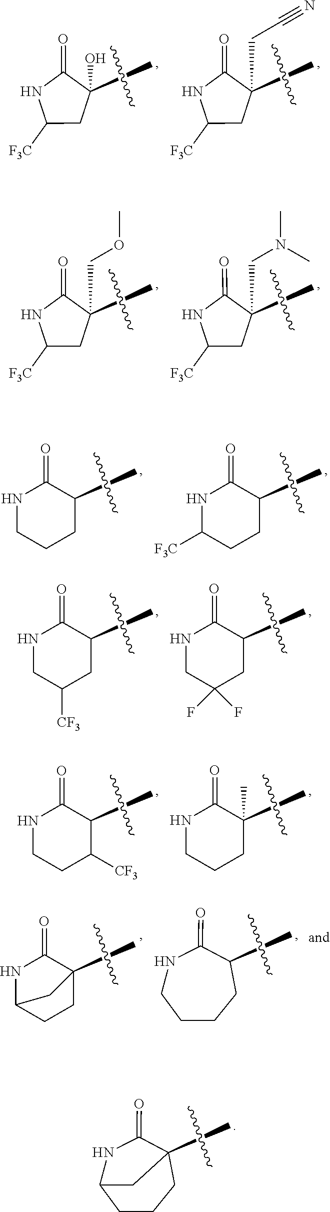

32-49. (canceled)

50. The compound of claim 1, having a formula of Formula Ik: 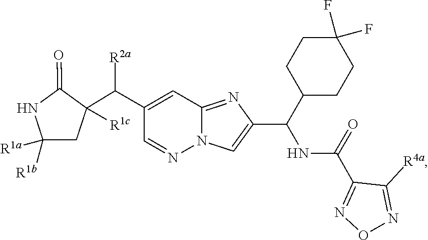
or a pharmaceutically acceptable salt thereof.

51-54. (canceled)

55. The compound of claim 1, selected from the group consisting of: 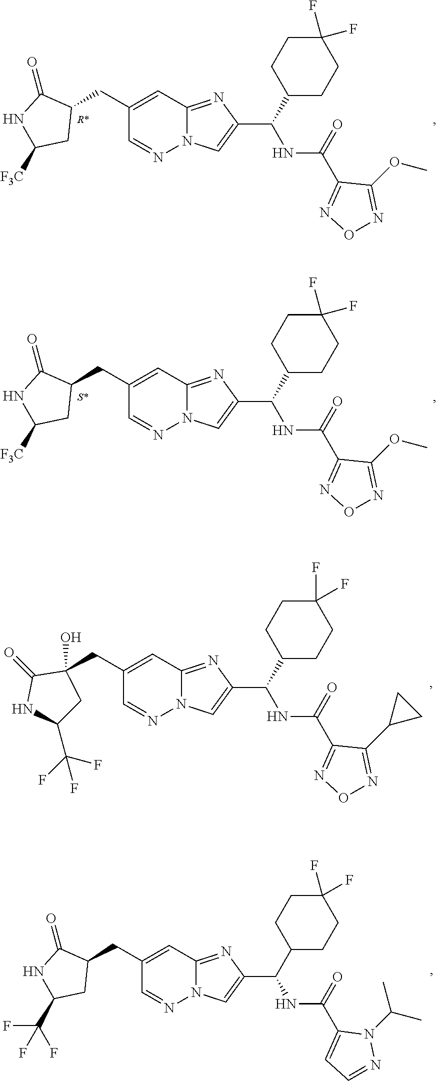 [SMILES: COc1nonc1C(=O)N[C@H](c1cn2ncc(C[C@@H]3C[C@@H](C(F)(F)F)NC3=O)cc2n1)C1CCC(F)(F)CC1 ; COc1nonc1C(=O)N[C@H](c1cn2ncc(C[C@H]3C[C@@H](C(F)(F)F)NC3=O)cc2n1)C1CCC(F)(F)CC1 ; O=C(N[C@H](c1cn2ncc(C[C@@]3(O)C[C@@H](C(F)(F)F)NC3=O)cc2n1)C1CCC(F)(F)CC1)c1nonc1C1CC1 ; CC(C)n1nccc1C(=O)N[C@H](c1cn2ncc(C[C@H]3C[C@@H](C(F)(F)F)NC3=O)cc2n1)C1CCC(F)(F)CC1] 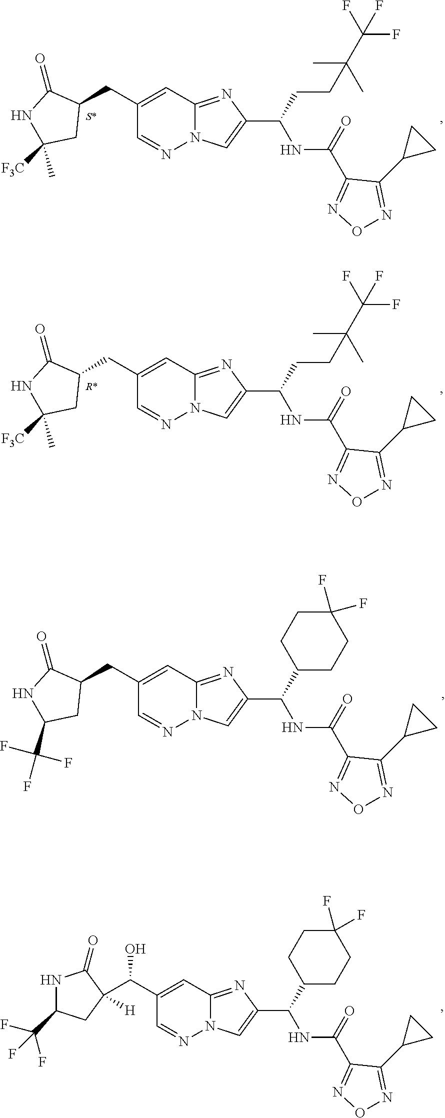 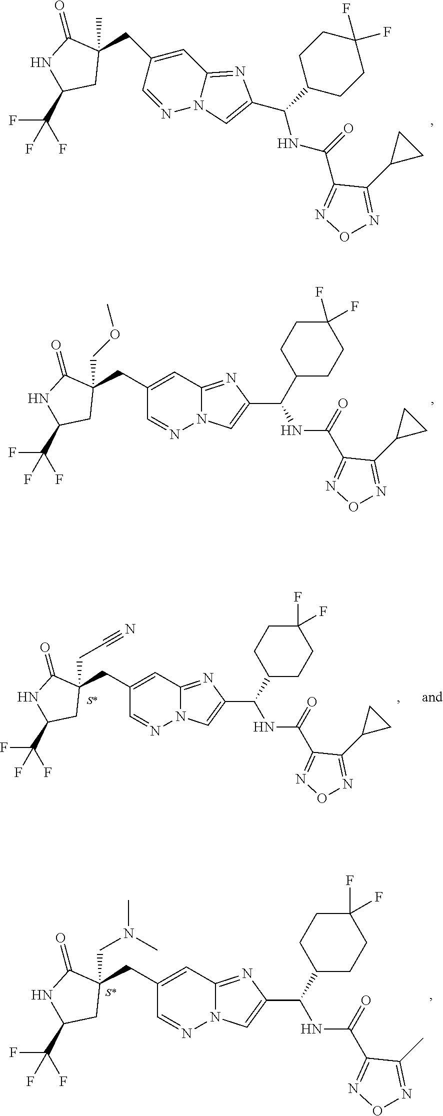 [SMILES: C[C@]1(Cc2cnn3cc([C@@H](NC(=O)c4nonc4C4CC4)C4CCC(F)(F)CC4)nc3c2)C[C@@H](C(F)(F)F)NC1=O ; COC[C@]1(Cc2cnn3cc([C@@H](NC(=O)c4nonc4C4CC4)C4CCC(F)(F)CC4)nc3c2)C[C@@H](C(F)(F)F)NC1=O ; N#CC[C@]1(Cc2cnn3cc([C@@H](NC(=O)c4nonc4C4CC4)C4CCC(F)(F)CC4)nc3c2)C[C@@H](C(F)(F)F)NC1=O ; Cc1nonc1C(=O)N[C@H](c1cn2ncc(C[C@@]3(CN(C)C)C[C@@H](C(F)(F)F)NC3=O)cc2n1)C1CCC(F)(F)CC1]
or a pharmaceutically acceptable salt thereof.

56-68. (canceled)

69. A pharmaceutical composition made by combining a compound of claim 1, or a pharmaceutically acceptable salt thereof, and a pharmaceutically acceptable carrier.

70. The pharmaceutical composition of claim 69, or a pharmaceutically acceptable salt thereof, which is administered orally.

71. The pharmaceutical composition of claim 70, or a pharmaceutically acceptable salt thereof, which is administered as a tablet or a capsule.

72. A process for making a pharmaceutical composition comprising combining a compound of claim 1, or a pharmaceutically acceptable salt thereof, and a pharmaceutically acceptable carrier.

73. A method for treating and/or ameliorating an IL-17A mediated inflammatory syndrome, disorder, or disease comprising administering to a subject in need thereof a therapeutically effective amount of a compound of claim 1, or a pharmaceutically acceptable salt thereof.

74. The method of claim 73, wherein the IL-17A mediated inflammatory syndrome, disorder, or disease is selected from the group consisting of: psoriasis, psoriatic arthritis, rheumatoid arthritis, ankylosing spondylitis, hidradenitis suppurativa, bullous pemphigoid, atopic dermatitis, vitiligo, multiple sclerosis, asthma, uveitis, chronic obstructive pulmonary disorder, multiple myeloma, and systemic lupus erythematosus.

75-92. (canceled)


SEQUENCE LISTING (2 sequences)

SEQ ID NO: 1 - protein, 182 aa, synthetic construct
MATGSRTSLLLAFGLLCLPWLQEGSAGSDYKDDDDKGSGSGSLEVLFQGPGITIPRNPGCPNSEDKNFPRTVMVNLNIHNRNTNTNPKRSSDYYNRSTSPWNLHRNEDPERYPSVIWEAQCRHLGCINADGNVDYHMNSVPIQQEILVLRREPPHCPNSFRLEKILVSVGCTCVTPIVHHVQ

SEQ ID NO: 2 - protein, 323 aa, synthetic construct
MKFLVNVALVFMVVYISYIYALRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWGSGGHHHHHHHHHH